SEQUENCE LISTING (122 sequences)

SEQ ID NO: 1 - DNA, 4830 bp, Mus musculus
  source 1..4830: mol_type = genomic DNA; note = Jhdm2a
accaagtcagagctagagtcggctgggcggctgggctctgttgccgccgtcgtgggtgactcagggaggcgggaggcgggggcgaggggacgaacacttaggcaaaagcactggcgccgcggctcagtcctcccttctctctctcagtgtccagctttgaaagggaggagcccttcctgctggcgtggaaaccatggtgctcacgctcggagaaagttggccagtattggtggggaagcgattcctcagtctgtccgcagccgaaggcaacgaaggcggccaggacaactgggacttggagcgcgttgccgagtggccctggctgtcggggaccattcgagctgtttcccacaccgacgttactaagaaagacttgaaggtgtgtgtggagtttgatggggagtcttggagaaagagaagatggatagatgtctacagccttcagagaaaagcatttttagtagagcataacctggttttggcagaacgaaaatcacctgaagttcctgagcaagttattcagtggcctgcaataatgtacaaatctcttctagacaaagctggcttgggagccataacttctgttcggtttcttggagatcaacaaagtgtatttgtttccaaagaccttttgaaacctatacaggatgttaacagtcttcggctttcccttactgataatcagacagtcagtaaggaatttcaagctttgattgtaaaacatttggatgaaagccatcttttacaaggtgacaagaaccttgttggttcagaagtaaaaatttatagcttggacccatctactcagtggttttcagcaactgttgtacatggaaacccatcatccaaaactcttcaagtcaactgtgaggagattccagcactgaaaattgtcgacccagcactgattcatgttgaagttgtacatgacaactttgtgacatgtggtaattctacaagaactggagctgtaaaacgcaagtcttctgagaataacggaagttcggtttctaaacaagcaaaatcttgttctgaggcctctcccagtatgtgtcctgtacagtctgttcccacaacagtgtttaaggagatcctgcttggctgtactgcagcaactccatctagcaaggacccaagacagcaaaatactccccaggcagccaattctccacctaacattggagcaaaacttcctcaaggatgtcataagcagaacttaccagaagaactttcttcctgtctaaacacaaaacctgaagtaccgagaacaaaaccagatgtctgcaaagaaggattactttcttcaaaatcttctcaggttggagctggagacttgaaaattctgagtgagcccaaaggtagctgtatccagcctaaaacaaacactgatcaggagagcagactggagtctgctccacagccagtcactggccttccaaaggagtgcttgcctgcaaagacttcctctaaggcagaactggacattgccaccactcctgaactgcagaagcatctagaacatgcagcttccacatccgatgacctttcagataagccagaagtgaaagcaggtgtcactagccttaatagttgtgcagaaaagaaggtcgaaccttcacatttaggttcccagtcacagaatttaaaggaaacttcagtaaaagtagataatgaaagctgttgtacaagaagcagtaataaaacccagactcccccagcccggaagtcagttttgacagacccagataaagtcaggaagctgcagcagagcggagaggcctttgttcaggatgactcctgtgttaacatcgtggcacagctgcccaagtgtcgggagtgtcgactagacagcctgcgcaaggataaggaccagcagaaggactctcctgtgttttgtcgctttttccacttcaggagattacaattcaacaagcatggtgtgttgcgggtagaaggcttcttaacaccaaacaagtatgacagtgaagcgattggcttgtggctgcctttgaccaaaaatgttgtggggactgatttggacacagcaaaatatatcctggccaatattggagaccacttctgtcaaatggtgatttctgagaaggaagctatgtcgactattgagccacacagacaggttgcttggaaacgagctgtcaaaggagttagagaaatgtgtgatgtgtgtgacacaaccattttcaacctgcactgggtgtgccctcggtgtgggtttggagtatgtgtagattgctaccggatgaagaggaagaactgccaacagggtgctgcctacaagactttctcttggataaggtgtgtgaagagtcagatacatgagcctgagaacctgatgcccacacagattattcctggcaaagccctctacgatgttggagacattgtgcattctgtcagagcaaaatggggcataaaggccaattgtccctgctccaacaggcagttcaagctcttctcaaagccagccttaaaggaagacctgaaacagacatccttgtctggagaaaaaccaactcttgggaccatggtccagcaaagttcccctgttttggagccagtggctgtgtgcggggaagcagcctccaagccagccagcagcgtgaagcccacctgtcccaccagcacttcacctttaaactggctagctgaccttaccagtgggaatgtcaacaaggagaataaggaaaaacagctgactatgccaattttaaagaatgaaatcaaatgccttccacccttgccccctctgaacaagcccagcacagtcctccatacttttaacagcaccatcttgacacctgtgagcaacaataattcaggtttccttagaaatcttttgaattcatccacagcaaagacagaaaatggattgaaaaacacacccaaaattcttgatgacatctttgcctctttggtgcaaaacaagacttcttctgattcatccaagaggcctcaaggactgacaatcaagcctagcattcttggctttgacactcctcactactggctgtgtgacaaccgcctgctgtgcttgcaagaccccaacaataagagcaattggaatgtttttagggaatgctggaaacaagggcagccagtgatggtgtcgggcgtgcatcataaattaaacactgaactctggaaacccgagtccttcagaaaagagtttggtgagcaggaagtagacctagtcaattgtaggaccaatgaaatcatcactggagccacagtcggagacttctgggatggatttgaagatgttccaaaccgtttgaaaaacgacaaagaaaaagaaccaatggtgttgaaacttaaggactggccgccaggagaagacttcagagacatgatgccttccaggtttgatgatctgatggccaacattccactgcctgagtacaccaggcgagatggcaaactgaacctggcttccagactgccaaactactttgtacggccagacctgggccccaagatgtacaatgcttatggattgatcactccagaggatcggaaatatgggaccacaaatcttcacttagatgtatctgatgcagccaatgtcatggtttatgtgggaattcccaaaggacagtgtgaacaagaagaagaagtccttagaaccatccaagatggagattctgatgaactcacaatcaagagatttattgaaggaaaagagaagccaggagccctttggcacatatatgctgctaaagacacagagaagataagagaattccttaaaaaggtatcagaggagcagggtcaagacaaccctgcagaccatgaccctatccacgatcagagctggtatttagaccgatcgctgagaaagcgcctctatcaagagtacggcgtgcaaggctgggctattgtacagtttcttggggatgtggtgtttatcccagcaggagcgccacatcaggttcataacttatacagctgtatcaaagtggctgaagactttgtgtctccagagcatgttaaacactgcttctggcttactcaggaattccgttacttgtcacagactcataccaaccatgaagataaattgcaggtgaaaaatgttatctaccatgcagtgaaagatgcagttgctatgctgaaagccagtgaatccagtttgggcaaaccttaactcttctctgcacaatggagatgaattattggcagctgatcaaactcttcaggcaggattcctgtggactttgagatttcctgttacctcatcttcttttttaaagtacacctgacttgggagggtactgtctctaatgtatatttctagtgtttacagacactaagtgtgtatatgtagtaactatttacagaccacgcatccttatactgtgacttcacctagatcttctaccaagctgaagaccctgctggctctgaaacaatccttgcagttactccccagctgttcgtctggacagctcattcaagtggatttttaactattagggatcactgcgaagtttcgttggattttattttatgtccttcagagcaccctcccccacccactagggtccagaagagcaatggaggaagtgacagctaatggtgcagttctaaatatatattgcataggactggcattatatagcagaaataactactgtataattcttggggttaaccatctttagttaatggaattttaatttaaatgaagctttgctaattttaagtggtaagcattttgcattaaaatattcctataatattttgtcgctgttctttgtcctttattctttgtttactctctcgaaaataaaagggctaaactattgaatt

SEQ ID NO: 2 - protein, 1323 aa, Mus musculus
  source 1..1323: note = Jhdm2a
MVLTLGESWPVLVGKRFLSLSAAEGNEGGQDNWDLERVAEWPWLSGTIRAVSHTDVTKKDLKVCVEFDGESWRKRRWIDVYSLQRKAFLVEHNLVLAERKSPEVPEQVIQWPAIMYKSLLDKAGLGAITSVRFLGDQQSVFVSKDLLKPIQDVNSLRLSLTDNQTVSKEFQALIVKHLDESHLLQGDKNLVGSEVKIYSLDPSTQWFSATVVHGNPSSKTLQVNCEEIPALKIVDPALIHVEVVHDNFVTCGNSTRTGAVKRKSSENNGSSVSKQAKSCSEASPSMCPVQSVPTTVFKEILLGCTAATPSSKDPRQQNTPQAANSPPNIGAKLPQGCHKQNLPEELSSCLNTKPEVPRTKPDVCKEGLLSSKSSQVGAGDLKILSEPKGSCIQPKTNTDQESRLESAPQPVTGLPKECLPAKTSSKAELDIATTPELQKHLEHAASTSDDLSDKPEVKAGVTSLNSCAEKKVEPSHLGSQSQNLKETSVKVDNESCCTRSSNKTQTPPARKSVLTDPDKVRKLQQSGEAFVQDDSCVNIVAQLPKCRECRLDSLRKDKDQQKDSPVFCRFFHFRRLQFNKHGVLRVEGFLTPNKYDSEAIGLWLPLTKNVVGTDLDTAKYILANIGDHFCQMVISEKEAMSTIEPHRQVAWKRAVKGVREMCDVCDTTIFNLHWVCPRCGFGVCVDCYRMKRKNCQQGAAYKTFSWIRCVKSQIHEPENLMPTQIIPGKALYDVGDIVHSVRAKWGIKANCPCSNRQFKLFSKPALKEDLKQTSLSGEKPTLGTMVQQSSPVLEPVAVCGEAASKPASSVKPTCPTSTSPLNWLADLTSGNVNKENKEKQLTMPILKNEIKCLPPLPPLNKPSTVLHTFNSTILTPVSNNNSGFLRNLLNSSTAKTENGLKNTPKILDDIFASLVQNKTSSDSSKRPQGLTIKPSILGFDTPHYWLCDNRLLCLQDPNNKSNWNVFRECWKQGQPVMVSGVHHKLNTELWKPESFRKEFGEQEVDLVNCRTNEIITGATVGDFWDGFEDVPNRLKNDKEKEPMVLKLKDWPPGEDFRDMMPSRFDDLMANIPLPEYTRRDGKLNLASRLPNYFVRPDLGPKMYNAYGLITPEDRKYGTTNLHLDVSDAANVMVYVGIPKGQCEQEEEVLRTIQDGDSDELTIKRFIEGKEKPGALWHIYAAKDTEKIREFLKKVSEEQGQDNPADHDPIHDQSWYLDRSLRKRLYQEYGVQGWAIVQFLGDVVFIPAGAPHQVHNLYSCIKVAEDFVSPEHVKHCFWLTQEFRYLSQTHTNHEDKLQVKNVIYHAVKDAVAMLKASESSLGKP

SEQ ID NO: 3 - DNA, 4597 bp, Bos taurus
  source 1..4597: mol_type = genomic DNA; note = Jhdm2a
atggtgctcacgctcggagaaagttggccagtactggtggggaagcgattcctcagtctgtctgcaggcgacggcggccacgacagctgggacgtggagcgcgtcgccgagtggccctggctctccgggaccattcgagccgtctcccacaccgacgttaccaagaaggatctgaaggtatgtgtggagtttgatggagaatcttggaggaagagaagatggatagacgtctacagcctcctaaggagagctttcttagtggaacataacttggttttggctgaacgaaagtctcctgaaatttctgagcgaattgttcagtggcctgcaataatgtacaaatctctactggacaaagctggtttggggtccataacatctgttcgctttttaggagatcaacaaagagtatttctttctaaagaccttttaaagcctatacaggatgtaaacaatcttcgtctttcactgatggataatcagagtgtcagtaaagaatttcaagctttgattgtgaaacatttagatgaaagccatcttttacaaggtgacaaaaatttagttggttcagaagtaaaaatttacagcttggacccatcaactcagtggttttctgcaaccattgtaaatggaaacccaatgtcaaaaactcttcaagtcaactgtgaggagattccagcactgaaaattgtggatccatcactgattcatgttgaagttgtacatgataacttcgtgacatgtggtaattctataagaatgggagctgtgaaacgcaagtcttcagagaataatggaaacctggtttccaaacaggcaaaatcatgctccgaggcctctcccagtgtgtgtcctgtacagtctgttcccacaacagtttttaaggagatactgcttggctgtactgcagctactccgccaagtaaggacccgagacagcagagcactccccaagccgccaactctccaccgaacctcggagcaaaaatccctcagggatgtcataaacagattttaccagaactttcttcctgtctaaatacaaagcctgaaatactgagaacaaaaccagatgttgcctgcaaagtagatttgctctcttcaaagtcctctcagattggaactggagacttaaaaaccctgaatgaaccaaaggaaagctgttcccagctgaagaccagcactgaagaggaaagcagtgtggaatctcttcctccaacatcgactgatcctcctaaggagtgcataccaataaaggcggcttctgtggcagaattggaaatcaccaacactcctgaactgcagaagcatttcgaacatgcaccttccacatctgatgtcctttcaaagaagccagaagagaaagcagctgtcaatagtaacagtcctgatagttgcatagaaaaaaggggagaaccttcaactttaggctgccagtcacagaatataaagtcatcttcagcaaaagtagatcctgaaagctgctgtacaagaagcaacaataaattccagaatgccccatctcggaagtcagtcttgacagacccagccaaactcaagaagctgcagcagagtggcgaggcctttgtccaggacgactcctgtgtgaacatcgtggcacagctgcccaagtgccgtgagtgccgcctggacagcctgcgcaaggacaaggagcagcagaaagactcgcctgtgttctgccgcttctttcacttcaggaggttacagttcaacaaacatggtgtattgcgggtagaaggcttcttaacaccaaacaagtatgacagtgaagcaattggcttgtggttgcctttaaccagaaatgttgtggggactgatttggacacagcaaagtatatcctggccaacattggagaccacttctgtcaaatggtgatttctgaaaaggaagctatgtcaactattgagccacacagacaggttgcttggaagcgagccgtcaaaggtgtgcgggaaatgtgtgacgtgtgtgacaccaccatcttcaacctgcactgggtgtgtcccaggtgtgggtttggagtgtgtgtggactgctatcggatgaagaggaaaaactgccaacaaggtgctgcctataagactttcaattggctgagatgtgtgaagagtcaaatacatgagccagagaacctcatgcctacacagatcattcctggaaaagccctctatgatgttggagacattgttcattctgtaagagcaaaatggggaataaaagcaaattgtccctgttcaaacaggcagttcaaactcttttcaaaaccagcctcaaaggaagacataaaacagacttccttaactggagaaaaagcaactctcggtgccatgctcccgcagaatccctcggggttggagccagtgactgtgggtggggaggccgcctccaaaccagccagcagtgtgaaacccgcctgtccggccagcacgtctcctttgaactggctggcagacctaaccagtggcaatgtcaacaaggagaacaaggaaaagcaaccagcgatgccaattttaaagaatgaaatcaaatgccttccacccctcccccccttgtgcaaatccagcacagtcctccacacctttaacagcacaattttgacacctgtaagcaacaacagttcgggtttcctccgaaatctcttgaattcctctacaggaaagacagaaaatggactcaagaatacaccaaaaatccttgatgacatctttgcctctttggtacaaaataagacctgctctgatttatcaaagaggccccaaggactgaccatcaagcccagcattctgggctttgacactccccactactggctgtgtgataaccgtctgctttgcctgcaagatcccaacaacaagagcaactggaatgtgttcagggagtgctggaaacaagggcagccagtgatggtgtcaggggtgcaccacaagctgaacactgaactctggaaaccggagtccttcaggagggagtttggaaaccaggaagttgacctcgttaactgcagaaccaatgaaatcatcacaggagcaacagtaggagacttctgggatggctttgaagatgttccgaaccgcttgaaaaatgaaaaagagccaatggtgttgaaacttaaggactggccaccaggagaagattttagagatatgatgccctccaggtttgatgatctgatggccaacattccactgcctgagtacacaaggagagacggcaaactaaacttggcctcacggctgccaaactactttgttaggccagatctgggtcccaagatgtataatgcttatggattaattactccagaagatcggaaatatggaacaacaaatcttcacctagatgtatctgatgcagctaatgtcatggtctatgtgggaattcccaaaggacagtgtgaccaagaagaagaagtccttaaaactatccaagatggagattctgatgaactcacaataaagcgatttattgaaggaaaagagaagccaggagctctctggcacatatatgctgctaaggacacagagaagatacgagaatttcttaaaaaggtgtcagaagagcaaggtcaggaaaaccccgccgaccacgatcccattcatgatcagagctggtatttagaccgatcactgaggaagcgtttgcatcaagagtatggggttcaaggctgggctattgtgcagtttcttggagatgtggtatttatcccagcaggagcgccacatcaggttcataacctatatagctgtatcaaggtggctgaagattttgtttctccagaacatgttaagcactgcttctggcttactcaggaattccgctatctgtcacagactcatacaaaccacgaagataaattacaggtgaagaatgttatctaccatgcagtaaaagatgcggttgctatgctgaaagccagtgaatccagttttggcaaaccctgagggtcccagaggcaccttacccctgcacattggaagtgaattactggcagctgttcaaactcttcaggcaggattcctgtggactttgagactcatgttacctcatcttcttttttaaactgtacccacctggtaagggggtactctgtctaatgtatatttctagtgtttacagacactaaatgtgtatatgtagtaactatttacagaacatgcatcctttaaaactgtgacttctcacctagtgcagagcttctacccacctgtaaagacagaaccccttctcagttttggaacaacatctgcagttactccccagctgtgtggttggacagtttaatcaaatggattataactcttaggaaccaccacacagttgatttgggttgtgttttgttgtgtctgtctgagtaccctctctcccagcccttagggttcagaagaacagtggaggaagcgaccaccaacaatgcagttcttatactgtattgcattaggactggcattatatagcagaagtcaactactgtacaattcttggggttaaccagctttagttaaatggagttttaatttaaattgaagctttgctaattttaagtgttaagcattttgcattaaagtattcatataa

SEQ ID NO: 4 - protein, 1320 aa, Bos taurus
  source 1..1320: note = Jhdm2a
MVLTLGESWPVLVGKRFLSLSAGDGGHDSWDVERVAEWPWLSGTIRAVSHTDVTKKDLKVCVEFDGESWRKRRWIDVYSLLRRAFLVEHNLVLAERKSPEISERIVQWPAIMYKSLLDKAGLGSITSVRFLGDQQRVFLSKDLLKPIQDVNNLRLSLMDNQSVSKEFQALIVKHLDESHLLQGDKNLVGSEVKIYSLDPSTQWFSATIVNGNPMSKTLQVNCEEIPALKIVDPSLIHVEVVHDNFVTCGNSIRMGAVKRKSSENNGNLVSKQAKSCSEASPSVCPVQSVPTTVFKEILLGCTAATPPSKDPRQQSTPQAANSPPNLGAKIPQGCHKQILPELSSCLNTKPEILRTKPDVACKVDLLSSKSSQIGTGDLKTLNEPKESCSQLKTSTEEESSVESLPPTSTDPPKECIPIKAASVAELEITNTPELQKHFEHAPSTSDVLSKKPEEKAAVNSNSPDSCIEKRGEPSTLGCQSQNIKSSSAKVDPESCCTRSNNKFQNAPSRKSVLTDPAKLKKLQQSGEAFVQDDSCVNIVAQLPKCRECRLDSLRKDKEQQKDSPVFCRFFHFRRLQFNKHGVLRVEGFLTPNKYDSEAIGLWLPLTRNVVGTDLDTAKYILANIGDHFCQMVISEKEAMSTIEPHRQVAWKRAVKGVREMCDVCDTTIFNLHWVCPRCGFGVCVDCYRMKRKNCQQGAAYKTFNWLRCVKSQIHEPENLMPTQIIPGKALYDVGDIVHSVRAKWGIKANCPCSNRQFKLFSKPASKEDIKQTSLTGEKATLGAMLPQNPSGLEPVTVGGEAASKPASSVKPACPASTSPLNWLADLTSGNVNKENKEKQPAMPILKNEIKCLPPLPPLCKSSTVLHTFNSTILTPVSNNSSGFLRNLLNSSTGKTENGLKNTPKILDDIFASLVQNKTCSDLSKRPQGLTIKPSILGFDTPHYWLCDNRLLCLQDPNNKSNWNVFRECWKQGQPVMVSGVHHKLNTELWKPESFRREFGNQEVDLVNCRTNEIITGATVGDFWDGFEDVPNRLKNEKEPMVLKLKDWPPGEDFRDMMPSRFDDLMANIPLPEYTRRDGKLNLASRLPNYFVRPDLGPKMYNAYGLITPEDRKYGTTNLHLDVSDAANVMVYVGIPKGQCDQEEEVLKTIQDGDSDELTIKRFIEGKEKPGALWHIYAAKDTEKIREFLKKVSEEQGQENPADHDPIHDQSWYLDRSLRKRLHQEYGVQGWAIVQFLGDVVFIPAGAPHQVHNLYSCIKVAEDFVSPEHVKHCFWLTQEFRYLSQTHTNHEDKLQVKNVIYHAVKDAVAMLKASESSFGKP

SEQ ID NO: 5 - DNA, 4780 bp, Gallus gallus
  source 1..4780: mol_type = genomic DNA; note = Jhdm2a
gcggggcgcagcggctgttgccgggggcggaggggaagcggcgcgttctcgggacccaggcgccttgtggaggcggaggggggccgggctgagggcggggggcagcagggaggcctcccgatgaggtaaaatggtgctgaggctcgaggggagctggtcgctgctggtcgggaagcgcttcctcagcctgtccggggacgacgacgggccgtgggacacggagcgcgtggcggaatggccgtggcaggcgggcaggatccgggcggtctcacacactgacatctccaaaccggacctgaagatttgtgtagaatttgatgatgagtcatgggaaaaaagaagatggatagaagtctacagtcccaaaatgaaggtattcctcgtggagcaaaagctggtactggcagaaagaagatctcatggtggttctatatcgcctgtccagtggcctgcaatgacttacaaatccttagtggacaaagccggtttgggaccaatggtttcaatacgctatcttggtgaagagaaatgtgtttttctttctaaagaccttttgacgcccattcaggacgtggagagttccaggcttcctctgaaggatgatcaaactgtcaatgaagaaattcaagctttagttaagaaacacctagatgaaacacgcttggtgcaaggtggaaaaaatataattggttcaaaaattagaatttatagcctggatccatctacccagtggttcacagcagttgttgtaaatggcaaccctgctacaagaactctagaagtcaactgtcaggagattccagctttaaaaactcttgatccagagttaattcatgtggaaataatatatgataataacggcaaatgtgataaatccaaaagaattgggggcgtgaaaaggaaaccatcagaaaatagtggaagtgttgatgccaaacacacgaagtcttctcctgaggtttctcagagtcagggacatgtgcactcagtgccaacagtgtttggtgaagcattgctgggttgtactcctgctaataaagaccaaaggcatcaaggcttgcccctgccagctaactctcctcctaatcttggtgcagaaactcctcagggcacatgtagacgaagcgtaccagaagtccatccctcttgtcttaatgctggaacaaaaccattgaaggaaaatctgacgctctttcctaaagtgacatatataactaaagaacaaacacagaatattaatgatcaaaatggtaaatatacttctctgatatcttcaagatcttcatctttgagtgattcaactaatggaaaagaaaccggcctgaaaaatataagcgaacctcttctgaagcccacaacaaactttccaaaagagtgtatttctgcaaaaccgttgcaacatcttaactcttccatagcaataccaagagtcaatagctctgttgaactgcagaggactttagattgtaggccctcaacatcagatgctcaccttctcccttttactgaggctggagttagatcacacagtggttctaacagccaaaaaggaaacaaggtggatgaacatgtggaaatggagttttttactaggagaaattcaaatgaggctttttatgagagaaatgaaaacgaaagcttttggactgggagtaccaagatccaggataatgtaatagttcgcaagtccattttagcagatgcttctaaagtgaagaagctgcagcagagtggagaagcatttgtacaggatgggtcctgcaacaatattgcacctcacttgcataaatgcagggaatgccgcttggacagctaccgaaagaacaaagaccagaaagattccactgtcttttgtcgattctttcacttcagaaggctgcagttcaataagcatgggatactgcgtgaggagggcttcttaactcccaataagtatgaccctgaggctattagcttgtggctgcctttatccaaaaacgttgtaggcctggatctggacacagcaaagtatatcctggccaacattggagaccacttttgtcagctggtaatatctgaaaaggaagttatgtctacaattgaaccacacagacaagtagcctggaagcgtgcagttcgtggagttcgagagatgtgtgatgtgtgtgacaccacaatcttcaaccttcgctgggtctgctctaagtgcggctttggcgtgtgtgtggattgctacaggatgaggaaaaaaggcatacatgaagatgatgactcggatactttctcctggtttaaatgtgtgaaggggcaggagcatgaaccagagaatctgatgcccacacaaataattcctggaagagcactctatgatgttggtgatattgttcactctgtaagaacaaaatggggaataaaggcaaactgcccctgtgcaaataaacagttcaaggcactgtcaaaaccgactctaaaggaggattcaaaacagaatctggttcctggagagaggaccagccttcagcaaagcaacttagtcctgagcccacagctccctacacatgagcctcctgtcaaaccagcagctggaagtaaacaaactgcttcagtaactacttctccttcgttaagttggctgtcaaatataacaagtggaaatgtgaataaagaaaacaaagagaagctcctcgtgcccatttccaagaatgaaaacaaagctcttcagacactccctagcttagctaagcctgctgcagccctgcagacattcaacagtgcaatactcacgcctgtgagcaacaacaacacaggtttcctgcggaatctcctgaattcttctggaggaaagacagacaatggactcaagagtacacccaaaatccttgatgacatttttgcttccctggtgcaaaatagaactgttacagacatgcctaagaagccacaaggactgactataaagccgacaataatgggctttgatacacctcattactggttatgtgataaccgcttactgtgtcttcaagatcccaacaatgaaagtaattggaatgtcttcagagagtgctggaagcagggacagcctgtaatggtgtcaggagtacatcacaaattaaatgcagacctctggagacctgagtccttcaggaaagagtttggccaacaggaagtagacctggttaactgcaggaccaatgaaatcatcactggcgctactgtaggagatttctgggatggatttgaagatatttcaagtcgcctgagaacagaagaaggagagccaatggtgttgaagcttaaagattggcctccgggagaagatttcagagatatgatgccttctcggtttgatgatttgatgaaaaatattccactgccagagtacacgaggagaggtggaaagctcaaccttgcctctcgacttcccaactattttgtacggccagatttgggccctaagatgtacaatgcctatggcttaataacaccagaagatagaaaatatggcacaacaaatctccacttggatgtgtctgatgcagctaatgttatggtgtatgtggggatccccaaaggccaggctgatcaagaagaagaagtacttaagaccatacaagatggtgattctgatgagctgacaattaagcgttttactgaaagtcgagaaaaacctggagctctgtggcacatctatgctgctaaggatacagagaagatccgggagttccttaaaaaggttgctgaagaacaaggtcaagaaaatcctgtagatcacgatcccattcatgatcaaagttggtacttggaccgatctctaagaaaacgccttcaccaggagtatggagttcaaggctgggctattgtacagtttctaggagatgtagttttcattccagcaggagctccgcatcaggttcataatttgtacagttgtattaaagttgcagaagactttgtatccccagagcacgtcaagcattgcttctggctcactcaggaatttagatatctgtcacatacgcatacaaaccacgaagataaattgcaggtgaagaatgtcatataccacgctgttaaagatgcagttgggatattgagagctaacgaatccagccttagcagatcataaagtagaaagccttaacacacactatgaaacagaagcgctggcagctgttttaactactcagtcaggacctctgtggactttgagattatatgaatgttacctcatcttcctttcagcagtaccagaggatcgtggtactatgttctgtgtattcttccaatgtttacaaacctgatatgtatatgtagtaatatgtatggactgtggcatactgtgaatgctcagttgcaatagaactttatatggagttgctctccaaactgtacaaaatacctcctatagaactgtcgggaattattccaaactactcatttgaaaactattatctgagacattgtaaagctccactttttaaaacttacttcagggtgggaggaaatacagtcacccaactaataaccaatttacgttaactaagaatgcggttctgtaacttcacattaacatagaactagcatttaaaagtagctttaagctattgtatagtaacatagagatggagattaaccatctttaggtaaatgtatttaaatttctaaatgttaaaagaaacttttaatcaaatgtcttctgtttgaaactgatctttaatttgaacttacgtttgattctctggtttttaacactgaatggtctacataaaattcttctatttcaaaaaaaaaaaaaaaa

SEQ ID NO: 6 - protein, 1325 aa, Gallus gallus
  source 1..1325: note = Jhdm2a
MVLRLEGSWSLLVGKRFLSLSGDDDGPWDTERVAEWPWQAGRIRAVSHTDISKPDLKICVEFDDESWEKRRWIEVYSPKMKVFLVEQKLVLAERRSHGGSISPVQWPAMTYKSLVDKAGLGPMVSIRYLGEEKCVFLSKDLLTPIQDVESSRLPLKDDQTVNEEIQALVKKHLDETRLVQGGKNIIGSKIRIYSLDPSTQWFTAVVVNGNPATRTLEVNCQEIPALKTLDPELIHVEIIYDNNGKCDKSKRIGGVKRKPSENSGSVDAKHTKSSPEVSQSQGHVHSVPTVFGEALLGCTPANKDQRHQGLPLPANSPPNLGAETPQGTCRRSVPEVHPSCLNAGTKPLKENLTLFPKVTYITKEQTQNINDQNGKYTSLISSRSSSLSDSTNGKETGLKNISEPLLKPTTNFPKECISAKPLQHLNSSIAIPRVNSSVELQRTLDCRPSTSDAHLLPFTEAGVRSHSGSNSQKGNKVDEHVEMEFFTRRNSNEAFYERNENESFWTGSTKIQDNVIVRKSILADASKVKKLQQSGEAFVQDGSCNNIAPHLHKCRECRLDSYRKNKDQKDSTVFCRFFHFRRLQFNKHGILREEGFLTPNKYDPEAISLWLPLSKNVVGLDLDTAKYILANIGDHFCQLVISEKEVMSTIEPHRQVAWKRAVRGVREMCDVCDTTIFNLRWVCSKCGFGVCVDCYRMRKKGIHEDDDSDTFSWFKCVKGQEHEPENLMPTQIIPGRALYDVGDIVHSVRTKWGIKANCPCANKQFKALSKPTLKEDSKQNLVPGERTSLQQSNLVLSPQLPTHEPPVKPAAGSKQTASVTTSPSLSWLSNITSGNVNKENKEKLLVPISKNENKALQTLPSLAKPAAALQTFNSAILTPVSNNNTGFLRNLLNSSGGKTDNGLKSTPKILDDIFASLVQNRTVTDMPKKPQGLTIKPTIMGFDTPHYWLCDNRLLCLQDPNNESNWNVFRECWKQGQPVMVSGVHHKLNADLWRPESFRKEFGQQEVDLVNCRTNEIITGATVGDFWDGFEDISSRLRTEEGEPMVLKLKDWPPGEDFRDMMPSRFDDLMKNIPLPEYTRRGGKLNLASRLPNYFVRPDLGPKMYNAYGLITPEDRKYGTTNLHLDVSDAANVMVYVGIPKGQADQEEEVLKTIQDGDSDELTIKRFTESREKPGALWHIYAAKDTEKIREFLKKVAEEQGQENPVDHDPIHDQSWYLDRSLRKRLHQEYGVQGWAIVQFLGDVVFIPAGAPHQVHNLYSCIKVAEDFVSPEHVKHCFWLTQEFRYLSHTHTNHEDKLQVKNVIYHAVKDAVGILRANESSLSRS

SEQ ID NO: 7 - DNA, 1502 bp, Mus musculus
  source 1..1502: mol_type = genomic DNA; note = Sun4
acggtgacaacagcacagcagcgggagggtcggccgagccgccgcggggctcacgcagggtgctaccctgcggcgcgaggcagctgaggaagggccgagggctttggctgcggcgacttcggcttcccagaggggtagcccaggtcaggatgcggcggagcccccgctcaggctcggccgcatcctctcataatcacacacccaacttctacagcgagaacagcaatagttcccacagcgcgacttcgggggacagcaatgggcgccggtccgctgggccggagctcggggagcccgagggcagaagggcccggggctcgagctgtggtgagcccgccttaagcccaggaatgcccggaggagacacatgggcaggaagctctcggccgaagctggcgcctcggagccacaatgggcagaccgcctgtggcgcggcaaccgtgaggggcggggcctcggaaccgtctggatcttctgtagttttggaggaacaactcaaccttctcccgatcctggatctgaggcaggagatgcctaccccgagggtgtccaagagcttcctgagcctcctctttcaggtgctgagcatggtgttatccctggcagtcgatgggctggtctgtgtgtgcagggaaatctgctccatccgcttcctgttcactgctgtgtcgctcctgagcatctttctggcagcactctggtgggggctcctgtacctgatccctcctttggagaacgaacctacggagatgctgactctaagccaataccaccaccgcgtgcattcccagggccagcaactgcagcagctccaggcagaactgaataaactccacaaggaggtgtccagcgttcgtgcagcccacagtgagagagtggccaagctcgtgttccagaggctgaatgaagacttcgtgcgcaaacctgactatgcactgagctctgtgggagcctccatcgacctggagaagacgtccagtgactacgaggaccagaacactgcctacttctggaaccgcttgagcttctggaactatgcccgcccgccttctgtcatattggagccagatgtgttccctggaaattgctgggcttttgaaggtgataaaggccaggtggtgatccgactgccaggtcatgtgcagttaagcgacatcaccctgcagcatcctccacccactgtggcacacaccggaggggccagcagcgcgcctcgggactttgcagtctatgggctccaagctgatgacgagactgaagtgttcttggggaaattcatctttgatgtgcagaaatctgaaattcagactttccacctccagaatgatcctccttctgccttccccaaggtgaagattcagattctaagcaactggggccatccccgcttcacgtgcttatatcgcgtccgtgctcatggtgtgcggacctcagagtgggcagacgataatgccacaggggtcacagggggaccccattaaaaatgccgatggttggagtag

SEQ ID NO: 8 - protein, 443 aa, Mus musculus
  source 1..443: note = Sun4
MRRSPRSGSAASSHNHTPNFYSENSNSSHSATSGDSNGRRSAGPELGEPEGRRARGSSCGEPALSPGMPGGDTWAGSSRPKLAPRSHNGQTACGAATVRGGASEPSGSSVVLEEQLNLLPILDLRQEMPTPRVSKSFLSLLFQVLSMVLSLAVDGLVCVCREICSIRFLFTAVSLLSIFLAALWWGLLYLIPPLENEPTEMLTLSQYHHRVHSQGQQLQQLQAELNKLHKEVSSVRAAHSERVAKLVFQRLNEDFVRKPDYALSSVGASIDLEKTSSDYEDQNTAYFWNRLSFWNYARPPSVILEPDVFPGNCWAFEGDKGQVVIRLPGHVQLSDITLQHPPPTVAHTGGASSAPRDFAVYGLQADDETEVFLGKFIFDVQKSEIQTFHLQNDPPSAFPKVKIQILSNWGHPRFTCLYRVRAHGVRTSEWADDNATGVTGGPH

SEQ ID NO: 9 - DNA, 1485 bp, Bos taurus
  source 1..1485: mol_type = genomic DNA; note = Sun4
agttacaaaggagagggttgcggcaggccacggggcccaccggctgcacgcagggttgaggggtccgggcggcgcgcaccagctggagggctgagggccttggaggccacgacggtgcctttggcgtcggaaggccacagccccgggtcaggatgcggcgaagccctcgcccgggttcagccgcgtcccagcacaagcacacgcctaacttctacagcgacaacagcaacagctctgtgagcgtcacctcgggggacagctgcgggcaccggtcagctgggccggggcccggggagcccgagggcagacgagcccggggctcgagctgcggtgagcccgccttgagcgctggagtgcccggaggaaccacatgggctggaagctctcggcagaagccggcgcctcggagccacaatgggcagaccgcctgtggcgcggcaaccgtgaggggcggggcctcggtctctgaggagcagctcgaccttctcccgactctggatctgaggcaggagatgccttccccgcaagtgtccaagagcttcctgagcctactcttccaggtgctgagcgtgttgttatccctggtaggagacgtgctggtcagtgtgtacagggaggtctgttccatccgcttcctgctcacggctgtgtcactgctgagcctctttctggcagcactctggtggggactcctatacctcgcccctcctttggaaaatgaacctaaggagatgctgactctaagcgaataccacgagcgcgtgcgctcccaggggcagcagctgcagcagctccaggctgaactggttaaactccacaaggagatgtccagcgttcgcgccgccaacagcgagagagtggcccaactcgtattccagaggctgagtgaggactttgtgcagaaacccgactatgcgctgagttctgtcggagcctccatcgacctagagaagacatcccaggactatgaggatgcgaacactgcctacttctggaatcgcttcagcttctggaacttcgcaagaccgcccacggttatcttggagccagacgtgttccctggaaattgctgggcttttgaaggcgaccagggccaggtggtgatccggttgccgggtcgtgtgcagctgagcgacatcactctgcagcatccaccgcccactgtggcacacacccggggagctaacagcgccccccgtgactttgcagtctatggcctccaagttgacggcgagactgaggtcttcttggggaaatttacctttgacgtggagaaatctgagattcagactttccacctacagaatgaccccccagctgcctttcccaaggtgaagatccagattctaagcaactggggccaccctcgtttcacatgcttgtatcgagtccgagcccatggtattcgaacctcagagggggcaggggacagtgccacagggggcgcccattaaacatgctgattgttgaaaaaaaaaaaaaaaa

SEQ ID NO: 10 - protein, 433 aa, Bos taurus
  source 1..433: note = Sun4
MRRSPRPGSAASQHKHTPNFYSDNSNSSVSVTSGDSCGHRSAGPGPGEPEGRRARGSSCGEPALSAGVPGGTTWAGSSRQKPAPRSHNGQTACGAATVRGGASVSEEQLDLLPTLDLRQEMPSPQVSKSFLSLLFQVLSVLLSLVGDVLVSVYREVCSIRFLLTAVSLLSLFLAALWWGLLYLAPPLENEPKEMLTLSEYHERVRSQGQQLQQLQAELVKLHKEMSSVRAANSERVAQLVFQRLSEDFVQKPDYALSSVGASIDLEKTSQDYEDANTAYFWNRFSFWNFARPPTVILEPDVFPGNCWAFEGDQGQVVIRLPGRVQLSDITLQHPPPTVAHTRGANSAPRDFAVYGLQVDGETEVFLGKFTFDVEKSEIQTFHLQNDPPAAFPKVKIQILSNWGHPRFTCLYRVRAHGIRTSEGAGDSATGGAH

SEQ ID NO: 11 - DNA, 1565 bp, Gallus gallus
  source 1..1565: mol_type = genomic DNA; note = Sun4
aaggtgacctcggaattagttttatttagcgaaggtgccaggaggtttcacaggagtagtctgaatttagcgagagaagtgggagcgaggctgccagggaagcctcattttctgatggaaatcggatgatgtccggagagtccgttttctgtcgaaggcggcagactgaagagtaagaaatctccctccatgcccctggatgcttggtgtgccccgctcatgcctgcgggtgtgccgctgccctctatgacatcacagctgccctttgtgacgtcacagctgcctccgtgggctgcagcgtggcagagccccagtgggctcagagcttttgggacggcagtgctgagaggacgtgctgcttccagcgctcatctgttgccaacatggcgagagcaaaggctcgacgcgccaggcgcccgaaggcatctgagcgctctgctgcgagcgaggctgttggtgaggcccgtcagcgccgcgtagaagaggcagcagcaatgagtcaaaggctccgtgtgctgatggcgttgtggatggcattatcagtacaagctcttgcctccgctggccggctttgcagaacgtggcgtgacaaagccacgggaacggcaggcaagctggctgctgctgcttcagggcacaagaagatgcttttcaggctgtccatcatagtgctcctggtgactttggccatggtgaatgtgtgtgtcttcaagacttgctgggcagctaggagaatttgggaagacagcacggcacaggagatcgagagactccaacaggcagcgtctgagctgagagcgcagctggactgctaccagacacctgactgggcgctgcaaagctttggtgaggacacaggcaggcaagccctgctaccctgtgcttctccacgtctgcggcctcttgcactcatctcctcccccatcattacaggtgccaccatcgacacgcggagaacttctcctatttacgagctgaggagctggtttagcagacactgcttctggtgctctgtgaaccctccggataccatattgcagccgggtgtctcccttggagaatgctggcccatggaaggacagcagggccaggtggtcatcaggctgcgtgccaaaatccggccctcttgtgtcactctggagcacatcaccccggagatgactccgagcggcacagccagcagtgcccccagggatgtcgctgtctttggactggatgcggacagcgaagaggaggttccccttgtgtcattcacctttgatgtgggggaagggcccacgcagaccttcttgctaaagaataatcattccagagcctttcgctacatcaaggttctggtgaagagcaactggggacacccccgttacacctgcctttaccgagtgcaggtgcacggaaaggtggtgaaaacagaagaccatagaaccgcagagtcattgaatggctaaggctggaaaagacccacaggatcatgcgatgcaaccagtcacccgtcaccagcggttctcatgaaaccaaggtctgtaattggatttgtcgttgaaaaaataaagaccttaaccaca

SEQ ID NO: 12 - protein, 354 aa, Gallus gallus
  source 1..354: note = Sun4
MARAKARRARRPKASERSAASEAVGEARQRRVEEAAAMSQRLRVLMALWMALSVQALASAGRLCRTWRDKATGTAGKLAAAASGHKKMLFRLSIIVLLVTLAMVNVCVFKTCWAARRIWEDSTAQEIERLQQAASELRAQLDCYQTPDWALQSFGEDTGRQALLPCASPRLRPLALISSPIITGATIDTRRTSPIYELRSWFSRHCFWCSVNPPDTILQPGVSLGECWPMEGQQGQVVIRLRAKIRPSCVTLEHITPEMTPSGTASSAPRDVAVFGLDADSEEEVPLVSFTFDVGEGPTQTFLLKNNHSRAFRYIKVLVKSNWGHPRYTCLYRVQVHGKVVKTEDHRTAESLNG

SEQ ID NO: 13 - DNA, 1157 bp, Mus musculus
  source 1..1157: mol_type = genomic DNA; note = Sun5
tcaatggtggtggcggcagtggcagccacttcctcctcattgactcccatagacctccatccttggccctttataaaaggaaatcaatgccccggacgaggaacatcggggccttgtgtaccctacctgaagacacaactcacagtggcaggcctaggaggggcgtccaaagaagctatatcagcagaatggcagagccagccccggcaaacatgacctggctcacctacctagcctgcttcctgaggactcagacccagcaagttttccttaacacctgcagatgcaagttgttctgccagaaagtcatggaaaagatgggccttctggtcctctgtgtttttggcttctggatgttttccatgcacttaccatcaaaagtggaagtctggcaggatgacagcataaatggtccattgcagagcctgaggatgtaccaggagaaggtacgacatcacactggggaaatccaggacctacgaggtagcatgaaccagctcattgccaaactccagaagatggaagccatatctgatgagcaaaaaatggcccagaaaataatgaagatgattcaaggtgattacattgaaaagccagactttgccctgaagtccataggggccagcatcgacttcgagcacacgtcagccacgtataaccacgacaaggctcgctcctactggaactggatccgactgtggaactacgcgcagcccccggatgtgatccttgagccaaacgtgacacctgggaactgctgggcctttgccagtgaccgcggccaggtgaccatccgtttggcccagaaagtctacctgtccaacattacactgcagcacattcctaagacaatctcgttgtcgggaagcctggacaccgcaccaaaggactttgtcatctatggcttggagagcctacccagggaggaagtgttcctaggggcattccagtttcagccagaaaacatcatccaaacgtttcagctccagaacctgccacccaggagctttgctgcagtcaaggtgaagatctccagcaactgggggaaccctcgcttcacctgcatgtaccgtgtgcgtgtgcacggctctgtaacccctcccaaagactcacacctagagcccctatcctagagtaaagtatattattcagtccaa

SEQ ID NO: 14 - protein, 348 aa, Mus musculus
  source 1..348: note = Sun5
MPRTRNIGALCTLPEDTTHSGRPRRGVQRSYISRMAEPAPANMTWLTYLACFLRTQTQQVFLNTCRCKLFCQKVMEKMGLLVLCVFGFWMFSMHLPSKVEVWQDDSINGPLQSLRMYQEKVRHHTGEIQDLRGSMNQLIAKLQKMEAISDEQKMAQKIMKMIQGDYIEKPDFALKSIGASIDFEHTSATYNHDKARSYWNWIRLWNYAQPPDVILEPNVTPGNCWAFASDRGQVTIRLAQKVYLSNITLQHIPKTISLSGSLDTAPKDFVIYGLESLPREEVFLGAFQFQPENIIQTFQLQNLPPRSFAAVKVKISSNWGNPRFTCMYRVRVHGSVTPPKDSHLEPLS

SEQ ID NO: 15 - DNA, 1269 bp, Bos taurus
  source 1..1269: mol_type = genomic DNA; note = Sun5
acactaggtgggagcagcagcagcagccacttccccctcacagactcccagagatccccaccgtcaatcccctgtcctagagaaggacatcgatgccccggtcctcaaggagccctgaggactcgtgtgaccaacccgaaaatgtggcccacgtctccagggaggtcaggcccaggaggatcatccaacgaggccggaacatctgcaggatgacagaggcgcctttgtcaaacgcaagtgatgccttcctgttgcctgtccgcatcaatgccccaggcctgacccagtgcatgctggaatgtgtgtcctggatcacctgcctggcctgcttcctaaggactcgggctcaccgggttctgttcaacacttgcagatacaagctgttcatccacaagctgatagaaaaagcaggcgttctgatcctctgtgcttttggattttggatgttttctgtgcacttaccgtccaaaatggacatctggcaggaagacagcatagatagtccactgcagagcttgaggatgtaccaagagaaggtgcggcatcatactggggaaatccaggacctccgaggccacttgaaccagctcattgccaagctccaagagatggaagccatgtccgatgagcaaaaaatgacccagaaaataataaagatgatccaaggagattacattgagaagccggactttgccctgaagtcaataggggcctccatcgactttgagcagacgtcagccacgtacaaccacgacaaggcctgctcctactggaactggatccggctgtggaactatgcacagcccccggacgtgatcctcgagcccaacgtgacacctggcaactgctgggccttctcgggtgaccgcggccaggtgaccatccgactggcccagaaggtctacctgtccaacctgaccctgcagcacatcccgaagaccatctcactgtcaggcagcctggacaccgcccccaaggacttcgtcatctacggcgtggaaggctcccccaaggaggaggtgttcctgggggcatttcagtttcagccagaaaatatcattcagacgttccagctccagaaccagcctcccaggacatttggggctgtcaaggtgaagatatccagcaactgggggaacccacgcttcacctgcctgtaccgagtacgtgtgcatggctctgtgactctacccagagagcagcctaactagagcccccaccccaagagagattaaagtttattcttcacgtccaaaaaaaaaaaaaaaa

SEQ ID NO: 16 - protein, 372 aa, Bos taurus
  source 1..372: note = Sun5
MPRSSRSPEDSCDQPENVAHVSREVRPRRIIQRGRNICRMTEAPLSNASDAFLLPVRINAPGLTQCMLECVSWITCLACFLRTRAHRVLFNTCRYKLFIHKLIEKAGVLILCAFGFWMFSVHLPSKMDIWQEDSIDSPLQSLRMYQEKVRHHTGEIQDLRGHLNQLIAKLQEMEAMSDEQKMTQKIIKMIQGDYIEKPDFALKSIGASIDFEQTSATYNHDKACSYWNWIRLWNYAQPPDVILEPNVTPGNCWAFSGDRGQVTIRLAQKVYLSNLTLQHIPKTISLSGSLDTAPKDFVIYGVEGSPKEEVFLGAFQFQPENIIQTFQLQNQPPRTFGAVKVKISSNWGNPRFTCLYRVRVHGSVTLPREQPN

SEQ ID NO: 17 - DNA, 4900 bp, Gallus gallus
  source 1..4900: mol_type = genomic DNA; note = Sun5
atggagaacatcctcctggatgagagagggcatctcaaactcgctgactttgggctctcccgacacctgcgctgtggtgagcgggcacacaccatctgtggcactctgcagtacatggccccagaggtgctgagtgggggtccctacagccacgcagctgactggtggtccctgggtgtcctgctctttgccctggccagtggggagtttccagtgccaccagcgagggaccacgtggccatgctggagcgtgtcaaagagagcagctatgcgagcccccctgcactcagccctgcgctgggccgactgctggctgagctgctatgccacaacccccttcgccgcctgcgctacctccaccacttccaggaccacccttttttccgtgggttggccttcgacgccgagctgctgcagaaggacccggtggccgttgccgtggccccacgccccaccgagcagcccctgcccgatcccaccacctttgatgacttcgactgcgacctcactgccgcccccccgaaccggccatggcccggctgagcctccgctggaggaacccccgcaccgcggagggacccccgcgccactcagggacaccccccgccctctgtacctctccccacgggagcaataaacccaaacccgatggtaatgctgtgggtgtctcctgcggggctgcgcaccgacggcataggctgcggggagtgcacggagctgagccccccacccagctcaggaccccccgcttagcgttgcggaccacgccggcgattcccgccgggtgggcgtggccgtcgagaaaccacgcccacgccgcacgtaaccacgcccacacgagacgtagccccgcccccatcgcgggaccaatcagagccgcgcgtggttttcaaatgggcggcggggagcggcgcggcgggagcgcggccatgcggggagcgcgcgtctctgccgcacaacggcggcccctgcaggagctgcggctgcagcagggagcggagcgtaccaacatcaccccgctgctccgccgcttgcggctcaaggaccacgatggaggcactcccgcaccccgcgcccggtgcccctctggccgcgttgccccggggctcccccgcactgctgtcccgtgcgccccctgcagcgccgacccggagcccctcggccccctgaagatcccctcaggcaatgccacccccgaccccatcagcatctcagagcccactggccacgccaccccggttacagatgccccagagcccccgggtgacgccacctcagggcctgacaccctggtgcccccacgtgatgctccccaagaggacagccccccagagacccccagcggggccccccgagagccccatggcagtgccccagtgcccatcttcccattctcccccagccacctccccccgctgcctccatctccggaccaccccagcagcactcccttggagcagcgtactcctgaccctgttggcagcacctctatagggccctgcgctctggacacccctggcagcccctctccaggcccccccggtacctccacccctcaggacaccccgttccgcaggtggagagtggccccccctgacctctcctgcagccccgtggcttcccaacccctgtctgagaatagagatgtgggtactcctccacatcgcagcaccctggggatggctggatccccgggcactgccccaccggagcagtgctccccccaggtcgaggcagtgggctccgagcctgtagagtcagcagtagcagcagcggccaccccggtgccttcgtctgagccgtccctgggggctgtgtcctggataatgccactggtgtggctggagaggaccctccctgcctcgtccctgttggagtccctgaggcacagcctgtccgtgtcgtggcgggatgctggcaccaacatcaccccagtgcccactgcagctgtcagtaccggagtcaccccagtgcccaccatacctgtggggagctgtgtcaccccagtgcccactgtatctacgggtaccaccgtaaccccggtgcccaccatacctgtggggagcaacgtcaccccagtgccaaccatggccacaggcaccagtgtcaccccagtgcccactggatccattgccaccaatgtcaccccagtgcccactggatccattgccaccaatgtcaccccagtgcccactggatccattggcaccagcgtcaccccggtgcccaccatgtctatgggcaccagcatcaccccagtgcccaccatgtctacgggcaccagtgtcaccccagtgctcaccctatctatgggcaccaccatgaccccagaggagaggagcgctggcacatccatgcctgctggtgccaaggacagcgctgccgagaccgactctctgctctggcactgtccccgggagcagctgcgttcgctgccacgagcagagctggaggggcggctggagagcaccctgatcatcatcgaggcgctgtcactgcagctgcgtgactggcaggactgccagcgcccgctccctgctgtggggccggcggggcagagggatgcacacacccagactgatgtcacccggccccggggggaggaggggatctaccacagcctctacgtggagctgcagaagaaggtgcaggctgtgcagcggcagcgtggagcagagcaggagttggcgcagcagctggcacgggcagcggaggcaatggatgcatgggccgggcagcgccgtgcgctgcgggacgtggcagacgccgctctgcagggcgtgcaggatgagcacgcggcgctggagcaggagaggctgcaggtgcgtgccatggtgtcccggtgcgaggcagtgctgcacggtgtccctgccaagctgcagagctgcctgcaggagcgggatgctgcacagcagcgagcggatgaagcccttcgtgctaagcaggagagtgactgtttcctggaggcgtttcgctcccacgctgctgcccagattggtgcccgcacgcagagcctggagctgcagcaggagctgagcgcactgctggcagctgccgtccagcagcaggtgtccctggctgcagaggctcagcctttccgggaattcatagacgtgacctttgccagcctccaggaggagcggggagcgctggacggagagcgggagcaggtgcgtgccctggtgtctcgctgcatgactgcgctgcgggacgtccccagcaagctgcacagctgcctgcaggagcgggatgctgcgctgcaacgggtggaggaagccctccaagccaagaaggaggtgtcccagcagctggaggagaccctggaggctctgcaggacgccgtggcccagggggagcagctggcagtcaccaactcacgcctcagcacagatctgagcacggtgatgaagcagctggccagcctgcagcaggagcaggacgcgctgcagcaggactacgaggagcagaaggagaagatgagctggctgaccaaggagagggatgctttgcagcgggagcgccgggagctgcaggaagcagtggagtgccgggagtttctggaccaggagaaccgcatgtcccgcaggcagctgatggaggtggaagccaagctgcaatccacgctggctgagctgcaggagcgcagcctgcagcacgaggagctgctggaagcccaccagagcctgcaggaggaacgagccgccctgcacaaggagctggagagcaccaaggcggagctgcaggacctgcagctcaagagggacagggtgtcctggtgctcagcaggcattgccgagagcaaggcgaggctgcaggagctggctgactgcctgcgagccgccctgccccaccaggatgacgatgacgaggtcccatcgaggagcaagacctggaccccaggctggaggaccccccgtgggccctggaccccccgtagcagtgcttggacccccgtgtgccgcacgcctgcccgccacacaccgcaccacgccagggcgtcctttgtaggcagtgttctgaaggcagtgtcaggaagagatggtgatgaaggcactgaaggtgagagcacatctgccagggatagggctgcatccacaccgaagcccccagagcccgaggacggtctgatggagcaggcagccgagctgcgggccgtggtgtccgacctctctgcgctgagctcccgcatccaggagttggagcagagcgagttcagggcgctgcaggcggagatctctgacctgcagctccagctggaggaggtgacgactgagagccaggagcggatggacgcgcaggctgccaccattgccaagctgaacaaggtgctgcagggcaagctgcagaacgagaaggagctgcaggatgttgtgaaacagcaggaagcaaagatgctgcaactcatcgataagagcggggaggtgacgaggctgaaggaggaggtgacgcagctgaagcgctcgctgcagcgtgcagagactgaggccaaggtgctgtgggaggagatgagaggccaggaggccaagggggacgcagcccacgtgcaggagcgcgtcctgctgcggcaggaggtggacaagctgcgcttgctgctgctggagaaggaggatgagaaggtggtggtgtcggggaattacctggagcaggtgcgagggttggagctgaggctcagccaagcacagaagatgctgcggagccaggaggagctgcaggagaaggtgaaggaggtcctgctggccctccccgaggtgcccccggagctccgtgccctcctgcagcacgtggggttgaagccaaacagcgagaaagctccggctcctctgtaacccacagcgccgaaacgttcttgtgcttaacgccgtcgttatttattgaataaagtttgttggcattcacccca

SEQ ID NO: 18 - protein, 1431 aa, Gallus gallus
  source 1..1431: note = Sun5
MARLSLRWRNPRTAEGPPRHSGTPPALCTSPHGSNKPKPDGNAVGVSCGAAHRRHRLRGVHGAEPPTQLRTPRLALRTTPAIPAGWAWPSRNHAHAARNHAHTRRSPAPIAGPIRAARGFQMGGGERRGGSAAMRGARVSAAQRRPLQELRLQQGAERTNITPLLRRLRLKDHDGGTPAPRARCPSGRVAPGLPRTAVPCAPCSADPEPLGPLKIPSGNATPDPISISEPTGHATPVTDAPEPPGDATSGPDTLVPPRDAPQEDSPPETPSGAPREPHGSAPVPIFPFSPSHLPPLPPSPDHPSSTPLEQRTPDPVGSTSIGPCALDTPGSPSPGPPGTSTPQDTPFRRWRVAPPDLSCSPVASQPLSENRDVGTPPHRSTLGMAGSPGTAPPEQCSPQVEAVGSEPVESAVAAAATPVPSSEPSLGAVSWIMPLVWLERTLPASSLLESLRHSLSVSWRDAGTNITPVPTAAVSTGVTPVPTIPVGSCVTPVPTVSTGTTVTPVPTIPVGSNVTPVPTMATGTSVTPVPTGSIATNVTPVPTGSIATNVTPVPTGSIGTSVTPVPTMSMGTSITPVPTMSTGTSVTPVLTLSMGTTMTPEERSAGTSMPAGAKDSAAETDSLLWHCPREQLRSLPRAELEGRLESTLIIIEALSLQLRDWQDCQRPLPAVGPAGQRDAHTQTDVTRPRGEEGIYHSLYVELQKKVQAVQRQRGAEQELAQQLARAAEAMDAWAGQRRALRDVADAALQGVQDEHAALEQERLQVRAMVSRCEAVLHGVPAKLQSCLQERDAAQQRADEALRAKQESDCFLEAFRSHAAAQIGARTQSLELQQELSALLAAAVQQQVSLAAEAQPFREFIDVTFASLQEERGALDGEREQVRALVSRCMTALRDVPSKLHSCLQERDAALQRVEEALQAKKEVSQQLEETLEALQDAVAQGEQLAVTNSRLSTDLSTVMKQLASLQQEQDALQQDYEEQKEKMSWLTKERDALQRERRELQEAVECREFLDQENRMSRRQLMEVEAKLQSTLAELQERSLQHEELLEAHQSLQEERAALHKELESTKAELQDLQLKRDRVSWCSAGIAESKARLQELADCLRAALPHQDDDDEVPSRSKTWTPGWRTPRGPWTPRSSAWTPVCRTPARHTPHHARASFVGSVLKAVSGRDGDEGTEGESTSARDRAASTPKPPEPEDGLMEQAAELRAVVSDLSALSSRIQELEQSEFRALQAEISDLQLQLEEVTTESQERMDAQAATIAKLNKVLQGKLQNEKELQDVVKQQEAKMLQLIDKSGEVTRLKEEVTQLKRSLQRAETEAKVLWEEMRGQEAKGDAAHVQERVLLRQEVDKLRLLLLEKEDEKVVVSGNYLEQVRGLELRLSQAQKMLRSQEELQEKVKEVLLALPEVPPELRALLQHVGLKPNSEKAPAPL

SEQ ID NO: 19 - DNA, 1060 bp, Mus musculus
  source 1..1060: mol_type = genomic DNA; note = Spem1
aggctagtgggcattaggggtagtgggctgggggcctaagggccctggtggccctagggaccaccatggccatggctgagcggcctcggcctgggtgggcctcgtatcaaaaccccaacatcaacaactgccaggacatgggaaactccattctgctgctcttgggtcttatcgtctgtattaacattggcatcaatttggtgaccctgctctggagccgaatccgcatcctcttacaccgaatgttccatatcatttgtgaaaaagaaaccagtacttcacttgggaagctggtccagcccttgaggaagcagacctaccccaaagtccatcttcgatgtaccatggaccctgtgaagatgactgtgacccccccacccactcgtcgtcgttaccgccatcgagcgcctccatctcgccgtgcccgctgccccatagcttgggcccctgatactgatgatgagaaacctccacatcagcaccctgcaatctgttcccgccactggaatgactctaaggattgggaaggctttcaatctatgcaggaggtgtgggaaccttggactcaggatggtctagagcaacctccccagaccatccgcttccagccagcagtagaggccaggcctctcaaatcggaaatccggtcagaccagggcctagaggcctatgtgtatactgtaaatcctccacctcccagtccagaggctttgagccataaaaacaatgcggcagggtcagggggatgtgttgagggtgagcaagcacagggccagcctgtctcaccatcctttctgggtccagcaaatgtccctgaaataccccggcgccattcatcaggccgaatagtctatgatgcccgagatgtgaggagaaggttacgagaactcacccaggaggtggaggctctgtcccactgttaccccttggtctctggatccagcgcagctgaggggacaggcaaagattgggtgtatcgtcccctgaagggaaggtgactggggacaaaaaaataaaaaataaaaaagagaaaaaaaaaaaaaaaaaaaaaaaaaaaaaa

SEQ ID NO: 20 - protein, 310 aa, Mus musculus
  source 1..310: note = Spem1
MAMAERPRPGWASYQNPNINNCQDMGNSILLLLGLIVCINIGINLVTLLWSRIRILLHRMFHIICEKETSTSLGKLVQPLRKQTYPKVHLRCTMDPVKMTVTPPPTRRRYRHRAPPSRRARCPIAWAPDTDDEKPPHQHPAICSRHWNDSKDWEGFQSMQEVWEPWTQDGLEQPPQTIRFQPAVEARPLKSEIRSDQGLEAYVYTVNPPPPSPEALSHKNNAAGSGGCVEGEQAQGQPVSPSFLGPANVPEIPRRHSSGRIVYDARDVRRRLRELTQEVEALSHCYPLVSGSSAAEGTGKDWVYRPLKGR

SEQ ID NO: 21 - DNA, 1058 bp, Bos taurus
  source 1..1058: mol_type = genomic DNA; note = Spem1
gggctggtggatactgtgggcacagtgggctgggggcctaggggccctggtggccctagggacccccatggccatggctgagcggccacggcccgggtgggcctcgtatcacaaccctaacaccaacagctgccaggacctgggcaactccatcctgctgctgctgggccttatcatttgcattaacattggcatcaacatggtgacgctgctctggcgcagactccgtggcttcttacaccaagtgttccgtgttatttgtgagaaagaagcttctaagttacgctcacctgggaagcagacccagccctcaaagcatagctctccagcagtccacctacgatgcaccatggacgctgtgaagatgacggtgacccccccacccactcgccgccgtcaccgtcgaggctcttcatcgcgccgtgcccgccgcccggtggcctgggctcctgacactgacgacgatgatgacgagaagcccccacatcagcacacagcggcctgctcccacaactgggactaccctgaggattgggagggtttacagactgcccagaggttctggactccctgggcccaggacaccttggagcctcctacccagaccatccgcttccagcagactatagagggaaggcccctcaagagagagatgcagtctgatctgggcctagaggcctacgtgtaccctgtgaatcccccactccccagccctcagatcctgagccacaagaacagtggagggggggcaggggcaggggcccaggcagaacaggagcagtgtgcaccagctgagccacccatcctgggcccggccaatgtcccggacatcccccggcgccgctcctcagggcgcgtcacctacgatgccagggatgtgaggcggcggctgcgggagctgaccagggaggtggaggccctgtctcactgttaccctctggcctctggatccagcacagctgaggggacgagaaaggactgggtataccgttccatgacagagaggtgactgggagaagaataaaaaaaagagaggaggtggaaaaaaaaaaaaaaaa

SEQ ID NO: 22 - protein, 313 aa, Bos taurus
  source 1..313: note = Spem1
MAMAERPRPGWASYHNPNTNSCQDLGNSILLLLGLIICINIGINMVTLLWRRLRGFLHQVFRVICEKEASKLRSPGKQTQPSKHSSPAVHLRCTMDAVKMTVTPPPTRRRHRRGSSSRRARRPVAWAPDTDDDDDEKPPHQHTAACSHNWDYPEDWEGLQTAQRFWTPWAQDTLEPPTQTIRFQQTIEGRPLKREMQSDLGLEAYVYPVNPPLPSPQILSHKNSGGGAGAGAQAEQEQCAPAEPPILGPANVPDIPRRRSSGRVTYDARDVRRRLRELTREVEALSHCYPLASGSSTAEGTRKDWVYRSMTER

SEQ ID NO: 23 - DNA, 2423 bp, Mus musculus
  source 1..2423: mol_type = genomic DNA; note = Crem
gataaataaggaaaacagggaaggaacaaagcattgattacacatgtaataataatgagcaaatgtggcaggaaaaagtatatgaggacaaatgtaaggcaaatgaccatggaaacagttgaatcacagcaggatcgaagtgtaacacgttctgtggcagagcatagctctgctcatatgcagactggtcaaatttctgttcctactctagctcaggtagcaacaattgcagagacagatgattctgcagactcagaagtaattgattcgcataaacgtagagaaattctttcacgaagaccctcatatagaaaaatactgaatgaactttcctctgatgtgcctggtattcccaagattgaagaagaaaaatcagaggaagaagggacaccacctaacattgctaccatggcagtaccaactagcatatatcagactagcacggggcaatacaatgaggagactgaccttgccccaagtcacatggctgctgccacaggtgacatgccaacttaccagatccgagctcctactactgctttgccacaaggtgtggtgatggctgcctcaccaggaagcctgcacagtccccagcaactagcagaagaagcaactcgcaagcgggagctgaggctgatgaaaaacagggaagctgcccgggagtgtcgcaggaagaagaaagaatatgtcaaatgtcttgaaaatcgtgtggctgtgcttgaaaatcaaaacaagaccctcattgaggaactcaaggccctcaaagacctttattgccataaagcagagtaactgtgtttgatttggaccttgttgactgtgaactctaatcggggcaggcgatgcagcatcctcataatggccatgtggacttgtagatgggtctcttaacccttgcttaagaatacagtctgctgtagagtgtgaattgggaatactgttccatgggttggaatgcagctcccctcacattaccaagcttgctctattgccaatagcatgcaacatatgttttgtttgcccttctgcttctacttttttcagggaagctgctaaagaatgtcgacgtcgaaagaaagagtatgtgaagtgtcttgagagtcgagtcgcagtgctggaagttcagaacaagaagcttatagaggagcttgaaactttgaaagacatttgctctcccaaaacagattagtagaaatatttaactatgaactgattacagcatgtacagttgcttttgaatgcaatacaatatatagccggcaagaattatggctttttcctttgtatcattcatctaactttctaaaactaacattcctaagatgctttgttgtatttaatttgctcttacctctaaggtcaattttttagaagagacaaactgtaaaaaatgtatgtaacaaattcttaaaatgaagtatttgtaagacttgttccagtgcaacatatttacagttcccagtctctctgtcatgaatagtgtcctatgcaataaaaattttgcaggttttaagaatcattttaggaaagggtgatcaaaggcagtgcatctctccagtagtaagataaaatcaacccatagagatacctcaggaaagaatgaaaggaagtgtatcctgatgacatgactacgtgagaatagcctacaaatgaatttatgcatttatagatttttataatcgtcactttgtaaagaaagtattgtattgctgtccttgggtgccacagttgaagacagttttaaatagaaccatgttggttgctctttgtactatttggtatttatttaagtatctgagcatttactacagcttcctactatgtatgtagtatgtgaatttctacaaaagtttgtgctgctttgctgttatttaatgaaagagacaacatattttcattatctggaatgagttccacaagtatgaatttattgctacactggatcagcagccttgcaaatactgggccatttcattagaggacaactgcagggctctaggagcagagttcagtgtggagcacttgcctggcatgctacatgttcagttgaaagggaagacctcaagctctgcaaatggaatggggtccaggggaagaggttagaggttagcctttgtgctgtactaggcttcttgctgatcgtctggagagtttctgctgatgaccctccattgtgaattcttgcaacctcaggaatgttaacgtttaaaaaacaacccaagatgtcatttttgattttacaacttggatcaattttgttttgctctttggaatatagctgtgtacatttgtcacgtaggtttaggctggccttaaactcacagttctcttgcctcagccttctgagtgcttggattacggatgtgggccagaatatccagtttgatcaagtattcttttataaaatattactttctttttaa

SEQ ID NO: 24 - protein, 245 aa, Mus musculus
  source 1..245: note = Crem
MSKCGRKKYMRTNVRQMTMETVESQQDRSVTRSVAEHSSAHMQTGQISVPTLAQVATIAETDDSADSEVIDSHKRREILSRRPSYRKILNELSSDVPGIPKIEEEKSEEEGTPPNIATMAVPTSIYQTSTGQYNEETDLAPSHMAAATGDMPTYQIRAPTTALPQGVVMAASPGSLHSPQQLAEEATRKRELRLMKNREAARECRRKKKEYVKCLENRVAVLENQNKTLIEELKALKDLYCHKAE

SEQ ID NO: 25 - DNA, 1499 bp, Bos taurus
  source 1..1499: mol_type = genomic DNA; note = Crem
aacgtgggccggcggcggtcccagtggggctcgtcgtcccacctcctcgcgtccgtaagcagtgacgaggtccgctacgtaaatcgcttcgctgcgggataaataaagaagaaaacaagaaggagcaaagcattgattataaatgtcttaataatgagcaaatgtggcaggaaaaaatatatgaagacaaatctaagacaaatgaccatggaaacaattgactctcagcaggatggaagtttaccagattctgtggcagagagcgaatctgctcacatgcagacgcagactggtcaaaattcaatccccactttagctcaggtttctgtagctggatccagcaccggaagaggctccccagctgtaactctagtacagttaccttccggccagaccgtccatgtccagggagtaattcagacaccacagccatccgttattcagtcaccacaaatacaaactgttcaggtagcaaccattgcagaaacagatgagtctgcagaatcagaaggtgtaattgattctcataaacgtagagaaatcctttcacgaagaccctcttacagaaaaatactgaatgaactttcctctgatgtgcctggtgtacccaagattgaagaagaaaaatcagaggaagagggaacaccacctaacatcgctgctatggcagtgccgactagcatatatcagactagcacggggcaatacattgctatagcccaaggtggaacaatccagatttctaacccaggatctgatggtgttcagggactgcaggcattaacaatgacaaattcaggagctcctccaccaggtgctacaattgtgcagtacgcagcacagtcagcagatggcacacagcagttctttgtcccaggcagccaggtggtggttcaagatgaggaaactgaacttgccccaagtcacatggctgctgccactggcgacatgccgacttaccagatccgagctccgactactgctttgccacagggagtggtgatggctgcctcccctggaagcctgcacagtccccagcagctagcagaggaagcgacacgcaaacgggagctgaggctaatgaaaaacagggaagctgctaaagaatgtcgacgtcgaaagaaagaatatgtcaagtgtctggagagtcgagtcgcggtgctggaagttcaaaacaagaagcttatagaggaactggaaaccttgaaagacatttgctctcccaaaacagattagtagaaatatttaactatgaactgattacaacctgtacagttgcttttgaaggcaatacaatatatagccagcaagaattacggcttttttttcctcctttgtatcattcagcgtattttctaatctctaacattcccaaactgcttcactgtacgtagttaactcttagctgtaacttcagttttttcttaaaagagacaaactgtaaaaaaaagtctgtaacagattcttaaaatgcagtatttccaaaaaaaaaaaaaaaa

SEQ ID NO: 26 - protein, 360 aa, Bos taurus
  source 1..360: note = Crem
MSKCGRKKYMKTNLRQMTMETIDSQQDGSLPDSVAESESAHMQTQTGQNSIPTLAQVSVAGSSTGRGSPAVTLVQLPSGQTVHVQGVIQTPQPSVIQSPQIQTVQVATIAETDESAESEGVIDSHKRREILSRRPSYRKILNELSSDVPGVPKIEEEKSEEEGTPPNIAAMAVPTSIYQTSTGQYIAIAQGGTIQISNPGSDGVQGLQALTMTNSGAPPPGATIVQYAAQSADGTQQFFVPGSQVVVQDEETELAPSHMAAATGDMPTYQIRAPTTALPQGVVMAASPGSLHSPQQLAEEATRKRELRLMKNREAAKECRRRKKEYVKCLESRVAVLEVQNKKLIEELETLKDICSPKTD

SEQ ID NO: 27 - DNA, 1720 bp, Gallus gallus
  source 1..1720: mol_type = genomic DNA; note = CREM
ttgctagtttggaacagttcatgctgagctctgggagaggagcaggggagctgtgcgtgcaggggctgagcatggctgttacaggagatgaaacagctgccactggagacatgccatcttaccagcttcggactcccactactaacttacctcagggagtggtaatggcagcctccccaggggctctgcatagtcctcagcaactggcagaagaggcaacgcgcaagagagagctgcgacttatgaaaaacagggaagctgccagagaatgtcgcaggaagaagaaagaatatgtcaaatgtcttgaaaatcgtgtggctgtgcttgaaaaccaaaacaagactctcattgaggaactcaaggccctcaaagatctttattgtcataaagcagaataactgtctttcattcggaccttgtttattatgaactttaatcaaggcatgagaggaagcaattctacagattgccatatgaacttgagaaagagacacttgaggcccttgtggaaccccagttttgcttaatgttttcagactgatttgggagatgctccaaaatttggaattcagttgtagtctccctactctgctgagctttctaatagcatgcaaatggctttttgtttgcactttttacttctgctttttgcagggaagctgctaaagaatgtcgacgtcggaagaaagaatacataaaatgtctggagagtcgtgttgcagtgctagaagttcagaaccagaaacttatagaggagcttgaaacccttaaagacatttgctcttccaaaacagattagtaaaaatatttattatactgtgaactgacctaaaatatgtccggttgctttataaggcaatacatggccaacatgaattatggctttctctgtgtcatttatattatattccaacttctaacattcctgaaatgctttcctgctttttgtcaattcatagctctgcttacaggttttccatgttgcacccttccacctcccaaggagccaaatggttttaaaaaaacaaagaagtatcaaagtgaaaaaaaaacataatttctaagagctgtttctttgccatatatttacatgctactttttacatgtactgctagtgttctgcacatgcaaaatattttacagctgtttcaaatgaatcataaagataatgcatccggtgatacgagaaaaatcaaataactcaaggtatctcaggaaagagtttataaaagaatgttatgattacatgtatgtattagcatactggtctacaaatgattttatgacatatttttatattagttgctttgtggaaagaaaaagtattgtattgctatcactgagtgccatggctaaagatagtttttaatagaatgatgttgtttaacctgtgtagtgtctgatttcctttgaacatctggttattggcgatgcttttaaacctgtgagagtttggagggggtggaagagaaagtaacactctgacatgacgctaagtatattgcaggtatctgaaaacgtctgttacgtggctgctgttgacttatattttctgtagtctgtgaaacatttggtatattagaagtctatgtataaaaattcaactttgtatcctgaataatttagattccggacagctgtaggcagttacccgtactgaaggctgatggcgtagttcttgttacacttcatccaaagtaccaagggaagtaaaggtga

SEQ ID NO: 28 - protein, 125 aa, Gallus gallus
  source 1..125: note = CREM
MLSSGRGAGELCVQGLSMAVTGDETAATGDMPSYQLRTPTTNLPQGVVMAASPGALHSPQQLAEEATRKRELRLMKNREAARECRRKKKEYVKCLENRVAVLENQNKTLIEELKALKDLYCHKAE

SEQ ID NO: 29 - DNA, 4295 bp, Mus musculus
  source 1..4295: mol_type = genomic DNA; note = GOPC
gtgtcgctgtcgctgcttcagccccgcttcctttgctgctctggagctgcgctgccggcgttggcggcgcgagggaagggaggggaccggcgggtgtgtgtctgtgtgtgtgtgcgtgtgtgtctgtgtgtgcgcgcgcgcggggggcgccgcggtgctagcagttcccctcgcgcggggcgggcggccagagtgagcagcagtcggcgaggccctggacggcgccatgtcggcgggtggcccatgcccggcgggagccggcggcggcccagggggctcctcctgccctgtgggggtctcccctggcggggtgtccatgttccgatggcttgaggtgctggagaaggagttcgacaaggccttcgtggatgtggatctgcttttgggcgagatcgatccggaccaggcggacatcacttatgaggggcgtcagaagatgaccagccttagctcctgctttgcgcagctatgccacaaagcccagactgtgtcccagatcaaccacaagcttgaggcacagttggtggatctgcgatcggaattgacagagactcaagcagagaaagttgttctggagaaagaagtccacgagcagcttttacagctgcactccactcagcttcagcttcatgccaagactggtcaaagtgttgactctggggccatcaaggcaaaattggaaagagaacttgaagcaaataaaacagaaaaggtaaaagaagctcgacttgaagctgaagtgaagctactaaggaaggagaacgaggctcttcgaaggcacatagctgttctccaggctgaagtttatggggcgaggttggctgccaagtacttggataaggagctggccggcagggtgcaacagatacaattactgggacgggatatgaagggacctgctcacgataagctctggaaccagttagaagctgaaatacatttgcatcgccacaaaactgtcatcagagcctgcagagggcgcaacgacttgaagcggcccatgcaagcacccccaggccacgatcaagactctttgaagaaaagccagggagtcggtccaattagaaaagttctcctccttaaggaagaccatgaaggcctgggcatttctatcacaggtgggaaagagcacggtgttcccatcctcatctctgagatccacccgggacagcctgcggatagatgtggagggctgcatgttggagatgctattctggcagtcaatggagtcaacctaagagacacaaagcataaagaagctgtaactatactctcccagcagagaggagagattgagtttgaagtagtctacgtggcacctgaagtggattctgacgatgaaaatgtagagtatgaagacgagagtggacaccgctatcgtctgtatcttgacgagttagaaggcagtgggaattctggtgctagttgcaaagacagcagtggcgagatgaaaatgttacaagggtataacaagaaggcagtgagggatgcgcatgagaatggggacgtgggggctgcaggtgagagccctctggatgacactgctgcgagagcggctcatctgcacagcctgcatcagaagaaggcctactaacccagacacgctgcttgtcagactcgtggagtgtggggcactagagaagacggtgacaaagactgcacaagatcagggaggctgtgtgttgcctagagaaaggcgggaacctcagggcttcatgtgaacaattctaagtgcataaacccctgttttctgtggtataccttaattagctattgcatggaaggcagttttgattttcctggaatctaagcaccaaagcatgtgtttcccaaaggggttttactaggctcttacaggccaagtggatgctatttcatttggctccttttcatgtataacaggacagtgctaactggaattctctaagtaccctttaaatacagcagagtaaatgataccatatattccctaaggataactgcggaatttcagatgacagaactttactcagtgaattgcaaaaatctaggttgacatttttgcttaattgaaaattttgtttcaggttttttttaaaaagtatcttgtacattcctgatatctaaattaattactatcttaaatcattatgatgagttcactctccaggtaactcaactgcagtacaaaacggttttctttaggcaatcctgtatcaatcaatgctgctctaggaatagtttgggaaaagttactgtgcatttgactttggacttttaaaggaaacgcagacaagtggaaaatggcctgttttcaattatgttgatctgtgtgcatggtattggagcattacactgttaatgtttagtcccaaatgttaattgtctggaatccacgaaaaacagctttatacatttttgagctgttcccaagctttgtcttgtgatagtcaggaacattactatgtcatgaaatacattgtgttcaaacagcagttggctttaagtacctgacttgccagtcatttaaaaagccgggggccaaggtaggcaagatgaggtaaagacattttggtacagtgagcatattggtttttatatcagtatatgtttgactaaagcaatactcgtgtgcaaactctgtaaatgtattagttctattgtatcagaactaagtacagagtgcctgctcccttctttcctgacctacagacaattgcaccctgcctgccgggcaaactgaaaatactgtacttaaagaggtgtgatttttgctaagtcactgttttaaaggatttacacctaagggaatcttatgtgggagaagcctacatatatcgggcttttacaatatcagtatcttctttcttggtatgcttgtttttcccagatgcattatatatttgttcaaagttaaaatctataaaatgtatatactttattttgtgattttgctatttataaattttctttttaactgttgctcatttatggtctctgggtggtttcgttcttcatagtatcaccatgttaactggtaactgaaagtgcacttcgtatgtatattgttagtgatgctgagataccttgtagtgtaacattgcctgtaaacaaggttagtattttaactgtacaaatgaataaacacgttacatattactgtttgctcctgataggctagacctcttaaaagaaagattgttttttcctcaagtattcctcccacaccaaacacataccccagtgcatgacttgagaccttcaaagtaggtggtagccattgtattcatttaaggaaaaaatgttcatggtaacagatctgaaatatttttgctttgacctgaaaccatgctgctcctatatcctttcagcattgcctgggaaggaccagggccattttattttcctttttattcatagattatatgccttatatactatgattgaatcgctaggaaatgtaagctatataaccctttgaattttttcaagaacttcacagcagatgtgaaagcaaaacttggatgagcattttcatgtgctgcctaggagggattgtgtgagatgggtacagaatatatttagaaagagtagtttatggttctacaggtccgtctttccatctctgctttctggtttggaagcataggagagtgctgtggcgtgaaacctgctctgagagcatcctggtactctcccaggcccactggagagttcacttcagagtgtgtggtcccaaagcactaagtgaaaagtagtctttatagttcatgttcataggaaagatgcccatataacactgactcttcttgaacaaagccaaaaaatggggggaaagctgttgcaaagccattacaatgaaaacgtgtagagcatgagaggtgtgtgcgtggacatactcacacttattgtgtgaaattgtcttctgcagttttctgttctgaacatgatgaaagtgacgtattcgggttagggattattgttaaattaaatttgagagagatgaagagcttgcaagtctgttgattagaattaaacaattacaaataacataacctgtccatggtgctggaatagaccccacagcaaggggcttatgttgttttcatattttaaaacaaaaaatatattaatttacatataaggggatttatgataattgtattatatctagttgagtttcatattcttaattagcctgtaaataaaaatgacatattctacattaaagcagtattgatcagatttgaaggaaaataaatgttttctctttcattcacattaaatattttggtttattatgggttctgagtaaa

SEQ ID NO: 30 - protein, 455 aa, Mus musculus
  source 1..455: note = GOPC
MSAGGPCPAGAGGGPGGSSCPVGVSPGGVSMFRWLEVLEKEFDKAFVDVDLLLGEIDPDQADITYEGRQKMTSLSSCFAQLCHKAQTVSQINHKLEAQLVDLRSELTETQAEKVVLEKEVHEQLLQLHSTQLQLHAKTGQSVDSGAIKAKLERELEANKTEKVKEARLEAEVKLLRKENEALRRHIAVLQAEVYGARLAAKYLDKELAGRVQQIQLLGRDMKGPAHDKLWNQLEAEIHLHRHKTVIRACRGRNDLKRPMQAPPGHDQDSLKKSQGVGPIRKVLLLKEDHEGLGISITGGKEHGVPILISEIHPGQPADRCGGLHVGDAILAVNGVNLRDTKHKEAVTILSQQRGEIEFEVVYVAPEVDSDDENVEYEDESGHRYRLYLDELEGSGNSGASCKDSSGEMKMLQGYNKKAVRDAHENGDVGAAGESPLDDTAARAAHLHSLHQKKAY

SEQ ID NO: 31 - DNA, 1743 bp, Bos taurus
  source 1..1743: mol_type = genomic DNA; note = GOPC
tctacgcatgcgcggcgggggcgggccgagaggtgggacggcccgcgtcttccgtgtgccaccgctgccgcctctgctctgctgctcttgcccctctggggcttcgttgccggcgttagccacgcgaggggaagagaggggacggacggagcgtgcgtgcgcggggcttcccgcagtcccagcagttcctctcgcggggtggacgacggggtcagcagcagtcgggggaggccctagacggcgccatgtcggcgggcggtccatgcccggcaggggccggagggggctcagggggagcctcttgcgccgtgggggtctcccctggcggagtatctatgttccggtggctggaggtgctggagaaggagttcgacaaggccttcgtggatgtggatctgctcctgggcgagattgatccggaccaggcggacatcacttatgaggggagacagaagatgaccagcctgagctcctgctttgcgcagctttgccacaaagcccagactgtgtcccaaatcaaccacaagctagaggcacagttggtggatctgaaatctgaactgacagagacccaagcagagaaagttgtattggagaaagaagtacatgatcaacttttacagttgcactctattcagcttcagcttcatgctaaaactggccaaagtgttgactctggtaccattaaggcaaaattgtctgtcccctctgtggaggagctggagagagaactcgaagcaaacaaaaaagagaaaatgaaagaagctcagcttgaagctgaagtgaagttattgagaaaagagaatgaagcccttcgcagacatatagctgttctccaggctgaagtttatggggcaagactggctgccaagtacttggataaggaactggcaggaagagtccaacaaatacagctacttggacgagatatgaagggacctgctcatgataagctttggaaccagttggaagctgaaatacacttgcatcgtcacaaaactgtgatcagagcctgtagaggacgcaatgacctgaagcgaccgatgcatgctccaccaggccatgatcaagactctttaaagaaaagccaaggtgttggtccaattagaaaagttctcctccttaaggaagatcatgaaggccttggcatttcaattacaggtggaaaggagcatggtgttccaatcctcatttctgagatccacccagggcaacctgctgatagatgtggagggctgcacgttggagatgctattctggctgttaatggagttaacctccgggacacaaagcataaagaagctgtaaccattctttcccagcagagaggagagattgaatttgaagtagtttacgtggctcctgaagtggattctgacgatgaaaatgtagagtatgaagatgagagtggacatcgttaccgtttgtaccttgatgaattagagggaagtagtaatcctggtgctagttgtaaagatacaagtggggaaatcaaaatgttacaaggatttaataagaaggcagttactgatggacacgaaaatggagacctaggagcttcaagtgaaactcctctagaagacaatgcttctaaactagatgatctacacagtctgtatcataaaaagtcttattaaattgactgtatcttcagacaagattgttaatcagactattctgaatttggggcactagggaagatggtgacaaagactatacaagatttaaggaggctgtttattg

SEQ ID NO: 32 - protein, 463 aa, Bos taurus
  source 1..463: note = GOPC
MSAGGPCPAGAGGGSGGASCAVGVSPGGVSMFRWLEVLEKEFDKAFVDVDLLLGEIDPDQADITYEGRQKMTSLSSCFAQLCHKAQTVSQINHKLEAQLVDLKSELTETQAEKVVLEKEVHDQLLQLHSIQLQLHAKTGQSVDSGTIKAKLSVPSVEELERELEANKKEKMKEAQLEAEVKLLRKENEALRRHIAVLQAEVYGARLAAKYLDKELAGRVQQIQLLGRDMKGPAHDKLWNQLEAEIHLHRHKTVIRACRGRNDLKRPMHAPPGHDQDSLKKSQGVGPIRKVLLLKEDHEGLGISITGGKEHGVPILISEIHPGQPADRCGGLHVGDAILAVNGVNLRDTKHKEAVTILSQQRGEIEFEVVYVAPEVDSDDENVEYEDESGHRYRLYLDELEGSSNPGASCKDTSGEIKMLQGFNKKAVTDGHENGDLGASSETPLEDNASKLDDLHSLYHKKSY

SEQ ID NO: 33 - DNA, 4920 bp, Gallus gallus
  source 1..4920: mol_type = genomic DNA; note = GOPC
tcagcggcgtcgcgcatgcgcgcgggcacattccgctcgcctggctgcgtggccgcgccgggagagccgggaccgccgcgagggcgcagggggacggccgaggcgggtgggggcgcaagccgggccctgtgcgaggcgaggccgggccgggcggtggccggcgggatgtcggcggcgggcggcgttggctcctgcgggccgggtggcgcggcctgcggcggaggggtgggcggcgcggcctcggcgccgggcggcggggtgtccatgttccgctggctggaggtgctggagaaggagttcgacaaggccttcgtggacgtggacctgctgctgggcgagatcgaccccgaccaggccgacatcacctacgaggggcggcagaagatgaccagcctgagctcctgcttcgcgcagctctgccacaaggcgcagaccgtgtcccagatcaaccacaaactggaggcacagttagttgatctgaaatctgaactgacagaaacccaggcagagaaagcagtactggaaaaggaagtccatgatcagcttctgcagcttcatgctgtccaacttcagctacatgcccaaactggtcagaatgtggattcgggggctattaaggcaaagctgtctgtcctttctgtggatgaattggagagagaacttgaagctaacaagaaggaaaaagtgaaggaagctcagctggaagctgaggtgaagttgctgaggaaggagaatgaagctcttcgtcgacacattgctgttcttcaggctgaggtttatggagcaagactggcagccaagtacttagataaggaactagctggaagagttcaacagattcagttactgggtcgagatatgaagggacctgctcatgacaagctctggaatcagcttgaagcagaaatacaccttcaccgtcacaaaactgttattagagcttgtcgaggtaggaatgatctaaaacgaccaatgcaagcaccaccaggacatgatccagatgccttaaagaagagtcaaggtgttggtccaatcagaaaagttttgctagtcaaagaagaccatgaaggactaggaatttcaatcacaggtgggaaggagcacggtgttccaatactgatctctgaaatccatcctgggcaacctgctgatcgatgtggaggactacatgttggtgatgctattctggcagtaaatggagttaatctaagagatgccaaacataaagaagctgtaactatcctttcccaacagagaggcgagattgaatttgaagtagtgtatgttgctccagaagttgattcggatgatgaaaacgtagaatatgaggatgagagtggacaccgttatcgtttgtatcttgatgaattggaagaaggagcaaattcaggttccaataggaaagaagtaaatgtggacgtcaaaccttcacaagtgtttgataagaaaccaagtattgatggacatgaaaatggagacctggggaattcagttgaaccttccttagaagatgctgcacccaaattaacccgttctgctgagtctttatcctaaagaaagaaagaaagaaacaaacaaaaacaactggacagatcccatctttgggaacaattgataatcaatattgactgctccgagttcgtgtatacaaatgtaggttgtgaaatggtagttaaaagtcaaagggaactgttattgttgcctagaaaaaaggcagttacctcagggcttcattgcgagcagttctaagtgtggaaaactgtcttttgcttgacacacagtggttacttaacacataacatgtgaaatgaattttcttttaataataagcaccaaagcatgggatttctaaaaacgataacctcaataccttcagcttttaatttcttttatatccccaaggaaggaaagtaagatgtgaccagcttcattttaacccctcgatgtataaaagggacaaccagcgttcagtttgttgtgaaacaagctttgactctggaaggtgagggggaattgtactgttgcatacctgaattttccacttggagattttgatttgtaactttcaaagataatgtggtttttgttttgttttgttttgtttttttcacagaacagtaaaaccttgttacttaaacaaggctatcaaaacccaaattctgcaatggaatacttgttagttatgatccatgatagttctatgggtgaactttttgatattattcttttaattaattcactgctaattgttccatatgaaaactgctccgagcgtatttaaacagggtattatggggttttacaaacaaaattagcttaaaaagacatccacatttatgtgtgccctttggtatttttttctgaacattatcagttcagtgtgctttcctgaatggatttggttactacctgtgcggagccatctgagtttgtcattattagcttgagcttaagagatgctgagtactctcaataggtactgaacatatttcagattaagataatctttaaagaagtgaaaataaggtgcacagtactttataggaggtgctcagaattgtacagtgtctggccctatctgttacttttgggcaggtgtattaacctgtaattgtcttactgttgaaagagaaaagatactgtttttgtaacagcagtgtatctgaaactagtttttaattagtgtacatctaaaggcccgtggcacggttgcaaatggaaatgttcggggtggcttcttggctagtttcttgtataactgtagatatacaagaactgaccttgtagatctttaggtgaaatgcaacttttttccatatacagactctctttgtaatcacatttattatttcagcctgctaagatagctttcctgttcactgctttgcaggcttttggactgtaatgtgaaaagattttattatttgacagtggaactaatgtgaaaagacatttttggaagtgttagtatgtaggatattacgtacctaagaaaaaacctggatggcctttagagtaacatcaattacttttaccttcaagattgtgatgtagaacacggttgcttgtttctcctaggagcactatatatatgttaatagaaaaatcctaaaaatgtatatactttattcaaagatttttctatttataaatcccttaactttgctatttacaagattgttttgtgtatgtgcttgtacgcatgagttccattcactccgtggagttatttggttcattttttgtaatagcccttgtcatatggcatactgtttgaaggcttttagaatagtcagagttgtacagttttcaccacacaggcactagtacttaaaaatgaatctgtgtaaagaagaaacaggtcaccagacatgtttctttttcttatgcatcaggaattacaggcatgttgggtacatattagcagataaatccactgctataacttcacattcagatttctgtgttttttttaattattattttttatattttttttccctcacagttagtagagtgcactctgaaatggtcagtggcctagaacgttctatgctctctggtagagagtgtaatttaaagccaagtagtaattttactgtctttgaaaatcatagctgtataaaatattgctgcagttctaagaagtgaggctggtccaactagtctaatagctttttaaaagcactgaatttgcctgcacagtttccctgtatctgagggaaagattaattttttttatttatttgtttttccttactttctggcttagctcattttccatttagtccattgagccatttcagcctgaggtgggagaatatttctggagttattttcctgacattccttgtccacctgtttgttaagatcctgtacgcttttctttatcatctggaagttgctggaccctttctctgttattctgtctatttgtaatctatggatgagatcaacagaaggagcaatggctgaggaaaatgactaggagcagacaaatcaggtatcagtaaatcatcactgtgtttctttccctgcagtttttataatctttgtttagaaagccctgaatgggaggaatttgcacagacactacagcatgactgtaccccatacttagtaccatctgatttaacatactcatgtgccgtagtcatactgagatgctttgagattaatatttaaattgagaaaacttttgcaagttgagatttccagattcagtagacgaatgtgtatttttccctttgtcagtaactggaagactcagtcctgctgatccccagaggttgaaagaaatagacttggaagaatttccgaggaaaggcagtattcagacttcatctttattttttaaagtgtaaaaccgtttactggcatgtatatatatttttacttcaagaataaaatagtattctaaagaaaactgtgcattttttttctagtagataattccttgcctattaaaaaaaaaaaaaacaacaaacttgattgagacaactgtattttaagctaaaggtagacagtgcttgttggcatctgggaccaatgttaattaagagcatgttagttaagggaagtgctaaatggcaataaatttaactctgatcaaagactgtatgtagtatttcctttgtagaagtgtatttgtggcagtgaagatggtttgtatccatctcaatatatgttaatcgaccattatatcaaatgaaatccaagtatttattgtatgtgtccaaacatctataaagtcattcctgtaggaggtataacatttaacaacaaatgtatcatatctagttaagctgaatatatttttattagcctgtaaatactcttctgtacttcagtgatttctgtacctaattcagactgatacttcattttcttctgtttttatatataaaatatagataccta

SEQ ID NO: 34 - protein, 512 aa, Gallus gallus
  source 1..512: note = GOPC
MRAGTFRSPGCVAAPGEPGPPRGRRGTAEAGGGASRALCEARPGRAVAGGMSAAGGVGSCGPGGAACGGGVGGAASAPGGGVSMFRWLEVLEKEFDKAFVDVDLLLGEIDPDQADITYEGRQKMTSLSSCFAQLCHKAQTVSQINHKLEAQLVDLKSELTETQAEKAVLEKEVHDQLLQLHAVQLQLHAQTGQNVDSGAIKAKLSVLSVDELERELEANKKEKVKEAQLEAEVKLLRKENEALRRHIAVLQAEVYGARLAAKYLDKELAGRVQQIQLLGRDMKGPAHDKLWNQLEAEIHLHRHKTVIRACRGRNDLKRPMQAPPGHDPDALKKSQGVGPIRKVLLVKEDHEGLGISITGGKEHGVPILISEIHPGQPADRCGGLHVGDAILAVNGVNLRDAKHKEAVTILSQQRGEIEFEVVYVAPEVDSDDENVEYEDESGHRYRLYLDELEEGANSGSNRKEVNVDVKPSQVFDKKPSIDGHENGDLGNSVEPSLEDAAPKLTRSAESLS

SEQ ID NO: 35 - DNA, 4252 bp, Mus musculus
  source 1..4252: mol_type = genomic DNA; note = HOOK1
ggcgcgggcgccaggcctggccgccgagctccagtggcgggcgcggaggtcgttgacgcgggcccggtcggaggcgcgtccgtcgctgtgagcgcggcgtcgaggttctcggaccatggaggacccgcagccgctgccacagtccgagctgccgctgtgtgacagcctcatcatctggctgcagacattcaagactgcctcaccttgtcaagatgtcaaacagctgactaatggagtgaccatggcacaagttcttcatcaaattgatgtagcttggttcagcgaatcttggttaagccgaattaaagatgatgttggagacaactggagaataaaggctagtaacctgaagaaggtcctccatggaattacaagttactatcatgagtttttggggcagcagatttctgaagaacttatccctgatttaaatcaaatcactgagtgtgcagatcctgtggagcttgggaggttgcttcagctaattctaggatgtgcagtcaactgtgaaaagaagcaagaacatattaaaaatataatgaccctggaagaatctgttcaacatgtggtcatgactgcaattcaggagctgatgagtaaagaaatagtgatctctcctgcaagtgacacggtgggagaattagaacagcagcttaaaagggctttagaagagcttcaggaggccatagcagaaaaggaagagctgaagcagaggtgccaggaactggatatgcaggtgactacacttcaggatgagaagaattcactggtctctgagaatgagatgatgaatgaaaagcttgaccagttggatggctcttttgatgatccaaatacaatggttgcaaagaaatattttcacgtacagctacaactagaacaattacaagaagaaaactataggcttgaagctgcaaaagatgattaccgtgttcactgtgaggaacttgaaaagcaactgattgaatttcagcacaggaatgatgagctgacgagccttgctgaagagaccagagccctaaaagatgagatagatgttcttagggctacgtcagacaaagcaaataaactggagtcaacagtggaggtatatcgtcagaagctacaagatttaaatgacctccgcaaacaggtgaaatctttacaggagacaaatatgatgtatatgcataacactgtgagcttggaagaagagctgaagaaggccaacgcagctcgggcacagttagagacctataagcgccaggttcaggaccttcacacaaagctttcctctgaatccaaaagggcagatacactagcatttgaaatgaagaggcttgaagaaaaacatgaaactttactcaaggaaaaagagcgattgatagaacagcgtgacactctgaaggagacgaatgaggagctgcggtgctcaaaagcacagcaagatcacttaaaccaagctgatgcatctgctacaaaaagttatgaaaatcttgctgctgagatcatgcccgtggaatacagagaggtgttcattcgactccagcatgaaaacaaaatgcttcgcctacagcaagaagggacggagaatgaacgcattgagcagctgcaggagcaactggagcagaagcaccgcaagatgaatgagctggaaaccgagcaaaggttgagcaaagagcgcatcggagaactacagcagcaaattgaggacctccagaagtcattacaagaacagggctctaagtctgaaggtgaaagttccagcaaattaaagcagaagttggaagctcatatggaaaaactaacagaagtccatgaagaattacagaagaaacaggagctcattgaagaccttcagccagatataagtcagaatgcccaaaagatcagtgaacttgaagcggctcttcagaagaaagatgaagacatgaaagcaatggaggagagatacaaaatgtacttagagaaagccaggaatgtaataaaaactttagatcccaaattaaatccagcatcagcagaaataatgttactcagaaagcagctggcggagaaggaaagaagaattgagattctagagagtgaatgtaaagtagcaaaattgagggattatgaggaaaaactcattgtttctgcctggtataacaagagcctggcgtttcagaaattgggcatggagtctcggctggtgagtggtgctagtgcttgcaaagacagcgttgcggctgctcctgcacgttccttcttagcgcagcagcggcacatcaccaacacccgaagaaacctctctgttaaggtcccagctgcagcatctgactagtctgcaaagcaaacacaacggaagtgcctcagaatatctgtaccccaagaaactaccagatagaaaacaaaggttaagatgcttggtagcagctagtcttattttggtttcactttgagacagggtctcataatcctgaactggcatagaaatgacagtgtgttcaaggatgaccttgaacagcggatcagtccttcctgcagtctctgcacgctggtgggattaacggaagtagttgtagaccacatggcaggtttgtatagttctaggggtcaaactcagaagctctgctcatcgagctgcattctcaacctcacaatcaactatatttaagtcagattatatcaaattaatattttcagcttacttagaaagcagagcacagtatcagctctgtctctgggcaggacattggagtagttggaagtgtgatgaagcgaatacttgtcttgaaatggcatcctgtgagggaaagcaaatgcatattccaactttatgtttttagttgtaacagcaagtcaagttaatttgcataaagtaatgggtagtttgtcatttaatatctagatttgctttatggaaaagtttcctgttacacatgtggagaaaaactgtgcaaggtgaattccttagtctggtgtgtatagtttcctctctttgcaaattctgccctctagaggatgcttcagcatgctgtagaagctataatgcttggaaaactgctgttgcagtgactttgtctgtaaaagtgactcattagaaatttgcagcttctctgagttgtatgccagtttgaacatgtactgctgtgaatgaggactgaactattaggataggaagtaaactgacatttgtgtttctgatgaaaagcaagctacactttgcaatagccacatgctttggagcatggtgttcttgtgggcattgctttctaagaaggagtatatcatgtataattttgcctctttattgaaaagaaatagtgatggaatagcttataaccattttggtttctgcttgcctctaaggagtgtctggagctgtatttgtggcattcggttcatttctgcccaacattgttttttccagaggtgttactttccaatttggggtcactttttaaagtcacagctttggaatattagttgttaaatcccattgcttaatgtagccacaggtggtttgctcttgagtcagtcacacatccaacactttgtagccacgtttccatcattgttgagagtttgttgagagttcatgtgcagcgagtcacaatcttggtttttggtaacttgaagaaattctttgatccttagactacagatttaagaagtgttacgtccatttcacttgtacagaggaatcagatggggaaaggagtgcagcagacacaagggagacaacagtgtgctttcccagaacattgccagtcattgggtgaaggtgtaaacttgcaggctgcatgacccttgttgggtcagcttctcacctctgccctcatcatagcatgaaagaagggatatgtaatatataaacaaaaagggggtggtggtggctgagtaagccagattgagctggcaggctgtggagctgtggtctacattttattcttagtccttcataaatatgagtattgttctatttgcagtgttaactcatttagataattgcctgtaaatccttgcccagtaattcttaaccacttaaaataacaagaatttcacaatgatttggggtctttgagattagtggcatctgccaggatattttgttttatcagggttccttctgttcactacctcttagattttgatgattcttgtctacattttacactgttggtttgtatttttgtatgtttatggtgtctgcttgcctgtgtcttttagtaaaataagactttaaaattacttagcatgctatttaacattttctaaaaaaaaaaaaaaa

SEQ ID NO: 36 - protein, 728 aa, Mus musculus
  source 1..728: note = HOOK1
MEDPQPLPQSELPLCDSLIIWLQTFKTASPCQDVKQLTNGVTMAQVLHQIDVAWFSESWLSRIKDDVGDNWRIKASNLKKVLHGITSYYHEFLGQQISEELIPDLNQITECADPVELGRLLQLILGCAVNCEKKQEHIKNIMTLEESVQHVVMTAIQELMSKEIVISPASDTVGELEQQLKRALEELQEAIAEKEELKQRCQELDMQVTTLQDEKNSLVSENEMMNEKLDQLDGSFDDPNTMVAKKYFHVQLQLEQLQEENYRLEAAKDDYRVHCEELEKQLIEFQHRNDELTSLAEETRALKDEIDVLRATSDKANKLESTVEVYRQKLQDLNDLRKQVKSLQETNMMYMHNTVSLEEELKKANAARAQLETYKRQVQDLHTKLSSESKRADTLAFEMKRLEEKHETLLKEKERLIEQRDTLKETNEELRCSKAQQDHLNQADASATKSYENLAAEIMPVEYREVFIRLQHENKMLRLQQEGTENERIEQLQEQLEQKHRKMNELETEQRLSKERIGELQQQIEDLQKSLQEQGSKSEGESSSKLKQKLEAHMEKLTEVHEELQKKQELIEDLQPDISQNAQKISELEAALQKKDEDMKAMEERYKMYLEKARNVIKTLDPKLNPASAEIMLLRKQLAEKERRIEILESECKVAKLRDYEEKLIVSAWYNKSLAFQKLGMESRLVSGASACKDSVAAAPARSFLAQQRHITNTRRNLSVKVPAAASD

SEQ ID NO: 37 - DNA, 3010 bp, Bos taurus
  source 1..3010: mol_type = genomic DNA; note = HOOK1
gctaccgagctttcctggtgggcgagcaggtcctcgacgtcggctcctgggggagcgcggggaggagggaggtgtcggcgtcagcggtgtagtcgacggcggtcctggccatggaagagacgcagccgcttccgcagtccgagctcccgctgtgcgacagcctcatcatctggctgcagacattcaatactgctgcaccctgtcaggatgtcaaacagctgactagtggagttgccatggcacaagttcttcatcaaattgatgtagcttggttcgatgaaccttggttaagccgaattaaagaagatactggtgacaactggagaataaaggctagtaatttaaagaaggtacttcaaggaattatgagttactacaatgagtttttggggcaacagatttctgaagaacttatccctgatttaaaccagataactgaatgttcagattctgtggagcttgggagattgctccagcttattttaggttgtgctgtcaactgtgaaaagaaacaagaacatattcagaatataatgactttggaagaatctgttcaacatgtggtcatgactgctattcaagagttgatgagtaaagaaattgtgagctctcccacaaatgatgctgttggagaattagaacaacagcttaaaagggccctagaagaacttcaggaagcactagcagaaaaagaagaactgaggcaaagatgccaagaactagatatgcaggtgacagcgcttcaagatgaaaagaattcactagtttctgagaacgagatgatgaatgaaaaacttgaccagttggatggatcttttgatgatccaaatacaatggttgcaaaaaaatattttcatgcacagttgcaactagaacaattacaggaagaaaacttcaggcttgaagctgcaaaagatgattaccgtgttcactgtgaagaacttgaaaagcaattaattgaatttcaacataggaatgatgaattgactagtcttgctgaggaaacaagagccctgaaagatgaaatagatgttcttagggctacctctgataaagcaaataaactggagtctacagttgagatatatcgtcagaagctacaagatctgaatgaccttcgcaagcaagtgaaaactttacaggaaacaaacatgatgtacatgcataatacagtcagcttagaagaggaattaaagaaggcaaatgctgcacgcacacaattagaaacatacaagagacaggttcaggatcttcatgttaaactttcctctgaatccaaaagggcggacacactagcatttgaaatgaagcggcttgaagagaaacacgaagctttacttaaggaaaaagagagactaatagaacagcgtgacactctgaaagaaacaaatgaagagcttcgatgttcacaagtacaacaggatcacctaaaccaagcagatgcgtccgctacaaaaagttatgagaatcttgctgctgagattatgccagtggaatatagggaggtgttcattcgactgcaacatgaaaataagatgcttcgattacagcaggaaggcactgagaacgagcgtattgaggagctgcaggagcagctggagcagaagcaccgcaagatgaatgagctggaaaccgagcagaggctgagcaaagagcgaatccgagagttgcagcagcagattgaggacctccagaagtctttacaagaacaaggctccaagtctgaaggtgaaagttccagcaaactaaagcagaagttggaagctcatatggaaaaactaacagaggtccatgaagaactacagaagaaacaagagctcattgaagacctccagccagatgtaaaccagaacgtacaaaagatcaatgagcttgaagctgctcttcagaagaaagatgaagatatgaaagcaatggaggaaagatacaaaatgtacttagagaaagcaagaaatgtaataaaaactttagatcccaaattaaatccagcatcagctgaaataatgctacttagaaagcagttggcagagaaagagagaagaattgagattctggagagtgaatgtaaagtagcaaaattccgtgattatgaagaaaagctcattgtttctgcctggtataataagagtctagcattccagaaattggggatggaatctagacttgtgagcagcagtggtgcttgcaaagacacgggggcctgtgctcctgcccggtctttcctagcacagcagcgccacatcaccagcaccagaagaagtctctctgttaaagttcctgctgcgacaactgattaaaccgcaaaagaacacataaacagaagtgcccttaaaatgttttgtatctttcaactaactgcatggttgaaaagaagtgtatgtttctggatagcagcttttttaaaatcaaaatgtatgcaattaatcttgccagattaatatggagacagaatttagaagtagctatctgggggagcacgtgtgagtaattggaagatataataaggtaaacatctcaaacgtttgtctttggagagtattatattatttcacattgacttagtatattttaatgttctcagttgtagcagtaactaatttgcaatgtgtaatcagttatatgcaatttaaaactactgttctggtccatataaaaatagtgaacaggtaggcacatggagaactatgcaagagttatattttttgattaaaaatacacagatattacctctgacctctcattctgtcgttgaatgggtatattttatcatgtaatggaaaataacatttggaagatttgactatattgaaatgacttctccatagtagagtgactatttggaaatgttgcagatttagacaaggtcatagttctcatatgtacctactataaacaaaggcttctgtatagcatgcttgaaatgttaagggtatggacttttgactttaagagatgactttcggacgggaagcaagctgaaaagtctactttgtgtgaaatgcccacacta

SEQ ID NO: 38 - protein, 728 aa, Bos taurus
  source 1..728: note = HOOK1
MEETQPLPQSELPLCDSLIIWLQTFNTAAPCQDVKQLTSGVAMAQVLHQIDVAWFDEPWLSRIKEDTGDNWRIKASNLKKVLQGIMSYYNEFLGQQISEELIPDLNQITECSDSVELGRLLQLILGCAVNCEKKQEHIQNIMTLEESVQHVVMTAIQELMSKEIVSSPTNDAVGELEQQLKRALEELQEALAEKEELRQRCQELDMQVTALQDEKNSLVSENEMMNEKLDQLDGSFDDPNTMVAKKYFHAQLQLEQLQEENFRLEAAKDDYRVHCEELEKQLIEFQHRNDELTSLAEETRALKDEIDVLRATSDKANKLESTVEIYRQKLQDLNDLRKQVKTLQETNMMYMHNTVSLEEELKKANAARTQLETYKRQVQDLHVKLSSESKRADTLAFEMKRLEEKHEALLKEKERLIEQRDTLKETNEELRCSQVQQDHLNQADASATKSYENLAAEIMPVEYREVFIRLQHENKMLRLQQEGTENERIEELQEQLEQKHRKMNELETEQRLSKERIRELQQQIEDLQKSLQEQGSKSEGESSSKLKQKLEAHMEKLTEVHEELQKKQELIEDLQPDVNQNVQKINELEAALQKKDEDMKAMEERYKMYLEKARNVIKTLDPKLNPASAEIMLLRKQLAEKERRIEILESECKVAKFRDYEEKLIVSAWYNKSLAFQKLGMESRLVSSSGACKDTGACAPARSFLAQQRHITSTRRSLSVKVPAATTD

SEQ ID NO: 39 - DNA, 2965 bp, Gallus gallus
  source 1..2965: mol_type = genomic DNA; note = HOOK1
gcgtgtggttcccgccgtaagcgcgctccgcgcatcggatgcttacgcagccgccctgagcggaggcggcgggcaccgacaggaggctgacgccatggaggggaaggcggcggacccgctgctctgtgacagcctcatcctgtggttgcagacgttcaatactgccgcaccttgcagaaatgttcaggacttgaccaatggtgttgccatggctcaggtgcttcatcaaatagatgttgcttggtttgatgcatcttggctaaatcgcattaaagaagatgttggtgacaactggaggataaagtccagtaatctgaagaaaatactgcaaggaattatggactactatcatgagtttttggatcagcagatttcagaagaacttattccggacttaaataagatttctgagaattcagatcctactgaactgggaaggcttatgcagcttattttaggctgtgcggtcaactgtgaaaggaaacaagaacacatacaaaatatcatgacccttgaagagtctgttcaacatgttgtcatgacagccattcaagagctcatgagcaaggaagcaatgggtccttctgcaagtgatgtgtcaagtgaaatggaacagcagcttaaaaaagctttggaggatcttcaagaagcaatagcagaaaaagaagagttggcacaaagatgtcaagagctagatttacaggtggctgcccttcaggatgaaaaaaacagcttggtgtcagaaaatgagattctgaatgacaggctggagcaattagatgactctcttgatgatccaaataccgtggtagctaagaagtattttcatgcgcagttgcagctagaacaacttcaagaagaaaacttcaggcttgaggctgccaaagatgattatcgtgtccattgtgaagatctagaaaaacaactgattgaactacaacatagaaacaacgaactaacctctttggccgaagaatctagagcgttgaaagatgaaaatgatattcttagagctgctgcagacaaagcaagtaagctggaatcaactgttgaagtgtatcgtaaaaagctgcaggatctgaacgacttccgaagacaagtcaaatctctgcaggaaactaacatgatgtacatgcacaatacagtcagtctggaagatgaactaagggaagccaatgcagcccgtgcccaattagaaacctacaaaagacaggtccaggagcttcataacaagctgtctgaagaatctaaaagagctgataagttagcatttgaaatgaaacgacttgaagaaaaacatgaagctttagtcaaagaaaaagagagactggtaatacagtgtgatgcattgaaagagactaatgaagagctccggtattcacagatgcaacaggatcatctgagtcgaacagatgcatctcgcattaaaagccatgataatcttgctgctgagcttttgcctgtggaatatagagaaatgtttattcagcttcagcatgaaaataagatgcttctactgcaacaggaaggatcagaaaatgaacgtattatggagctccagaaacagctggagcaaaagcagtggacagtaaatgaactaggaactgaaaaaaggctaaacaaggagcgtattggagagttacaacagcagattgaagatctgcagaagactttacaggagcaaggttccaagacagaaggatctagcaacctgaagcagaaattggcagcacatatggaaaaactgagtgaagttcatgatgaattacagaagaaagaggctgctcttgcagaactacagcctgatgttagtcagaacccccagaagattggggagctggaagcagctttgcgaaaaaaagatgaggacatgaaagcaatggaagaaagatataaaatgtaccttgagaaagcaagaaatgtgataaaaaccttggaccccaagctgaatccagcatcagcagaaattatgttgcttagaaaacagataacagagagagacaaaaaaattgaagcactggaggctgaatacaaacttgctaagttacgtgattatgaggaaaatttaattgtaactgcatggtataacaagagcctaacgctccagaaattagggatggaagctagactgcttggcagtggtggtgcctgcagggatggcccaggacgttccttcctagctcaacagcgccatgtcaccaatactagaaggaatcttcctgttaaagtaccaagtgcaacatctgattaaaccataaggaccgtgagggaagcttacatgctgaagtgtccttaaatgttctatagcttccacttcaactgtttggtgaaagatgatgttacagtggtgggcatctgtgaattcagcatcagaatttatcaagaacttaacttgattaacttgatcagtggtatctagaaggtagcacggtaaaagtagatttaataactacagtatgtatctacaagcacaacttaagatttatatttgccttgaagatatattgtaaaaataaaaattggcactatatatttaaaactaatcaatgtattagggctagagtgttgactttcgtaatagaaagatgttatgcaaaaatatacttcaggaaactggcaatatttgacatttttgctggcctcttctaaaatgaagtttaaaaaatactttaaaaagttttaaatactttaaaaatacttcttatataatataaaatgcaattttagaagcgagaaaatatttgcatttatttaaaaataaaattttaaaattgcattactagttgacctgtataacaggaggcaggctttgtcatctcaagtcagagacacaattatgcagactatttaaataaagatcaagttagtgcttatatacactaaggaaatcctatctttaagatgttgaattcaccaaagccaaataagctgtcttttttcctgtttaagaaaaaaaaaaaaaaaa

SEQ ID NO: 40 - protein, 718 aa, Gallus gallus
  source 1..718: note = HOOK1
MEGKAADPLLCDSLILWLQTFNTAAPCRNVQDLTNGVAMAQVLHQIDVAWFDASWLNRIKEDVGDNWRIKSSNLKKILQGIMDYYHEFLDQQISEELIPDLNKISENSDPTELGRLMQLILGCAVNCERKQEHIQNIMTLEESVQHVVMTAIQELMSKEAMGPSASDVSSEMEQQLKKALEDLQEAIAEKEELAQRCQELDLQVAALQDEKNSLVSENEILNDRLEQLDDSLDDPNTVVAKKYFHAQLQLEQLQEENFRLEAAKDDYRVHCEDLEKQLIELQHRNNELTSLAEESRALKDENDILRAAADKASKLESTVEVYRKKLQDLNDFRRQVKSLQETNMMYMHNTVSLEDELREANAARAQLETYKRQVQELHNKLSEESKRADKLAFEMKRLEEKHEALVKEKERLVIQCDALKETNEELRYSQMQQDHLSRTDASRIKSHDNLAAELLPVEYREMFIQLQHENKMLLLQQEGSENERIMELQKQLEQKQWTVNELGTEKRLNKERIGELQQQIEDLQKTLQEQGSKTEGSSNLKQKLAAHMEKLSEVHDELQKKEAALAELQPDVSQNPQKIGELEAALRKKDEDMKAMEERYKMYLEKARNVIKTLDPKLNPASAEIMLLRKQITERDKKIEALEAEYKLAKLRDYEENLIVTAWYNKSLTLQKLGMEARLLGSGGACRDGPGRSFLAQQRHVTNTRRNLPVKVPSATSD

SEQ ID NO: 41 - DNA, 2880 bp, Mus musculus
  source 1..2880: mol_type = genomic DNA; note = TBPL1
gagaaccggaagtgagtggctagccgggatccccggagctcctgggtccggcggcgctctagccgcctcgtgccgcctctatgcccccgtgcgtctagtctatttattgtcgcgggggaagcggcggccgccctgtacccggaacaacaaagcgagaaaccgagctcgagccttggggggctcctagcaacgggcctgggcgggagttccatggagactggggagcggacccgttttatcttcatccttgtcctccagcttcttctccgcgtccgacgcaaccagcagcagcgctgccgccgtgtcctttacgaccgccccgtcttcccacggatgtgatcttcgtggtgggaaccaagtttctaaactaccccaatggatgcagacagtgatgttgcattggacattttaattacaaatgtagtctgtgtttttagaacaagatgccatttgaacttaaggaagattgctttggagggagcaaatgtaatttataagcgtgatgttgggaaagtattaatgaagcttagaaaacctagaattacagctacaatttggtcctcaggaaaaattatttgcactggagcaacaagtgaagaagaagctaaatttggtgccagacgtttagcccgtagtctgcagaaactaggttttcaggtcatcttcacagattttaaggtggtgaatgttttggcagtgtgtaacatgccctttgagatccgtttgccagaatttacaaagaacaatagacctcatgccagctatgaacctgaacttcatcctgccgtgtgctatcggataaagtctctaagagctacattacagatattttcaacaggaagcatcacagtgacagggcccaatgtaaaggctgtggcgactgccgtggaacagatctacccgttcgtgtttgaaagcaggaaggagattttataactcaaacttggttggttagtctccacctgagcaccgtttgagacctgctgcacattggactcaaaacacaaggaaaactggaccaacagtgaccgaggacagactcggactcacagctgcagtgtaagctccagtccctttggattttacttcaaagtttgctgtaatataaaaaggaagtttacaagacatgacattgctgcttttacaaaaggacgttctatttattttcgcagtaatctccatgtcccacaggcagagctgtcgcagtgtgcacttcccgacagttgttttattgatgtcattatttttccagttggagctaatgtgtttgatttgtgaatagtcttttacatttttgtatgctgaatatgggcaccaaagaacctgtaaacgttatctttttaaattgaatgtgcacaaataaaagtttggaaaagatctctcttcatatccattctgtaacttttattcaccatttttttcttttaaacaactgtattcataattttttttaaatctatgcaggcttagactttggctaaagtgtgtgggcttctttttgaagtgagtttgtatatatttgtgtgtatgctacatgtatatagtatcttttcatgctctgttaccaaatatcaattagaatgttctcctcgcagattggaagtaaaaaccatgtatataagctctaggtaactagggtagaactccacaggtaaatgataatgtttgtgttactaatttaatatgatagaaaatgttaaatgcgtttgaaaagctaagaggttgtagttttgaagagaggccttttcctgtctaggtggatctgcagtagccccgtaacccttgttattctggaaagctggctgtagatgaacgagccagcagaggttcttaactacccttaggtcggtgctcaaggctatgctggtcttgtcagaaattgactgacattgtgttttaacaagaagtggctcgacttgacccaaagcctctggcaagctggtctctgtcctcgccgtgtctgagtagtgcatgtgaagggtgactggaggattaagagaggccactcttaaacttcagttccatgagacagggcctcagccgtgcatgggctctgggtggaaaggcatggtcggacaggtgcttagtgtagccaggagtggctttacctaccaataaactaggtggagtcaaaacttagattctctgtacagacgaaactatttcccaccaagctctgacttactgttgtatttttgaaatgtttgcaaaggcttgtgacatgtgcaccatattacagaccttccaaggctaacttaagcactggctgggcagtgcataggagtctgtgttcagttctgttttcttaaagcctcctcatcccctgcccctcagctccttcagaacgtgggagcaaaggtgagctttgcacttgcttttcccataattcattgtgactttttaaagccttcaattttttaaaaattgtttttatatttgggttttggtttttttttttttttttttgcttgttttaagattatttttaaaaaaccagaggtagaagtagacggctttaaaaccgtgaggatttaccacttgttttaacagtgaactcctggtttgcagtgttcagaaacatgtatgtgaggcagaaacttgggtagggaaggtgcagatccattcgctgtaacatgttcagtgttcagtgacctgaaagccaggcagagctggagctctgggtaaatgagtgtctaaggagggaagagtgagcatggccgagtctggggtcacctgtgaggccactgtctggcagcttcctgggcagtccccacaaaaatgctgggaaggagtgagtgggtgttgcgtcgcctggttttctacaagtactgtaaaattttttatataaataaaaactgtgaatatatacatccaaaaaaa

SEQ ID NO: 42 - protein, 186 aa, Mus musculus
  source 1..186: note = TBPL1
MDADSDVALDILITNVVCVFRTRCHLNLRKIALEGANVIYKRDVGKVLMKLRKPRITATIWSSGKIICTGATSEEEAKFGARRLARSLQKLGFQVIFTDFKVVNVLAVCNMPFEIRLPEFTKNNRPHASYEPELHPAVCYRIKSLRATLQIFSTGSITVTGPNVKAVATAVEQIYPFVFESRKEIL

SEQ ID NO: 43 - DNA, 1194 bp, Bos taurus
  source 1..1194: mol_type = genomic DNA; note = TBPL1
gagttccatggagactggggagcgcgctcgcctcatcatcatccttatcctccagcttatccttcgcgtccgccgcaaccggcagcagcgctgccgccgcgtcctctgccgccgcccgctcttcccacggatgtgatcttcgtggtgggaagccaagtttttaaactaccccaatggatgcagacagtgatgttgcactggacattctaattacaaatgtagtatgtgtttttagaacaagatgccatttgaacttaaggaagattgctttagagggagcaaacgtaatttataagcgtgatgttggaaaagtattaatgaagcttagaaagcctagaattacagctacaatttggtcctcaggaaaaattatttgcactggagcaacaagtgaagaagaagctaaatttggtgccagacgtttagcccgtagtctccagaaactaggatttcaggtaatatttacagattttaaggttgttaacgttttggcagtgtgtaacatgccgtttgaaatccgtttgccagaattcacaaagaacaatagacctcatgccagttacgaacctgagcttcatcctgctgtgtgctatcggataaaatcgctaagagctacattacagattttttcaacaggaagtatcacagtaacaggacccaatgtaaaggctgttgctactgctgtggaacagatttacccatttgtgtttgaaagcaggaaagagattttataattcaccacttaattggttagaatccttaactgagcaccttttaaaacctgctgcacattggactcaaaaggaaaaccggaccaacagtaactgaggaaatagactcttttattctttcacggctacagtgtaagctccaggccctttggattttatgtcaaaccttgctgtaatataaaaaggaagtttacaagacatgacattgctgcttttacaaaaggacatttctatttattttcgcagtaattttcatgtccccgtgagcagagctgtcaccgtgtgcactaccgcagatcgttttgttgttgtcattatttttttccagtttgagctaatgtgttttatttgtgaatagtcttttacatttttgtatgctgaatatgggcaccaaagaacctgtaaaagttacctttttcaattgaatgtgcacaaataaaagtttggaaaaaaaaaaaaaaaa

SEQ ID NO: 44 - protein, 186 aa, Bos taurus; identical to SEQ ID NO: 42
  source 1..186: note = TBPL1

SEQ ID NO: 45 - DNA, 1049 bp, Gallus gallus
  source 1..1049: mol_type = genomic DNA; note = TBPL1
gatcttcatggtgaactggtgtgagaactgagttgtaaatcaccccaatggatgcggacagtgatgttgcattggacattttaatcacaaatgtagtgtgtgtttttagaacaagatgtcatttaaacttgaggaagattgcattagagggagcaaacgtgatatacaagcgtgatgttgggaaggtattaatgaaacttagaaaacctaggattacagccacaatttggtcctcaggaaaagttatttgcacaggagccacaagtgaagaagaagctaaatttggtgccagacgattagctcgttgtctacagaaactaggttttcaggtaattttcacagattttaaagttgtgaatgttttagcagtgtgcaacatgccctttgagatcagattgccagaatttacgaagaataacagacctcatgctagttatgaaccagaacttcatcctgctgtgtgctacagaataaaaactctcagagctaccttacagattttttcaactggcagtatcacagttacagggccaaacgtaaaggctgttgccagtgctgtggagcagatttacccgttcgtgtttgaaagcagaaaataaattttataattcaccacttgatggttaggttccctaaccgagcacctttaaagactgctgcacattggactaaaagcaaaaaggaaaactggaccaactatagcagaggaatacagacttttttcttgttcatggccacagtataaactccagtccttttggattttactcttaacagtgctgtattgtaaaaacagaagtttacaagatatgaaattgctgcttttaaaaagacattctatttatttttgcagtaatttctgtgtattcataagcaaagctgtcacgatgtgcactacctttaaaacatttttttttgtttagtttgagcttgtgttttatttgtgaatagtcttttacatttttgtatgctgaatattgggcaccaaagaagctgtaaaagttatctttttcacctgatgaatgtgcacaaataaaagtttggaaact

SEQ ID NO: 46 - protein, 183 aa, Gallus gallus
  source 1..183: note = TBPL1
MDADSDVALDILITNVVCVFRTRCHLNLRKIALEGANVIYKRDVGKVLMKLRKPRITATIWSSGKVICTGATSEEEAKFGARRLARCLQKLGFQVIFTDFKVVNVLAVCNMPFEIRLPEFTKNNRPHASYEPELHPAVCYRIKTLRATLQIFSTGSITVTGPNVKAVASAVEQIYPFVFESRK

SEQ ID NO: 47 - DNA, 4107 bp, Streptococcus pyogenes
  source 1..4107: mol_type = genomic DNA; note = Cas9 protein id: AAK33936.1; note = M1 GAS
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactga

SEQ ID NO: 48 - protein, 1368 aa, Streptococcus pyogenes [sequence omitted]

SEQ ID NO: 49 - DNA, 4335 bp, synthetic construct
  source 1..4335: mol_type = other DNA; note = Cas9 adapted for mammalian expression
atggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagggaagcggagccactaacttctccctgttgaaacaagcaggggatgtcgaagagaatcccgggcca

SEQ ID NO: 50 - protein, 1445 aa, synthetic construct
  source 1..1445: note = Cas9 adapted for mammalian expression
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKGSGATNFSLLKQAGDVEENPGP

SEQ ID NO: 51 - DNA, 24620 bp, synthetic construct
  source 1..24620: mol_type = other DNA; note = VB201210-1271bpw (for constructing mice#2)
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtggcaactttgtatagaaaagttgaaaaaagcaccgactcggtgccactttttcaagttgataacggactagccttattttaacttgctatttctagctctaaaacggtcacaatgaggttggggcggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaactggtcagggatggagggtgttagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaactggctgagcggcctcggcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaactggtggccctagggaccaccaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaacctaatgcccactagcctgccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattggccaccatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaactttattatacatagttgtaactttaaataattggcattatttaaagttatccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtaaatataaaatttttaagtgtataatgtgttaaactactgattctaattgtttgtgtattttaggtgtctatggtcgagattatgcaggaagggacatggaagaagcctgaaacactgatgcggtatctcagaaggggcggagtgtccgtgggagccaattctcgactgatggatagctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcttcagtaactttaaataattggcattatttaaagttacaattgaagaaatttaaatgaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttgttaattaattgattcgaggctgctaacaaatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggattaattaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtccgtacgggcgcgccgcggccgc

SEQ ID NO: 52 - DNA, 4646 bp, synthetic construct
  source 1..4646: mol_type = other DNA; note = Homology left arm of the VB201210-1271bpw (for constructing mice#2)
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtgg

SEQ ID NO: 53 - DNA, 10282 bp, synthetic construct
  source 1..10282: mol_type = other DNA; note = The KI insert of the VB201210-1271bpw (for constructing mice#2)
caactttgtatagaaaagttgaaaaaagcaccgactcggtgccactttttcaagttgataacggactagccttattttaacttgctatttctagctctaaaacggtcacaatgaggttggggcggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaactggtcagggatggagggtgttagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaactggctgagcggcctcggcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaactggtggccctagggaccaccaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaacctaatgcccactagcctgccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattggccaccatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaactttattatacatagttg

SEQ ID NO: 54 - DNA, 3673 bp, synthetic construct
  source 1..3673: mol_type = other DNA; note = The SDN sequence of the VB201210-1271bpw (for constructing mice#2)
taactttaaataattggcattatttaaagttatccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtaaatataaaatttttaagtgtataatgtgttaaactactgattctaattgtttgtgtattttaggtgtctatggtcgagattatgcaggaagggacatggaagaagcctgaaacactgatgcggtatctcagaaggggcggagtgtccgtgggagccaattctcgactgatggatagctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcttcagtaactttaaataattggcattatttaaagttacaattgaagaaatttaaat

SEQ ID NO: 55 - DNA, 2550 bp, synthetic construct
  source 1..2550: mol_type = other DNA; note = The homology right arm sequence of the VB201210-1271bpw (for constructing mice#2)
gaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttg

SEQ ID NO: 56 - DNA, 657 bp, synthetic construct
  source 1..657: mol_type = other DNA; note = The DTA sequence of the VB201210-1271bpw (for constructing mice#2)
atggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaa

SEQ ID NO: 57 - DNA, 20 bp, synthetic construct
  source 1..20: mol_type = other DNA; note = The spem1 gRNA #5 of KI insert sequence of plasmid (for constructing mice#2)
ggtcacaatgaggttggggc

SEQ ID NO: 58 - DNA, 248 bp, synthetic construct
  source 1..248: mol_type = other DNA; note = The U6 promoter of KI insert sequence of plasmid (for constructing mice#2)
ggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctc

SEQ ID NO: 59 - DNA, 20 bp, synthetic construct
  source 1..20: mol_type = other DNA; note = The spem1 gRNA #4 of KI insert sequence of plasmid (for constructing mice#2)
tggtcagggatggagggtgt

SEQ ID NO: 60 - DNA, 227 bp, synthetic construct
  source 1..227: mol_type = other DNA; note = The H1 promoter of KI insert sequence of plasmid (for constructing mice#2)
tagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatt

SEQ ID NO: 61 - DNA, 20 bp, synthetic construct
  source 1..20: mol_type = other DNA; note = The spem1 gRNA #3 of KI insert sequence of plasmid (for constructing mice#2)
tggctgagcggcctcggcct

SEQ ID NO: 62 - DNA, 243 bp, synthetic construct
  source 1..243: mol_type = other DNA; note = The h7SK promoter of KI insert sequence of plasmid (for constructing mice#2)
gaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcag

SEQ ID NO: 63 - DNA, 20 bp, synthetic construct
  source 1..20: mol_type = other DNA; note = The spem1 gRNA #2 of KI insert sequence of plasmid (for constructing mice#2)
tggtggccctagggaccacc

SEQ ID NO: 64 - DNA, 255 bp, synthetic construct
  source 1..255: mol_type = other DNA; note = The mouse U6 promoter of KI insert sequence of plasmid (for constructing mice#2)
aaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgag

SEQ ID NO: 65 - DNA, 20 bp, synthetic construct
  source 1..20: mol_type = other DNA; note = The spem1 gRNA#1 of KI insert sequence of plasmid (for constructing mice#2)
ctaatgcccactagcctgcc

SEQ ID NO: 66 - DNA, 249 bp, synthetic construct
  source 1..249: mol_type = other DNA; note = The human U6 promoter of KI insert sequence of plasmid (for constructing mice#2)
ggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctc

SEQ ID NO: 67 - DNA, 424 bp, synthetic construct
  source 1..424: mol_type = other DNA; note = The tetracycline response element (TRE) of KI insert sequence of plasmid (for constructing mice#2)
ggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattg

SEQ ID NO: 68 - DNA, 300 bp, synthetic construct
  source 1..300: mol_type = other DNA; note = The KRAB domain of KI insert sequence of plasmid (for constructing mice#2)
atgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctc

SEQ ID NO: 69 - DNA, 4104 bp, synthetic construct
  source 1..4104: mol_type = other DNA; note = The dCas9 of KI insert sequence of plasmid (for constructing mice#2)
atggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 70 - DNA, 1733 bp, synthetic construct
  source 1..1733: mol_type = other DNA; note = The CAG promoter of KI insert sequence of plasmid (for constructing mice#2)
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 71 - DNA, 747 bp, synthetic construct
  source 1..747: mol_type = other DNA; note = The tTa of KI insert sequence of plasmid (for constructing mice#2)
atgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 72 - DNA, 24863 bp, synthetic construct
  source 1..24863: mol_type = other DNA; note = Plasmid sequence (for constructing mice#4)
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtggcaactttgtatagaaaagttgaaaaaagcaccgactcggtgccactttttcaagttgataacggactagccttattttaacttgctatttctagctctaaaaccaacaaggcgtcctcctccacggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaaccctcattgactcccatagactagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaaccagggtagcaccctgcgtgagaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaacccctgagtcacccacgacggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaactagggaccaccatggccatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattggccaccatgactagtcagattttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtggcgacgctagtaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaactttattatacatagttgtaactttaaataattggcattatttaaagttatccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtaaatataaaatttttaagtgtataatgtgttaaactactgattctaattgtttgtgtattttaggtgtctatggtcgagattatgcaggaagggacatggaagaagcctgaaacactgatgcggtatctcagaaggggcggagtgtccgtgggagccaattctcgactgatggatagctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcttcagtaactttaaataattggcattatttaaagttacaattgaagaaatttaaatgaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttgttaattaattgattcgaggctgctaacaaatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggattaattaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtccgtacgggcgcgccgcggccgc

SEQ ID NO: 73 - DNA, 4646 bp, synthetic construct; identical to SEQ ID NO: 52
  source 1..4646: mol_type = other DNA; note = Homology left arm sequence of plasmid (for constructing mice#4)

SEQ ID NO: 74 - DNA, 10525 bp, synthetic construct
  source 1..10525: mol_type = other DNA; note = The KI insert sequence of plasmid (for constructing mice#4)
caactttgtatagaaaagttgaaaaaagcaccgactcggtgccactttttcaagttgataacggactagccttattttaacttgctatttctagctctaaaaccaacaaggcgtcctcctccacggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaaccctcattgactcccatagactagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaaccagggtagcaccctgcgtgagaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaacccctgagtcacccacgacggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaactagggaccaccatggccatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgacctatataagcagagctcgtttagtgaaccgtcagatcgcctggagacgccatccacgctgttttgacctccatagaagacaccgggaccgatccagcctccgcggccccgaattggccaccatgactagtcagattttcgtgaagaccctgaccggcaagaccatcaccctagaggtggagcccagtgacaccatcgagaacgtgaaggccaagatccaggataaagagggcatcccccctgaccagcagaggctgatctttgccggcaagcagctggaagatggccgcaccctctctgattacaacatccagaaggagtcaaccctgcacctggtccttcgcctgagaggtggcgacgctagtaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtctagactggacaagagcaaagtcataaactctgctctggaattactcaatgaagtcggtatcgaaggcctgacgacaaggaaactcgctcaaaagctgggagttgagcagcctaccctgtactggcacgtgaagaacaagcgggccctgctcgatgccctggcaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggatcaggagcatcaagtagcaaaagaggaaagagagacacctaccaccgattctatgcccccacttctgagacaagcaattgagctgttcgaccatcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaactttattatacatagttg

SEQ ID NO: 75 - DNA, 3673 bp, synthetic construct; identical to SEQ ID NO: 54
  source 1..3673: mol_type = other DNA; note = The SDN sequence of plasmid (for constructing mice#4)

SEQ ID NO: 76 - DNA, 2550 bp, synthetic construct; identical to SEQ ID NO: 55
  source 1..2550: mol_type = other DNA; note = The sequence of the right homology arm of plasmid (for constructing mice#4)

SEQ ID NO: 77 - DNA, 657 bp, synthetic construct; identical to SEQ ID NO: 56
  source 1..657: mol_type = other DNA; note = The DTA sequence for plasmid (for constructing mice#4)

SEQ ID NO: 78 - DNA, 24620 bp, synthetic construct; identical to SEQ ID NO: 51
  source 1..24620: mol_type = other DNA; note = Plasmid sequence (for constructing mice#6)

SEQ ID NO: 79 - DNA, 10282 bp, synthetic construct; identical to SEQ ID NO: 53
  source 1..10282: mol_type = other DNA; note = The KI insert sequence of plasmid (for constructing mice#6)

SEQ ID NO: 80 - DNA, 23551 bp, synthetic construct
  source 1..23551: mol_type = other DNA; note = Plasmid sequence (for constructing mice#7)
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtggcaactttgtatagaaaagttgaaaaaagcaccgactcggtgccactttttcaagttgataacggactagccttattttaacttgctatttctagctctaaaaccaacaaggcgtcctcctccacggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaaccctcattgactcccatagactagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaaccagggtagcaccctgcgtgagaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaacccctgagtcacccacgacggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaactagggaccaccatggccatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtggcaactttattatacatagttgtaactttaaataattggcattatttaaagttatccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtaaatataaaatttttaagtgtataatgtgttaaactactgattctaattgtttgtgtattttaggtgtctatggtcgagattatgcaggaagggacatggaagaagcctgaaacactgatgcggtatctcagaaggggcggagtgtccgtgggagccaattctcgactgatggatagctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcttcagtaactttaaataattggcattatttaaagttacaattgaagaaatttaaatgaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttgttaattaattgattcgaggctgctaacaaatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggattaattaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtccgtacgggcgcgccgcggccgc

SEQ ID NO: 81 - DNA, 9213 bp, synthetic construct
  source 1..9213: mol_type = other DNA; note = The KI insert sequence of plasmid (for constructing mice#7)
caactttgtatagaaaagttgaaaaaagcaccgactcggtgccactttttcaagttgataacggactagccttattttaacttgctatttctagctctaaaaccaacaaggcgtcctcctccacggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaaccctcattgactcccatagactagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaaccagggtagcaccctgcgtgagaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaacccctgagtcacccacgacggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaactagggaccaccatggccatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtggcaactttattatacatagttg

SEQ ID NO: 82 - DNA, 23548 bp, synthetic construct
  source 1..23548: mol_type = other DNA; note = Plasmid sequence (for constructing mice#8)
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtggcaactttgtatagaaaagttgaaaaaagcaccgactcggtgccactttttcaagttgataacggactagccttattttaacttgctatttctagctctaaaacggtcacaatgaggttggggcggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaactggtcagggatggagggtgttagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaactggctgagcggcctcggcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaactggtggccctagggaccaccaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaacctaatgcccactagcctgccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtggcaactttattatacatagttgtaactttaaataattggcattatttaaagttatccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtaaatataaaatttttaagtgtataatgtgttaaactactgattctaattgtttgtgtattttaggtgtctatggtcgagattatgcaggaagggacatggaagaagcctgaaacactgatgcggtatctcagaaggggcggagtgtccgtgggagccaattctcgactgatggatagctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcttcagtaactttaaataattggcattatttaaagttacaattgaagaaatttaaatgaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttgttaattaattgattcgaggctgctaacaaatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggattaattaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtccgtacgggcgcgccgcggccgc

SEQ ID NO: 83 - DNA, 9210 bp, synthetic construct
  source 1..9210: mol_type = other DNA; note = The KI insert sequence of plasmid (for constructing mice#8)
caactttgtatagaaaagttgaaaaaagcaccgactcggtgccactttttcaagttgataacggactagccttattttaacttgctatttctagctctaaaacggtcacaatgaggttggggcggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaactggtcagggatggagggtgttagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaactggctgagcggcctcggcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaactggtggccctagggaccaccaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaacctaatgcccactagcctgccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtggcaactttattatacatagttg

SEQ ID NO: 84 - DNA, 22812 bp, synthetic construct
  source 1..22812: mol_type = other DNA; note = Plasmid sequence (for constructing mice#11)
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtggcaactttgtatagaaaagttgaaaaaagcaccgactcggtgccactttttcaagttgataacggactagccttattttaacttgctatttctagctctaaaaccaacaaggcgtcctcctccacggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaaccctcattgactcccatagactagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaaccagggtagcaccctgcgtgagaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaacccctgagtcacccacgacggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaactagggaccaccatggccatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctttagaaggtaggtggagggtggagagggtacccagctctggttgtacctgtcttgttagaagggggtgggaagaagtgcccaacccttttcagaaaataactcttaagatctgaggagaaatggcaaagggaaaagtctccatcctgacgggattggatggttaagatagagaccaggatgggattggatggttaacatagagaccaggatggacaaatagtcagaaacacttataatcatagcattcaggccactgcggggggtggggtggggcaggaactcaagaccagcctgagctaaatacgagatcccatctttaaaataaaagtgctggctgggcagtgttggcacatgcctttaatcccagcacacgggaggcagaggcaggctgatctcttgagtttgaggccagcctggtctacaaagtgagttccaggacagccagggatacacagagaaaccctgtcttgaaaataacaacaaaaaagataaaaatgttggacatggtgtatgttgtaattctaggactggagtagtaatgacagggaacacgggagttcaaggtcactcctgcctacatagaaagtttgaggccatcttaggctacatgagaccctgtctcaagtcaaacaaaacaaaacaaaaggtcagcaactatgtttgtgtggtccagggcctggtgctttagaagacataaaaactgatgagggcagatgcccaatagattagaaagccagtgtgttcacccaggaggctggctctaagaaaggatgcaaagaaagggtctggctcctgccgaggcctgggctaaggctggggggggggggcaaccctgggccttgcaccttgagagtatcatcttagcacattatgagggaaagccccaacctcattgtgacccggtcactctcaggactttctgggtcagactggcaggctagtgggcattaggggtagtgggctgggggcctaagggccctggtggccctagggaccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtggcaactttattatacatagttgtaactttaaataattggcattatttaaagttatccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtaaatataaaatttttaagtgtataatgtgttaaactactgattctaattgtttgtgtattttaggtgtctatggtcgagattatgcaggaagggacatggaagaagcctgaaacactgatgcggtatctcagaaggggcggagtgtccgtgggagccaattctcgactgatggatagctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcttcagtaactttaaataattggcattatttaaagttacaattgaagaaatttaaatgaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttgttaattaattgattcgaggctgctaacaaatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggattaattaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtccgtacgggcgcgccgcggccgc

SEQ ID NO: 85 - DNA, 8474 bp, synthetic construct
  source 1..8474: mol_type = other DNA; note = The KI insert sequence of plasmid (for constructing mice#11)
caactttgtatagaaaagttgaaaaaagcaccgactcggtgccactttttcaagttgataacggactagccttattttaacttgctatttctagctctaaaaccaacaaggcgtcctcctccacggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaaccctcattgactcccatagactagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaaccagggtagcaccctgcgtgagaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaacccctgagtcacccacgacggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaactagggaccaccatggccatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctttagaaggtaggtggagggtggagagggtacccagctctggttgtacctgtcttgttagaagggggtgggaagaagtgcccaacccttttcagaaaataactcttaagatctgaggagaaatggcaaagggaaaagtctccatcctgacgggattggatggttaagatagagaccaggatgggattggatggttaacatagagaccaggatggacaaatagtcagaaacacttataatcatagcattcaggccactgcggggggtggggtggggcaggaactcaagaccagcctgagctaaatacgagatcccatctttaaaataaaagtgctggctgggcagtgttggcacatgcctttaatcccagcacacgggaggcagaggcaggctgatctcttgagtttgaggccagcctggtctacaaagtgagttccaggacagccagggatacacagagaaaccctgtcttgaaaataacaacaaaaaagataaaaatgttggacatggtgtatgttgtaattctaggactggagtagtaatgacagggaacacgggagttcaaggtcactcctgcctacatagaaagtttgaggccatcttaggctacatgagaccctgtctcaagtcaaacaaaacaaaacaaaaggtcagcaactatgtttgtgtggtccagggcctggtgctttagaagacataaaaactgatgagggcagatgcccaatagattagaaagccagtgtgttcacccaggaggctggctctaagaaaggatgcaaagaaagggtctggctcctgccgaggcctgggctaaggctggggggggggggcaaccctgggccttgcaccttgagagtatcatcttagcacattatgagggaaagccccaacctcattgtgacccggtcactctcaggactttctgggtcagactggcaggctagtgggcattaggggtagtgggctgggggcctaagggccctggtggccctagggaccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtggcaactttattatacatagttg

SEQ ID NO: 86 - DNA, 22809 bp, synthetic construct
  source 1..22809: mol_type = other DNA; note = Plasmid sequence (for constructing mice#12)
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtggcaactttgtatagaaaagttgaaaaaagcaccgactcggtgccactttttcaagttgataacggactagccttattttaacttgctatttctagctctaaaacggtcacaatgaggttggggcggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaactggtcagggatggagggtgttagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaactggctgagcggcctcggcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaactggtggccctagggaccaccaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaacctaatgcccactagcctgccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctttagaaggtaggtggagggtggagagggtacccagctctggttgtacctgtcttgttagaagggggtgggaagaagtgcccaacccttttcagaaaataactcttaagatctgaggagaaatggcaaagggaaaagtctccatcctgacgggattggatggttaagatagagaccaggatgggattggatggttaacatagagaccaggatggacaaatagtcagaaacacttataatcatagcattcaggccactgcggggggtggggtggggcaggaactcaagaccagcctgagctaaatacgagatcccatctttaaaataaaagtgctggctgggcagtgttggcacatgcctttaatcccagcacacgggaggcagaggcaggctgatctcttgagtttgaggccagcctggtctacaaagtgagttccaggacagccagggatacacagagaaaccctgtcttgaaaataacaacaaaaaagataaaaatgttggacatggtgtatgttgtaattctaggactggagtagtaatgacagggaacacgggagttcaaggtcactcctgcctacatagaaagtttgaggccatcttaggctacatgagaccctgtctcaagtcaaacaaaacaaaacaaaaggtcagcaactatgtttgtgtggtccagggcctggtgctttagaagacataaaaactgatgagggcagatgcccaatagattagaaagccagtgtgttcacccaggaggctggctctaagaaaggatgcaaagaaagggtctggctcctgccgaggcctgggctaaggctggggggggggggcaaccctgggccttgcaccttgagagtatcatcttagcacattatgagggaaagccccaacctcattgtgacccggtcactctcaggactttctgggtcagactggcaggctagtgggcattaggggtagtgggctgggggcctaagggccctggtggccctagggaccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtggcaactttattatacatagttgtaactttaaataattggcattatttaaagttatccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtaaatataaaatttttaagtgtataatgtgttaaactactgattctaattgtttgtgtattttaggtgtctatggtcgagattatgcaggaagggacatggaagaagcctgaaacactgatgcggtatctcagaaggggcggagtgtccgtgggagccaattctcgactgatggatagctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcttcagtaactttaaataattggcattatttaaagttacaattgaagaaatttaaatgaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttgttaattaattgattcgaggctgctaacaaatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggattaattaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtccgtacgggcgcgccgcggccgc

SEQ ID NO: 87 - DNA, 8471 bp, synthetic construct
  source 1..8471: mol_type = other DNA; note = The KI insert sequence of plasmid (for constructing mice#12)
caactttgtatagaaaagttgaaaaaagcaccgactcggtgccactttttcaagttgataacggactagccttattttaacttgctatttctagctctaaaacggtcacaatgaggttggggcggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaactggtcagggatggagggtgttagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaactggctgagcggcctcggcctgaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaactggtggccctagggaccaccaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaacctaatgcccactagcctgccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctttagaaggtaggtggagggtggagagggtacccagctctggttgtacctgtcttgttagaagggggtgggaagaagtgcccaacccttttcagaaaataactcttaagatctgaggagaaatggcaaagggaaaagtctccatcctgacgggattggatggttaagatagagaccaggatgggattggatggttaacatagagaccaggatggacaaatagtcagaaacacttataatcatagcattcaggccactgcggggggtggggtggggcaggaactcaagaccagcctgagctaaatacgagatcccatctttaaaataaaagtgctggctgggcagtgttggcacatgcctttaatcccagcacacgggaggcagaggcaggctgatctcttgagtttgaggccagcctggtctacaaagtgagttccaggacagccagggatacacagagaaaccctgtcttgaaaataacaacaaaaaagataaaaatgttggacatggtgtatgttgtaattctaggactggagtagtaatgacagggaacacgggagttcaaggtcactcctgcctacatagaaagtttgaggccatcttaggctacatgagaccctgtctcaagtcaaacaaaacaaaacaaaaggtcagcaactatgtttgtgtggtccagggcctggtgctttagaagacataaaaactgatgagggcagatgcccaatagattagaaagccagtgtgttcacccaggaggctggctctaagaaaggatgcaaagaaagggtctggctcctgccgaggcctgggctaaggctggggggggggggcaaccctgggccttgcaccttgagagtatcatcttagcacattatgagggaaagccccaacctcattgtgacccggtcactctcaggactttctgggtcagactggcaggctagtgggcattaggggtagtgggctgggggcctaagggccctggtggccctagggaccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtggcaactttattatacatagttg

SEQ ID NO: 88 - DNA, 16082 bp, synthetic construct
  source 1..16082: mol_type = other DNA; note = Plasmid sequence (for constructing mice#19)
gttctcatgagcctggcctgaatatttattgttgttatcttataaaattgagcagtctgtcatctttccccaaaacatgaactaaaatttgctttatagaacttacaaaaatgctttttttgtaaaatactctttgaatattcctttgtatgatacaaatgcccaatttctatttttgttactaaatgtcctaatataaatattaagtgaatgttaagcaggtttttttagctctctaattttttaaagaaattgccataatgtttcacaaatttttctgattatgaaaaaaaatacatgtgtatttaagaataaatcagtattctgcatgctgcaggttcttgtgaacagacctaaaaataaataaaattctatcaaacattgagctcaaaatagaccttttgtgactaaacagagagacagacagactatacaacttttaatttttttctctgggagaaacctcaaaatatatctgaattctgagctgggtgttgacagtcccagtacttggaaagtagaagaagggttgtgagtttgaaaccagcatgagctatgcaatgcaattccatctcaaaaacaatggcaagtgaattctgcaaagaacgtaaacttatgtaattttaatttttttcaaagtcaaaccacttgtgacgaaagcacctaataaaacgttaactgaaaggaagaactctgtatgaattcacagatatgatgaaatttagttatgatttggccaatattccttgaaaactgccagagcgtagacaaccaggtcttgtagtctattcggtttagaggattttttgggatgggggcaggagctctctatgtaggtcaccttggcctttaactcgtgattatccctgcctttgtacccgagtgctggcattagagatgtgtgacaccatgtgcagcttgatgctttagttcttacaacaccgtgccagggactgctgcttcttagatggggcttaaaagttgggaatgtcatgtaagattgagtagtctaattcttcctggcttatttctctcaataagtttttttaaaaagatttttatttattattatagataagtacactgttggctgtcttcagacacaccagaagggggcatcagatctcattccagatgattgtgagccaccgtgaggttgctgggatttgaactcaggaccttcggaagagcagtcagtgttcttacccgctgagccatctctccagcccctctcattaagttttgattaatagattaacttatgatcttttaagcttttcttgtagtttagtttttgttttgcttttagtgtaaagagggctggagatgagcttggttgctggagtgcttgcccggtatgcatgaaaccctgggtttgacacctagcatcgcataaactgggtgtgatatactcatgctcataatctaaaagaaggagaagcttcagaagttgaaggtcaactgcatagtgagttttaggccagtttaggatccatgacacccccccgccccccatatctcactataattcactgtcaaatggcttttatgatttggagtttggggatacttttaaagacagggtcttggtagctcctagatggcttgggattcacttagtaacccttgctggcctaaaattcaggaacttctacctcatcttcccaagtactgggttagtagggatgtccagcatatttagcttgctttaatgattttgctgtacttttttctgtgtgtacaggtgcatgtatatttataaaactaacataatatattttacccccaaagtaccataaccctaattaatgctattaaatgttttgtatgctgggctctgggcatactaatataaccaatattatacaatgcaaaacaattttggaatatcttctgtaaaattatcttattagtaagttgtaaccatctcagacttctaagtttgagtttgtaaaggatcagttttttttggtgacctacacacacacacacacacacacacacacacacacacacacacacacacacaggggggtggattctgagaatttagagaatttagatggttaataccagtttttatttttattctgtgtgatatttatttattaagatttatttatttattacatgtaggtacactgtatctgtcttcagacacaccagaagagggcatcagatctcgttacagatggttgtgagtcaccatgtggttgctgggatttgaactcaggacctctggaagaacagtcagtgctcttaaccgttgagccatctcaccagccccgtgatattttatttaacttactagaatatactgtgttaaaacatattttaggggattggggagatggcccattctgtaaagtgtctgccatgtcatcttcaggacctgcgtttgtaaccccagcatggagaagacagagacagggagacccctgggcttcctggcaagctagactagcaaatttggttaactaggttcaatgagagactcagtctcaaaacataagaagtgaattttttagacccaatttcaatctctgaactccatacgtatgcacatgtacctgcatataaatatatatacacacccataaacacatgcacacacaaaaaatggttcaaaagttcttaaaatgtataagagtagaaagtgtagagccgggagtggtggcgcacgcctttaatcccagcacttgggagcagaggcaggtggatttctgagttcgaggccagcctggtctacagagtgagttccaggacagccagagctatacagagaaatcctgcctcaaaaaaccaaaaataaagtgtatatactaatctttcatgcctaaaatatttatttttgccacatttttcatacctctttaatatttttctttttctctttgtgcttcaaaggaaattttgtgtcatgttttacttttcattcttgactctattcagcagtaagacgcagcatggctttcttagagaagtataatctattacaaaacggtaacatttaactgctctacataccctcttgtagctaaattcctctgttaaactaagacttattatctaaacgtaattaaatgttgatatcttataatgctccactttgaaacagctgtctttcttttgcagattagcgatgatgaaccaggttatgacctagatttgttttgtatacctaatcattatgccgaggacaactttgtatagaaaagttgacccagctttcttgtacaaagtgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacatcaacgatctgatccgggagatcaagaacaaggactacaccgtgaagctgagcggcaccgatagcaacagcatcacccagctgattatccgcgtgaacaacgacggcaacgagtacgtgatctccgagagcgagaacgagagcatcgtggagaagttcatcagcgccttcaagaacggttggaaccaggagtacgaggacgaggaggagttctacaacgacatgcagaccatcaccctgaagagcgagctgaactaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaactttattatacatagttgtaactttaaataattggcattatttaaagttatccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtaaatataaaatttttaagtgtataatgtgttaaactactgattctaattgtttgtgtattttaggtgtctatggtcgagattatgcaggaagggacatggaagaagcctgaaacactgatgcggtatctcagaaggggcggagtgtccgtgggagccaattctcgactgatggatagctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcttcagtaactttaaataattggcattatttaaagttacaattgaagaaatttaaattttggaaaaagtgtttattcctcatggactgattatggacaggttagtaagacctcgattgaagttatttttagagggtgtgtgtgtggtgtgtgtgtgtgtgtgtgtgtgtgtgtgtgtgtgtgtgtgtgtgtgtgtgtaagagagagaggagaggcatgtgtgatatatatgtgaagtgtattgcatgtacatggaatccagagaaggacattaggtgtcctactctgttgctctcttattcccttgaatcagtgtctcttgctgaacctggggcatacatactgatttggctaggctgcctggctagcaagacccagggatctgcttgtctctgccccccagtgctgacgttacaagcaggtgcaaccacatctggtttttatatgggtactgggggatctgaacttagggcctgatgcgtgcccaagtgctctcacccgccaagccacctctccagccaaagggttttttttcttgtattaattctaacttagcaactgttgagtagttgatgtttgaatataagctaagccaacttattagaataaatgttttctattttttacttgtatgtgtgagtgcctgcatgtatgtatgtgaagcatatgtattccaggatgtcagaattgtgatggattccctggaactggagttacagaccattgtgaaccactgtgtatgggtgctgggaactgaactttaggtcctctgcaaaaacagtaaggatcttaactgataaactatctttctagccctaaggctttctacttttaaagttcgtttctctttctctctctctctctctctctctctctctctctctctccctctccctccctcctcctcctcctcctctttcccccctctcgcctcctctttctctctcccctccctctctctctctcacacacacacacagacagagagaggagagagaaaggaagccttactaattaattacctagttttagacataactagtgggaatccatagtaagtaaatcaacattcttgaagagaggttagaagaattggctctcgttgtccatcgaggctgtataagattattcttctgttttctcagcttaccatacatattttctacgtagacaataccaattcaaatacactgtacgtaaagtatatgcgcacacacacacacacacacacacacacacacacacacacaagttacatattgtcatatagtactaaaatttatggactttttcatattaatacatctagagctatcatgttttttatcttttaaaaattactttacggggctacagagatgtctcagcagttaggagcactgactgctcttccagaggtcctgagttcaattcccagcaaccacatggtggctcacaaccatctgtaatgggatctgatccactcttatggtgtgtctgaagacagtgacagtgtactcatatacatgaaataaatctttaaaaaattactttaccatcagggtatatatatatatatatatatgtgtgtgtgtgtgtgtgtgtgtatgaagtatttgctatttgagagggtctcactgtttagctcagtctggcttggaacgttctgtatagcccagactggcctcaactcagcagtccccctgaaaggctctcatatgtggagattacaggtatgtgctactgtgcccagccctacatgttactttgtttttgaaatagggtatcaaatagcttagactggcttctaagtcagtgtgtagctaaggatggtctgagcttctgaacctgctacttcaactcctggtgtgccatcatggccagtttgcacagttgtgagagtagaactcaggacctcctatgtgttactagacatgttctctaccagctgagctacatcccaattcgcctacatttacttttattttttaaaagatttatttatgtatacagcactctgcctgcatgtacatctgcatgccagaagagggcaccagatctcatcatacatagttgtgagccaccatgtggttgctggggattgaactcagggcctctggaagagcagtcagtgctcttaacctctgagccatctctgtagccccctacatttattttaaagattataggctattctattcatagatgtgccataactgatttaaccagatcttcattattttgtacttagtagagtatattagagggaatgaatcttaaatacacattttgacacttgctcagctttagacaagtaaaaatcactggtcaaggaaatggtgcttagtggatgcttctgaactgtcaaattgtcctccaacaagttacatcattttatacttccaacgttatgtgagagttcatttttctttaggctcttctagtattttatatttgagaaataaaatttaaccaaaatttgccaatctagtaagcttgtttttatttgtatctttaaattttatgatagatgttatagtgtactctcctctcccctcccctcccctctcctcctcagcaggatcttgttatgtagccctaagggatctaatacttactctgtagtccaggctgtccttcagctcatgagacacaccatcatgcttggcatgtttcttgttctgaaaggtatctggttttaattttggttgagctggtgtgttggtgtatatctgtaatctcagcacaggcaagtagagacaaggattatgattacagtccagcctgggctatttagagggattctctttaatctttcctcatgccccaaaatcttacctttggtatatgaaaaatagtctccacttctgcaaaatattgctttatgaagtaagaattcccttcatagagacaaggaatgtgtcctgtaaaagtttaatgtgtaagaagtatttgttataaaagataaatattcagaatcttctttttaattcctgattttatttctataggactgaaagacttgctcgagatgtcatgaaggagatgggaggccatcacattgtggccctctgtgtgctcaaggggggctataagttctttgctgacctgctggattacattaaagcactgaatagaaatagtgatagatccattcctatgactgtagattttatcagactgaagagctactgtttaattaattgattcgaggctgctaacaaatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggattaattaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtccgtacgggcgcgccgcggccgc

SEQ ID NO: 89 - DNA, 2624 bp, synthetic construct
  source 1..2624: mol_type = other DNA; note = The KI insert sequence of plasmid (for constructing mice#19)
caactttgtatagaaaagttgacccagctttcttgtacaaagtgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacatcaacgatctgatccgggagatcaagaacaaggactacaccgtgaagctgagcggcaccgatagcaacagcatcacccagctgattatccgcgtgaacaacgacggcaacgagtacgtgatctccgagagcgagaacgagagcatcgtggagaagttcatcagcgccttcaagaacggttggaaccaggagtacgaggacgaggaggagttctacaacgacatgcagaccatcaccctgaagagcgagctgaactaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaactttattatacatagttg

SEQ ID NO: 90 - DNA, 23 bp, synthetic construct
  source 1..23: mol_type = other DNA; note = 3'arm PCR primer F1
cctgaatgaactgcaagacgagg

SEQ ID NO: 91 - DNA, 23 bp, synthetic construct
  source 1..23: mol_type = other DNA; note = 3'arm PCR primer R1
gagttccaggccagtctatggtc

SEQ ID NO: 92 - DNA, 21 bp, synthetic construct
  source 1..21: mol_type = other DNA; note = KI PCR primer F2 for plasmid 1
ggttgttgcagcctcttgtga

SEQ ID NO: 93 - DNA, 19 bp, synthetic construct
  source 1..19: mol_type = other DNA; note = KI PCR primer R2 for plasmid 1
cgccccaacctcattgtga

SEQ ID NO: 94 - DNA, 21 bp, synthetic construct
  source 1..21: mol_type = other DNA; note = Neo-L PCR primer F3 for plasmid 1
cttagacatgctcccagccga

SEQ ID NO: 95 - DNA, 20 bp, synthetic construct
  source 1..20: mol_type = other DNA; note = Neo-L PCR primer R3 for plasmid 1
ggcccacaacagcaccattg

SEQ ID NO: 96 - DNA, 22 bp, synthetic construct
  source 1..22: mol_type = other DNA; note = Wildtype PCR primer F4
ggttgttgcagcctcttgtgat

SEQ ID NO: 97 - DNA, 25 bp, synthetic construct
  source 1..25: mol_type = other DNA; note = Wildtype PCR primer R4
tggtaaaactaccctgtttcctttc

SEQ ID NO: 98 - DNA, 27 bp, synthetic construct
  source 1..27: mol_type = other DNA; note = 5'-arm probe F
cagggataacctttgtgagggactgtt

SEQ ID NO: 99 - DNA, 24 bp, synthetic construct
  source 1..24: mol_type = other DNA; note = 5'-arm probe R
aagttcccagaatcggtgctcctt

SEQ ID NO: 100 - DNA, 26 bp, synthetic construct
  source 1..26: mol_type = other DNA; note = 3'-arm probe F
gaaatagagggaggagaggggaaatg

SEQ ID NO: 101 - DNA, 30 bp, synthetic construct
  source 1..30: mol_type = other DNA; note = 3'-arm probe R
gggaatacttttcattaacctgtacttgct

SEQ ID NO: 102 - DNA, 21 bp, synthetic construct
  source 1..21: mol_type = other DNA; note = PCR1 primer TKIE100-F1
ggttgttgcagcctcttgtga

SEQ ID NO: 103 - DNA, 23 bp, synthetic construct
  source 1..23: mol_type = other DNA; note = PCR2 primer TKIE100-R2
aactgtttcatttcccctctcct

SEQ ID NO: 104 - DNA, 20 bp, synthetic construct
  source 1..20: mol_type = other DNA; note = Neo-F primer
tcatctcaccttgctcctgc

SEQ ID NO: 105 - DNA, 20 bp, synthetic construct
  source 1..20: mol_type = other DNA; note = Neo-R primer:
aaggcgatagaaggcgatgc

SEQ ID NO: 106 - DNA, 468 bp, synthetic construct
  source 1..468: mol_type = other DNA; note = Prob for Neo
tcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgcctt

SEQ ID NO: 107 - DNA, 9530 bp, synthetic construct
  source 1..9530: mol_type = other DNA; note = Intron 1, exon 2, intron 2, exon 3 of the uty gene (NCBI Reference Sequence: NM_009484.3) of mus musculus
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtgggaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttgtgagtttgagactagcctggtctacagagaaaatttcaggatagtcagagtaacagagaaaaagttcaaaaacaaaaaaacagaagagtcatatgacagtgttttaaatctgcaagtaagcaagtattttcaggtgatatgctgatactttattgtaacggcagaattcagagggctgaaaaagtcgttttttgagtcagacctgtggcaagtgccaagtgcttttacttcctgagttatcaagtggctaataataataataacaataataaaaataataaaatataattattaaaataataaagttgttgttgttcttcttccttctttctcctccttcttcttccttcttcttcctccctccttcctctcttcttcttcttccttcttccttcttcttcttccttcttcctcctccctccttcctccttccttctccctccttcctcacttcttcttcttcttctattattattttggggtagagtctcactttgtagttagtgtagaccatagactggcctggaactcagtacaggaaagaaagctgtatgccaggggctggagagatggctcagtggttaagagcactgactgctcttctgaaggtcctgagtttataccagcaaccacattgtggcttacaagcatctgtaatgggatctaatgccctcttatggtgtgcctgaagacagctacagtgtacttagatataataataaataaatctttaggccagaacaaagtgagcagaaatcctgaattcaaattcccagcaaccacatgaagggaagagactctggttgcacacattgactttcttttttttttttttttttttttggtttttcaagacagggtttctctgtgtagtcctagctgtcctggaactcactctgtagaccaggatagcctcaaactcagagatccatctgctctgcctcccaagtgctgggattaaaggcatgcaccaccactgcctggctaaaatggtcctaaaatgtctgtaaagacatttccaattattttagtctcaggctccaattagtcagggtctgattattaataagtgtgaagtctggttttatttttcaccccactcccttttgtttttcctgtatatttgtgtatattcagtttatagtgttagactatttgatttatgataaaaagagtaggtattaaacaattgaaatttgtggtgcttaaatgctgaaaacagtttctgataaaattatatctcaagttttcgtaaataggcaagtacttagtagttgaagattgttcattagccttagaaaccacaaaagatgcctggaaccgtataccttctttaagatgacacctgcaaaacagttccactgactaaagatcaagcatttaaatgtagccaaaagcactattctctctctttttttttttcccccagagctgaggacggaacccagggccttgtgcttgctaggcaagccctctaccactgagctaaatccccatcccccaaaagcactattctcattcagattactatagtgtgtgtgtgtgtgggggggggttgttttgttttggttgtgtttctttttttctttttttttttttttttgctatgtagcttagttggctcttactgtctaaggtcattcatttttgggtctctaaatgctactgttagagaaataaagtaaaatttaaaaatatgataaactgtaaaatatgttgtttaaaaattgaatttacataatctcagtttggatttaccatatatttaaaaaattaccttgagtatatggattctgaatttaatatatttagtaatgcactcctttgttactttattgagagtttttgtttctttttaaaatacattgaaaaataatatgtatttggtgtgtttcagtatatgtgtatgtctttatgtgggtgtgtgcaatagaggagactcagtggttaagagacaactcttccagagatcctgagttcaattccctgcaaccacatggtggctcacaaccatctgtaatgggatctgatgctttcttctggtgtgtctgaagtaagctacagtgtactcatatacatgaaataaatcttaaaaatacagagaatatacttatgaattatgatttccatagtcataaactgtatgtaatcttggagatagagttttactaattctaatgtagagttttattttaattgtaaatgtttctatgtttcatttttaggcaattcacttctatgagtctttaatagtaaaagctgaaggaaaagtggaatcagatttcttttgtcaattaggtcacttcaatctcttactggaagattattcaaaag

SEQ ID NO: 108 - DNA, 5336 bp, synthetic construct
  source 1..5336: mol_type = other DNA; note = Intron 2 of the uty gene (NCBI Reference Sequence: NM_009484.3) of mus musculus
gtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtgggaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttgtgagtttgagactagcctggtctacagagaaaatttcaggatagtcagagtaacagagaaaaagttcaaaaacaaaaaaacagaagagtcatatgacagtgttttaaatctgcaagtaagcaagtattttcaggtgatatgctgatactttattgtaacggcagaattcagagggctgaaaaagtcgttttttgagtcagacctgtggcaagtgccaagtgcttttacttcctgagttatcaagtggctaataataataataacaataataaaaataataaaatataattattaaaataataaagttgttgttgttcttcttccttctttctcctccttcttcttccttcttcttcctccctccttcctctcttcttcttcttccttcttccttcttcttcttccttcttcctcctccctccttcctccttccttctccctccttcctcacttcttcttcttcttctattattattttggggtagagtctcactttgtagttagtgtagaccatagactggcctggaactcagtacaggaaagaaagctgtatgccaggggctggagagatggctcagtggttaagagcactgactgctcttctgaaggtcctgagtttataccagcaaccacattgtggcttacaagcatctgtaatgggatctaatgccctcttatggtgtgcctgaagacagctacagtgtacttagatataataataaataaatctttaggccagaacaaagtgagcagaaatcctgaattcaaattcccagcaaccacatgaagggaagagactctggttgcacacattgactttcttttttttttttttttttttttggtttttcaagacagggtttctctgtgtagtcctagctgtcctggaactcactctgtagaccaggatagcctcaaactcagagatccatctgctctgcctcccaagtgctgggattaaaggcatgcaccaccactgcctggctaaaatggtcctaaaatgtctgtaaagacatttccaattattttagtctcaggctccaattagtcagggtctgattattaataagtgtgaagtctggttttatttttcaccccactcccttttgtttttcctgtatatttgtgtatattcagtttatagtgttagactatttgatttatgataaaaagagtaggtattaaacaattgaaatttgtggtgcttaaatgctgaaaacagtttctgataaaattatatctcaagttttcgtaaataggcaagtacttagtagttgaagattgttcattagccttagaaaccacaaaagatgcctggaaccgtataccttctttaagatgacacctgcaaaacagttccactgactaaagatcaagcatttaaatgtagccaaaagcactattctctctctttttttttttcccccagagctgaggacggaacccagggccttgtgcttgctaggcaagccctctaccactgagctaaatccccatcccccaaaagcactattctcattcagattactatagtgtgtgtgtgtgtgggggggggttgttttgttttggttgtgtttctttttttctttttttttttttttttgctatgtagcttagttggctcttactgtctaaggtcattcatttttgggtctctaaatgctactgttagagaaataaagtaaaatttaaaaatatgataaactgtaaaatatgttgtttaaaaattgaatttacataatctcagtttggatttaccatatatttaaaaaattaccttgagtatatggattctgaatttaatatatttagtaatgcactcctttgttactttattgagagtttttgtttctttttaaaatacattgaaaaataatatgtatttggtgtgtttcagtatatgtgtatgtctttatgtgggtgtgtgcaatagaggagactcagtggttaagagacaactcttccagagatcctgagttcaattccctgcaaccacatggtggctcacaaccatctgtaatgggatctgatgctttcttctggtgtgtctgaagtaagctacagtgtactcatatacatgaaataaatcttaaaaatacagagaatatacttatgaattatgatttccatagtcataaactgtatgtaatcttggagatagagttttactaattctaatgtagagttttattttaattgtaaatgtttctatgtttcatttttag

SEQ ID NO: 109 - DNA, 4646 bp, synthetic construct; identical to SEQ ID NO: 52
  source 1..4646: mol_type = other DNA; note = Homology left arm sequence of the VB201210-1273huu (for constructing mice#20)

SEQ ID NO: 110 - DNA, 11441 bp, synthetic construct
  source 1..11441: mol_type = other DNA; note = The KI sequence of the VB201210-1273huu (for constructing mice#20)
caactttgtatagaaaagttgaaaaaagcaccgactcggtgccactttttcaagttgataacggactagccttattttaacttgctatttctagctctaaaaccaacaaggcgtcctcctccacggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaaccctcattgactcccatagactagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaaccagggtagcaccctgcgtgagaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaacccctgagtcacccacgacggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaactagggaccaccatggccatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacatcaacgatctgatccgggagatcaagaacaaggactacaccgtgaagctgagcggcaccgatagcaacagcatcacccagctgattatccgcgtgaacaacgacggcaacgagtacgtgatctccgagagcgagaacgagagcatcgtggagaagttcatcagcgccttcaagaacggttggaaccaggagtacgaggacgaggaggagttctacaacgacatgcagaccatcaccctgaagagcgagctgaactaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaactttattatacatagttg

SEQ ID NO: 111 - DNA, 2550 bp, synthetic construct; identical to SEQ ID NO: 55
  source 1..2550: mol_type = other DNA; note = The sequence of the right homology arm for the VB201210-1273huu (for constructing mice#20)

SEQ ID NO: 112 - DNA, 20 bp, synthetic construct
  source 1..20: mol_type = other DNA; note = The Crem gRNA of KI insert sequence of plasmid
caacaaggcgtcctcctcca

SEQ ID NO: 113 - DNA, 20 bp, synthetic construct
  source 1..20: mol_type = other DNA; note = The sun5 gRNA of KI insert sequence of plasmid
cctcattgactcccatagac

SEQ ID NO: 114 - DNA, 20 bp, synthetic construct
  source 1..20: mol_type = other DNA; note = The sun4 gRNA of KI insert sequence of plasmid (for constructing mice#20)
cagggtagcaccctgcgtga

SEQ ID NO: 115 - DNA, 20 bp, synthetic construct
  source 1..20: mol_type = other DNA; note = The Jhdm2a gRNA of KI insert sequence of plasmid (for constructing mice#20)
ccctgagtcacccacgacgg

SEQ ID NO: 116 - DNA, 20 bp, synthetic construct
  source 1..20: mol_type = other DNA; note = The spem1 gRNA of KI insert sequence of plasmid (for constructing mice#20)
tagggaccaccatggccatg

SEQ ID NO: 117 - DNA, 324 bp, synthetic construct
  source 1..324: mol_type = other DNA; note = The destabilizing domain (DD) of KI insert sequence of plasmid (for constructing mice#20)
atgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaa

SEQ ID NO: 118 - DNA, 270 bp, synthetic construct
  source 1..270: mol_type = other DNA; note = The AcrIIA4 of KI insert sequence of plasmid (for constructing mice#20)
ggatctggaaacatcaacgatctgatccgggagatcaagaacaaggactacaccgtgaagctgagcggcaccgatagcaacagcatcacccagctgattatccgcgtgaacaacgacggcaacgagtacgtgatctccgagagcgagaacgagagcatcgtggagaagttcatcagcgccttcaagaacggttggaaccaggagtacgaggacgaggaggagttctacaacgacatgcagaccatcaccctgaagagcgagctgaactaa

SEQ ID NO: 119 - DNA, 25779 bp, synthetic construct
  source 1..25779: mol_type = other DNA; note = VB201210-1273huu (for constructing mice#20)
accaccgcccagctagctttcttaaattaagcagtttctgtcagtgttagggatttttacctgcaattccatcatatttgaatactctgtgactgagactgttagggtccagtaatagccatacaaacccctcagaagccaatctcagtcatgtatcaatggtttattgaacacattcctagactgattagtcaggagtggggatcgaaaacttcaataaggcattcctcagaaccaatttttatatgggaaaaacaaggacttggtacatttgcaatgtttataacatttgcaacatttatgctatctagacaaagcagtgctgtctggcctttaagctgataggctatgtatagagacaaggagacaaggagacctccaaagttcacaaggctcagtgaaaaagtataacaactttttttcagggcaagaaacaataggtcaagttaaatttatgagctacaaaatgaagattgagggaatatggcagaattttaatatttaacagagaaatgtccttaaacccccaaagagtaatttaaaagggtttttattatcaaacacctttttctgttttaagattctctatgtagagtaggttgcattggacaattatctttctggctttacttactgagttctgaaatttgaaaggattcaccattatttctttgtggcaaatatttgtcaaccccagaactcagcagatacaatagtggaagaaataagtcagctcttgctccctcccttacctctattctctagctcttttgctcccctgctccccctctctccccatccccttccttgctctctctatgtggtcatggctgccttctgcttctctactatctcacactctttgcctttctacaataaatgccttaaaaccaaaaaataaaaaataagaaaaaagtcagcagaggagaatgaaaggatggactactactacttcagaactgtttaaacctgagaattttgaggagaaagattgtttagtttcaacccttacaagggactgaggtacatattttacatattaaagcagtcctggcctccagattctcctagcatccctcagtcccaacctggcattctgtctccaaacctaaacttttcaggtcagaggctgggctacctttcccccagaagcttctccctatataatccagacatttggacaatgactcccccccccatgtccccctccttttccttttcctcctcttcttcctcttcctccccaccttgtcttatcctctttctctctctctgtctatctctctctctctcttctcccttctctgtgtacatgagctttctctctccatggtaacttccctggcctcagttattggggccagtgaacttgcccaagagcagcttcccaataagcctgcctatatataaaatattaaagacctgttccacaatatccaactaaagcaagctttatttaacactgactaggaagttcaacactgactacattcatgccaggattccaagagaaatggctttgaattaaaatcagtcatgtgatcatgcaaagaaacctctacaaatctatgaactttccatatttgtccaattaggggtaagcatacatcctcactgacttcctgtctgcatatttctcacctgtgtgtgatcaagcatatcctgagcacttggggcaacaaaccttgctattgaactaaaacacaacctccagtattctgcagttatttgtccttaggtaagtgtagcatacacattaactttaaagaattcccagagctgattttttgtttgtttgtttgtttgtttgtttgtttgtttttgagagaaagtttttctgtgtagccctggctgtcctggaactcactctgtagaccaggcttcctcaaactcagaaatccacctgcctctgcctcccaagtgctgggattaaaggcgtgcaccaccacggctggctctgactttttttttttttaagagttactttatttatatgagtacactgttactgttttcagacaccccagaagagggcattggatcccattacagatggttgtgaaccaccatgttggtggtgggaattgaactcaggacctctggaagagcagtcagtgctcttaaccataccatgcagttagtatctgagagtaaagtagcagaagctatacaccgtctgagaatcaattcttgtagtgtaggcttctggagtcccagaaactgcttttcaagttaaattgggtgcagggagggagggggagaataggtctaattgtttctgaaaccttttggcagaagggtaaaagataagtcagattgcctgttttttgcatcttatggcttccaggtctgcaaagtgttttagaagcttcctagaattttgttatggaagcttttgaaaagacttagacatgttttgtggacctggtttgggggtagggaatttacttgaagagggattaggtagtgtttgctagccagtcttcaatacaattcaagtttaaggtcatcttagatttataaatatttgtgttggagacttgtgatactggtttctgagatggggaactaggtttccttgtgaaggtccaggcccagtaagtaatggtaatacaagtcaattcaaaactcctgagaacagcaaaattggaacttgtcttattatattttttattattgtattatatataaatgaatgctcctcttacaaagtgttggatcatatttacaaattttagctacaacaaccaaaaggtcaaaagaaatggtattgttctcaaatggttactgcatcttcaaaatgcaatgagggattcccccgtccttcccccccccccgccccctcacgtcactccttgcccccgcagggataacctttgtgagggactgttcattcactatgatctgttgtttaaggacaaagtcatgtgacttttataaagtttttttggtttgatgtggttgagtggatgggtaattaatgagtttagctatggtggtgacaaaaaaacacgacccagttactcattgaggatgatttcaattttagctgaatacattttgttttcccaacgtgtgtgtcattacttcaagatcttaattttaaagtttgctagaattagtattaaggagcaccgattctgggaacttaacagaaggtgttctgtaaggcaagaggaaaatttttttttttaactatttggtctctaattggctttcatgtgccctaaaaaccgatgtttgatgtattttttcttagtgctctttgttccttgtgagatcagtatagcaattaaatgaaagggtctggatatctagtgtggaataagaaagagttacaaagcatacctaaaactaatttcactgtggtcggtaggtagagctcttaggctgacatcacgagctagcgaagtagcaagtttagctgcactggtgatgacgcaagtcaacaaaagtcacgtgggataaaacggttgcttccattggaaagcctggctttagggagaggttatggacgttacgtgcggttcacactgacggtattataacaactttatgcggatgctagcgaagtttgatattctaagaacttcgcttaggttctgcaacaatgagttcttcttgcgttcaccatctgcgtggtaatgtgaccagatagcctctgccgctttctcctttgccagtttccatgaaatcttacggattatctctcactactgctgccctaggtaatgaggaaaagaaaatggcggcggaaaaggctagaggcgagggcgaagaggggtccttcagtctcacagtcgaagagaagaaggcgctttgtggattagataggtacgcactgctaatacttacaggttgtctttctctctccgggttcaaaagactctccaggaggcctgactgcatcttgcttgcggtgacgactcgctttcgcctagcactgttaacaatgttttgggagattttttactgatgtctttctgtgatcattaacaagagccatgtatttcttccacagcagcttcttcgggttcttgacccgatgcaaagatggcgccaagatgaagacgctgttgaacaaggtcagcaactgtttaaacataattacttatatttttctcctaagcttttcactgttccaagcctggagcctctccctactctgaaagtgctcattatagcttttagggatctttgaaactttttttttccttccccgtttctcctttcttggcttactttcctgatagtttgaggccctgcctcagcatacagcccagagcactgcttattttgcagtttttctgaggtcagctatatcctccagttactcatctcaagttatgagaagacgcttggttgttgcagcctcttgtgatatacacacgttttattattttcccataagggacaagttataggtaaggagataaagagtttccgtacttaaaaagaattttttacgatgcagtagtaatactatgcagaaataatactgaattccagtttggaatgttaatttttttttttttttacttccttgtagtttattttccagagagtgagagagcacacggcatttcctttgtgaaactttgcattgtcatttagttattggctgctggtatcaatcaagatggtggcaactttgtatagaaaagttgaaaaaagcaccgactcggtgccactttttcaagttgataacggactagccttattttaacttgctatttctagctctaaaaccaacaaggcgtcctcctccacggtgttggtaacttgccagaagatatataaaggcaagaaatagaaatactttacacttacggcaagtatgtcatggtccattttaaaacataattttaaaattacaaactacccaagaaattattactttctgcatcaatattttgtactaatatctttatgtttatagccaaattaattctaattatctctctaacattcttgcatcgtatatgcaaatatgaaggactcatgggaaataggccctccgtaatgggataggttacgttggtgtagatgggcgcatcgtaaccgtgcatctgccagtttgaggggacgacgacagtatcggcctcaggaagatcgcacaaaaaagcaccgactcggtgccactttttcaagttgataacggactagccttattttaacttgctatttctagctctaaaaccctcattgactcccatagactagatctgtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgttcgaatttccagccagctttccggcaccgcttctggtgccggaaaccaggcaaagcgccattcgccattcaggctgcgcaactgttgggaagggcgatcggtgcgggaaaaaagcaccgactcggtgccactttttcaagttgataacggactagccttattttaacttgctatttctagctctaaaaccagggtagcaccctgcgtgagaggtacccaggcggcgcacaagctatataaacctgaaggaaatctcaactttacacttaggtcaagttacttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcagttaaaaggcacaaatgtaggaataacaaatgttgtaggttttcctctaggtgcatgcacaacagctacaaaagcattcgaagtttctgaagcaattaaagaaaaaagcaccgactcggtgccactttttcaagttgataacggactagccttattttaacttgctatttctagctctaaaacccctgagtcacccacgacggcaaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatcggccggcccctcttcgctattacgccagctggcgaaagggggatgtgctgcaaggcgattaagttgggtaacgccagggttttcccagtcacgacgttgtaaaacgacaaaaaagcaccgactcggtgccactttttcaagttgataacggactagccttattttaacttgctatttctagctctaaaactagggaccaccatggccatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctccaagtttgtacaaaaaagcaggctgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgaacaattcccagggaagagtgaccttcgaggatgtcactgtgaacttcacccagggggagtggcagcggctgaatcccgaacagagaaacttgtacagggatgtgatgctggagaattacagcaaccttgtctctgtgggacaaggggaaaccaccaaacccgatgtgatcttgaggttggaacaaggaaaggagccatggttggaggaagaggaagtgctgggaagtggccgtgcagaaaaaaatggggacattggagggcagatttggaagccaaaggatgtgaaagagagtctctaccctaccttcctgtacaaggtggtgggtggatctggaggttctcccaagaagaagcgcaaggtgggacgcgtctgcaggatatcaagcttgcggtaccgcgggcccgggatcgccaccatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggacggctagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaacccagctttcttgtacaaagtgggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgggagtgcaagtggaaaccatctccccaggagacgggcgcaccttccccaagcgcggccagacctgtgtggtgcactacaccgggatgcttgaagatggaaagaaagtcgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaacctgaaggatctggaaacatcaacgatctgatccgggagatcaagaacaaggactacaccgtgaagctgagcggcaccgatagcaacagcatcacccagctgattatccgcgtgaacaacgacggcaacgagtacgtgatctccgagagcgagaacgagagcatcgtggagaagttcatcagcgccttcaagaacggttggaaccaggagtacgaggacgaggaggagttctacaacgacatgcagaccatcaccctgaagagcgagctgaactaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaactttattatacatagttgtaactttaaataattggcattatttaaagttatccatgggccaggcaaatatcccttaccagcctcacagagacctcccccaccccccgcaaccctagagttcttttactagtgagggacaagtggacaatggtgctgttgtgggccccaccctgtgtcccctgtgcccacagtggtcactctgcttggcaggcaggtgttgcaggctggctgctccaggccctggcaggaggtactgaaggacctggtaggctcagatgccctggatgccaaggcactgctggagtacttccaaccggtcagccagtggctggaagagcagaatcagcggaatggcgaagtcctaggctggccagagaatcagtggcgtccaccgttacccgacaactatccagagggcattggtaaagctctgagtgagggtggactgggaccaagagaagtcctggcctctggcctctggcttctgggtcaaagcctcagcatcctggtcactttgctgccagctgagccccagtgtcctttgcttcagtgccaagccacccctgggctcatcctcagggccctaagcagaaatgggtatgtctttctctcagggtcctagagacagtgtgcccaagcctgagggcccttggggtcaggctggctggcacattgctctatgaggtcacactgcaggcttggctcttattggccggtgatgggagcttcagggctctgctttcctgcggccatgcctaagaagaagaggaaggtttctgagctgattattagtggttcatctggtggattcctgcgaaacatcggcaaagagtatcaggaggccgctgaaaacttcatgaggtttatgaatgaccagggggcgtacgctcctaacactttgagggatttgaggttggtctttcatagctgggccagatggtgccatgctcggcagcttgcatggtttccaattagtcctgaaatggcacgcgaatactttcttcagttgcacgatgcagacctggcctccactaccatcgacaagcactatgctatgcttaatatgcttctgtcccactgcggactgccacccttgtccgacgacaagtcagtgagtcttgccatgagaagaattagaagagaagccgcaaccgaaaagggtgagaggacaggacaggcaatccccctgcgctgggacgacctgaagctgctggatgtgctgctcagcaggagcgagcggctggtcgacctgcgcaacagggctttcctgttcgtagcctataacaccctcatgagaatgtctgaaatatcacgcatcagggttggggacttggatcagacaggagacacagtgaccctgcacatcagtcacactaagacaatcaccacagctgcgggccttgacaaagtgctctcccggcgaaccacagcagtgctcaatgactggctggacgtcagtgggcttagagaacatccagacgctgtgctcttcccacctatacaccggtcaaacaaagcccgcattactaccacgcccctgaccgcccctgccatggagaagattttcagtgatgcctgggtgctgctgaacaaacgggacgccacccccaataaagggaggtataggacctggaccggccattccgccagggtgggtgccgcaatagacatggccgagaaacaggtaaatataaaatttttaagtgtataatgtgttaaactactgattctaattgtttgtgtattttaggtgtctatggtcgagattatgcaggaagggacatggaagaagcctgaaacactgatgcggtatctcagaaggggcggagtgtccgtgggagccaattctcgactgatggatagctaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagaattcttcagtaactttaaataattggcattatttaaagttacaattgaagaaatttaaatgaaatagagggaggagaggggaaatgaaacagttttagaaaggaaacagggtagttttaccaagaaaatattaaacctagcaagtacaggttaatgaaaagtattccccattacttaaaaggagaattatcaagtctttttaaacctttaaaaaaagattcttagccgggcggggtggcacatgcctttaatcccagcacttgggaggcagaggcaggcagatttctgagtttgaggccagtctggtctacagagtgagttccaggacagccagggctacacagagataccctgtctcaaaaaaaaaaaaaaaattcttaaaggtaacatatttagatacttgtaaaccatagcaaaaaaagcacagatctgtgacatacatacttgttaccattatatagtttccgttctaatgaaaatcagtgattataaacttgataatttcataccaccaatgaagtattaatagtattcttttttggtttttggagacagggtttctcagtatagctctggttgctatacagttaatagtgttcttaacatatatgtgtatatataaatttatgtatgtatgtatatatatatatacatatatatatatgcatattatgcacacataatttttggtttggtttggttgtgagcctagcctttaaggactgagtccaagatttaaacactcttcaggtgatatgtatagaaacatatttctgtttactagtaggttgctaggtttctggtgtacagtgggtaattttaaatttgatgttaacctccacactcttttaagatttcatggtaaaagcttaaagaaatgattttgtctagtttttatttttattttttaattttttttatttttttggtttttggagacagggtttctctgtatagctctggctgtcctggaactcactttgtagaccaggctggcctcaaactcagaaatctgcctgtctctccctcctgagtgctgggattaaaggcatgcaccaccacgcctgacgtggtttttaaagatataattatttaactatatcttagcatttcttttttttttttagagtagtaattcttatattataaaaaagacttggtgttgaatgtatttttggagtgactacaacaactatgtgtgatcctgtttgtcctagaactcacagacagaaatatcactttctctgctttctcattgctcattgctaagattaaagccctgttttcctaaaactggcttcttttaaatttaataattttgtatatattcatgtctggaagttataggacaaattgtggaagtcagttttcttcttctatcatgtcagggctggaggcaagtccttttactagctaagccacttccttgaactttcagtttatgtgtatagtgtttctactgcttgtaggtctattccacttgagtgtctggttctactaaggagtttagaagagacagacctatggaactggaattagtgaaggttgtgagcttcactaatcaaaccagtgttctcttgcagagctctttagtttatttaagtagcatatatttttcaggtgctttgtaatatcaatttatatgttagtaaactgttttcaaatattgaaataacaggtattttttaactaattgcaacagcattttaaaaatgagaagttaaaaacaaaatatgagaaactatcaagtgccactgttcttagaacatttatgagttgtgcctttattaaacattgagtaatatgtatttttgttttagatcagtctcagcctttctaatgcagtttctcatgttgtgatgacccccaaccataaaattatttttgttgttattcataactaattttggtgctgttatgaatcattatgtaaatatctgaatagtttattagtgaccaaaaaggtattttgacccataggttgagaaccattattttagacactgtcttagtctatattttgttgcccagaatagctttaaatttgaagaagtactcatctctcagcctccaagtattgagatttcaggcatgagccactactctttaacagttattaaaatgtattacaattataatttaaaatgacaagtattaaaaactgaaatatagggctggtgagatggctcagtgggtaagagcacccgactgctcttccgaaggtccagagttcaaatcccagcaaccacatggtggctcacaaccatccataacgagatctgactccctcttctggagtgtctgaagacagctacagtgtacttacatataataaataaatctttaaaaaaaaaaaaacaaaaaaaaacctgaaatatataaagggcaattgatggatggtggtggaatatccctttatcctagcactgtgcagaggcagttgggtctcttataagtttgaggccagcctgatctacatagcaagtttcaggacagcaagattagacagagaaacctgcctcaaaaaaggacaatttgtttttaagcctgacagtggtggcatacacctttaaacccagcacttgggggacataggcagatagatctttgttaattaattgattcgaggctgctaacaaatcgagcagtgtggttttcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcttggtggcgtgaaactcccgcacctcttcggccagcgccttgtagaagcgcgtgccatggatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcagatcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggattaattaaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagatccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtccgtacgggcgcgccgcggccgc

SEQ ID NO: 120 - DNA, 3200 bp, synthetic construct
  source 1..3200: mol_type = other DNA; note = Hprt (hypoxanthine phosphoribosyltransferase 1) of mus musculus homology left arm)
gttctcatgagcctggcctgaatatttattgttgttatcttataaaattgagcagtctgtcatctttccccaaaacatgaactaaaatttgctttatagaacttacaaaaatgctttttttgtaaaatactctttgaatattcctttgtatgatacaaatgcccaatttctatttttgttactaaatgtcctaatataaatattaagtgaatgttaagcaggtttttttagctctctaattttttaaagaaattgccataatgtttcacaaatttttctgattatgaaaaaaaatacatgtgtatttaagaataaatcagtattctgcatgctgcaggttcttgtgaacagacctaaaaataaataaaattctatcaaacattgagctcaaaatagaccttttgtgactaaacagagagacagacagactatacaacttttaatttttttctctgggagaaacctcaaaatatatctgaattctgagctgggtgttgacagtcccagtacttggaaagtagaagaagggttgtgagtttgaaaccagcatgagctatgcaatgcaattccatctcaaaaacaatggcaagtgaattctgcaaagaacgtaaacttatgtaattttaatttttttcaaagtcaaaccacttgtgacgaaagcacctaataaaacgttaactgaaaggaagaactctgtatgaattcacagatatgatgaaatttagttatgatttggccaatattccttgaaaactgccagagcgtagacaaccaggtcttgtagtctattcggtttagaggattttttgggatgggggcaggagctctctatgtaggtcaccttggcctttaactcgtgattatccctgcctttgtacccgagtgctggcattagagatgtgtgacaccatgtgcagcttgatgctttagttcttacaacaccgtgccagggactgctgcttcttagatggggcttaaaagttgggaatgtcatgtaagattgagtagtctaattcttcctggcttatttctctcaataagtttttttaaaaagatttttatttattattatagataagtacactgttggctgtcttcagacacaccagaagggggcatcagatctcattccagatgattgtgagccaccgtgaggttgctgggatttgaactcaggaccttcggaagagcagtcagtgttcttacccgctgagccatctctccagcccctctcattaagttttgattaatagattaacttatgatcttttaagcttttcttgtagtttagtttttgttttgcttttagtgtaaagagggctggagatgagcttggttgctggagtgcttgcccggtatgcatgaaaccctgggtttgacacctagcatcgcataaactgggtgtgatatactcatgctcataatctaaaagaaggagaagcttcagaagttgaaggtcaactgcatagtgagttttaggccagtttaggatccatgacacccccccgccccccatatctcactataattcactgtcaaatggcttttatgatttggagtttggggatacttttaaagacagggtcttggtagctcctagatggcttgggattcacttagtaacccttgctggcctaaaattcaggaacttctacctcatcttcccaagtactgggttagtagggatgtccagcatatttagcttgctttaatgattttgctgtacttttttctgtgtgtacaggtgcatgtatatttataaaactaacataatatattttacccccaaagtaccataaccctaattaatgctattaaatgttttgtatgctgggctctgggcatactaatataaccaatattatacaatgcaaaacaattttggaatatcttctgtaaaattatcttattagtaagttgtaaccatctcagacttctaagtttgagtttgtaaaggatcagttttttttggtgacctacacacacacacacacacacacacacacacacacacacacacacacacacaggggggtggattctgagaatttagagaatttagatggttaataccagtttttatttttattctgtgtgatatttatttattaagatttatttatttattacatgtaggtacactgtatctgtcttcagacacaccagaagagggcatcagatctcgttacagatggttgtgagtcaccatgtggttgctgggatttgaactcaggacctctggaagaacagtcagtgctcttaaccgttgagccatctcaccagccccgtgatattttatttaacttactagaatatactgtgttaaaacatattttaggggattggggagatggcccattctgtaaagtgtctgccatgtcatcttcaggacctgcgtttgtaaccccagcatggagaagacagagacagggagacccctgggcttcctggcaagctagactagcaaatttggttaactaggttcaatgagagactcagtctcaaaacataagaagtgaattttttagacccaatttcaatctctgaactccatacgtatgcacatgtacctgcatataaatatatatacacacccataaacacatgcacacacaaaaaatggttcaaaagttcttaaaatgtataagagtagaaagtgtagagccgggagtggtggcgcacgcctttaatcccagcacttgggagcagaggcaggtggatttctgagttcgaggccagcctggtctacagagtgagttccaggacagccagagctatacagagaaatcctgcctcaaaaaaccaaaaataaagtgtatatactaatctttcatgcctaaaatatttatttttgccacatttttcatacctctttaatatttttctttttctctttgtgcttcaaaggaaattttgtgtcatgttttacttttcattcttgactctattcagcagtaagacgcagcatggctttcttagagaagtataatctattacaaaacggtaacatttaactgctctacataccctcttgtagctaaattcctctgttaaactaagacttattatctaaacgtaattaaatgttgatatcttataatgctccactttgaaacagctgtctttcttttgcagattagcgatgatgaaccaggttatgacctagatttgttttgtatacctaatcattatgccgagga

SEQ ID NO: 121 - DNA, 2500 bp, synthetic construct
  source 1..2500: mol_type = other DNA; note = Hprt (hypoxanthine phosphoribosyltransferase 1) of mus musculus homology right arm)
tttggaaaaagtgtttattcctcatggactgattatggacaggttagtaagacctcgattgaagttatttttagagggtgtgtgtgtggtgtgtgtgtgtgtgtgtgtgtgtgtgtgtgtgtgtgtgtgtgtgtgtgtgtaagagagagaggagaggcatgtgtgatatatatgtgaagtgtattgcatgtacatggaatccagagaaggacattaggtgtcctactctgttgctctcttattcccttgaatcagtgtctcttgctgaacctggggcatacatactgatttggctaggctgcctggctagcaagacccagggatctgcttgtctctgccccccagtgctgacgttacaagcaggtgcaaccacatctggtttttatatgggtactgggggatctgaacttagggcctgatgcgtgcccaagtgctctcacccgccaagccacctctccagccaaagggttttttttcttgtattaattctaacttagcaactgttgagtagttgatgtttgaatataagctaagccaacttattagaataaatgttttctattttttacttgtatgtgtgagtgcctgcatgtatgtatgtgaagcatatgtattccaggatgtcagaattgtgatggattccctggaactggagttacagaccattgtgaaccactgtgtatgggtgctgggaactgaactttaggtcctctgcaaaaacagtaaggatcttaactgataaactatctttctagccctaaggctttctacttttaaagttcgtttctctttctctctctctctctctctctctctctctctctctctccctctccctccctcctcctcctcctcctctttcccccctctcgcctcctctttctctctcccctccctctctctctctcacacacacacacagacagagagaggagagagaaaggaagccttactaattaattacctagttttagacataactagtgggaatccatagtaagtaaatcaacattcttgaagagaggttagaagaattggctctcgttgtccatcgaggctgtataagattattcttctgttttctcagcttaccatacatattttctacgtagacaataccaattcaaatacactgtacgtaaagtatatgcgcacacacacacacacacacacacacacacacacacacacaagttacatattgtcatatagtactaaaatttatggactttttcatattaatacatctagagctatcatgttttttatcttttaaaaattactttacggggctacagagatgtctcagcagttaggagcactgactgctcttccagaggtcctgagttcaattcccagcaaccacatggtggctcacaaccatctgtaatgggatctgatccactcttatggtgtgtctgaagacagtgacagtgtactcatatacatgaaataaatctttaaaaaattactttaccatcagggtatatatatatatatatatatgtgtgtgtgtgtgtgtgtgtgtatgaagtatttgctatttgagagggtctcactgtttagctcagtctggcttggaacgttctgtatagcccagactggcctcaactcagcagtccccctgaaaggctctcatatgtggagattacaggtatgtgctactgtgcccagccctacatgttactttgtttttgaaatagggtatcaaatagcttagactggcttctaagtcagtgtgtagctaaggatggtctgagcttctgaacctgctacttcaactcctggtgtgccatcatggccagtttgcacagttgtgagagtagaactcaggacctcctatgtgttactagacatgttctctaccagctgagctacatcccaattcgcctacatttacttttattttttaaaagatttatttatgtatacagcactctgcctgcatgtacatctgcatgccagaagagggcaccagatctcatcatacatagttgtgagccaccatgtggttgctggggattgaactcagggcctctggaagagcagtcagtgctcttaacctctgagccatctctgtagccccctacatttattttaaagattataggctattctattcatagatgtgccataactgatttaaccagatcttcattattttgtacttagtagagtatattagagggaatgaatcttaaatacacattttgacacttgctcagctttagacaagtaaaaatcactggtcaaggaaatggtgcttagtggatgcttctgaactgtcaaattgtcctccaacaagttacatcattttatacttccaacgttatgtgagagttcatttttctttaggctcttctagtattttatatttgagaaataaaatttaaccaaaatttgccaatctagtaagcttgtttttatttgtatctttaaattttatgatagatgttatagtgtactctcctctcccctcccctcccctctcctcctcagc

SEQ ID NO: 122 - DNA, 6316 bp, synthetic construct
  source 1..6316: mol_type = other DNA; note = exon 2, intron 2, exon 3 of the Hprt (hypoxanthine phosphoribosyltransferase 1) of mus musculus ID: MGI:96217, NCBI Gene: 15452, ENSMUSG00000025630
gttctcatgagcctggcctgaatatttattgttgttatcttataaaattgagcagtctgtcatctttccccaaaacatgaactaaaatttgctttatagaacttacaaaaatgctttttttgtaaaatactctttgaatattcctttgtatgatacaaatgcccaatttctatttttgttactaaatgtcctaatataaatattaagtgaatgttaagcaggtttttttagctctctaattttttaaagaaattgccataatgtttcacaaatttttctgattatgaaaaaaaatacatgtgtatttaagaataaatcagtattctgcatgctgcaggttcttgtgaacagacctaaaaataaataaaattctatcaaacattgagctcaaaatagaccttttgtgactaaacagagagacagacagactatacaacttttaatttttttctctgggagaaacctcaaaatatatctgaattctgagctgggtgttgacagtcccagtacttggaaagtagaagaagggttgtgagtttgaaaccagcatgagctatgcaatgcaattccatctcaaaaacaatggcaagtgaattctgcaaagaacgtaaacttatgtaattttaatttttttcaaagtcaaaccacttgtgacgaaagcacctaataaaacgttaactgaaaggaagaactctgtatgaattcacagatatgatgaaatttagttatgatttggccaatattccttgaaaactgccagagcgtagacaaccaggtcttgtagtctattcggtttagaggattttttgggatgggggcaggagctctctatgtaggtcaccttggcctttaactcgtgattatccctgcctttgtacccgagtgctggcattagagatgtgtgacaccatgtgcagcttgatgctttagttcttacaacaccgtgccagggactgctgcttcttagatggggcttaaaagttgggaatgtcatgtaagattgagtagtctaattcttcctggcttatttctctcaataagtttttttaaaaagatttttatttattattatagataagtacactgttggctgtcttcagacacaccagaagggggcatcagatctcattccagatgattgtgagccaccgtgaggttgctgggatttgaactcaggaccttcggaagagcagtcagtgttcttacccgctgagccatctctccagcccctctcattaagttttgattaatagattaacttatgatcttttaagcttttcttgtagtttagtttttgttttgcttttagtgtaaagagggctggagatgagcttggttgctggagtgcttgcccggtatgcatgaaaccctgggtttgacacctagcatcgcataaactgggtgtgatatactcatgctcataatctaaaagaaggagaagcttcagaagttgaaggtcaactgcatagtgagttttaggccagtttaggatccatgacacccccccgccccccatatctcactataattcactgtcaaatggcttttatgatttggagtttggggatacttttaaagacagggtcttggtagctcctagatggcttgggattcacttagtaacccttgctggcctaaaattcaggaacttctacctcatcttcccaagtactgggttagtagggatgtccagcatatttagcttgctttaatgattttgctgtacttttttctgtgtgtacaggtgcatgtatatttataaaactaacataatatattttacccccaaagtaccataaccctaattaatgctattaaatgttttgtatgctgggctctgggcatactaatataaccaatattatacaatgcaaaacaattttggaatatcttctgtaaaattatcttattagtaagttgtaaccatctcagacttctaagtttgagtttgtaaaggatcagttttttttggtgacctacacacacacacacacacacacacacacacacacacacacacacacacacaggggggtggattctgagaatttagagaatttagatggttaataccagtttttatttttattctgtgtgatatttatttattaagatttatttatttattacatgtaggtacactgtatctgtcttcagacacaccagaagagggcatcagatctcgttacagatggttgtgagtcaccatgtggttgctgggatttgaactcaggacctctggaagaacagtcagtgctcttaaccgttgagccatctcaccagccccgtgatattttatttaacttactagaatatactgtgttaaaacatattttaggggattggggagatggcccattctgtaaagtgtctgccatgtcatcttcaggacctgcgtttgtaaccccagcatggagaagacagagacagggagacccctgggcttcctggcaagctagactagcaaatttggttaactaggttcaatgagagactcagtctcaaaacataagaagtgaattttttagacccaatttcaatctctgaactccatacgtatgcacatgtacctgcatataaatatatatacacacccataaacacatgcacacacaaaaaatggttcaaaagttcttaaaatgtataagagtagaaagtgtagagccgggagtggtggcgcacgcctttaatcccagcacttgggagcagaggcaggtggatttctgagttcgaggccagcctggtctacagagtgagttccaggacagccagagctatacagagaaatcctgcctcaaaaaaccaaaaataaagtgtatatactaatctttcatgcctaaaatatttatttttgccacatttttcatacctctttaatatttttctttttctctttgtgcttcaaaggaaattttgtgtcatgttttacttttcattcttgactctattcagcagtaagacgcagcatggctttcttagagaagtataatctattacaaaacggtaacatttaactgctctacataccctcttgtagctaaattcctctgttaaactaagacttattatctaaacgtaattaaatgttgatatcttataatgctccactttgaaacagctgtctttcttttgcagattagcgatgatgaaccaggttatgacctagatttgttttgtatacctaatcattatgccgaggatttggaaaaagtgtttattcctcatggactgattatggacaggttagtaagacctcgattgaagttatttttagagggtgtgtgtgtggtgtgtgtgtgtgtgtgtgtgtgtgtgtgtgtgtgtgtgtgtgtgtgtgtgtaagagagagaggagaggcatgtgtgatatatatgtgaagtgtattgcatgtacatggaatccagagaaggacattaggtgtcctactctgttgctctcttattcccttgaatcagtgtctcttgctgaacctggggcatacatactgatttggctaggctgcctggctagcaagacccagggatctgcttgtctctgccccccagtgctgacgttacaagcaggtgcaaccacatctggtttttatatgggtactgggggatctgaacttagggcctgatgcgtgcccaagtgctctcacccgccaagccacctctccagccaaagggttttttttcttgtattaattctaacttagcaactgttgagtagttgatgtttgaatataagctaagccaacttattagaataaatgttttctattttttacttgtatgtgtgagtgcctgcatgtatgtatgtgaagcatatgtattccaggatgtcagaattgtgatggattccctggaactggagttacagaccattgtgaaccactgtgtatgggtgctgggaactgaactttaggtcctctgcaaaaacagtaaggatcttaactgataaactatctttctagccctaaggctttctacttttaaagttcgtttctctttctctctctctctctctctctctctctctctctctctccctctccctccctcctcctcctcctcctctttcccccctctcgcctcctctttctctctcccctccctctctctctctcacacacacacacagacagagagaggagagagaaaggaagccttactaattaattacctagttttagacataactagtgggaatccatagtaagtaaatcaacattcttgaagagaggttagaagaattggctctcgttgtccatcgaggctgtataagattattcttctgttttctcagcttaccatacatattttctacgtagacaataccaattcaaatacactgtacgtaaagtatatgcgcacacacacacacacacacacacacacacacacacacacaagttacatattgtcatatagtactaaaatttatggactttttcatattaatacatctagagctatcatgttttttatcttttaaaaattactttacggggctacagagatgtctcagcagttaggagcactgactgctcttccagaggtcctgagttcaattcccagcaaccacatggtggctcacaaccatctgtaatgggatctgatccactcttatggtgtgtctgaagacagtgacagtgtactcatatacatgaaataaatctttaaaaaattactttaccatcagggtatatatatatatatatatatgtgtgtgtgtgtgtgtgtgtgtatgaagtatttgctatttgagagggtctcactgtttagctcagtctggcttggaacgttctgtatagcccagactggcctcaactcagcagtccccctgaaaggctctcatatgtggagattacaggtatgtgctactgtgcccagccctacatgttactttgtttttgaaatagggtatcaaatagcttagactggcttctaagtcagtgtgtagctaaggatggtctgagcttctgaacctgctacttcaactcctggtgtgccatcatggccagtttgcacagttgtgagagtagaactcaggacctcctatgtgttactagacatgttctctaccagctgagctacatcccaattcgcctacatttacttttattttttaaaagatttatttatgtatacagcactctgcctgcatgtacatctgcatgccagaagagggcaccagatctcatcatacatagttgtgagccaccatgtggttgctggggattgaactcagggcctctggaagagcagtcagtgctcttaacctctgagccatctctgtagccccctacatttattttaaagattataggctattctattcatagatgtgccataactgatttaaccagatcttcattattttgtacttagtagagtatattagagggaatgaatcttaaatacacattttgacacttgctcagctttagacaagtaaaaatcactggtcaaggaaatggtgcttagtggatgcttctgaactgtcaaattgtcctccaacaagttacatcattttatacttccaacgttatgtgagagttcatttttctttaggctcttctagtattttatatttgagaaataaaatttaaccaaaatttgccaatctagtaagcttgtttttatttgtatctttaaattttatgatagatgttatagtgtactctcctctcccctcccctcccctctcctcctcagcaggatcttgttatgtagccctaagggatctaatacttactctgtagtccaggctgtccttcagctcatgagacacaccatcatgcttggcatgtttcttgttctgaaaggtatctggttttaattttggttgagctggtgtgttggtgtatatctgtaatctcagcacaggcaagtagagacaaggattatgattacagtccagcctgggctatttagagggattctctttaatctttcctcatgccccaaaatcttacctttggtatatgaaaaatagtctccacttctgcaaaatattgctttatgaagtaagaattcccttcatagagacaaggaatgtgtcctgtaaaagtttaatgtgtaagaagtatttgttataaaagataaatattcagaatcttctttttaattcctgattttatttctataggactgaaagacttgctcgagatgtcatgaaggagatgggaggccatcacattgtggccctctgtgtgctcaaggggggctataagttctttgctgacctgctggattacattaaagcactgaatagaaatagtgatagatccattcctatgactgtagattttatcagactgaagagctactgt

(19) United States
(12) Patent Application Publication
(10) Pub. No.: US 2026/0053121 A1
(43) Pub. Date: Feb. 26, 2026

(54) GENETICALLY ENGINEERED EUKARYOTIC ORGANISMS, SYSTEMS AND METHODS FOR IN VIVO BIASING SEX-RATIO OF POPULATIONS

(71) Applicant: RAMOT AT TEL-AVIV UNIVERSITY LTD., Tel Aviv (IL)

(72) Inventors: Ehud QIMRON, Tel Aviv (IL); Ido YOSEF, Tel Aviv (IL); Mordechay GERLIC, Tel Aviv (IL); Ariel MUNITZ, Tel Aviv (IL)

(73) Assignee: RAMOT AT TEL-AVIV UNIVERSITY LTD., Tel Aviv (IL)

(21) Appl. No.: 19/105,448

(22) PCT Filed: Aug. 21, 2023
(86) PCT No.: PCT/IL2023/050881

§ 371 Date: Feb. 21, 2025

Related U.S. Application Data
(60) Provisional application No. 63/373,135, filed on Aug. 22, 2022.

(51) Int. Cl. A01K 67/0275; C12N 9/22; C12N 15/11; C12N 15/82; C12N 15/90

(52) U.S. Cl. CPC A01K 67/0275; C12N 9/226; C12N 15/11; C12N 15/8213; C12N 15/8287; C12N 15/902; C12N 15/907; A01K 2217/072; A01K 2217/075; A01K 2227/101; A01K 2227/105; A01K 2227/30; C12N 2310/20

(57) ABSTRACT

The present disclosure provides genetically modified heterogametic organism that comprises a genetically modified first sex chromosome comprising at least one exogenous nucleic acid sequence integrated therein. In more specific embodiments, the exogenous nucleic acid sequence comprises (i) at least one nucleic acid sequence encoding at least one nucleic acids modifier component, and further (ii) at least one nucleic acid sequence encoding and/or forming at least one target recognition element for the at least one nucleic acids modifier component of (i). The present disclosure further comprises systems comprising the discussed genetically modified heterogametic organism, methods and uses thereof in biasing sex-ratio of populations.

### FIELD OF THE INVENTION

The present invention relates to genetically engineered heterogametic eukaryotic organisms, systems and methods thereof, for in vivo biasing the sex-ratio of populations of said organisms, and/or for selection and/or production of sexed population of gamete cells of these organisms.

### BACKGROUND ART

References considered to be relevant as background to the presently disclosed subject matter are listed below:
[1] Ido Yosef, Liat Edry-Botzer, Rea Globus, Inbar Shlomovitz, Ariel Munitz, Motti Gerlic &Udi Qimron A genetic system for biasing the sex ratio in mice, EMBO reports e48269 (2019).
[2] WO 2019/135230 (PCT/IL2019/050015).
[3] WO 02/38748.
[4] US 2014/0359796.
[5] Windbichler, N., P. A. Papathanos, and A. Crisanti. “Targeting the X Chromosome during Spermatogenesis Induces Y Chromosome Transmission Ratio Distortion and Early Dominant Embryo Lethality in Anopheles Gambiae.” PLOS Genetics 4 (12): el000291 (2008).
[6] Galizi, R., L. A. Doyle, M. Menichelli, F. Bernardini, A. Deredec, A. Burt, B. L. Stoddard, N. Windbichler, and A. Crisanti. “A Synthetic Sex Ratio Distortion System for the Control of the Human Malaria Mosquito.” Nature Communications 5:3977 (2014).
[7] Galizi, R., A. Hanunond, K. Kyrou. C. Taxiarchi, F. Bernardini, S. M. O'Loughlin, P. A. Papathanos, T. Nolan, N. Windbichler, and A. Crisanti. “A CRISPR-Cas9 Sex-Ratio Distortion System for Genetic Control.” Scientific Reports 6:31139 (2016).
[8] WO 2018/204722.
[9] Douglas, C. et al. Nature Communications 12, 6926, doi: 10.1038/s41467-021-27227-2 (2021). US 2020/0399661.

Acknowledgement of the above references herein is not to be inferred as meaning that these are in any way relevant to the patentability of the presently disclosed subject matter.

### BACKGROUND OF THE INVENTION

Biasing the sex ratio of populations of different organisms, including plants, insects, crustacean, and fish has been demonstrated by genetic and non-genetic approaches.

The most widely used technology for selection of a desired sex is sorted sexed, in which the desired sexed semen is obtained by flow cytometer-based sorting procedures. Sorted sexed semen undergoes about twenty-one processes for providing about 90% probability for the desired sex progeny. Multiple processing steps of the semen reduce its fertility, are laborious, expensive and result only in a partial desired outcome. There is therefore need for effective technology that provides 100% sex selection of desired progeny.

Biasing the sex ratio of mammalian populations has not been demonstrated genetically until recently. The inventors have recently provided the first proof of concept for a such a genetic system in mammals by crossing two genetically-engineered mouse lines [1, 2]. The maternal line encoded a functional Cas9 protein on an autosomal chromosome, whereas the paternal line encoded guide RNAs on the Y chromosome targeting vital mouse genes. After fertilization, the presence of both the Y-encoded guide RNAs from the paternal sperm and the Cas9 protein from the maternal egg target the vital genes in males. It was shown that genes are specifically targeted in males and that this breeding consequently self-destructs solely the males in utero [1, 2]. These results paved the way for a genetic system that allows biased sex production of livestock. Despite the pioneering nature of this approach, there are three main shortcomings that need to be addressed, particularly, for mammalian organisms: (a) the abortion of males in utero reduces the litter size by half, (b) a single genetic line may be superb over maintaining two lines; and (c) the resulting females are genetically modified organisms (GMOs) which is a regulatory burden. The principles shown in the inventor's previous work were also followed successfully with some improvements by the Turner group, which showed complete sex bias for both males and females [9]. In these systems, the fertilized egg continued to develop in the uterus only in the absence of the Y chromosome. The male sperm carried on its Y chromosome or autosomal chromosome CRISPR guides that along with the CRISPR-Cas9 protein on the autosomal or sex chromosomes of the female, resulted in death of the developing embryo. As indicated above, this system required two mouse lines: the female carrying Cas9 and the male carrying CRISPR guides, and vice versa. The litter size of the mating was reduced by approximately half the normal size due to abortion of the male progeny. Moreover, the born progeny were genetically modified organisms (GMO), as they carried the Cas9 gene in their genome. A novel approach that enables to overcome the abovementioned shortcomings, is therefore needed.

WO 02/38748 [3], suggests methods of sex-selection of non-human animals, based on the provision of a genetically modified male comprising on either the X or Y chromosomes thereof (in the HPRT locus or the SRY locus, respectively), a direct or indirect mediator that is active post-meiotically and destroy gametes of the undesired sex. More specifically, the RNAse Barnase, is suggested as a direct mediator, under the control of the post-meiotic regulatory region such as transition protein 1 promoter (TP1). Alternatively, the herpes simplex virus thymidine kinase gene that forms a toxic metabolite in the presence of ganciclovir, is suggested as an indirect inducible mediator, under the control of TP1. However, the mediators used by this system are non-specific and therefore this system may lead to reduction in fertility. Thus, a highly specific and controlled system is needed to prevent any reduction in viability and function of the resulting sperm. US 2014/0359796 [4], discloses genetically modified livestock animal comprising a genetic modification to disrupt a target gene selectively involved in gametogenesis, thereby preventing formation of functional gametes. The disruption of the target gene resulted in sterile animals. More specifically, the genetically engineered male comprises nucleases such as Transcription activator-like effector nucleases (TALEN), or the CRISPR/cas9 system, specifically directed against genes involved in gametogenesis, for example, the Deleted in azoospermia-like (DAZL) gene that is connected with fertility in vertebrates, under the control of a promoter selectively activated in gametogenesis. This publication further suggests selection of a desired sex by directing the nuclease to a target gene necessary for the development of the undesired sex.

This system may be effective for disturbing the gametogenesis and creation of sterile animals, however, due to non-specific off target cleavage of these nucleases, this method may lead to reduction in fertility.

Windbichler et al., [5], disclose methods and systems for sex ratio distortion in Anophieles gambiae by targeting X chromosome during spermatogenesis. This system is based on the transgenic Anophieles mosquitoes that encode a transgenic I-PopI homing endonuclease (fused to GFP) that targets and cleaves ribosomal rDNA repeats that are exclusively located at the centromeric region of chromosome X. The expression of this transgenic endonuclease under the control of the B2 promoter that is exclusively activated in male gonads. However, this method resulted in male sterility, due to cleavage of male X chromosomes. Galizi et al., [6], suggested further improvement of this approach by providing destabilized I-PpoI variants in an attempt to restrict the activity of the nuclease to spermatogenesis. Galizi et al., [7], disclose an additional approach for sex distortion, based on CRISPR-Cas9 that targets ribosomal sequences restricted to the member species of the Anopheles gambiae complex that are exclusive to the female sex chromosome. Expression of Cas9 during spermatogenesis resulted in RNA-guided shredding of the X-chromosome during male meiosis and produced male bias among progeny. These approaches are mainly used for exterminating wild populations of pests and not for maintaining stable animal lines for reproduction. An approach and means ensuring that the desired sex chromosome will not be destroyed, are therefore essential to prevent reduction in fertility or even sterility. Still further, WO 2018/204722 [8], discloses methods for manipulation of populations and sex ratio in populations. This method is based however on insertion of two components into one chromosome of transgenic insects. The first component is a gene expressing an enzyme that modifies and inactivates a target gene essential for viability or fertility of the organism. An example for such component is the CRISPR/Cas9 that targets a specific target essential gene located in another chromosome. The second component is a rescue transgene that includes functional version of the target essential gene that is resistant to modification by the first components. Linking these two components together in one plasmid, ensures survival of an organism that carry the rescue gene, as well as transmission of these transgenes to progenies. Progenies that carry only the modified chromosome targeted by the first component, will not survive. This method however requires the presence of a chromosome that carry both components and also results in viable progenies that carry a modified target gene. Moreover, rescuing a particular target gene may not be sufficient to ensure a viable progeny. As such, this approach, although addressing the need to modify populations, cannot be applied effectively for the purpose of selection of a desired sex. Moreover, for producing both sexes, two transgenic animals are needed.

Furthermore, some of the above approaches rely on physical separation of the X and Y chromosome-encoding gametes. While this physical separation indeed partially occurs, there are still some cytoplasmic bridges between the gametes until an advance stage of maturation. West et al. [10] discloses methods for preventing the transmission of an undesired chromosome, e.g., an undesired sex chromosome, by providing transgenic male non-human animals comprising in the undesired chromosome exogeneous nucleic acid sequence comprising an untranslated region (UTR) that tethers a transcript transcribed from the attached sequence to a cytoskeletal structure of the sperm cells, operably linked to a sequence encoding siRNA directed at a target the Gnat3 and/or Tas1r3 genes. As indicated above, relying on separation alone may not suffice. For example, genes encoded on the Y chromosome may still be present in the X gamete at later stage due to their stability. A controllable degradation domain, which determines the protein half-life, is not present in the above prior inventions, which may be critical for success of these approaches. Therefore, effective and highly specific methods for in vivo production of gamete cells composed of a desired sex with no reduction in fertility, are needed.

### SUMMARY OF THE INVENTION

As indicated herein above, an efficient genetic system that produces non-GMO progeny should constitute a huge advance in the field, as it uses the natural way of reproduction without hurdles, sophisticated technology, hormone usage, or other costly or complex means, and results in the desired outcome. This is in fact a system long waited for in the industry. The present disclosure provides for the first proof of concept in mammals showing non-GMO females produced from genetically modified males. The constructed males produce mostly females that are all non-GMO. The present inventors engineered on the Y chromosome of the mouse, a controllable cassette encoding dCas9 and guides silencing specific gamete-maturation genes. Due to the separation of the gametes, in the desired X gamete the dCas9 and the guides would not be expressed at the critical gamete maturation step. In contrast, in the Y gamete, in which they are encoded, they would be expressed, thus eliminating the gamete. In this approach, the litter size of the progeny is normal because the sperm with the eliminated Y gametes is sufficient to fertilize all the available eggs and, in contrast to prior art systems, no offspring are lost after fertilization in utero. Furthermore, this approach, which shows a significant sex biasing (prior to further optimization with controllable degradation domain to determine the protein half-life), is achievable without the need to control the cytoplasmic bridges between the gametes, in contrast to West et al. [10]. Lastly, the disclosed system is simpler because it is composed of only a single animal male line, compared to two in the prior art systems.

Thus, a first aspect of the present disclosure relates to a genetically engineered eukaryotic heterogametic organism comprising exogenous nucleic acid sequences in one sex chromosome. More specifically, the genetically engineered heterogametic organism of the invention comprises: A first sex chromosome (a), comprising at least one exogenous nucleic acid sequence integrated therein. In more specific embodiments, the exogenous nucleic acid sequence comprises the following sequences:

In a first sequence (i), at least one nucleic acid sequence encoding at least one nucleic acids modifier component. In a second sequence (ii), at least one nucleic acid sequence encoding or forming at least one target recognition element for the at least one nucleic acids modifier component of (i). In some optional embodiments, the exogenous nucleic acid sequence may further comprise a third sequence (iii), at least one nucleic acid sequence encoding at least one inhibitor or repressor for the at least one nucleic acids modifier component of (i).

In some optional embodiments, the genetically engineered heterogametic organism of the invention may comprise exogenous nucleic acid sequences also in the second sex chromosome thereof. According to these optional embodiments, the genetically engineered eukaryotic heterogametic organism further comprises a second sex chromosome (b), comprising at least one of the following sequences: In a fourth sequence (iv), at least one exogenous nucleic acid sequence encoding at least one inhibitor or repressor for the at least one nucleic acids modifier component of (i); and/or as a fifth sequence (v), at least one endogenous or exogenous nucleic acid sequence encoding at least one transcription factor or repressor, that specifically bind at least one transcription regulatory element operably linked to the nucleic acid sequence of at least one of (i), (ii) and (iii).

In some optional embodiments, at least one of the first, second and the optional third, fourth and/or fifth nucleic acid sequences of (i), (ii) and the optional (iii), (iv) and/or (v), respectively, may be operably linked to at least one regulatory element. More specifically, such regulatory element may be at least one of: at least one inducible or repressible regulatory element, at least one transcriptional regulatory element, at least one post-translationally regulatory element and at least one temporally or spatially regulatory element.

A further aspect of the invention relates to a system comprising at least one genetically engineered eukaryotic heterogametic organism and at least one homogametic eukaryotic organism. In more specific embodiments, the heterogametic genetically engineered organism of the disclosed systems is as defined by the present disclosure. In some specific embodiments, the homogametic organism of the disclosed systems may be a wild-type homogametic organism. Alternatively, the homogametic organism of the disclosed systems may be a genetically engineered organism.

It should be noted that optionally, the exogenous nucleic acid sequence/s are operably linked to at least one of: at least one inducible or repressible regulatory element, at least one transcriptional regulatory element, at least one post-translationally regulatory element and/or at least one temporally or spatially regulatory element.

In yet some further aspect, the invention provides at least one progeny or cell produced or prepared by at least one genetically engineered eukaryotic heterogametic organism provided by the present disclosure, specifically, as disclosed above, or any product thereof.

Still further aspect of the invention relates to an in vivo or ex vivo population of gamete cells predominantly composed of one desired sex, or any composition or preparation thereof. It should be noted that in some embodiments, the population of cells is produced by at least one genetically engineered eukaryotic heterogametic organism as defined and provided by the present disclosure, specifically, as disclosed above.

A further aspect of the invention relates to a method for preparing gamete cell population predominantly composed of one desired sex. More specifically, the method comprising the step of obtaining gamete cell population from at least one genetically engineered eukaryotic heterogametic organism as defined by the present disclosure.

In yet another aspect, the present disclosure provides a method for selecting a desired sex of a eukaryotic organism. More specifically, the method comprising the steps of: contacting at least one genetically engineered heterogametic organism or any gamete cells thereof with at least one homogametic eukaryotic organism or any gamete cells thereof, thereby obtaining a progeny predominantly composed of the one desired sex. In some specific embodiments, the homogametic organism of the disclosed methods may be a wild-type homogametic organism. Alternatively, the homogametic organism of the disclosed methods may be a genetically engineered organism. In more specific embodiments, the genetically engineered eukaryotic heterogametic organism is as defined by the invention herein above.

These and other aspects of the invention will become apparent by the hand of the following description.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1B: Constructs design for the GMO and non-GMO strains

FIG. 1A. dCas9 strains. Schematic representation of the cassette introduced to the Y chromosome for producing females only, or the X chromosome for producing males only. dCas9-KRAB-MeCP2 (indicated in the figure as dCas9) or Cas9 is operably linked to a promoter (e.g. constitutive promoter (CAGG), an inducible promoter (Tet-off), a natural promoter of one of the gamete development genes (Spem1/Akap4)) and optionally, to at least one degron. The nucleic acid sequence further comprises a sequence encoding gRNA or shRNA and optionally Tet trans-activator (Table 4, mice #1-16). It should be understood that the present illustrative construct reflects only one possible arrangement of the various elements, and the order of the elements may change (e.g., placement of the sequence encoding gRNA, either upstream the Cas9 promoter, or downstream the degron, or any other appropriate position).

FIG. 1B. Anti-dCas9 strains. Schematic representation of the cassette introduced X and Y chromosomes for crossing with the respective GMO dCas9 strain. The sequence encoding the anti-CRISPR-ACRIIA4 is operably linked to a constitutive promoter and optionally, to at least one degron (Table 5, mice #17-19). It should be understood that the present illustrative construct reflects only one possible arrangement of the various elements, and the order of the elements may change.

FIG. 2. Linearized targeting vector

The figure generally illustrates the linearized targeting vector for insertion into intron 2 of the mouse uty gene, highlighting the key components involved in the gene targeting process. The targeting vector includes an HA left arm sequence, a knock-in (KI) sequence, an SDN (Self-deleting Neo Cassette) to allow the excision of the Neo cassette, a HA right arm sequence and a diphtheria toxin A (DTA) sequence for negative selection.

FIG. 3. Plasmid VB201210-125bpw for strain #2

Figure shows the plasmid VB201210-125bpw used for preparing strain #2. The plasmid comprises the dCas9-KRAB and four gRNAs directed at the Spem1 gene (Spem1-gRNA #1, Spem1-gRNA #2, Spem1-gRNA #3, Spem1-gRNA #4). The insert is flanked by a right and a left homology arms that are specific for the target location (the uty gene).

FIG. 4A-4C. Southern blot analysis of PCR positive clones of ES TKN-201123-CAA-01-TAC cells (uty (Plasmid 1) Constitutive Knockin)

Six positive clones (1B4, 1C2, 1D2, 1E3, 1G2 and 1H3) from the PCR screening were expanded and further characterized by Southern blot analysis.

FIG. 4A. The genomic DNA was digested with KpnI and hybridized using a Neo probe. The Neo probe is expected to detect the following DNA fragment from the targeted allele in the Southern analysis: ˜ 17.54 kb (with KpnI digestion).

FIG. 4B. The genomic DNA was digested with ScaI and hybridized using a 5′ arm probe. The 5′ arm probe is expected to detect the following DNA fragment from the targeted allele in the Southern analysis: WT—˜8.83 kb, Mutant—11.48 kb.

FIG. 4C. The genomic DNA was digested with AvrII and hybridized using a 3′ arm probe. The 3′ arm probe is expected to detect the following DNA fragment from the targeted allele in the Southern analysis: WT—˜7.68 kb, Mutant—10.08 kb.

All six ES PCR positive clones (1B4, 1C2, 1D2, 1E3, 1G2 and 1H3) were confirmed correct by Southern blot analysis.

FIG. 5A-5C. Crossing F0 line 2 male with a non-modified female

F0 line 2 males with a non-modified female resulted in a female bias progeny consisting of 11 females out of 12 pups. Pup number 1 was confirmed positive for PCR 1 and 2. All other 11 mice were confirmed as negative for PCR 1, 2 and 3, confirming they are females.

FIG. 5A. PCR 1. Expected PCR Product: Targeted Y-Chromosome: 410 bp.

FIG. 5B. PCR 2. Expected PCR Product: Targeted Y-Chromosome: 431 bp.

FIG. 5C. PCR 3. Expected PCR Product: Wildtype Y-Chromosome: 348 bp.

FIG. 6. Schematic representation of primers for PCR of the Y chromosome for Determining the sex of the offspring

The figure schematically illustrates the Wild type targeted allele (first from top), indicating the homology arm targeted by the targeting vector. The targeting vector (second from top) illustrates all parts of the constructs that comprises the transgene (KI), and the DTA and Neo element, the targeted allele (third from top) illustrates the incorporation of the transgene in the target allele, and the lower construct (forth from top) indicates the final integrated transgene after deletion of the neo. The open numbered frames indicate the location of the PCR reactions 1, 2 and 3. Sex determination is performed by PCR reactions PCR1, PCR2, PCR3, using the three sets of primers [SEQ ID NOs: 102, 93 (PCR1), SEQ ID NOs: 94, 103 (PCR3), and SEQ ID NOs: 96, 97 (PCR3)), as described in the Experimental procedures in Tables 1 and 2A, 2B, and in Example 15. As defined in Table 3, if at least one of the PCRs amplifies the genomic DNA, the pup is considered a male. If all PCRs are negative, the pup is considered a female. The sexing is subsequently validated by observing the mouse genitals at 2 weeks or older.

### DETAILED DESCRIPTION OF THE INVENTION

The present disclosure provides the first demonstration of a genetic system that manipulates the sex litters at the gamete level and produces sexed semen in-vivo. This demonstration is superior in all aspects to the current state-of-the-art of sexed semen to produce biased-sex litters. Sexed semen technology allows production of offspring of a single sex in mammals. This technology has been successfully applied in various species, mainly cattle and humans. The accuracy of sexing sperm using flow cytometry/cell sorting is around 90% in most species, but fertility of sexed sperm is greatly affected. In the demonstrated system the percentage of the desired progeny is also around 90%. The labor and the apparatuses required for the sexed semen in the genetic system are minimal after the animal has been engineered. The animal lines can be maintained indefinitely, and thus only a single animal should only once be constructed. The litter size does not seem to be affected by using the system, and as with the sorted sexed semen, the progeny is non-GMO.

The mouse line produced in this study can already be used in facilities that produce laboratory mice to better control demands for females. Use of this line will reduce the labor associated with separating the animals after weaning. A mouse line producing 100% female progeny would remove the separation requirement completely. Such a line could be constructed by several possible modifications in the construction cassette. For example, additional targets could be designed to better guide the dCas9 to eliminate the Y gamete production. Moreover, a stabilizing domain may be added to dCas9, to increase its stability in the gamete. Such modification may prove useful in increasing the efficiency to single-sex litters. In addition, it is clearly feasible to construct a line producing biased- or single-sex litters of males, by simply transferring the construct to the X chromosome of the male. Constructing the same cassette on the opposite sex chromosome to produce a single litter sex of the other sex.

The described system is applicable in other mammals such as cows in the dairy industry. A similar cassette can be constructed with minor modifications to yield similar results. The sperm from the engineered bull may then be used to fill insemination straws, or the bull can be used for direct mating of the cows. Likewise, the system can be used in the swine industry to produce the desired sex of the pigs and other domestic mammals. Still further, it should be appreciated that in general, if males are not preferred for a specific reason (e.g., more meat etc.), then biasing the sex toward females would be advantageous. Females are the bottleneck in a population expansion, as their number dictates the maximal possible pregnancies whereas only one or few males are sufficient for mating all of them. Therefore, for a farmer, the more females, the faster one can expand the animal reservoir.

The disclosed systems may be further applied to other organisms in which the female is the heterogametic organism, such as chickens. In chickens, the female carries the Z and W sex chromosomes, while the male carries two copies of the Z chromosome. In these cases, the Z chromosome of the maternal line can be engineered to encode a cassette that eliminates the W-chromosome bearing eggs in the ovaries. This would result in the production of only female progeny, which could save billions of male chicks from being culled annually. It would also save resources, as less labor would be required to separate male and female chicks, and less food and space would be needed to raise them. Additionally, because the male elimination is carried out in the ovaries, as opposed to other proposed genetic or spectroscopic methods for eliminating fertilized eggs, there would be no loss of fertilized eggs, and no need to separate and grow them. For male-only progeny, the maternal W chromosome is engineered to encode the cassette instead. This system can thus be manipulated to accommodate changing requirements in different organisms and for both sexes.

More specifically, the present invention provides methods and systems for in vivo sexing gamete cell population. The underlying principle of this approach is that during sperm production (spermatogenesis), at the stage were cells become haploid, the two sex chromosomes, for example, the Y chromosome is separated from the X chromosome and this naturally occurring separation is utilized for directed distraction of the undesired gamete.

The present disclosure discloses the first genetic system biasing the male/female ratio in animals which is composed of a single animal line, with no reduction in litter size, and, in some cases, the offspring of the desired sex (e.g., female) will be non-GMO. The product saves resources in growing animals that are of low economic value such as dairy-male cattle and swine. This dramatically increase the total yield of desired animals while reducing costs associated with exterminating unwanted animals. The disclosed technology bears an additional added benefit in the field of ethical conduct using farm animals since it will save lives of billions of females/males. Thus, the first aspect of the invention relates to a genetically engineered eukaryotic heterogametic organism comprising exogenous nucleic acid sequences in one sex chromosome, and in some optional embodiments, in both sex chromosomes thereof. More specifically, the genetically engineered heterogametic organism of the invention comprises:

A first sex chromosome (a), comprising at least one exogenous nucleic acid sequence integrated therein. In more specific embodiments, the exogenous nucleic acid sequence comprises the following sequences:

At least one sequence (i) (may be also referred to herein for clarity as “first”), comprising at least one nucleic acid sequence encoding at least one nucleic acids modifier component. The at least one sequence (ii) (may be also referred to herein for clarity as “second”), comprises at least one nucleic acid sequence encoding or forming at least one target recognition element for the at least one nucleic acids modifier component of (i). In some optional embodiments, the exogenous nucleic acid sequence may further comprise at least one sequence (iii) (may be also referred to herein for clarity as “third”), comprising at least one nucleic acid sequence encoding at least one inhibitor or repressor for the at least one nucleic acids modifier component of (i).

In some optional embodiments, at least one of the first, second and the optional third nucleic acid sequences of (i), (ii) and (iii), respectively, may be operably linked to at least one regulatory element. More specifically, such regulatory element may be at least one of: at least one inducible or repressible regulatory element [for example, repressible promoters (Tet-off)], at least one post translationally inducible elements, for example, degron/s (DD and LID)], at least one transcriptional regulatory element, at least one post-translationally regulatory element (e.g., degron/s) and at least one temporally or spatially regulatory element (e.g., promoters, enhancers or repressors that are specific to a particular cell, tissue, organ, or cellular compartment, or alternatively, specific to at least one developmental or cellular stage or condition).

In some optional embodiments, the genetically engineered heterogametic organism of the present disclosure may further comprise exogenous nucleic acid sequences also in the second sex chromosome thereof. According to these optional embodiments, the genetically engineered eukaryotic heterogametic organism may further comprise a second sex chromosome (b), comprising at least one of the following sequences: at least one sequence (iv) (may be also referred to herein for clarity as “fourth”), that comprise at least one exogenous nucleic acid sequence encoding at least one inhibitor or repressor for the at least one nucleic acids modifier component of (i), and as a sequence (v) (may be also referred to herein for clarity as “fifth”), at least one sequence comprising at least one endogenous or exogenous nucleic acid sequence encoding at least one transcription factor or repressor, that specifically bind at least one transcription regulatory element operably linked to the nucleic acid sequence of at least one of (i), (ii) and (iii).

It should be noted that optionally, the exogenous nucleic acid sequence/s are operably linked to at least one of: at least one inducible or repressible regulatory element, at least one transcriptional regulatory element, at least one post-translationally regulatory element and at least one temporally or spatially regulatory element.

The present disclosure provides genetically engineered and/or genetically modified eukaryotic organism. A “genetically engineered” or “transgenic organism” generally refers to an organism that comprise and encodes a heterologous nucleic acid sequence, and/or exogenously added nucleic acid sequence, and/or non-naturally occurring nucleic acid sequence, or one or more additional DNA sequences that are not normally endogenous to the organism (collectively referred to herein as “transgenes”). These exogeneous elements and sequences may be in some embodiments chromosomally integrated into the germ cells of the organism. As a result of such transfer and integration, the transferred sequence may be transmitted through germ cells to the offspring of a genetically engineered organism. The genetically engineered organisms (including its progeny) also have the transgene integrated into the sex chromosomes of somatic cells. Germ cells are embryonic cells that undergo meiosis, followed by cellular differentiation into a mature gamete. A gamete is a haploid cell that fuses with another haploid cell during fertilization (conception) in organisms that sexually reproduce. In species that produce two morphologically distinct types of gametes, and in which each individual produces only one type, a female is any individual that produces the larger type of gamete, called an ovum (or egg), and a male produces the smaller tadpole-like type, called a sperm.

In such organisms, the sex i.e., male or female is dictated by a specific sex-determination system. A sex-determination system is a biological system that determines the development of sexual characteristics in an organism. The two main sex-determination systems in eukaryotic species are the XY sex-determination system and the ZW sex-determination system (e.g., XY or ZW).

The term “sex-chromosome”, that may be also referred to as a “gender chromosome”, refers to a chromosome that when paired with another sex chromosome, determine the sex of an organism (e.g., XX, XY or ZZ, WZ). In some organisms (insects such as flies), the number of a specific sex chromosome may also determine the sex of the organism. More specifically, in such cases one sex (e.g., females) have at least one more sex chromosome than the other sex (e.g., males). For example, one sex carry two X chromosomes (XX) and the other sex carry only one (XO) sex chromosome. Thus, it should be understood that the invention further encompasses such options for sex selection.

As noted above, the genetically engineered organism provided by the present disclosure is a heterogametic organism. The term “heterogametic” refers to the sex of a species in which the sex-chromosomes are not the same. For example, an organism containing the X and Y sex chromosomes, or alternatively, the Z and W chromosomes. As indicated above, heterogametic organism may also carry only one sex chromosome, or at least one less sex chromosome as compared to the other sex (e.g., XO vs. XX).

The term “homogametic” refers to the sex of a species in which the sex-chromosomes are the same. More specifically, an organism having at least two copies of one sex chromosome, for example, two copies of the X chromosome, or two copies of the Z chromosome. Similarly, in some embodiments, the homogametic sex may carry at least two copies of one sex chromosome (e.g., XX vs., XO).

Thus, a heterogametic organism is an organism having two different sex chromosomes (W and Z or X and Y), or alternatively, only one copy of one sex chromosome and a homogametic organism carry at least two copies of the same sex chromosome.

In the XY sex-determination system, the male is the heterogametic organism and the sex-chromosome specific for the heterogametic sex is the Y chromosome while the sex-chromosome specific for the homogametic sex is the X chromosome. Therefore, in the XY sex-determination system concerning the sex-chromosomes of the male (heterogametic organism), the sex-chromosome determining for a male progeny is the Y chromosome and the sex-chromosome determining for a female progeny is the X chromosome.

In the WZ sex-determination system, the female is the heterogametic organism and the sex-chromosome specific for the heterogametic sex is the W chromosome while the sex-chromosome specific for the homogametic sex is the Z chromosome. Therefore, in the WZ sex-determination system concerning the sex-chromosomes of the female (heterogametic organism), the sex-chromosome determining for a male progeny is the Z chromosome and the sex-chromosome determining for a female progeny is the W chromosome.

Still further, as indicated above, according to the present disclosure, the first sex chromosome comprises at least one exogenous nucleic acid encoding at least one nucleic acid modifier component. In some embodiments, “the nucleic acid modifier” component may be any component, element or specifically protein, polypeptide or nucleic acid sequence or oligonucleotide that upon direct or indirect interaction with a target nucleic acid sequence, modify or modulate the structure, function (e.g., expression), activity, or stability thereof. Such modification may include the modification of at least one functional group, addition or deletion of at least one chemical group by modifying an existing functional group or introducing a new one such as methyl group. The modifications may include cleavage, methylation, demethylation, deamination, transcription activation or repression, and the like. Specific modifier component applicable in the present invention may include but are not limited to a protein-based modifier, for example, a nuclease, a methyltransferase, a methylated DNA binding factor, a transcription factor, a chromatin remodeling factor, a polymerase, a demethylase, an acetylase, a deacetylase, a kinase, a phosphatase, an integrase, a recombinase, a ligase, a topoisomerase, a girase, a helicase, any combinations thereof or any fusion proteins comprising at least one of the modifier proteins disclosed by the invention.

As will be elaborated herein below, “activity” of the nucleic acid modifier protein referred to herein may relate in some embodiments to any modification performed in any nucleic acid molecule or sequence, for example, any sequence encoding a product, or alternatively any non-coding sequences. Such modification in some embodiments may result (specifically in case performed on a coding sequence), in modulation of the expression, stability or activity of the encoded product. Non-limiting examples for such modification may be nucleolytic distraction, methylation, demethylation, acetylation, transcription activation or repression (e.g., binding and recruitment of transcription factors or repressors) and the like. In some specific embodiments, such nucleic acid modifier protein may be a nuclease, and the activity referred to herein may be the nucleolytic activity of the nuclease. However, in some alternative embodiments, the invention further encompasses other activities that do not relate to nucleolytic activity.

More specifically, as the invention may further encompass the use of an inactive nuclease or any fusion proteins thereof, “activity” may refer to any additional non-nucleolytic activity that may in some embodiments include repression or alternatively, activation of gene expression. More specifically, in case a non-active nuclease is used as part of a fusion protein, such modulation of gene expression may be achieved by including proteins having methylation or de-methylation activity in such fusion protein or alternatively, by recruiting either transcription factors or transcription suppressors to the non-active nuclease. In more specific embodiments, demethylation and/or recruitment of transcription factors may increase the expression of the encoded product, whereas methylation or recruitment of transcription repressors may inhibit or reduce the expression of the encode product. The use of a modifier that recruits transcription repressor/s is exemplified in the disclosed genetically engineered lines #1 to #5, #7 to #12 and #20 to #27, as also disclosed in the Examples and in Tables 4-6.

In yet some further embodiments, the nucleic acid modifier component encoded by the exogenous nucleic acid sequence integrated within the first sex chromosome of the heterogametic genetically engineered organism of the invention, may be a modifier based on at least one nucleic acid molecule. Such modifier may include any nucleic acid molecule that may participate directly or indirectly in gene silencing or expression. In some embodiments, such modifier component may be nucleic acid based molecules that participate in gene expression and silencing, specifically of particular target genes. In some embodiments, such molecules may be any nucleic acid molecules that participate in RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene or RNA sequence. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example, RNA duplexes comprising paired strands, as well as precursor RNAs from which such small non-coding RNAs can be generated. Exemplary RNA silencing agents include dsRNAs such as siRNAs, miRNAs and shRNAs. In some embodiments, the RNA silencing agent is capable of inducing RNA interference. In other embodiments, the RNA silencing agent is capable of mediating translational repression. More specifically, the nucleic acid modifier according to the invention may be an “antisense RNA”, which is a single strand RNA (ssRNA) molecule that is complementary to an mRNA strand of a specific target gene product. Antisense RNA may inhibit the translation of a complementary mRNA by base-pairing to it and physically obstructing the translation machinery. By “complementary”, it is meant the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes.

In yet some specific embodiments, the nucleic acid modifier applicable by the invention, may encode an RNA, specifically, dsRNA molecule participating in RNA interference. RNA interference (RNAi), as indicated above, is a general conserved eukaryotic pathway which down regulates gene expression in a sequence specific manner. It is the process of sequence-specific, post-transcriptional gene silencing in animals and plants, initiated by siRNA that is homologous in its duplex region to the sequence of the silenced gene. Gene silencing is induced and maintained by the presence of partly or perfectly double-stranded RNA (dsRNA). The silenced genes may be endogenous or exogenous to the organism, integrated into a chromosome or present in a transfection vector that is not integrated into the genome. The expression of the gene is either completely or partially inhibited.

More specifically, the dsRNA modifier encoded by the exogenous nucleic acid sequence of the genetically engineered organism encompassed by the invention may be selected from the group consisting of small interfering RNA (siRNA), MicroRNA (miRNA), short hairpin RNA (shRNA), PIWI interacting RNAs (piRNAs).

As known in the art RNAi is a multistep process, specifically, in a first step, there is cleavage of large dsRNAs into 21-23 ribonucleotides-long double-stranded effector molecules called “small interfering RNAs” or “short interfering RNAs” (siRNAs). These siRNAs duplexes then associate with an endonuclease-containing complex, known as RNA-induced silencing complex (RISC). The RISC specifically recognizes and cleaves the endogenous mRNAs containing a sequence complementary to one of the siRNA strands. One of the strands of the double-stranded siRNA molecule comprises a nucleotide sequence that is complementary to a nucleotide sequence of the target gene, or a portion thereof, and the second strand of the double-stranded siRNA molecule comprises a nucleotide sequence substantially similar to the nucleotide sequence of the target gene, or a portion thereof.

In more particular embodiments, siRNAs comprise a duplex, or double-stranded region, of about 18-25 nucleotides long. Often, siRNAs contain from about two to four unpaired nucleotides at the 3′ end of each strand. At least a portion of one strand of the duplex or double-stranded region of a siRNA is substantially homologous to or substantially complementary to a target sequence within the gene product molecule as herein defined.

The strands of a double-stranded interfering RNA (e.g., siRNA) may be connected to form a hairpin or stem-loop structure (e.g., shRNA). Thus, the RNA silencing agent used as the modifier and encoded by the exogenous nucleic acid sequence of the genetically engineered organisms of the present invention, may also be a short hairpin RNA (shRNA). The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including about 3 to 23. Some of the nucleotides in the loop can be involved in base-pair interactions with other nucleotides in the loop. According to other embodiments the RNA silencing agent encoded by the exogenous nucleic acid sequence of the genetically engineered organism of the invention may be a micro-RNA (miRNA). More specifically, miRNAs are small RNAs made from genes encoding primary transcripts of various sizes. The primary transcript (termed the “pri-miRNA”) is processed through various nucleolytic steps to a shorter precursor miRNA, or “pre-miRNA.” The pre-miRNA is present in a folded form so that the final (mature) miRNA is present in a duplex, the two strands being referred to as the miRNA (the strand that will eventually base pair with the target). It should be understood that in some embodiments, where the nucleic acid modifier encoded by the exogeneous nucleic acid sequence is a nucleic acid-based modifier as indicated herein above, the target recognition element (ii), may form a part of the modifier. Specifically, in some embodiments, the target recognition element is part of the modifier (e.g., in case of shRNA, siRNA, and the like). Still further, non-limiting embodiments for a nucleic acid modifier such as shRNA are provided by the present disclosure in genetically engineered organisms of lines #9 and #10, as disclosed in the examples and also in Table 4.

The nucleic acid modifiers, upon acting in the absence of any repressors or control elements, enable targeted destruction of genes responsible or participating in the viability and function of gamete cells. Thus, gamete cells that carry a sex chromosome with the active modifier (e.g., shRNA), are destroyed or not developed. This allows the selection of gametes that carry sex chromosomes of the desired sex (sex chromosome that do not carry the modifier component), during gametogenesis (e.g., spermatogenesis or oogenesis).

Still further, in some optional embodiments, the exogeneous nuclei acid sequence may further comprise (iii) additionally, or alternatively, at least one repressor or inhibitor of the nucleic acid modifier component of (i). The use of such repressors is exemplified foe Example, by the genetically engineered organisms of lines #20 to #27, as disclosed in Table 5. The alternative use of such repressor or inhibitor of the nucleic acid modifier component of (i) is also exemplified by the genetically engineered lines #17 to #19, although inserted in these specific lines in the homogametic organism.

Suitable repressor/s or inhibitor/s of the nucleic acid modifier component of (i), will be further defined herein after.

As noted above, the exogenous nucleic acid sequence integrated in at least one of the sex chromosomes of the heterogametic organisms of the invention may be operably linked to at least one regulatory element, to allow controlled expression of the transgene (the exogenous nucleic acid sequence of at least one of (i), (ii) and the optional (iii)). Such elements may control expression, stability and timing of the encoded transgene or product thereof. In certain embodiments the at least one inducible and/or non-inducible regulatory elements may be inducible or constitutive promotors, enhancers, repressors, transcription regulatory elements, and/or post translationally regulatory elements such as for example degron/s.

As used herein, a “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. The promoter sequence includes a transcription initiation site, as well as protein binding domains responsible for the binding of RNA polymerase. Eukaryotic promoters often, but not always, contain “TATA” boxes and “CAT” boxes. Various promoters, including inducible or repressible promoters, may be used to drive the various vectors of the present invention.

A “constitutive promoter” refers to a promoter that allows for continual transcription of the coding sequence or gene under its control. Examples of constitutive promoters include, without limitation, the cytomegalovirus (CMV) early enhancer fused to the chicken β actin promoter (CAG), the retroviral Rous sarcoma virus (RSV) LTR promoter (optionally with the RSV enhancer), the CMV promoter (optionally with the CMV enhancer), the SV40 promoter, the dihydrofolate reductase promoter, the beta-actin promoter, the phosphoglycerol kinase (PGK) promoter, and the EFla promoter. Non-limiting embodiments for constitutive promoters used in the genetically engineered organism lines of the present disclosure include the CAGG promoter used in lines #7, #8, #17, #19, and #20 to #27 (as disclosed in Tables 4, 5, and 6). In some specific and non-limiting embodiments, such promoter may comprise the nucleic acid sequence as denoted by SEQ ID NO: 70. Additional embodiments for constitutive promoters useful in the genetically engineered organisms of the present disclosure may include the SV40 promoter as used in line #18.

In yet some further embodiments, a promoter suitable for the exogenous nucleic acid sequence of the genetically engineered organism of the invention may be an inducible promoter. An “inducible or repressible promoter” refer to a regulatory region that is operably linked to one or more genes, wherein expression of the gene(s) is increased or decreased, respectively, in the presence of an inducer or repressor of said regulatory region. An “inducible promoter” refers to a promoter that initiates increased levels of transcription of the coding sequence or gene under its control in response to a stimulus or an exogenous environmental condition.

Inducible or repressible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible or repressible promoters and systems are available from a variety of commercial sources. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system, the ecdysone insect promoter, the tetracycline-inducible system, the RU486-inducible system and the rapamycin-inducible system. Still other types of inducible promoters which may be useful in this context are those which are regulated by a specific physiological state, e.g., temperature, acute phase, a particular differentiation state of the cell, or in replicating cells only. Examples for repressible promoters including the tetracycline-repressible system, are described in more detail herein after.

It should be appreciated that the promoters suitable for the present invention may be either endogenous or heterologous. The phrase “endogenous promoter” includes a promoter that is naturally associated, e.g., in a wild-type organism, with an endogenous gene. Thus, in some specific embodiments, the nucleic acid sequence of the invention may comprise or be operably linked to an endogenous promoter. It should be appreciated that such endogenous promoter may be either ectopically added or may be used in its original endogenous location.

In yet some further embodiments, the nucleic acid sequence of the invention may comprise heterologous promoter. The term “heterologous” includes a promoter from a different source or gene. It should be understood that in some embodiments, a promoter comprised within the nucleic acid cassette of the invention may be located 5′ to the nucleic acid sequence of interest. In some embodiments, relevant promoters that may be used by the methods and cassettes of the invention may include but are not limited to CMV promoter, SFFV promoter, EF1alpha promoter, AAT promoter, BgH promoter and any appropriate promoter.

In yet some further embodiments, transcriptional regulatory elements (enhancers, or alternatively, repressors), may be used for regulating the expression of the exogenous nucleic acid sequence of the invention. A transcription enhancer is a short (50-1500 bp) region of DNA that can be bound by transcription factors to increase the likelihood that transcription of a particular gene will occur. Enhancers are generally cis-acting, but can also be trans-acting and can be located far away from the gene, and/or can be located upstream or downstream from the start site, and either in the forward or backward direction.

Still further, the exogenous (and in some embodiments even endogenous regulatory elements) comprised within at least one of the sex chromosomes of the heterogametic genetically engineered organism of the present disclosure may include at least one transcriptional control elements, at least one post translational regulators, for example regulators that affect the stability or activity of the protein encoded by the exogenous or endogenous nucleic acid sequences of the genetically engineered organism. In some specific embodiments, degrons may be used. Degrons are readily understood by one of ordinary skill in the art to be amino acid sequences that control the stability of the protein of which they are part. Degrons that are specifically applicable in the present invention are described in more detail herein after.

It should be noted that the exogenous sequences incorporated within at least one of the sex chromosomes of the heterogametic genetically engineered organism of the present disclosure are in some embodiments, operably linked to regulatory elements as described above, and/or in some embodiments may be also operably linked together. The term “operably linked”, as used in reference to a regulatory sequence and a structural nucleotide sequence, means that the nucleic acid sequences are linked in a manner that enables regulated expression of the linked structural nucleotide sequence.

In some embodiments, the first sex chromosome (a), is a sex chromosome characterizing an undesired sex. Accordingly, the second sex chromosome (b), that may be either modified, or unmodified, is a chromosome characterizing a desired sex.

It should be noted that in some embodiments, the desired sex is of the heterogametic organism, or alternatively, of the homogametic organism. In yet some further embodiments, the undesired sex is of the heterogametic organism, or alternatively, of the homogametic organism.

As discussed above, in yet some further embodiments, the nucleic acids modifier component encoded by the nucleic acid sequence of the first sex chromosome (a) (i), may be at least one modifier protein or any fusion protein thereof. Alternatively, the modifier may be at least one modifier nucleic acid sequence. It should be appreciated that in some embodiments, the “first” nucleic acid sequences integrated within the “first” sex chromosome, may encode both, a protein-based modifier (e.g., protein or fusion protein) and a nucleic acid-based modifier (e.g., RNA interfering molecules). In some embodiments, the nucleic acid modifier is a protein. In yet some further embodiments, such modifier protein is a nuclease. More specifically, such nuclease according to some embodiments, may be at least one of: (i) a nuclease having a nucleolytic activity and/or a fusion protein thereof; (ii) a non-active nuclease and/or a fusion protein thereof.

More specifically, as used herein, the term “nuclease” refers to an enzyme that in some embodiments display a nucleolytic activity, specifically, capable of cleaving the phosphodiester bonds between monomers of nucleic acids (e.g., DNA and/or RNA). Nucleases variously effect single and double stranded breaks in their target molecules. There are two primary classifications based on the locus of activity. Exonucleases digest nucleic acids from the ends. Endonucleases act on regions in the middle of target molecules. They are further subcategorized as deoxyribonucleases and ribonucleases. The former acts on DNA, the latter on RNA. A nuclease must associate with a nucleic acid before it can cleave the molecule, providing a degree of recognition. The nucleases belong just like phosphodiesterase, lipase and phosphatase to the esterases, a subgroup of the hydrolases. This subgroup includes the Exonucleases are enzymes that work by cleaving nucleotides one at a time from the end (exo) of a polynucleotide chain. A hydrolyzing reaction that breaks phosphodiester bonds at either the 3′ or the 5′ end occurs. Eukaryotes and prokaryotes have three types of exonucleases involved in the normal turnover of mRNA: 5′ to 3′ exonuclease (Xrn1), which is a dependent decapping protein; 3′ to 5′ exonuclease, an independent protein; and poly (A)-specific 3′ to 5′ exonuclease. Members of this family include Exodeoxyribonucleases producing 5′-phosphomonoesters, Exoribonucleases producing 5′-phosphomonoesters, Exoribonucleases producing 3′-phosphomonoesters and Exonucleases active with either ribo- or deoxy-. Members of this family include exonuclease, II, III, IV, V, VI, VII, and VIII. As noted above, Endonucleases are enzymes that cleave the phosphodiester bond within a polynucleotide chain. Some endonucleases, such as deoxyribonuclease I, cut DNA relatively nonspecifically (without regard to sequence), while many, typically called restriction endonucleases or restriction enzymes, cleave only at very specific nucleotide sequences.

In some embodiment, the nuclease may be an active enzyme having a nucleolytic activity as specified above. In some alternative embodiments, the nuclease may be a defective enzyme. A defective enzyme (e.g., a defective mutant, variant or fragment) may relate to an enzyme that display an activity reduced in about 1%, 2%, 3%, 4%, 5% to about 100%, specifically, about 5% to about 10%, about 10% to about 15%, about 15% to about 20%, about 20% to about 25%, about 25% to about 30%, about 35% to about 40%, about 40% to about 45%, about 45% to about 50%, about 50% to about 55%, about 55% to about 60%, about 65% to about 70%, about 75% to about 80%, about 80% to about 85%, about 85% to about 90%, about 90% to about 95%, about 95% to about 99.9%, more specifically, reduced activity of about 98% to about 100% as compared to the active nuclease. Still further, the at least one exogeneous nucleic acid sequence integrated in the first sex chromosome of the genetically engineered organism disclosed herein, may comprise (ii) at least one nucleic acid sequence encoding or forming a target recognition element. As used herein a “target recognition element” is a nucleic acid sequence (either RNA or DNA) that will direct the nucleic acid-modifier component (e.g., protein or nucleic acid sequence that directly or indirectly modify the target sequence) encoded by the exogenous nucleic acid sequence of the genetically engineered organism of the invention. For example, in case of a nucleic acid-modifier that is a protein such as a nuclease, the target recognition element may be a nucleic acid guide that targets the nuclease to a specific target position within a nucleic acid sequence. The recognition of the target by the target recognition element is facilitated in some embodiments by base-pairing interactions. These target recognition elements are specifically relevant in case of guided nucleases. In yet some further alternative embodiments, the target recognition element itself may be a sequence within the target site that is recognized by the nuclease (e.g., a restriction site). In some embodiments, for nucleases displaying a nucleolytic activity, directing the nuclease to a specific site may result in cleaving the phosphodiester bonds between monomers of nucleic acids (e.g., DNA and/or RNA) that may lead in some embodiments to specific destruction thereof. In yet some alternative embodiments, where a non-active nuclease is used, and specifically, a fusion protein thereof, directing such defective nuclease to, or alternatively, by a target recognition element, may result in targeted modulation (e.g., activation or repression, methylation or demethylation and the like) of the target nucleic acid sequence that comprises, or is targeted by the target recognition element. Still further, in case the nucleic acid-modifier component is a nucleic acid molecule (e.g., shRNA), the target recognition element, may be in accordance with some embodiments of the invention, an endogenous or exogenous target sequence within the target site that is recognized by the shRNA encoded by the genetically engineered organism of the invention. It should be understood that in such case, binding of the shRNA to its target site (target recognition element), further involves requirement of cellular components (e.g., RISC) that may directly or indirectly modify the target nucleic acid sequence. It should be noted that a target recognition element may comprise between about 1 nucleotide to about 10 nucleotides, specifically, 1, 2, 3, 4, 5, 6, 7, 8, 9, 10 or more. More specifically between about 10 nucleotides to 70 nucleotides or more.

Still further, in some embodiments a restriction enzyme may be used as the nucleic acid modifier component. A restriction enzyme is an endonuclease that cleaves DNA into fragments at or near its specific recognition sites within the molecule. To cut DNA, most restriction enzymes make two incisions, through each sugar-phosphate backbone (i.e. each strand) of the DNA double helix. Restriction enzymes with long recognition sites (recognition site of at least 10 nucleotides) may be in some embodiments, suitable nuclease for the invention (mega nucleases/homing endonucleases). Homing endonucleases constitute a family of very rare-cutting endonucleases. They have recognition sequences that span 12-40 bp of DNA, whereas “classical” restriction enzymes recognize much shorter stretches of DNA, in the 3-8 bp range (up to 12 bp for rare-cutter). In such embodiments, the restriction site may be incorporated (e.g., as a target recognition element) either into the sex chromosome of the heterogametic genetically engineered organism, or alternatively, into a chromosomal or mitochondrial DNA of the homogametic genetically engineered organism of the invention. Non-limiting examples of such restriction enzymes may include, but are not limited to I-Sce I, I-Chu I, I-Dmo I, I-Cre I, I-Csm I, Pl-Sce I, PI-Tli I, PI-Mtu I, I-Ceu I, I-Sce II, I-Sce III, HO, Pi-Civ I, PI-Ctr I, PI-Aae I, PI-Bsu I, PI-Dha I, PI-Dra I, PI-Mav I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, Pl-Pfu I, PI-Rma I, Pl-Spb I, PI-Ssp I, PI-Fac I. In yet some further embodiments “nucleases” as referred to herein, also relates to nucleases that cut ribonucleic acids, specifically, RNA molecules. In some specific embodiments, PNAzymes that specifically cut RNAs or any artificial restriction systems such as argonautes with guides may serve as non-limiting examples for such nucleases. More specifically, in some embodiments, Argonaute protein taken from Pyrococcus furiosus (PfAgo) along with guide DNA, may be used as artificial restriction enzyme. A PNA-based system, called PNAzymes, has a Cu (II)-2, 9-dimethylphenanthroline group that mimics ribonucleases for specific RNA sequence and cleaves at a non-base-paired region (RNA bulge) of the targeted RNA formed when the enzyme binds the RNA. This enzyme shows selectivity by cleaving only at one site that either does not have a mismatch or is kinetically preferred out of two possible cleavage sites. As indicated above, in some further embodiments, the nuclease used by the invention may be either a guided or non-guided nuclease. A guided nuclease is according to some embodiments a nuclease targeted to its specific target site by a nucleic acid sequence that specifically interacts with the target site by base-pairing interactions between nucleotides of the guide nucleic acid sequence and the nucleotide sequence of the target site. A non-guided nuclease is a nuclease that achieves sequence specificity without the use of guiding nucleic acid sequences, for example, using protein-nucleic acid sequence interactions. In more specific embodiments, the non-guided nuclease may be a classical restriction enzyme (e.g., having a restriction site of up to 10 bp), or any derivative or fusion protein thereof, and the guided nuclease may be a TALEN, a ZFN, a CRISPR/Cas system, Fok-1-based systems and the like. In such embodiments, the target recognition element may be an element endogenously comprised within the chromosomal or mitochondrial DNA of both, the heterogametic organisms of the invention.

In yet some specific embodiments, the nuclease may be at least one Transcription activator-like effector nucleases (TALEN). TALEN are restriction enzymes that can be engineered to cut specific sequences of DNA. They are made by fusing a TAL effector DNA-binding domain to a DNA cleavage domain of a nuclease. More specifically, TALENs are artificial endonucleases designed by fusing the DNA-binding domain (multiples of nearly identical repeats each comprised of ˜ 34 amino acids) obtained from TAL (transcription activator-like) effector (TALE) protein to the cleavage domain of the FokI endonuclease. Each TALE repeat independently recognizes its corresponding nucleotide (nt) base with two variable residues [termed the repeat variable di-residues (RVDs)] such that the repeats linearly represent the nucleotide sequence of the binding site.

In yet some further alternative embodiments, the guided nuclease that may be used by the systems of the invention may be at least one Zinc-finger nucleases (ZFNs).

ZFNs are artificial restriction enzymes generated by fusing a zinc finger DNA-binding domain to a DNA-cleavage domain. Zinc finger domains can be engineered to target specific desired DNA sequence/s and this enables zinc-finger nucleases to target unique sequences within complex genomes. More specifically, the ZFNs are artificial endonucleases that have been generated by combining a small zinc finger (ZF; about 30 amino acids) DNA-binding/recognition domain (Cys₂His₂) to a type IIS nonspecific DNA-cleavage domain from the FokI restriction enzyme. However, the cleavage activity of the FokI endonuclease demands dimerization. As a ZF module recognizes a 3 bp sequence, there is a requirement for multiple fingers in each ZFN monomer for recognizing and binding to longer DNA target sequences.

In yet some further alternative embodiments, the artificial zinc-finger protein (AZP)-staphylococcal nuclease (SNase) hybrid (AZP-SNase) may be also used.

In some embodiments, the nuclease encoded by the first nucleic acid sequence incorporated into the first sex chromosome of the genetically engineered heterogametic organism of the invention may be an RNA guided nuclease. Accordingly, the first sex chromosome may therefore further comprise at least one target recognition element for such nuclease. In some embodiments, such recognition element is at least one ribonucleic acid guide (guide RNA) directed against at least one target sequence within at least one of coding and/or non-coding sequences, and/or any product/s thereof, of at least one gene involved in viability, stability and/or function (e.g., motility) of gamete cells of the organism. In some embodiments, at least one gene involved in viability, stability and/or function of gamete cells of the organism may be expressed at a late stage of gametogenesis.

“Gametogenesis” is a biological process by which diploid or haploid precursor cells undergo cell division and differentiation to form mature haploid gametes. Depending on the biological life cycle of the organism, gametogenesis occurs by meiotic division of diploid gametocytes into various gametes, or by mitosis. Animals produce gametes directly through meiosis from diploid mother cells in gonads (testis in males and ovaries in females). In mammalian germ cell development, sexually gametes dimorphic differentiates into primordial germ cells from pluripotent cells during initial mammalian development

Still further, in some embodiments, gametogenesis as used herein specifically refers to spermatogenesis. “Spermatogenesis” is the male version of gametogenesis, where haploid spermatozoa develop from germ cells in the seminiferous tubules of the testis. Sperm maturation is divided into several steps. The primordial germ cells are first going mitosis, and one of the daughter cells becomes spermatogonium. Spermatogonia (plural of spermatogonium) are diploid cells that become larger as they divide through mitosis.

The spermatogonium further differentiates into a primary spermatocyte, and after the first meiosis (Meiosis I) becomes a secondary spermatocyte. At this stage, the X and Y chromosomes are separated from each other but are found in duplicates. The second meiotic division (Meiosis II) forms spermatids containing one copy of each chromosome set and these haploid spermatids then mature into spermatozoa, also known as sperm cells. Thus, the primary spermatocyte gives rise to two cells, the secondary spermatocytes, and the two secondary spermatocytes by their subdivision produce four haploid spermatozoa cells.

The earliest theoretical point that a gamete selection can be successful is at the secondary spermatocytes. Due to cytoplasmic bridges even at the spermatid stage, which can transfer organelles and proteins between the gametes, later stages might be preferable.

In yet some further embodiments, an appropriate target gene may be a gene characterized by at least one of the following features. In some embodiments, appropriate target genes in accordance with the present disclosure may be by genes expressed solely in the testis. If this gene is essential in other tissues, it will be silenced there, and consequently there might be developmental defects in the animal. Spem1 addresses this requirement, as it is indeed expressed solely in the testis. This feature is not unique to Spem1, as there are many other testis-specific genes.

In yet some further embodiments, appropriate target genes may be gene/s essential for gamete formation. Accordingly, inactivation of such genes should eliminate the undesired gamete. Spem1 absence leads to deformed sperm with a bent head wrapped around the neck and middle piece of the tail, resulting in male infertility. In yet some further characterizing feature of the appropriate target gene, is a temporal expression. More specifically, appropriate target genes may be those is expressed in the testis after the separation of the gametes. Spem1 is exclusively expressed in the late stages of spermatid formation, and is thus an optimal non-limiting candidate, for targeting in accordance with the present disclosure.

Still further, as used herein, an RNA guided DNA binding protein nuclease is a nuclease which is guided to its cleavage site (or alternatively, a site for any other alternative activity), by an RNA molecule. This RNA molecule is referred to herein as a guide RNA, or gRNA.

In some more specific embodiments, the RNA guided DNA binding protein nuclease of the genetically engineered organisms and systems of the present disclosure may be any one of a clustered regularly interspaced short palindromic repeat (CRISPR) Class 2 or Class 1 system. The Clustered Regularly Interspaced Short Palindromic Repeats (CRISPR) system is a bacterial immune system that has been modified for genome engineering. CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I. III, and IV and class 2 may be divided into types II, V, and VI. As used herein, CRISPR arrays also known as SPIDRs (Spacer Interspersed Direct Repeats) constitute a family of DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition element may be designed separately to recognize and target any desired target.

In some specific embodiment, the RNA guided DNA binding protein nuclease of the genetically engineered organisms, systems and methods of the present disclosure may be a CRISPR Class 2 system. In yet some further particular embodiments, such class 2 system may be a CRISPR type II system. In a more specific embodiment, the RNA guided DNA binding protein nuclease may be CRISPR-associated endonuclease 9 (Cas9) system. The type II CRISPR-Cas systems include the ‘HNH’-type system (Streptococcus-like; also known as the Nmeni subtype, for Neisseria meningitidis serogroup A str. Z2491, or CASS4), in which Cas9, a single, very large protein, seems to be sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage. Still further, it should be noted that type II system comprise at least one of cas9, cas1, cas2 csn2, and cas4 genes. It should be appreciated that any type II CRISPR-Cas systems may be applicable in the present invention, specifically, any one of type II-A or B. Thus, in yet some further and alternative embodiments, at least one cas gene used in the genetically engineered organisms, methods and systems of the present disclosure may be at least one cas gene of type II CRISPR system (either typeII-A or typeII-B). In more particular embodiments, at least one cas gene of type II CRISPR system used by the methods and systems of the present disclosure may be the cas9 gene.

Thus, according to such embodiments, the RNA guided DNA binding protein nuclease is a CRISPR-associated endonuclease 9 (Cas9) system. It should be appreciated that such system may further comprise at least one of cas1, cas2, csn2 and cas4 genes. Double-stranded DNA (dsDNA) cleavage by Cas9 is a hallmark of “type II CRISPR-Cas” immune systems. The CRISPR-associated protein Cas9 is an RNA-guided DNA endonuclease that uses RNA: DNA complementarity to a target site (proto-spacer). After recognition between Cas9 and the target sequence double stranded DNA (dsDNA) cleavage occur, creating the double strand brakes (DSBs).

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding (also named trans-activating CRISPR RNA or tracrRNA) and about 20 nucleotide long “spacer” or “targeting” sequence, which defines the genomic target to be modified. Guide RNA (gRNA), as used herein refers to a synthetic fusion of the endogenous tracrRNA with a targeting sequence (also named crRNA), providing both scaffolding/binding ability for Cas9 nuclease and targeting specificity. Also referred to as “single guide RNA” or “sgRNA”.

CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy (may be used in some particular embodiments in monitoring and counting the gametes produced by the genetically engineered organisms of the present disclosure.

In most of CRISPR systems, the target sequence within the genome to be edited, should be present immediately upstream of a Protospacer Adjacent Motif (PAM). In other systems, such as type III, there is no PAM. In CRISPR systems based on PAM sequence recognition like CRISPR Type II, the PAM is necessary for target binding and the exact sequence is dependent upon the species of Cas9 (5′ NGG 3′ for Streptococcus pyogenes Cas9). In certain embodiments, Cas9 from S. pyogenes may be used in the methods, genetically engineered organisms and systems of the invention. Nevertheless, it should be appreciated that any known Cas9 may be applicable. Non-limiting examples for Cas9 useful in the present disclosure include but are not limited to Streptococcus pyogenes (SP), also indicated herein as SpCas9, Staphylococcus aureus (SA), also indicated herein as SaCas9, Neisseria meningitidis (NM), also indicated herein as NmCas9, Streptococcus thermophilus (ST), also indicated herein as StCas9 and Treponema denticola (TD), also indicated herein as TdCas9. In some specific embodiments, the Cas9 of Streptococcus pyogenes M1 GAS, specifically, the Cas9 of protein id: AAK33936.1. may be applicable in the genetically engineered organisms, methods and systems of the invention. In some embodiments, the Cas9 protein may be encoded by the nucleic acid sequence as denoted by SEQ ID NO: 47. In further specific embodiments, the Cas9 protein may comprise the amino acid sequence as denoted by SEQ ID NO: 48, or any derivatives, mutants, variants or any fusion proteins thereof. In yet some further embodiments, Cas9 adapted for mammalian use, may be also applicable in the present invention. Non-limiting embodiments for such Cas9 is disclosed by SEQ ID NO: 50 and the encoding nucleic acid sequence of said adapted Cas9 is denoted by SEQ ID NO: 49.

It should be noted that any CRISPR/Cas proteins may be used by the invention, in some embodiments of the present disclosure, the endonuclease may be a Cas9, Cas13, Cas6, Cpf1. CMS1 protein, or any variant thereof that is derived or expressed from Methanococcus maripaludis C7, Corynebacterium diphtheria, Corynebacterium efficiens YS-314, Corynebacterium glutamicum (ATCC 13032), Corynebacterium glutamicum (ATCC 13032), Corynebacterium glutamicum R. Corynebacterium kroppenstedtii (DSM 44385), Mycobacterium abscessus (ATCC 19977), Nocardia farcinica IFM10152, Rhodococcus erythropolis PR4, Rhodococcus jostii RFIA1, Rhodococcus opacus B4 (uid36573), Acidothermus cellulolyticus 11 B, Arthrobacter chlorophenolicus A6, Kribbella flavida (DSM 17836), Thermomonospora curvata (DSM43183), Bifidobacterium dentium Bd1, Bifidobacterium longum DJO10A, Slackia heliotrinireducens (DSM 20476), Persephonella marina EX H 1, Bacteroides fragilis NCTC 9434, Capnocytophaga ochracea (DSM 7271), Flavobacterium psychrophilum JIP02 86, Akkermansia muciniphila (ATCC BAA 835), Roseiflexus castenholzii (DSM 13941), Roseiflexus RS1, Synechocystis PCC6803, Elusimicrobium minutum Pei191, uncultured Termite group 1 bacterium phylotype Rs D17, Fibrobacter succinogenes S85, Bacillus cereus (ATCC 10987), Listeria innocua, Lactobacillus casei, Lactobacillus rhamnosus GG, Lactobacillus salivarius UCC118, Streptococcus agalactiae-5-A909, Streptococcus agalactiae NEM316, Streptococcus agalactiae 2603. Streptococcus dysgalactiae equisimilis GGS 124, Streptococcus equi zooepidemicus MGCS10565, Streptococcus gallolyticus UCN34 (uid46061), Streptococcus gordonii Challis subst CH1, Streptococcus mutans NN2025 (uid46353), Streptococcus mutans, Streptococcus pyogenes M1 GAS, Streptococcus pyogenes MGAS5005, Streptococcus pyogenes MGAS2096, Streptococcus pyogenes MGAS9429, Streptococcus pyogenes MGAS 10270, Streptococcus pyogenes MGAS6180, Streptococcus pyogenes MGAS315, Streptococcus pyogenes SSI-1, Streptococcus pyogenes MGAS10750, Streptococcus pyogenes NZ131, Streptococcus thermophiles CNRZ1066, Streptococcus thermophiles LMD-9, Streptococcus thermophiles LMG 18311, Clostridium botulinum A3 Loch Maree, Clostridium botulinum B Eklund 17B, Clostridium botulinum Ba4 657, Clostridium botulinum F Langeland, Clostridium cellulolyticum H10, Finegoldia magna (ATCC 29328), Eubacterium rectale (ATCC 33656), Mycoplasma gallisepticum, Mycoplasma mobile 163K. Mycoplasma penetrans, Mycoplasma synoviae 53, Streptobacillus, moniliformis (DSM 12112), Bradyrhizobium BTAil, Nitrobacter hamburgensis X14, Rhodopseudomonas palustris BisB18, Rhodopseudomonas palustris BisB5, Parvibaculum lavamentivorans DS-1, Dinoroseobacter shibae. DFL 12, Gluconacetobacter diazotrophicus Pal 5 FAPERJ, Gluconacetobacter diazotrophicus Pal 5 JGI, Azospirillum B510 (uid46085), Rhodospirillum rubrum (ATCC 11170), Diaphorobacter TPSY (uid29975), Verminephrobacter eiseniae EF01-2, Neisseria meningitides 053442, Neisseria meningitides alpha14, Neisseria meningitides Z2491, Desulfovibrio salexigens DSM 2638, Campylobacter jejuni doylei 269 97, Campylobacter jejuni 81116, Campylobacter jejuni, Campylobacter lari RM2100, Helicobacter hepaticus, Wolinella succinogenes, Tolumonas auensis DSM 9187, Pseudoalteromonas atlantica T6c. Shewanella pealeana (ATCC 700345), Legionella pneumophila Paris, Actinobacillus succinogenes 130Z. Pasteurella multocida, Francisella tularensis novicida U 112, Francisella tularensis holarctica, Francisella tularensis FSC 198, Francisella tularensis, Francisella tularensis WY96-3418, or Treponema denticola (ATCC 35405).

Once expressed, the Cas9 protein and the gRNA provided by the “first” sex chromosome of the heterogametic genetically engineered organism of the present disclosure, form a riboprotein complex through interactions between the gRNA “scaffold” domain and surface-exposed positively-charged grooves on Cas9. Cas9 undergoes a conformational change upon gRNA binding that shifts the molecule from an inactive, non-DNA binding conformation, into an active DNA-binding conformation. Importantly, the “spacer” sequence of the gRNA remains free to interact with target DNA. The Cas9-gRNA complex binds any target genomic sequence with a PAM, but the extent to which the gRNA spacer matches the target DNA determines whether Cas9 will cut, or alternatively, perform any other manipulation in case a fusion protein comprising a catalytically inactive cas9 is used. Once the Cas9-gRNA complex binds a DNA target, a “seed” sequence at the 3′ end of the gRNA targeting sequence begins to anneal to the target DNA. If the seed and target DNA sequences match, the gRNA continues to anneal to the target DNA in a 3′ to 5′ direction. Cas9 will only cleave the target if sufficient homology exists between the gRNA spacer and target sequences. Sufficient homology is meant between about 10% to about 99.9% homology or identity between the target site and the gRNA, that is complementary to the complementary strand. Still further, the Cas9 nuclease has two functional endonuclease domains: RuvC and HNH. Cas9 undergoes a second conformational change upon target binding that positions the nuclease domains to cleave opposite strands of the target DNA. The end result of Cas9-mediated DNA cleavage is a double strand break (DSB) within the target DNA that occurs about 3 to 4 nucleotides upstream of the PAM sequence. The resulting DSB may be then repaired by one of two general repair pathways, the efficient but error-prone Non-Homologous End Joining (NHEJ) pathway and the less efficient but high-fidelity Homology Directed Repair (HDR) pathway.

Programmable engineered nucleases (PEN) strategies for genome editing, may be based either on cell activation of the HDR mechanism following specific double stranded DNA cleavage (knock-in system) or on NHEJ mechanism (knock-out system). In some specific embodiments, the targeted genes to be knockout, are repaired through the NHEJ pathway, resulting in most cases in dysfunction of the target genes (deletions/insertions/non-sense mutations etc.). As discussed previously, Cas9 generates double strand breaks (DSBs) through the combined activity of two nuclease domains, RuvC and HNH. The exact amino acid residues within each nuclease domain that are critical for endonuclease activity are known (D10A for HNH and H840A for RuvC in S. pyogenes Cas9) and modified versions of the Cas9 enzyme containing only one active catalytic domain (called “Cas9 nickase”) have been generated. Cas9 nickases still bind DNA based on gRNA specificity, but nickases are only capable of cutting one of the DNA strands, resulting in a “nick”, or single strand break, instead of a DSB. DNA nicks are rapidly repaired by HDR (homology directed repair) using the intact complementary DNA strand as the template. Thus, two nickases targeting opposite strands are required to generate a DSB within the target DNA (often referred to as a “double nick” or “dual nickase” CRISPR system). This requirement dramatically increases target specificity, since it is unlikely that two off-target nicks will be generated within close enough proximity to cause a DSB. It should be therefore understood, that the invention further encompasses the use of the dual nickase approach to create a double nick-induced DSB for increasing specificity and reducing off-target effects, in the genetically engineered organisms, systems and methods of the invention. Additional examples of increasing specificity is the use of a nuclease such as Fok1 fused to dCas9 that serves as a linker to the targeting gRNA.

In yet some other embodiments, the invention further encompasses the option of providing and incorporating within the “first” sex chromosome, at least one nucleic acid sequence encoding at least one pre-crRNA that can be processed to several final gRNA products that may target identical or different targets, or plurality of targets. In yet some more specific embodiments, the crRNA comprised within the gRNA of the invention may be a single-stranded ribonucleic acid (ssRNA) sequence complementary to a target genomic DNA sequence. In some specific embodiments, the target genomic DNA sequence (e.g., gene essential for gametogenesis, gametes viability, stability and function) may be located immediately upstream of a protospacer adjacent motif (PAM) sequence and further. Specific targets applicable in the present invention will be discussed herein after. As indicated herein, the gRNA transcribed by the transgene of the invention may be complementary, at least in part, to the target genomic DNA. More specifically, the gRNA encoded by the transgene, may comprising sequences identical, at least in part, to the target sequence, that are complementary to the complementary strand. In certain embodiments, “Complementarity” refers to a relationship between two structures each following the lock-and-key principle. In nature complementarity is the base principle of DNA replication and transcription as it is a property shared between two DNA or RNA sequences, such that when they are aligned antiparallel to each other, the nucleotide bases at each position in the sequences will be complementary (e.g., A and T or U, C and G). As indicated above in some particular embodiments, the genomic DNA sequence targeted by the gRNA of the system of the invention may be located immediately upstream to a PAM sequence.

Thus, in some embodiments, the genetically engineered organism of the present disclosure may comprise, specifically, within the first sex chromosome thereof, sequences that encode crRNA, specifically, spacers. As used herein, the term “spacer” refers to either a non-repetitive or repetitive spacer sequence that is designed to target a specific sequence. In some specific embodiments, spacers may comprise between about 15 to about 50 nucleotides, specifically, about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more nucleotides. More specifically, spacers may comprise between about 20-35 nucleotides.

The guide or targeting RNA encoded by the CRISPR system of the invention may comprise a CRISPR RNA (crRNA) and a trans activating RNA (tracrRNA). However, it should be noted that in some specific CRISPR system, the guide RNA does not include a tracrRNA, such as CPF1 based CRISPR-Cas systems and CRISPR type I-E. The sequence of the targeting RNA encoded by the CRISPR spacers is not particularly limited, other than by the requirement for it to be directed to (i.e., having a segment that is the same as or complementarity to) a target sequence in genomic DNA that is also referred to herein as a “proto-spacer”. Such proto-spacers comprise nucleic acid sequence having sufficient identity to a targeting RNA encoded by the CRISPR spacers comprised within the nucleic acid sequence encoding the gRNA of the methods and systems of the invention. In some embodiments, a crRNA comprises or consists of 20, 21, 22, 23, 24, 25, 26, 27, 28, 29, 30, 31, 32, 33, 34, 35, 36, 37, 38, 39 or 40 nt of the spacer (targeting) sequence. In specific and non-limiting embodiments, the targeting spacer may comprise or consist of a segment that targets any one of the genomic DNA target sequences for which representative spacer sequences are indicated herein.

In some other embodiment, the RNA guided DNA binding protein nuclease of the genetically engineered organism of the invention may be any one of a clustered regularly interspaced short palindromic repeat (CRISPR) of a newly identified system. In addition, a recent study demonstrated that Cas9 can be split into two distinct fragments, which will reconstitute a functional full-length Cas9 nuclease when brought back together. Examples of efficient N and C terminal fragments of Cas9 being able to perform auto-assembly are described in Zetche B et al. (2015) Nat Biotechnol; 33 (2): 139-142. Non-limiting embodiments for genetically engineered organisms that encode Cas9 as the nucleic acid modifying component are provided by genetically engineered organisms of lines #6 and #13 to #16 (as disclosed in Table 4).

As indicated above, the nucleases, and specifically, the guided nucleases such as Cas9 encoded by the genetically engineered organisms, systems and methods of the present disclosure may be in some embodiments, catalytically inactive nucleases. In such cases, only the targeting properties of these guided nucleases are used (e.g., targeting a target nucleic acid sequence using gRNAs as targeting guides), for targeted manipulation of a target sequence, and the nucleolytic activity in such cases is undesired. Thus, in some embodiments, a guided nuclease with no nucleolytic activity may be used.

In some particular and non-limiting embodiments, the Cas9 enzyme encoded by the genetically engineered organisms of the present disclosure may be a Cas9 devoid of any nucleolytic activity, for example, a defective enzyme such as dCas9.

dCas9 is a mutant Cas9 that lacks endonucleolytic activity. A non-limiting example for such mutant may be a mutant that carries a point mutation in at least one of D10A (aspartic acid to alanine in position 10) and H840A (histidine to alanine in position 840). Such mutant can be used as a modular RNA-guided platform to recruit different protein effectors to DNA in a highly specific manner in cells (Qi et al., Cell 152:1173-1183 (2013)). Both repressive and activating effectors can be fused to dCas9 to repress or alternatively, activate gene expression, respectively (e.g., using methylases (methyl transferases), demethylases, transcription factors or transcription repressors etc.). Thus, in some embodiments, a fusion protein of dCas9 and activating effectors (e.g., transcription factors or enzymes that perform de methylation) or a fusion of dCas9 with repressors, may be used by the genetically engineered organisms and systems of the invention. More specifically, the activation or repression of a specific target sequence may be determined by the gRNAs that recognize the target DNA sequences based only on homologous base pairing. The use of dCas9 fusion protein to repress genes that are essential for development of normal gametes, can be harnessed to determine viability, stability and/or function of gamete cells. In fact, such use may be safer than the use of the natural Cas9 as the changes made do not alter the DNA, but only the transcription level. Thus, regulatory-wise, it may be more acceptable.

Repression by dCas9 is achieved when the naked mutated protein is guided to the target. This repression is more efficient when the guide targets dCas9 to the promoter region of the desired gene and when the guide sequence is complementary to the non-template strand of the gene, and therefore is identical, at least in part to the template strand, e.g., that includes the protospacer. However, this is not absolutely essential, and in some instances, guides to different regions in the gene or the opposite template can also repress efficiently.

Repression by dCas9 can be enhanced by fusing the dCas9 to known repressors. A non-limiting example for such repressor may be the Krüppel associated box (KRAB) domain, which enhances repression of the targets (Gilbert et al., Cell 154:442-451 (2013)). In yet some further embodiments, the repression may be further enhanced by combining with the transcription repression domain of methyl CpG-binding protein 2 (MeCP2). Thus, in some embodiments, the nuclease may be dCas9-KRAB-MeCP2 fusion protein. Non-limiting use of such fusion protein as the nucleic acid modifier component encoded by the genetically engineered organism of the present disclosure has been demonstrated in the genetically engineered organisms of lines ##1 to #5, #7, #8, #11, #12, and #20 to #27 (as disclosed in Tables 4 and 6).

Activation by dCas9 is achieved when a transcriptional activator is fused to it. A non-limiting example for such activator may be the Herpes simplex virus protein vmw65, also known as VP16 (Gilbert et al., Cell 154:442-451 (2013)). Alternatively, the guide RNAs themselves can be engineered (instead or in addition to dCas9) to recruit either activators or repressors, and thus recruit naked dCas9 and dictate the outcome (Zalatan et al., Cell 160:339-350 (2015)). For example, the guides can encode an RNA-domain that recruits a specific RNA-binding protein. This RNA-binding protein may be fused to an activator (e.g., VP16) or a repressor (e.g., Krab) and thus the entire recruitment of dCas9 along with the repressor or activator results in a desired outcome, specifically, the manipulation of the target sequence.

In some further embodiments, the nuclease of the invention may be any other programmed-regulator of DNA.

As noted above, the nucleic acid modifier nuclease component, specifically, the Cas9, or alternatively, the nucleic acid molecule modifier (e.g., shRNA), may target a gene encoding a product or a nucleic acid sequence, that are directly or indirectly involved or essential to viability, function and stability of gamete cells produced by the genetically engineered organism of the invention, or to any stage of gametogenesis (spermatogenesis or oogenesis).

In yet some alternative or additional embodiments, the modifier protein or nucleic acid sequence may target non-coding elements or regions that control a specific gene/s involved in gametogenesis or viability and activity of gamete cells as discussed herein.

Thus, both, coding and non-coding regions may be targeted by the modifier component of the present disclosure. “Coding region” as used herein refer to nucleic acid sequences, specifically, DNA that are transcribed to RNA and translated into a protein product. “Non-coding region” as used herein, refers to components of an organism's DNA that do not encode protein sequences. Some noncoding DNA region is transcribed into functional non-coding RNA molecules, other functions of noncoding DNA regions include the transcriptional and translational regulation of protein-coding sequences, scaffold attachment regions, origins of DNA replication, centromeres and telomeres.

In yet some specific and non-limiting embodiments, the target genes or sequences may be nucleic acid sequences or products thereof that are directly or indirectly essential or involved in viability, stability and/or function of gamete cells or of any process for production thereof. As used herein, gene/s involved in viability, stability and/or function of gamete cells refer to gene allowing the formation or function of the different parts of the ovum or the spermatozoa for example in the case of the male gamete the flagella, the manchette, etc.

In some embodiments, genes and nucleic acid sequences involved in viability, stability and/or function of gamete cells may include but are not limited to at least one of Gopc, Akap4, Sepp1, Vdac3, Tekt4, Tekt2, Hlfnt, Prm2, Prml, Tnp2, Tnpl, Crem, Sun4, Sun5, Spag16, Meig1, IFT88, Hook1, Tbpl1, Tpap, Spem1, Jhdm2a and/or Piwil1, as also disclosed in Table 7, and in more detail herein after in connection with other aspects of the present disclosure.

In yet some further embodiments, the genetically engineered eukaryotic heterogametic organism of the invention may comprise a genetically engineered nucleic acid sequence encoding an inhibitor of the modifier protein, specifically, an inhibitor of the nuclease. In more specific embodiments, such nuclease may be Cas9, accordingly, an inhibitor of such nuclease, may be an inhibitor of Cas9. In some specific and non-limiting embodiments, Cas9 inhibitor applicable in the present invention, may be at least one of anti-CRISPR (Acr) IIA4 (AcrIIA4), AcrIIA2 AcrIIC3, AcrIIA1, AcrIIA3, AcrIIC1, AcrIIC2, AcrE1, AcrE2, AcrE3, AcrE4, AcrF1, AcrF2, AcrF3, AcrF4, AcrF5, AcrF6, AcrF7, AcrF8, AcrF9, AcrF10.

As used herein, the term inhibitor of Cas9 refers to a set of bacteriophage proteins, anti-CRISPRs (Acrs) that inactivate certain CRISPR systems. Acrs (Anti-Clustered Regularly Interspaced Short Palindromic Repeats) can inhibit CRISPR activity by a variety of mechanisms and with varying promiscuity, but predominantly specifically inhibit the binding of a small set of Cas proteins to DNA. In some embodiments, the genetically engineered organism of the present disclosure, as well as systems, cells compositions and methods thereof may apply the AcrIIA4 as an ant-Cas inhibitor. In some embodiments such AcrIIA4 may be disclosed by AcrIIA4Lmo (L. monocytogenes). Non-limiting embodiments for genetically engineered organisms that encode the AcrIIA4 are demonstrated by lines #17 to #27 (Tables 5 and 6).

As indicated above, the nucleic acid sequences of the genetically engineered eukaryotic heterogametic organism of the invention, specifically, (i), (ii) and the optional (iii), may be operably linked to at least one regulatory element. In some embodiments, such elements may be (I) an inducible and/or repressible element. In more specific embodiments, a repressible element (a), may be for example a repressible promoter such as the Tetracycline (Tet)-off, Tetracycline (Tet)-on, Alcohol dehydrogenase 1 (ADH1), GAL4/UAS, LexA/lexAop, and the Q system. More specifically, Tetracycline-Controlled Transcriptional Activation is a method of inducible gene expression where transcription is reversibly turned on or off in the presence of the antibiotic tetracycline or one of its derivatives (e.g. doxycycline).

Tetracycline-controlled gene expression is based upon the mechanism of resistance to tetracycline antibiotic treatment found in Gram-negative bacteria. In nature, the Ptet promoter expresses TetR, the repressor, and TetA, the protein that pumps tetracycline antibiotic out of the cell.

In response to tetracycline or doxycycline (Dox, a more stable tetracycline analogue), the Tet-Off system prevents binding of the tTA transcription factor to the promoter, thereby inhibiting gene expression.

The GAL4-UAS system relates to a biochemical method used to study gene expression that has two parts: the Gal4 gene, encoding the yeast transcription activator protein Gal4, and the UAS (Upstream Activation Sequence), an enhancer to which GAL4 specifically binds to activate gene transcription.

In the LexA/LexAop systems, the E. coli protein LexA works as a repressor by binding to the lexA operator (lexAop). A fusion protein of LexA and a transcription activator (such as the activator domain of Gal4) can induce expression of the gene downstream of the lexAop sequence.

The Q-system utilizes genes from the qa cluster of the bread fungus Neurospora crassa, and consists of four components: the transcriptional activator (QF/QF2/QF2^{W}), the enhancer QUAS, the repressor QS, and the chemical de-repressor quinic acid. Similarly, to GAL4/UAS and LexA/LexAop, the Q-system is a binary expression system that allows expressing reporters or effectors (e.g. fluorescent proteins, ion channels, toxins and other genes) in a defined subpopulation of cells.

It should be noted that the present disclosure encompasses the use of at least one of these systems, specifically, GAL4/UAS, LexA/LexAop and the Q-system, or any combinations thereof.

In yet some further embodiments, an inducible or repressible element may be (b), at least one degron. In more specific embodiments, such degron may be at least one of: destabilization domain (DD), ligand-induced degradation (LID), and auxin-inducible degron (AID). Still further, in some embodiments, the regulatory element operably linked to the nucleic acid sequences of the genetically engineered heterogametic organism of the invention may be a post-translationally regulatory element (II), specifically, degron. Specifically, such degron may be any one of DD, UbK, UbD, and UbM. A degron is a portion of a protein that is important in regulation of protein degradation rates. Known degrons include short amino acid sequences, structural motifs and exposed amino acids (often Lysine or Arginine) located anywhere in the protein. Degrons are present in a variety of organisms, from the N-degrons first characterized in yeast to the PEST sequence of mouse ornithine decarboxylase. While there are many types of different degrons, and a high degree of variability even within these groups, degrons are all similar for their involvement in regulating the rate of a protein's degradation. Some degrons destabilize the protein, and some stabilize it. Their mechanisms are categorized by their dependence or lack thereof on Ubiquitin. Degrons may also be referred to as “Ubiquitin-dependent” or “Ubiquitin-independent”.

Ubiquitin-dependent degrons are so named because they are implicated in the polyubiquitination process for targeting a protein to the proteasome. In some cases, the degron itself serves as the site for polyubiquitination, also denoted herein as Ub tags, such as for example UbR, UbP, UbW, UbH, UbI, UbK, UbQ, UbV, UbL, UbD, UbN, UbG, UbY, UbT, UbS, UbF, UbA, UbC, UbE, UbM. Non-limiting embodiments for the use of degrons in the genetically engineered organism of the present disclosure have been demonstrated in the genetically engineered organisms of lines #3 to #5, and #22 to #27 (as disclosed in Tables 4 and 6).

Still further, in some embodiments, regulation of the targeted protein degradation of the genetically engineered organism of the invention, may use the destabilizing domain (DD) system and the auxin-based protein degradation systems.

In the destabilizing domain (DD) system, a DD based on the FK506- and rapamycin-binding protein FKBP12 was fused to the protein of interest, in this case, the modifier protein or any activator or repressor thereof, encoded by the transgene introduced to the genetically engineered organism of the invention. Since the DD instability is conferred to the fused protein, the protein modifier of the invention is constitutively degraded. However, binding of the small molecule Shield-1 to the DD prevented from degradation and stabilized the fusion protein. Non-limiting embodiments for the use of DD in the genetically engineered organism of the present disclosure have been demonstrated in the genetically engineered organisms of lines #7, #8, #11 to #16 (Table 4), #19 (Table 5) and #20 to #27 (as Table 6).

In yet some further embodiments, the LID system may be used. the ligand-induced degradation (LID) system, is based on the fusion of a LID domain comprising a mutated FKBP12 protein and a carboxy-terminal cryptic degradation sequence (degron) to the protein of interest, specifically, the protein modifier of the invention or any activator or repressor thereof. In the absence of Shield-1, the degron binds the active site of FKBP12 thus preventing degradation, while binding of Shield-1 to the active site displaced the degron resulting in degradation of the LID domain and the fused modifier or any activator or repressor thereof. Since the DD and LID system are utilizing the same inducer molecule, Shield-1 could be used to simultaneously stabilize and destabilize specific target proteins in the same cell. Non-limiting embodiments for the use of LID in the genetically engineered organism of the present disclosure have been demonstrated in the genetically engineered organisms of lines #15 and #16 (as disclosed in Table 4).

The Shield1 compound stabilizes a protein of interest tagged with the destabilization domain (DD), which is based on a mutated version of the FKBP protein. It is used to protect the DD fusion protein of interest from proteasomal degradation. The amount of Shield1 can be varied to tune the amount of stabilized protein of interest within the cell.

In yet some further embodiments, the auxin-inducible degron (AID) system may be used for regulating the translated modifier encoded by the genetically engineered organism of the invention, at the protein level. The auxin-based protein degradation system is based on the genetically engineered expression of Oryza sativa TIR1 and a fusion protein of Arabidopsis thaliana IAA transcription repressor 17 (Aid) and the protein of interest. Addition of auxin induces the interaction between Aid and a multi-protein complex comprising the auxin receptor TIR1 and the endogenous mammalian SCF complex. Since the SCF complex contains a ubiquitylation system, the protein of interest (the modifier protein of the invention or any activator or repressor thereof), is subsequently ubiquitinated (U) leading to rapid degradation by the proteasome. In yet some further embodiments, the regulatory element operably linked to the nucleic acid sequences of the genetically engineered heterogametic organism of the invention may be at least one temporally or spatially regulatory element, specifically, a regulatory element that affects specific tissue or developmental stage. In more specific embodiments, such element may be the Spermatid maturation protein 1 (spem 1) promoter. In other specific embodiments, such element may be the A-Kinase Anchoring Protein 4 (Akap4) promoter.

In some specific embodiments, the genetically engineered eukaryotic heterogametic organism of the present disclosure may comprise a first sex chromosome comprising: (i), at least one nucleic acid sequence encoding at least one Cas9 having a nucleolytic activity and/or a fusion protein thereof; or at least one non-active Cas9 and/or a fusion protein thereof; and (ii), at least one nucleic acid sequence encoding at least one gRNA directed against at least one target sequence.

In some optional embodiments, at least one of said nucleic acid sequences of (i) and (ii), may be operably linked to at least one of at least one inducible regulatory element, at least one transcriptional regulatory element, at least one post-translationally regulatory element and at least one temporally or spatially regulatory element. It should be noted that according to some embodiments, the first sex chromosome characterizes the undesired sex.

In some embodiments, the disclosed genetically modified organism may comprise an exogeneous nucleic sequence encoding a non-active Cas9 and/or a fusion protein thereof. In some embodiments, such non-active Cas9, may be the dCas, as disclosed by the present disclosure. In yet some further embodiments, the dCas encoded by the disclosed exogeneous nucleic acids may be a chimeric protein of dCas with KRAB. Thus, in some embodiments, the genetically engineered eukaryotic heterogametic organism of the present disclosure may comprise a first sex chromosome comprising: (i) at least one nucleic acid sequence encoding dCas9-KRAB-MeCP2 fusion protein. In some embodiments, the dCas9-KRAB-MeCP2 may be expressed under the control of an inducible control element. In more specific embodiments, under an inducible promoter. Still further specific and non-limiting embodiments, the dCas9-KRAB-MeCP2 of the disclosed genetically modified organism may be expressed under the control a Tet-off promoter. The first chromosome of the disclosed genetically modified organism further comprises (ii), at least one nucleic acid sequence encoding at least one gRNA directed against at least one target sequence within at least one of coding and non-coding sequences, and/or any product/s thereof, of at least one gene involved in viability, stability and/or function of gamete cells of said organism. In some embodiments, such genes may be any of the genes disclosed by the present disclosure. In some specific and non-limiting embodiments, the target gene may be a gene expressed at late stage of gametogenesis. It should be noted that sperm maturation is divided into several steps. The primordial germ cells are first going mitosis, and one of the daughter cells becomes spermatogonium. The spermatogonium further differentiates into a primary spermatocyte, and after the first meiosis becomes a secondary spermatocyte. At this stage, the X and Y chromosomes are separated from each other but are found in duplicates. The second meiotic division forms spermatids containing one copy of each chromosome set, and these spermatids then mature into sperm. The earliest theoretical point that a gamete selection can be successful is at the secondary spermatocytes. However, due to cytoplasmic bridges even at the spermatid stage, which can transfer organelles and proteins between the gametes. Spem1 is exclusively expressed in the late stages of spermatid formation. Thus, in some embodiments, the target gene may be the spermatid maturation 1 (Spem1) gene. Spem1 absence leads to deformed sperm with a bent head wrapped around the neck and middle piece of the tail, resulting in male infertility. In some particular and non-limiting embodiments, the genetically engineered eukaryotic heterogametic organism of the present disclosure may comprise a first sex chromosome comprising: (i), at least one nucleic acid sequence encoding dCas9-KRAB-MeCP2 fusion protein under a Tet-off promoter; and (ii), at least one nucleic acid sequence encoding at least one gRNA directed against at least one target sequence within the Spem1 gene. It should be understood that the first sex chromosome that comprises the discussed exogeneous nucleic acid sequences, is the chromosome characterizing the undesired sex. It should be noted that particular embodiments for such genetically engineered organism are provided in the present disclosure by the genetically engineered organism of line #2 as disclosed in Table 4, and in Examples 6, and 15.

In some other specific embodiments, the genetically engineered eukaryotic heterogametic organism of the invention may comprise:

A first sex chromosome (a), comprising the following sequences:

In a first sequence (i), at least one nucleic acid sequence encoding at least one Cas9, any derivative, variant or any fusion protein thereof; and as the second sequence (ii), at least one nucleic acid sequence encoding at least one gRNA directed against at least one target sequence.

According to some optional embodiments, at least one of the nucleic acid sequences of (i) and (ii), is operably linked to at least one of at least one inducible and/or repressible regulatory element, at least one transcriptional regulatory element, at least one post-translationally regulatory element and at least one temporally or spatially regulatory element. It should be noted that in some embodiments, this first sex chromosome, characterizes the undesired sex.

The genetically engineered heterogametic organism comprises a second sex chromosome (b), comprising at least one exogenous nucleic acid sequence encoding at least one inhibitor of the Cas9. According to some optional embodiments, the Cas9 inhibitor may be at least one of AcrIIA4 and AcrIIA2, or at least one of AcrIIC3, AcrE1, AcrE2, AcrE3, AcrE4, AcrF1, AcrF2, AcrF3, AcrF4, AcrF5, AcrF6, AcrF7, AcrF8, AcrF9, AcrF10, AcrIIA1, AcrIIA2, AcrIIA3, AcrIIA4, AcrIIC1, AcrIIC2, AcrIIC3. It should be noted that in some embodiments, the second sex chromosome characterizes the desired sex.

As disclosed in Example 1, in some particular and non-limiting embodiments of the invention, a mammalian genetically engineered heterogametic organism, specifically, a male that produces female gametes (X chromosome gametes) only is produced. This male comprises at least one exogeneous nucleic acid sequence encoding a functional dCas9 system and guide RNAs on the Y chromosome. The X chromosome encodes an anti-dCas9 protein (AcrII4A) under a constitutive promoter. The guides are directed against and therefore repress genes that are essential for sperm morphogenesis and motility (e.g., Gopc, Hook1, Tbpl1, etc.). These genes are repressed only in the Y gametes, as these gametes do not have the anti-dCas9. Thus, only X gametes are produced in this male.

To enable production of gametes containing the Y chromosome (and thereby male progeny for future breeding) from this genetically engineered male, the dCas9 in the above construct is further controlled by a tet-off promoter. Thus, upon supplement of doxycycline in the drinking water, constantly repress the dCas9 and therefore, the Y-chromosome containing gamete are preserved. For producing female-only progeny, doxycycline is removed from the water.

In yet some further embodiments, the present disclosure provides a genetically engineered eukaryotic heterogametic organism according to the invention that comprises:

A first sex chromosome (a), comprising the following sequences: (i), at least one nucleic acid sequence encoding at least one Cas9, any variant, derivatives or any fusion protein thereof; and (ii), at least one nucleic acid sequence encoding at least one gRNA directed against at least one target sequence.

It should be noted that at least one of the nucleic acid sequences of (i) and (ii), are operably linked to at least one transcription regulatory element. It should be noted that at least one of these regulatory elements is an element being specific for a repressor endogenously encoded by a nucleic acid sequence comprised within the second sex chromosome. In yet some optional embodiments, at least one of the nucleic acid sequences of (i), (ii), is operably linked to at least one of: at least one inducible and/or repressible regulatory element, at least one transcriptional regulatory element, at least one post-translationally regulatory element and at least one temporally or spatially regulatory element. It should be noted that in some embodiments this first sex chromosome characterizes the undesired sex.

The genetically engineered organism of the invention further comprises a second sex chromosome (b), comprising at least one endogenous nucleic acid sequence encoding at least one repressor specific for the transcription regulatory element operably linked to at least one of the nucleic acid sequences of (i) and (ii). It should be noted that in some embodiments, this second sex chromosome characterizes the desired sex. In some particular embodiments presented by Example 2, a mammalian heterogametic organism (male) is provided. In this case, the genetically-engineer male comprises at least one exogeneous nucleic acid sequence encoding a functional CRISPR-Cas9 system and guide RNAs under a tet-off promoter. In these males, a sequence repressed by an X-encoded repressor, such as Nkrf, is cloned upstream of the CRISPR-Cas9 system. Thus, the CRISPR-Cas9 system is repressed as long as the X chromosome is present. Upon separation of the X chromosome, the Y gametes are exterminated. These males produce gametes that carry only an X chromosome. For producing males of these genetically engineered males, doxycyclines used in the drinking water of the genetically engineered organism to constantly repress the Cas9 system. For producing female-only progeny, doxycyclines removed from the water.

Still further, in other specific embodiments, the invention provides a genetically engineered eukaryotic heterogametic organism that comprise:

A first sex chromosome (a), comprising: First (i), at least one nucleic acid sequence encoding at least one Cas9, or any derivatives, variants or fusion protein thereof; second (ii), at least one nucleic acid sequence encoding at least one gRNA directed against at least one target sequence; and third (iii), at least one nucleic acid sequence encoding at least one inhibitor of the Cas9 or any derivatives, variants or fusion proteins thereof. In some optional embodiments the Cas9 inhibitor is at least one of AcrIIA4 and AcrIIA2. In yet some further embodiments, the Cas9 inhibitor may be at least one of AcrIIC3, AcrE1, AcrE2, AcrE3, AcrE4, AcrF1, AcrF2, AcrF3, AcrF4, AcrF5, AcrF6, AcrF7, AcrF8, AcrF9, AcrF10, AcrIIA1, AcrIIA2, AcrIIA3, AcrIIA4, AcrIIC1, AcrIIC2, AcrIIC3. It should be noted that this third nucleic acid sequence is operably linked to at least one transcription regulatory element. Such regulatory element being specific for a transcription factor endogenously encoded by a nucleic acid sequence comprised within the second sex chromosome. Optionally, at least one of the nucleic acid sequences of (i), (ii) and (iii), is operably linked to at least one of at least one inducible and/or repressible regulatory element, at least one transcriptional regulatory element, at least one post-translationally regulatory element and at least one temporally or spatially regulatory element. It should be noted that in some embodiments, the first sex chromosome characterizes the undesired sex. This genetically engineered organism of the present disclosure further comprises a second sex chromosome (b), comprising at least one endogenous nucleic acid sequence encoding at least one transcription factor specific for the transcription regulatory element operably linked to the nucleic acid sequences of (iii). It should be noted that the second sex chromosome characterizes the desired sex. In some non-limiting embodiments of such genetically engineered organism, as disclosed in Example 3, the heterogametic organism is a genetically engineered mammalian male. According to such embodiments, a functional CRISPR-Cas9 system and guide RNAs under a tet-off promoter as well as an anti-CRISPR protein under an X-encoded transcription factor promoter is encoded on the same cassette on the Y-chromosome. The X-encoded transcription factor that activates the anti-CRISPR protein, may be any one of Sox3, Foxp3 and/or Tfe3. Thus, the anti-CRISPR protein is expressed as long as the X chromosome is present. Once the X is separated, the CRISPR-Cas9 system is not repressed, and the Y encoding gametes are exterminated. In this case, all gametes produced by the male contain the X chromosome. For producing males of these genetically engineered males, doxycyclines used in the drinking water of the genetically engineered organism to constantly repress the Cas9 system. For producing female-only progeny, doxycycline removed from the water. Semen extracted from the engineered organism, tested for female-only zygotes and further used as needed.

In yet some additional embodiments, the invention provides a genetically engineered eukaryotic heterogametic organism comprising a first sex chromosome (a), comprising: (i), at least one nucleic acid sequence encoding at least one Cas9, or any derivative, variant or fusion protein thereof; and (ii), at least one nucleic acid sequence encoding at least one gRNA directed against at least one target sequence. It should be noted that at least one of the nucleic acid sequences of (i) and (ii), is operably linked to at least one of: at least one inducible and/or repressible regulatory element, at least one transcriptional regulatory element, at least one post-translationally regulatory element and at least one temporally or spatially regulatory element. It should be noted that this first sex chromosome characterizes the undesired sex. As demonstrated by Example 4, in some non-limiting embodiments, mammalian genetically engineered male is provided. In this male, the Y chromosome is genetically-engineered to encode a functional dCas9 and guide RNAs. This cassette is driven by a sperm-specific gene (e.g., Spem1) promoter, which is expressed only in the gamete stage. Thus, dCas9-mediated repression of genes required for spermatogenesis will only occur in gametes carrying the Y-chromosome, and not in gametes carrying the X chromosome. This male thus produce only females (gametes that carry the X chromosome), and since the X chromosome is unmodified, the resulting progeny is non-GMO.

In this construction of males producing non-GMO progeny, the dCas9 is controlled at the protein level by a synthetic chemical. The dCas9 is fused to a Ligand-induced degradation (LID) domain that leads to the entire protein degradation in the presence of the synthetic chemical Shield1. Thus, the parental male lines are able to produce male progeny for future breeding upon supplement of Shield1 in their drinking water. The supplement of Shield1 constantly degrade the dCas9 and therefore, the Y-chromosome containing gamete is preserved. For producing female-only progeny, Shield 1 is removed from the water.

It should be appreciated that any regulatory element disclosed by the present disclosure may be applicable for any of the genetically engineered heterogametic organisms of the present disclosure, as well as for any aspect disclosed herein after.

In certain embodiments, the genetically engineered eukaryotic heterogametic organism of the invention produces gamete cells predominantly composed of one desired sex.

As used herein the term “predominantly” refers to a gamete cells wherein is the desired sex or selected sex represents 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% of the total gamete cells. In yet some further embodiments, at least 80% of the gamete cells produced by the genetically engineered organisms of the invention, are of the desired sex. In yet some further embodiments, at least 85%, at least 90%, at least 95%, at least 96%, at least 97%, at least 98%, at least 99%, or 100% of the gamete cells produced by the genetically engineered organisms of the invention, are of the desired sex.

In some specific embodiments of the genetically engineered eukaryotic heterogametic organism of the invention, in cases where the desired sex is of the homogametic organism (for example, female XX), and the undesired sex is of the heterogametic organism (for example a male XY), the first sex chromosome is a chromosome characterizing the heterogametic organism, that is the undesired sex (for example, the Y chromosome) and the second sex chromosome is a chromosome characterizing the homogametic organism (for example, the X chromosome).

In yet some alternative embodiments, where the desired sex is of the heterogametic organism (for example, male YX), and the undesired sex is of the homogametic organism (for example a male XX), the first sex chromosome is a chromosome characterizing the homogametic organism, that is the undesired sex (for example, the X chromosome) and the second sex chromosome is a chromosome characterizing the heterogametic organism (for example, the Y chromosome).

It should be understood that the genetically engineered organisms, systems as well as the methods of the invention are suitable to any eukaryotic species possessing a heterogametic and homogametic organisms. Such organisms are present both in the Animalia and Plantae biological kingdoms.

In some embodiments, the genetically engineered eukaryotic heterogametic organism of the invention is of the biological kingdom Animalia.

In other embodiments, the eukaryotic heterogametic organism and homogametic organism of the system of the invention may be any one of a vertebrate or an invertebrate.

Invertebrates are animals that neither possess nor develop a vertebral column (commonly known as a backbone or spine), derived from the notochord. This includes all animals apart from the subphylum Vertebrata. Familiar examples of invertebrates include insects; crabs, lobsters and their kin; snails, clams, octopuses and their kin; starfish, sea-urchins and their kin; jellyfish and worms. Vertebrates comprise all species of animals within the subphylum Vertebrata (chordates with backbones). Vertebrates represent the overwhelming majority of the phylum Chordata, with currently about 66,000 species described. Vertebrates include the jawless fish and the jawed vertebrates, which include the cartilaginous fish (sharks, rays, and ratfish) and the bony fish. Still further, in some embodiments, the genetically engineered eukaryotic heterogametic organism of the invention may be any one of a non-human mammal, an avian, an insect, a fish, an amphibian, a reptile, a crustacean, a crab, a lobster, a snail, a clam, an octopus, a starfish, a sea-urchin, jellyfish, and worms.

In more specific embodiments, the genetically engineered eukaryotic heterogametic organism of the invention may be a mammal, specifically, a non-human mammal. In yet some further embodiments, such mammalian organisms may include any member of the mammalian nineteen orders, specifically, Order Artiodactyla (even-toed hoofed animals), Order Carnivora (meat-eaters), Order Cetacea (whales and purpoises), Order Chiroptera (bats), Order Dermoptera (colugos or flying lemurs), Order Edentata (toothless mammals), Order Hyracoidae (hyraxes, dassies), Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses), Order Primates (primates), Order Proboscidea (elephants), Order Rodentia (gnawing mammals), Order Sirenia (dugongs and manatees), Order Tubulidentata (aardvarks). In some specific embodiment, such mammal may be at least one of a Cattle, domestic pig (swine, hog), sheep, horse, goat, alpaca, lama and Camels.

In some embodiments, the present disclosure provides genetically engineered heterogametic mammalian organisms of the Order Artiodactyla, including members of the family Suidae, subfamily Suinae and Genus Sus, and members of the family Bovidae, subfamily Bovinae including ungulates. More specifically, domestic cattle, bison, African buffalo, the water buffalo, the yak. Of particular interest in the present disclosure are domestic cattle being the most widespread species of the genus Bos, and are most commonly classified collectively as Bos taurus. More specifically, the present genetically engineered organisms, systems of the present disclosure as well as the methods disclosed herein above offer great economic advantage for any industrial or agricultural use of animals, specifically, livestock. Thus, in some specific embodiments, the invention (systems, genetically engineered organisms and methods thereof) may be applicable for mammalian livestock. Livestock are domesticated animals raised in an agricultural setting to produce labor and commodities such as meat, eggs, milk, fur, leather, and wool. The term includes but is not limited to Cattle, sheep, domestic pig (swine, hog), horse, goat, alpaca, lama and Camels. Of particular interest are cattle applicable in the meat and milk industry, as well as in the leather industry. More specifically, in certain embodiments, the genetically engineered animals of the invention, as well as the animals provided and used by the systems and methods of the present disclosure may be Cattle, colloquially cows, that are the most common type of large, domesticated ungulates, that belong to the Bovidae family.

Thus, in yet some specific embodiments, the mammalian genetically engineered eukaryotic heterogametic organism of the invention is a domestic Cattle. According to such embodiments, the heterogametic organism is a male (YX). Thus, in cases (a), where the desired sex is female, the first sex chromosome is the Y chromosome, and the second sex chromosome is the X chromosome. The genetically engineered male of the invention may therefore produce sperm predominantly composed of gamete cells predominantly comprising the X chromosome/s.

In alternative embodiments, where the desired sex is male (b), the first sex chromosome as described by the invention herein above, is the X chromosome, and the second sex chromosome is the Y chromosome. According to such particular embodiments, such genetically engineered male produces sperm predominantly composed of gamete cells predominantly comprising the Y chromosome.

Still further, the Bovidae are the biological family of cloven-hoofed, ruminant mammals that includes bison, African buffalo, water buffalo, antelopes, wildebeest, impala, gazelles, sheep, goats, muskoxen. The biological subfamily Bovinae includes a diverse group of ten genera of medium to large-sized ungulates, including domestic cattle, bison, African buffalo, the water buffalo, the yak, and the four-horned and spiral-horned antelopes. Of particular interest in the present invention may be the domestic cattle are the most widespread species of the genus Bos, and are most commonly classified collectively as Bos taurus. More specifically, Bos is the genus of wild and domestic cattle. Bos can be divided into four subgenera: Bos, Bibos, Novibos, and Poephagus. Subgenus Bos includes Bos primigenius (cattle, including aurochs), Bos primigenius primigenius (aurochs), Bos primigenius taurus (taurine cattle, domesticated) and Bos primigenius indicus (zebu, domesticated).

In yet some further embodiments, the genetically engineered organisms of the invention are of particular relevance to rodent since it represents the most popular and commonly accepted animal model in research.

Still further, in some embodiments, the present disclosure may be applicable as the genetically modified organism of the present disclosure. The pig (Sus domesticus), is of the mammalian family Equidae, and the order Artiodactyla. Pigs may be also referred to herein as swine, hog, or domestic pig when distinguishing from other members of the genus Sus, is an omnivorous, domesticated, even-toed, hoofed mammal. It is variously considered a subspecies of Sus scrofa (the wild boar or Eurasian boar) or a distinct species.

When used as livestock, pigs are farmed primarily for the production of meat. The animal's bones, hide, and bristles are also used in products. Pigs, especially miniature breeds, are also kept as pets. The present disclosure therefore encompasses any genetically modified pig that carry the exogenous nucleic acid sequences in one of its sex chromosomes, as disclosed herein.

Still further, in some embodiments, the present disclosure may be applicable for the mammalian family Equidae, more specifically a horse. The “horse” (Equus ferus caballus) is a domesticated, one-toed, hoofed mammal. It belongs to the taxonomic family Equidae and is one of two extant subspecies of Equus ferus. Horses and humans interact in a wide variety of sport competitions and non-competitive recreational pursuits as well as in working activities such as police work, agriculture, entertainment, and therapy.

It should be further understood that domestic animals may further encompass also pets. Thus, in some embodiments, the disclosed genetically modified organisms, systems and methods may be applicable for biasing sex ratio in pets. In yet some further embodiments, the disclosure may be used for pets. The term “pet” or “companion animal” refers to an animal kept primarily for a person's company or entertainment rather than as a working animal, livestock, or a laboratory animal. Pets as used herein include dogs, cats, rabbits; ferrets; pigs; rodents such as gerbils, hamsters, chinchillas, rats, mice, and guinea pigs; birds such as parrots, passerines, and fowls; reptiles such as turtles, lizards, snakes, and iguanas; aquatic pets such as fish, freshwater snails, and saltwater snails; amphibians such as frogs and salamanders; and arthropod pets such as tarantulas and hermit crabs.

Thus, in some further embodiment, the mammal of the system of the invention may be a rodent. Rodents are mammals of the order Rodentia, which are characterized by a single pair of continuously growing incisors in each of the upper and lower jaws. Rodents are the largest group of mammals. In some embodiments, the genetically engineered organisms of the invention, as well as the systems and methods disclosed herein after, may be any rodent. Non-limiting examples for such rodents that are applicable in the present invention, appear in the following list of rodents, arranged alphabetically by suborder and family. Suborder Anomaluromorpha includes the anomalure family (Anomaluridae) [anomalure (genera Anomalurus, Idiurus, and Zenkerella)], the spring hare family (Pedetidae) [spring hare (Pedetes capensis)]. The suborder Castorimorpha includes the beaver family (Castoridae) [beaver (genus Castor), giant beaver (genus Castoroides; extinct)], the kangaroo mice and rats (family Heteromyidae) [kangaroo mouse (genus Microdipodops), kangaroo rat (genus Dipodomys), pocket mouse (several genera)], the pocket gopher family (Geomyidae) [pocket gopher (multiple genera)]. Suborder Hystricomorpha, includes the agouti family (Dasyproctidae), acouchy (genus Myoprocta) [agouti (genus Dasyprocta)], the American spiny rat family (Echimyidae), the American spiny rat (multiple genera), the blesmol family (Bathyergidae) [blesmol (multiple genera)], the cane rat family (Thryonomyidae) [cane rat (genus Thryonomys)], the cavy family (Caviidae) [capybara (Hydrochoerus hydrochaeris), guinea pig (Cavia porcellus) mara (genus Dolichotis)], the chinchilla family (Chinchillidae) [chinchilla (genus Chinchilla), viscacha (genera Lagidium and Lagostomus)], the chinchilla rat family (Abrocomidae) [chinchilla rat (genera Cuscomys and Abrocoma)], the dassie rat family (Petromuridae) [dassie rat (Petromus typicus)], the degu family (Octodontidae) [degu (genus Octodon)], the diatomyid family (Diatomyidae), the giant hutia family (Heptaxodontidae), the gundi family (Ctenodactylidae) [gundi (multiple genera)], the hutia family (Capromyidae) [hutia (multiple genera)], the New World porcupine family (Erethizontidae) [New World porcupine (multiple genera)], the nutria family (Myocastoridae) [nutria (Myocastor coypus)], the Old World porcupine family (Hystricidae) [Old World porcupine (genera Atherurus, Hystrix, and Trichys)], the paca family (Cuniculidae) [paca (genus Cuniculus)], the pacarana family (Dinomyidae) [pacarana (Dinomys branickii)], the tuco-tuco family (Ctenomyidae) [tuco-tuco (genus Ctenomys)]. The suborder Myomorpha that includes the cricetid family (Cricetidae) [American harvest mouse (genus Reithrodontomys), cotton rat (genus Sigmodon), deer mouse (genus Peromyscus), grasshopper mouse (genus Onychomys), hamster (various genera), golden hamster (Mesocricetus auratus), lemming (various genera) maned rat (Lophiomys imhausi), muskrat (genera Neofiber and Ondatra), rice rat (genus Oryzomys), vole (various genera), meadow vole (genus Microtus), woodland vole (Microtus pinetorum), water rat (various genera), woodrat (genus Neotoma), dipodid family (Dipodidae), birch mouse (genus Sicista), jerboa (various genera), jumping mouse (genera Eozapus, Napaeozapus, and Zapus)], the mouselike hamster family (Calomyscidae), the murid family (Muridae) [African spiny mouse (genus Acomys), bandicoot rat (genera Bandicota and Nesokia), cloud rat (genera Phloeomys and Crateromys), gerbil (multiple genera), sand rat (genus Psammomys), mouse (genus Mus), house mouse (Mus musculus), Old World harvest mouse (genus Micromys), Old World rat (genus Rattus), shrew rat (various genera), water rat (genera Hydromys, Crossomys, and Colomys), wood mouse (genus Apodemus)], thenesomyid family (Nesomyidae), African pouched rat (genera Beamys, Cricetomys, and Saccostomus)], the Oriental dormouse family (Platacanthomyidae) [Asian tree mouse (genera Platacanthomys and Typhlomys)], the spalacid family (Spalacidae) [bamboo rat (genera Rhizomys and Cannomys), blind mole rat (genera Nannospalax and Spalax), zokor (genus Myospalax), suborder Sciuromorpha], the dormouse family (Gliridae) [dormouse (various genera), desert dormouse (Selevinia betpakdalaensis)], the mountain beaver family (Aplodontiidae) [mountain beaver (Aplodontia rufa)], the squirrel family (Sciuridae) [chipmunk (genus Tamias), flying squirrel (multiple genera), ground squirrel (multiple genera), suslik (genus Spermophilus), marmot (genus Marmota), groundhog (Marmota monax), prairie dog (genus Cynomys), tree squirrel (multiple genera)]. In yet some further embodiments, the system of the invention may be applicable for mice. A mouse, plural mice, is a small rodent characteristically having a pointed snout, small, rounded ears, a body-length scaly tail and a high breeding rate. The best-known mouse species is the common house mouse (Mus musculus). Species of mice are mostly found in Rodentia and are present throughout the order. Typical mice are found in the genus Mus. It should be understood that in some embodiments, sex biasing in laboratory animals may be of particular interest as it facilitates and reduce costs of maintenance and separation of animals in single sex cages.

As noted above, in more particular embodiments, the genetically engineered eukaryotic heterogametic rodent provided by the invention is a mouse. Thus, according to some embodiments, the heterogametic organism is a male mouse, that carry both X and Y sex chromosomes. Therefore, in cases where the desired sex is female, the first sex chromosome of the genetically engineered muse of the invention is the Y chromosome, and the second sex chromosome is the X chromosome. In such case, the male produces sperm predominantly composed of gamete cells comprising the X chromosome/s.

In yet some alternative embodiments, where the desired sex is male, the first sex chromosome is the X chromosome, and the second sex chromosome is the Y chromosome. Accordingly, such male produces sperm predominantly composed of gamete cells comprising the Y chromosome.

In some specific embodiments, the genetically engineered eukaryotic heterogametic mammal of the present disclosure may comprise a first sex chromosome comprising: (i), at least one nucleic acid sequence encoding at least one Cas9, or any fusion protein thereof; and (ii), at least one nucleic acid sequence encoding at least one gRNA directed against at least one target sequence.

In some optional embodiments, at least one of said nucleic acid sequences of (i) and (ii), may be operably linked to at least one of at least one inducible regulatory element, at least one transcriptional regulatory element, at least one post-translationally regulatory element and at least one temporally or spatially regulatory element. It should be noted that according to some embodiments, the first sex chromosome characterizes the undesired sex.

In some embodiments, wherein the desired sex is female, the genetically engineered eukaryotic heterogametic organism may be a mammalian male comprising a Y chromosome comprising: (i), at least one nucleic acid sequence encoding at least one Cas9 having a nucleolytic activity and/or a fusion protein thereof; or at least one non-active Cas9 and/or fusion protein thereof; and (ii), at least one nucleic acid sequence encoding at least one gRNA directed against at least one target sequence. In some optional embodiments, the target sequence is at least one protospacer comprised within at least one of the Spem1, JmjC-containing H3K9 demethylase (Jhdm2a), SUN domain-containing protein 4 (Sun4), SUN domain-containing protein 5 (Sun5), Crem, Golgi-associated PDZ and coiled-coil motif-containing protein gene (Gopc), the hook microtubule tethering protein 1 (Hook1) and/or the TATA box-binding protein-like protein 1 (Tbpl1) genes, and/or any orthologs or homologs thereof. still further, in some optional embodiments, the nucleic acid sequences of (i) and (ii), may be operably linked to at least one of at least one inducible regulatory element, at least one transcriptional regulatory element, at least one post-translationally regulatory element and at least one temporally or spatially regulatory element.

In some particular and non-limiting embodiments, the genetically engineered eukaryotic heterogametic mammal of the present disclosure may comprise a first sex chromosome comprising: (i), at least one nucleic acid sequence encoding dCas9-KRAB-MeCP2 fusion protein under a Tet-off promoter; and (ii), at least one nucleic acid sequence encoding at least one gRNA directed against at least one target sequence within the Spem1 gene. It should be understood that the first sex chromosome that comprises the discussed exogeneous nucleic acid sequences, is the chromosome characterizing the undesired sex. It should be noted that particular embodiments for such genetically engineered organism are provided in the present disclosure by the genetically engineered organism of line #2 as disclosed in Table 4, and in Examples 6, and 15.

In more particular embodiments, the genetically engineered eukaryotic heterogametic organism provided by the invention may be a mammal as described above, specifically, any of the mammals disclosed by the invention. In some specific embodiments in cases the desired sex is female (XX), the genetically engineered organism of the invention is the heterogametic organism, specifically, a male (YX). Such genetically engineered male comprises:

A Y chromosome (also referred by the invention as the first sex chromosome) comprising the following exogenous sequences: (i) at least one nucleic acid sequence encoding at least one Cas9, or any fusion protein thereof; and (ii), at least one nucleic acid sequence encoding at least one gRNA directed against at least one target sequence. In some embodiments, the target sequence resides in genes essential for viability and function of gamete cells.

In some optional embodiments, at least one of the nucleic acid sequences of (i) and (ii), is operably linked to at least one of: at least one inducible regulatory element, at least one transcriptional regulatory element, at least one post-translationally regulatory element and at least one temporally or spatially regulatory element. Still further, this genetically engineered mammal may further comprise an X chromosome (also referred to herein as the second sex chromosome) comprising at least one exogenous nucleic acid sequence encoding at least one inhibitor of the Cas9. In some optional embodiments, the Cas9 inhibitor is at least one of AcrIIA4 and AcrIIA2. Such particular embodiments are further illustrated by Example 1.

In yet some further particular embodiments, the genetically engineered eukaryotic heterogametic organism provided by the invention may be a mammal as described above. In some specific embodiments in cases the desired sex is female (XX), the genetically engineered organism of the invention is the heterogametic organism, specifically, a male (YX). Such genetically engineered male comprises:

A Y chromosome (also referred by the invention as the first sex chromosome) comprising the following exogenous sequences: (i), at least one nucleic acid sequence encoding at least one Cas9, or any fusion protein thereof; and (ii) at least one nucleic acid sequence encoding at least one gRNA directed against at least one target sequence. Optionally, the target sequence is at least one protospacer comprised within at least one of Jhdm2a, Sun4, Sun5, Spem1, Crem, Gopc, Hook1, and the Tbpl1 genes or any orthologs or homologs thereof.

According to these embodiments, at least one of the nucleic acid sequences of (i) and (ii), is operably linked to at least one transcription regulatory element, such regulatory element being specific for a repressor endogenously encoded by a nucleic acid sequence comprised within the X chromosome. In yet some optional embodiments, at least one of the nucleic acid sequences of (i), (ii), is operably linked to at least one of: at least one inducible regulatory element, at least one transcriptional regulatory element, at least one post-translationally regulatory element and at least one temporally or spatially regulatory element (for example the Spem1, that restrict expression to spermatogenesis stage). Still further, this genetically engineered male further comprises an X chromosome (also referred to herein as the second sex chromosome) comprising at least one endogenous nucleic acid sequence encoding at least one repressor specific for the transcription regulatory element operably linked to at least one of the nucleic acid sequences of (i) and (ii) in the Y chromosome as described above. This particular embodiment is illustrated by Example 2. In yet some further specific embodiments, the genetically engineered eukaryotic heterogametic organism provided by the invention may be a mammal as described above. In some specific embodiments in cases the desired sex is female (XX), the genetically engineered organism of the invention is the heterogametic organism, specifically, a male (YX). Such genetically engineered male comprises:

A Y chromosome (also referred by the invention as the first sex chromosome) comprising the following exogenous sequences: (i), at least one nucleic acid sequence encoding at least one Cas9, or any fusion protein thereof; (ii), at least one nucleic acid sequence encoding at least one gRNA directed against at least one target sequence. Optionally, the target sequence is at least one protospacer comprised within at least one of the Jhdm2a, Sun4, Sun5, Spem1, Crem, Gopc, Hook1, and the Tbpl1 genes or any orthologs or homologs thereof. The Y chromosome of this genetically engineered male may further comprise (iii), at least one nucleic acid sequence encoding at least one inhibitor of said Cas9, optionally, said Cas9 inhibitor is at least one of AcrIIA4 and AcrIIA2.

It should be noted that this sequence is operably linked to at least one transcription regulatory element specific for a transcription factor endogenously encoded by a nucleic acid sequence comprised within the X chromosome. In some optional embodiments, at least one of the nucleic acid sequences of (i), (ii) and (iii), is operably linked to at least one of: at least one inducible regulatory element, at least one transcriptional regulatory element, at least one post-translationally regulatory element and at least one temporally or spatially regulatory element (e.g., Spem 1). The X chromosome of such genetically engineered male (also referred to herein as the second sex chromosome) comprises at least one endogenous nucleic acid sequence encoding at least one transcription factor specific for the transcription regulatory element operably linked to the nucleic acid sequences of (iii) in the Y chromosome. This particular embodiment is illustrated by Example 3.

In yet some further particular embodiments, the genetically engineered eukaryotic heterogametic organism provided by the invention may be a mammal as described above. In some specific embodiments, in cases the desired sex is female (XX), the genetically engineered organism of the invention is the heterogametic organism, specifically, a male (YX). Such genetically engineered male comprises:

A Y chromosome (also referred by the invention as the first sex chromosome) comprising the following exogenous sequences: (i), at least one nucleic acid sequence encoding at least one Cas9, or any fusion protein thereof; (ii), at least one nucleic acid sequence encoding at least one gRNA directed against at least one target sequence. Optionally, the target sequence is at least one protospacer comprised within at least one of the Jhdm2a, Sun4, Sun5, Spem1, Crem, Gopc, Hook1, and the Tbpl1 genes or any orthologs or homologs thereof. It should be noted that at least one of said nucleic acid sequences of (i) and (ii), is operably linked to at least one of at least one of: at least one inducible regulatory element, at least one transcriptional regulatory element, at least one post-translationally regulatory element and at least one temporally or spatially regulatory element. This particular embodiment is illustrated by Example 4.

Still further, in some embodiments, the target sequence targeted by the gRNAs may be at least one protospacer comprised within at least one of the JmjC-containing H3K9 demethylase (Jhdm2a), SUN domain-containing protein 4 (Sun4), SUN domain-containing protein 5 (Sun5), Spem1, Crem, Golgi-associated PDZ and coiled-coil motif-containing protein gene (Gopc), the hook microtubule tethering protein 1 (Hook1) and the TATA box-binding protein-like protein 1 (Tbpl1) genes, or any orthologs or homologs thereof.

More specifically, JmjC-containing H3K9 demethylase (Jhdm2a) also named lysine (K)-specific demethylase 3A (Kdm3a) encodes a zinc finger protein that contains a jumonji domain and may play a role in hormone-dependent transcriptional activation. Alternative splicing results in multiple transcript variants. It has been shown that JHDM2A plays a role in spermatogenesis and transition nuclear protein and protamine genes are direct targets of JHDM2A. In some embodiments, the target sequence may be comprised within the Jhdm2a gene. In some embodiments, the Jhdm2a gene may be of Mus musculus and may comprise the nucleic acid sequence as denoted by SEQ ID NO: 1 (Accession number: NM_001038695.3), encoding a protein comprising the amino acid sequence as denoted by SEQ ID NO: 2 (Accession number: NP_001033784.2). In some embodiments, this target may be applicable in case of a genetically engineered mouse. In yet some further embodiments, the Jhdm2a gene may be of Bos taurus and may comprise the nucleic acid sequence as denoted by SEQ ID NO: 3 (Accession number: NM_001192872.3), encoding a protein comprising the amino acid sequence as denoted by SEQ ID NO: 4 (Accession number: NP_001179801.1). In some embodiments, this target may be applicable in case of a genetically engineered cattle. In some embodiments, the Jhdm2a gene may be of Gallus gallus and may comprise the nucleic acid sequence as denoted by SEQ ID NO: 5 (Accession number: NM_001012891.1), encoding a protein comprising the amino acid sequence as denoted by SEQ ID NO: 6 (Accession number: NP_001012909.1). In some embodiments, this target may be applicable in case of a genetically engineered chicken.

Still further, SUN domain-containing protein 4 (Sun4), also named sperm associated antigen 4 (Spag4) is a spermatid nuclear membrane protein that has an essential function in coupling the manchette to the nuclear periphery. In the absence of SUN4, manchette microtubules appear highly disorganized and the nucleus itself fails to elongate. It is a member of the SUN (Sad1 and UNC84 domain containing)-domain proteins which are reported to reside on the nuclear membrane playing distinct roles in nuclear dynamics. In some embodiments, the target sequence may be comprised within the Sun4 gene. In some embodiments, the Sun4 gene may be of Mus musculus and may comprise the nucleic acid sequence as denoted by SEQ ID NO: 7 (Accession number: NM_139151.4), encoding a protein comprising the amino acid sequence as denoted by SEQ ID NO: 8 (Accession number: NP_631890.3). In some embodiments, this target may be applicable in case of a genetically engineered mouse. In some embodiments, the Sun4 gene may be of Bos taurus and may comprise the nucleic acid sequence as denoted by SEQ ID NO: 9 (Accession number: NM_001076507.2), encoding a protein comprising the amino acid sequence as denoted by SEQ ID NO: 10 (Accession number: NP_001069975.1). In some embodiments, this target may be applicable in case of a genetically engineered cattle. In some embodiments, the Sun4 gene may be of Gallus gallus and may comprise the nucleic acid sequence as denoted by SEQ ID NO: 11 (Accession number: XM_025143566.1), encoding a protein comprising the amino acid sequence as denoted by SEQ ID NO: 12 (Accession number: XP_024999334.1). In some embodiments, this target may be applicable in case of a genetically engineered chicken.

SUN domain-containing protein 5 (Sun5) is a member of the SUN family, that has an essential role in anchoring sperm head to the tail. In some embodiments, the target sequence may be comprised within the Sun5 gene. In some embodiments, the Sun5 gene may be of Mus musculus and may comprise the nucleic acid sequence as denoted by SEQ ID NO: 13 (Accession number: NM_029599.2), encoding a protein comprising the amino acid sequence as denoted by SEQ ID NO: 14 (Accession number: NP_083875.1). In some embodiments, this target may be applicable in case of a genetically engineered mouse. In some embodiments, the Sun5 gene may be of Bos taurus and may comprise the nucleic acid sequence as denoted by SEQ ID NO: 15 (Accession number: NM_001046165.2), encoding a protein comprising the amino acid sequence as denoted by SEQ ID NO: 16 (Accession number: NP_001039630.1). In some embodiments, this target may be applicable in case of a genetically engineered cattle. In some embodiments, the Sun5 gene may be of Gallus gallus and may comprise the nucleic acid sequence as denoted by SEQ ID NO: 17 (Accession number: XM_015296077.1), encoding a protein comprising the amino acid sequence as denoted by SEQ ID NO: 18 (Accession number: XP_015151563.1). In some embodiments, this target may be applicable in case of a genetically engineered chicken.

Spermatid maturation 1 (Spem1), as used herein, encodes a protein exclusively expressed in the cytoplasm of steps 14-16 elongated spermatids in the mouse testis. It has been shown that proper cytoplasm removal is a genetically regulated process requiring the participation of Spem1 and that lack of Spem1 causes sperm deformation and male infertility. In some embodiments, the target sequence may be comprised within the Spem1 gene. In some embodiments, the Spem1 gene may be of Mus musculus and may comprise the nucleic acid sequence as denoted by SEQ ID NO: 19 (Accession number: NM_028855.1), encoding a protein comprising the amino acid sequence as denoted by SEQ ID NO: 20 (Accession number: NP_083131.1). In some embodiments, this target may be applicable in case of a genetically engineered mouse. In some embodiments, the Spem1 gene may be of Bos taurus and may comprise the nucleic acid sequence as denoted by SEQ ID NO: 21 (Accession number: NM_001079586.2), encoding a protein comprising the amino acid sequence as denoted by SEQ ID NO: 22 (Accession number: NP_001073054.2). In some embodiments, this target may be applicable in case of a genetically engineered cattle. It should be noted that the Spem1 gene is not found in fly, zebrafish, and chicken. But is highly conserved in mammals.

Transcription activator CREM (cyclic AMP-responsive element modulator) is highly expressed in postmeiotic cells and is responsible for the activation of several haploid germ cell-specific genes involved in the structuring of the spermatozoon. In some embodiments, the target sequence may be comprised within the Crem gene. In some embodiments, the Crem gene may be of Mus musculus and may comprise the nucleic acid sequence as denoted by SEQ ID NO: 23 (Accession number: NM_013498.3), encoding a protein comprising the amino acid sequence as denoted by SEQ ID NO: 24 (Accession number: NP_038526.2). In some embodiments, this target may be applicable in case of a genetically engineered mouse. In some embodiments, the Crem gene may be of Bos taurus and may comprise the nucleic acid sequence as denoted by SEQ ID NO: 25 (Accession number: NM_001034710.3), encoding a protein comprising the amino acid sequence as denoted by SEQ ID NO: 26 (Accession number: NP_001029882.2). In some embodiments, this target may be applicable in case of a genetically engineered cattle. In some embodiments, the Crem gene may be of Gallus gallus and may comprise the nucleic acid sequence as denoted by SEQ ID NO: 27 (Accession number: NM_204387.2), encoding a protein comprising the amino acid sequence as denoted by SEQ ID NO: 28 (Accession number: NP_989718.2). In some embodiments, this target may be applicable in case of a genetically engineered chicken. Golgi-associated PDZ and coiled-coil motif-containing protein gene (Gopc) encodes a Golgi-associated protein that is predominantly localized at the trans-Golgi region in round spermatids. It has been shown that this protein plays an important role in acrosome formation during spermatogenesis. In some embodiments, the target sequence may be comprised within the Gopc gene. In some embodiments, the Gopc gene may be of Mus musculus and may comprise the nucleic acid sequence as denoted by SEQ ID NO: 29 (Accession number: NM_053187.3), encoding a protein comprising the amino acid sequence as denoted by SEQ ID NO: 30 (Accession number: NP_444417.2). In some embodiments, this target may be applicable in case of a genetically engineered mouse. In some embodiments, the Gopc gene may be of Bos taurus and may comprise the nucleic acid sequence as denoted by SEQ ID NO: 31 (Accession number: NM_001206157.1), encoding a protein comprising the amino acid sequence as denoted by SEQ ID NO: 32 (Accession number: NP_001193086.1). In some embodiments, this target may be applicable in case of a genetically engineered cattle. In some embodiments, the Gopc gene may be of Gallus gallus and may comprise the nucleic acid sequence as denoted by SEQ ID NO: 33 (Accession number: XM_015284489.1), encoding a protein comprising the amino acid sequence as denoted by SEQ ID NO: 34 (Accession number: XP_015139975.1). In some embodiments, this target may be applicable in case of a genetically engineered chicken.

Hook microtubule tethering protein 1 (Hook1) encodes a protein that plays an important role in Nuclear and manchette development in spermatids. In some embodiments, the target sequence may be comprised within the Hook1 gene. In some embodiments, the Hook1 gene may be of Mus musculus and may comprise the nucleic acid sequence as denoted by SEQ ID NO: 35 (Accession number: NM_030014.2), encoding a protein comprising the amino acid sequence as denoted by SEQ ID NO: 36 (Accession number: NP_084290.1). In some embodiments, this target may be applicable in case of a genetically engineered mouse. In some embodiments, the Hook1 gene may be of Bos taurus and may comprise the nucleic acid sequence as denoted by SEQ ID NO: 37 (Accession number: NM_001206870.2), encoding a protein comprising the amino acid sequence as denoted by SEQ ID NO: 38 (Accession number: NP_001193799.2). In some embodiments, this target may be applicable in case of a genetically engineered cattle. In some embodiments, the Hook1 gene may be of Gallus gallus and may comprise the nucleic acid sequence as denoted by SEQ ID NO: 39 (Accession number: NM_001006542.1), encoding a protein comprising the amino acid sequence as denoted by SEQ ID NO: 40 (Accession number: NP_001006542.1). In some embodiments, this target may be applicable in case of a genetically engineered chicken. Still further, TATA box-binding protein-like protein 1 (Tbpl1) gene is highly expressed in a finely regulated pattern in the testis during spermatogenesis and was shown to be essential for spermatogenesis. In some embodiments, the target sequence may be comprised within the Tbpl1 gene. In some embodiments, the Tbpl1 gene may be of Mus musculus and may comprise the nucleic acid sequence as denoted by SEQ ID NO: 41 (Accession number: NM_011603.5), encoding a protein comprising the amino acid sequence as denoted by SEQ ID NO: 42 (Accession number: NP_035733.1). In some embodiments, this target may be applicable in case of a genetically engineered mouse. In some embodiments, the Tbpl1 gene may be of Bos taurus and may comprise the nucleic acid sequence as denoted by SEQ ID NO: 43 (Accession number: NM_001076290.2), encoding a protein comprising the amino acid sequence as denoted by SEQ ID NO: 44 (Accession number: NP_001069758.1). In some embodiments, this target may be applicable in case of a genetically engineered cattle. In some embodiments, the Tbpl1 gene may be of Gallus gallus and may comprise the nucleic acid sequence as denoted by SEQ ID NO: 45 (Accession number: NM_204755.1), encoding a protein comprising the amino acid sequence as denoted by SEQ ID NO: 46 (Accession number: NP_990086.1). In some embodiments, this target may be applicable in case of a genetically engineered chicken.

It should be note that a “target sequence” comprised within the target gene as indicated above, may be according to some embodiments, at least one target sequence that forms at least one protospacer recognized and targeted by the at least one gRNA encoded by the “first” sex chromosome of the transgenic heterogametic organism of the invention.

In some embodiments, the transgenic organisms of the invention, as well as the systems and methods disclosed herein after, may be from the mammalian family Mustelidae, more specifically a mink. “Mink” are dark-colored, semiaquatic, carnivorous mammals of the genera Neogale and Mustela and part of the family Mustelidae, which also includes weasels, otters, and ferrets. There are two extant species referred to as “mink”: the “American mink” (Neogale vison) and the “European mink” (Mustela lutreola). The extinct sea mink was related to the American mink but was much larger.

The American mink's fur has been highly prized for use in clothing and is the most popular fur traded worldwide. There are differences between female fur and male fur: female mink fur is generally lighter in weight, softer and more supple than male mink fur. Additionally, female pelts have higher luster, are very silky and soft and are more adaptable to tailoring and draping. Male mink is often used to create a different fashion look and is also of excellent quality. Selection of male/female ratio in mink may thus be desired.

It yet some further embodiments, the genetically engineered eukaryotic heterogametic organism provided by the invention may be an avian organism. In more specific embodiments, such avian organism may be any one of a domesticated and an undomesticated bird.

In yet some further embodiments, the genetically engineered organisms provided herein, as well as in the systems and methods of the invention, may be applicable for avian organisms. In yet some further specific embodiments, the invention may provide birds as the genetically engineered organisms. More specifically, domesticated and undomesticated birds are also suitable organisms for the invention.

Therefore, in certain embodiments, the avian organism of the invention may be any one of a domesticated and an undomesticated bird. In more specific embodiment, the avian organism may be any one of a poultry or a game bird. In some specific embodiments, the avian organism may be of the order Galliformes which comprise without limitation, chicken, quail, turkey, duck, Gallinacea sp, goose, pheasant and other fowl. The term “avian” relates to any species derived from birds characterized by feathers, toothless beaked jaws, the laying of hard-shelled eggs, a high metabolic rate, a four-chambered heart, and a lightweight but strong skeleton. The term “hen” includes all females of the avian species. In yet some specific embodiments, the genetically engineered organisms, systems and methods of the invention may be also suitable for chicken. Chimeric avian are generated which are derived in part from the modified embryonic stem cells or zygote cells, capable of transmitting the genetic modifications through the germ line. Mating avian strains containing exogenous sequences should result in progenies displaying the desired sex. Still further, genetically engineered avian can be produced by different methods, some of which are discussed below and in the examples section. Among the avian cells suitable for transformation for generating genetically engineered animals are primordial germ cells (PGC), sperm cells and zygote cells (including embryonic stem cells). Sperm cells can be transformed with DNA constructs by any suitable method, including electroporation, microparticle bombardment, lipofection and the like. The sperm can be used for artificial insemination of avian. Progeny of the inseminated avian can be examined for the exogenous sequence as described above. The developmental stage of chicken is as detailed in the following: the chicken germ-line develops from a small population of primordial germ cells (PGCs), migrating to the genital ridge from an extragonadal site, while undergoing phases of active migration, as well as passive circulation in the bloodstream. PGCs are located in the center of the epiblast of freshly laid, un-incubated egg, a developmental stage referred as stage X. During incubation PGCs migrate anteriorly and accumulate at the germinal crescent of stage 10HH embryo (approximately 33-38 hours of incubation), considered the main site for intravasation. Later, PGCs can be found in the circulation from stage 12HH to 17HH (approximately from 45 to 64 hours of incubation), reaching a peak concentration in stage 14HH (approximately 50-53 hours of incubation). PGCs leave the circulation at a site adjacent to the gonad anlage at the intermediate mesoderm, where they can be found as early as stage 15HH (55-55 hours of incubation). PGCs reach the genital ridge by active migration along the dorsal mesentery, and colonize both gonads, where they later differentiate into spermatogonia or oogonia. “Primordial germ cells (PGCs)”, as used herein relates to germline stem cells that serve as progenitors of the gametes and give rise to pluripotent embryonic stem cells. The cells in the gastrulating embryo that are signaled to become PGCs during embryogenesis, migrate into the genital ridges which becomes the gonads, and differentiate into mature gametes.

Newly hatched avian can be tested for the presence of the target construct sequences, for example by examining a biological sample thereof, e.g., a blood sample. After the avian have reached maturity, they are bred, and their progeny may be examined to determine whether the exogenous integrated sequences are transmitted through the germ line.

In yet some more specific embodiments, the genetically engineered eukaryotic heterogametic organism provided by the invention is a domesticated bird, specifically, a chicken. In such case, the heterogametic organism is a female (ZW).

Thus, in cases where the desired sex is female (ZW), the first sex chromosome is the Z chromosome, and the second sex chromosome is the W chromosome. Such female produces predominantly gamete cells comprising the W chromosomes.

However, in cases where the desired sex is male (ZZ), the first sex chromosome is the W chromosome, and the second sex chromosome is the Z chromosome. In such case the genetically engineered hen provided by the invention produces predominantly gamete cells comprising the Z chromosome.

In some additional and specific embodiments, the present disclosure may be applicable for predatory birds. “Predatory birds” or “Birds of prey”, also known as “raptors”, are hypercarnivorous bird that species actively hunt and feed on other vertebrates (mainly mammals, reptiles and other smaller birds). In addition to speed and strength, these predators have keen eyesight for detecting prey from a distance or during flight, strong feet with sharp talons for grasping or killing prey, and powerful, curved beaks for tearing off flesh. Raptors include for example Eagles, Falcon, kestrel, Kite, Buzzard, harrier, Owls, vulture and seriemas. Biasing the sex ratio (e.g., towards females) in non-domesticated birds may be useful particularly in protection of in endangered species.

Still further, in some embodiments, the invention may be applicable for insects and thus, may provide genetically engineered insects, systems, methods and uses thereof. Insects or Insecta are hexapod invertebrates and the largest group within the arthropod phylum. Definitions and circumscriptions vary; usually, insects comprise a class within the Arthropoda. As used here, the term Insecta is synonymous with Ectognatha. Insects have a chitinous exoskeleton, a three-part body (head, thorax and abdomen), three pairs of jointed legs, compound eyes and one pair of antennae. Insects are the most diverse group of animals; they include more than a million described species and represent more than half of all known living organisms. Insects can be divided into two groups historically treated as subclasses: wingless insects, known as Apterygota, and winged insects, known as Pterygota. The Apterygota consist of the primitively wingless order of the silverfish (Zygentoma). Archaeognatha make up the Monocondylia based on the shape of their mandibles, while Zygentoma and Pterygota are grouped together as Dicondylia. The Zygentoma themselves possibly are not monophyletic, with the family Lepidotrichidae being a sister group to the Dicondylia (Pterygota and the remaining Zygentoma). Paleoptera and Neoptera are the winged orders of insects differentiated by the presence of hardened body parts called sclerites, and in the Neoptera, muscles that allow their wings to fold flatly over the abdomen. Neoptera can further be divided into incomplete metamorphosis-based (Polyneoptera and Paraneoptera) and complete metamorphosis-based groups. It should be noted that the present invention is applicable for any of the insects of any of the groups and species disclosed herein.

Still further, many insects are considered pests by humans. Insects commonly regarded as pests include those that are parasitic (e.g. lice, bed bugs), transmit diseases (mosquitoes, flies), damage structures (termites), or destroy agricultural goods (locusts, weevils). Insects considered pests of some sort occur among all major living orders with the exception of Ephemeroptera (mayflies), Odonata, Plecoptera (stoneflies), Embioptera (webspinners), Trichoptera (caddisflies), Neuroptera (in the broad sense), and Mecoptera (also, the tiny groups Zoraptera, Grylloblattodea, and Mantophasmatodea). Of particular interest of this group is the Mosquito. More specifically, in some embodiments, the invention may be suitable for insects such as mosquito for example. Mosquitoes are a group of about 3500 species of small insects that are a type of fly (order Diptera). Within that order they constitute the family Culicidae. Superficially, mosquitoes resemble crane flies (family Tipulidae) and chironomid flies (family Chironomidae). It should be appreciated that in some embodiments, the term mosquito, as used herein includes all genera encompassed by the subfamilies Anophelinae and Culicinae. In yet some further embodiments, mosquito as used herein include, but is not limited to any mosquito of the following genera, Aedeomyia, Aedes, Anopheles, Armigeres, Ayurakitia, Borachinda, Coquillettidia, Culex, Culiseta, Deinocerites, Eretmapodites, Ficalbia, Galindomyia, Haemagogus, Heizmannia, Hodgesia, Isostomyia, Johnbelkinia, Kimia, Limatus, Lutzia, Malaya, Mansonia, Maorigoeldia, Mimomyia, Onirion, Opifex, Orthopodomyia, Psorophora, Runchomyia, Sabethes, Shannoniana, Topomyia, Toxorhynchites, Trichoprosopon, Tripteroides, Udaya, Uranotaenia, Verrallina, and Wyeomyia. Females of most species are ectoparasites, whose tube-like mouthparts (called a proboscis) pierce the hosts' skin to consume blood. Though the loss of blood is seldom of any importance to the victim, the saliva of the mosquito often causes an irritating rash that is a serious nuisance. Much more serious though, are the roles of many species of mosquitoes as vectors of diseases. In passing from host to host, some transmit extremely harmful infections such as malaria, yellow fever, Chikungunya, West Nile virus, dengue fever, filariasis, Zika virus and other arboviruses, rendering it the deadliest animal family in the world. Therefore, reducing the population of mosquitoes and particularly of female mosquitoes is of great relevance. Sex is determined in most mosquito by heterogamety, males being XY and females being XX.

In yet some further embodiments, the invention may be applicable for Aedes aegypti mosquito. More specifically, the dengue, yellow fever, chikungunya and zika vector, Aedes aegypti, has a dominant male-determining sex locus (M) on chromosome 1, for which males are heterozygous (Mm). This locus is primarily responsible for sex determination, however male and female chromosomes are also cytologically distinct. The male-determining factor (M factor) nix, an M-linked myosin heavy chain gene, myo-sex, and two sex determination transcription factors have been characterized but little else is known about the specific genes contributing dimorphic phenotypes in aedine mosquitoes. Thus, in some specific embodiments, since the male is the heterogametic organism, and the M gene may be equivalent to the heterogametic chromosome, where the mm homozygotes are equivalent to the homogametic organism (female).

Still further, some insects, like wasps, bees, butterflies and ants, pollinate flowering plants. Pollination is a mutualistic relationship between plants and insects. This greatly increases diversity in plants and significantly affects agriculture. The present invention provides genetically engineered organisms, systems and methods for selecting insect sex by producing gametes of only the desired sex, and therefore may be of a high economic value, when applied for insects useful in agriculture. Of particular interest in some embodiments of the present invention is the bee. Bees are flying insects closely related to wasps and ants, known for their role in pollination and, in the case of the best-known bee species, the western honey bee, for producing honey and beeswax. Bees are a monophyletic lineage within the superfamily Apoidea and are presently considered a clade, called Anthophila. There are nearly 20,000 known species of bees in seven recognized biological families, specifically, Andrenidae, Apidae, Colletidae, Halictidae, Megachilidae, Melittidae, Stenotritidae. Some species including honey bees, bumblebees, and stingless bees live socially in colonies. It should be understood that the present invention encompasses any of the bee species of any of the bee families indicated herein.

It should be understood that the present invention further provides any of the genetically engineered mosquitos, bees, or insects of the invention, any cells, progenies and embryos thereof, and any use or product thereof.

In yet some further embodiments, the present invention may be also applicable to the aquaculture industry. Aquaculture, also known as aquafarming, is the farming of fish, crustaceans, molluscs, aquatic plants, algae, and other organisms. Aquaculture involves cultivating freshwater and saltwater populations under controlled conditions, and can be contrasted with commercial fishing, which is the harvesting of wild fish. It should be noted that the present invention further pertains to Mariculture that refers to aquaculture practiced in marine environments and in underwater habitats. Unisex populations are often preferred by the aquaculture industry. More specifically, male fingerlings are preferred by tilapia growers and females by salmonid fish arms. Sex chromosomes were identified in both salmonid and in tilapia. All salmonids have the XX/XY mode of sex determination while different tilapine species may have the XX/XY or ZZ/ZW mode (reviewed in Cnaani et al. 2008 Sex Dev; 2:43-54).

More specifically, in the Nile tilapia O. niloticus, the male is heterogametic (XX/XY system), while in O. aureus from Israel, O. karongae and Tilapia mariae, the female is heterogametic (WZ/ZZ system). Thus, the present invention in some embodiments thereof also encompasses genetically engineered fish, as well as systems and methods producing and using these fish. Fish, as used herein refer to gill-bearing aquatic craniate animals that lack limbs with digits. They form a sister group to the tunicates, together forming the olfactores. Included in this definition are the living hagfish, lampreys, and cartilaginous and bony fish as well as various related groups. It should be noted that the present invention relates to any group, class, subclass or any family of fish. Specifically, any fish of the following families, specifically, Cyprinidae, Gobiidae, Cichlidae, Characidae, Loricariidae, Balitoridae, Serranidae, Labridae, and Scorpaenidae.

In some specific embodiments, the genetically engineered fish of the invention may be of the genus tilapia. Tilapia, as used herein is the common name for nearly a hundred species of cichlid fish from the tilapiine cichlid tribe. Tilapia are mainly freshwater fish inhabiting shallow streams, ponds, rivers, and lakes, and less commonly found living inbrackish water. It should be noted that the invention relates to any species of tilapia.

In more specific embodiments, the tilapia fish may be of the Oreochromis niloticus species. In yet other embodiment, the tilapia fish may be of any one of the species Oreochromis aureus, Oreochromis karongae or Pelmatolapia mariae.

Still further, the invention may be useful for crustaceans organisms. Crustaceans, as used herein, form a large, diverse arthropod taxon which includes crabs, lobsters, crayfish, shrimp, krill, woodlice, and barnacles, that are all encompassed by the present invention. The crustacean group is usually considered as a paraphyletic group and comprises all animals in the Pancrustacea clade other than hexapods. Some crustaceans are more closely related to insects and other hexapods than they are to certain other crustaceans. In some embodiments, such crustaceans may be shrimp. The term shrimp is used to refer to decapod crustaceans and covers any of the groups with elongated bodies and a primarily swimming mode of locomotion i.e. Caridea and Dendrobranchiata. In shrimps, the female heterogamety of the ZZ/ZW type enables sex determination. The female shrimps are preferred since they are brighter in color and are larger compared to the males.

As mentioned above, the invention concerns any eukaryotic organism and as such may be also applicable for members of the biological kingdom Plantae. Particularly, in certain plants that display heterogametic sex determination. Thus, in some further embodiments, the eukaryotic heterogametic organisms of the invention may be of the biological kingdom Plantae.

In more specific embodiments, the genetically engineered eukaryotic heterogametic organism may be a dioecious plant.

More specifically, plants presenting biparental reproduction. In dioecious plants the male and female reproductive systems occur on separate plants. While both plants produce flowers, one plant has the male reproductive parts, and the other plant has the female parts. This is unlike a monoecious plant, which has both male and female flowers.

Still further, in some embodiments, the genetically engineered eukaryotic heterogametic organism may be a dioecious plant, specifically, of the family Cannabaceae.

In some specific embodiments, the plant of the family Cannabaceae may be any one of Cannabis (hemp, marijuana) and Humulus (hops). In more specific embodiments, the plant of the family Cannabaceae may be Cannabis (hemp, marijuana). Thus, in some embodiments, the invention provides at least one heterogametic genetically engineered Cannabis plant, that is a male plant. More specifically, Cannabis is a dioecious plant producing either male or female reproductive organs i.e., cannabis grows as either a male or female plant. Removing male plants from crop allows female plants to grow large, an unfertilized flower named sensimilla. Sensimilla is a highly concentrated type of cannabis and does not contain seeds. Sensimilla refers to many strains of marijuana where the female plant is allowed to only produce flowers but is left unfertilized so does not progress on to produce seeds. Its unfertilized state contributes to the plant's ability to produce higher levels of tetrahydrocannabinol (THC) and other cannabinoids.

Currently, one way to obtain only female plant is by using feminized cannabis seeds. These seeds are produced by causing the monoecious, or hermaphrodite condition in a female cannabis plant. This is achieved through several methods such as spraying the plant with a solution of colloidal silver, or with gibberellic acid. Feminized seeds produce plants that are nearly identical to this self-pollinated female parent plant, as only one set of genes is present and will not produce any male plants. Another way is by collecting pollens may for fertilizing other females. In both cases, only feminized seeds will be produced because only the X chromosome is found in the pollen and the female gamete. However, most feminized seeds end up being hermaphrodites, which results in flowers possessing seeds and reduces yields. Genetically, the cannabis plant sex is regulated by two chromosomes the X and Y chromosomes. A plant with two X chromosomes becomes female. A plant with an X and Y chromosome turns into a male.

In yet some further embodiments, the plant of the family Cannabaceae may be Humulus (hops). More specifically, Hops are the flowers (also called seed cones or strobiles) of the hop plant Humulus lupulus. They are used primarily as a flavoring and stability agent in beer, to which they impart bitter, zesty, or citric flavors, though they are also used for various purposes in other beverages and herbal medicine. Male and female flowers of the hop plant usually develop on separate plants (dioecious plant), although fertile monoecious individuals appear occasionally. Because viable seeds are undesirable for brewing beer, only female plants are grown in hop fields, thus preventing pollination. Female plants are propagated vegetatively, and male plants are culled if plants are grown from seeds. The plant Humulus lupulus has heteromorphic sex chromosomes is hop. The genotypes carrying XX or XY chromosomes correspond to female and male plants, respectively.

As indicated herein above, the present disclosure provides various genetically modified heterogametic organisms. In some specific and non-limiting embodiments, the disclosed genetically modified organisms may carry one sex chromosome that carry exogeneous nucleic acid sequence. Some specific and non-limiting embodiments for the genetically modified organisms are provided by the following Examples.

Lines #1-5 disclosed in the present disclosure all rely on the same principle. The Y gamete separates from the X gamete. Therefore, after separation, only the engineered gamete produces the nucleic acid modifier (e.g., dCas9). Because it produces it at an essential stage of gamete formation and targets genes essential for gamete cell formation, stability, viability and function, the gamete cannot mature. When the Y gamete contains the construct (indicated herein as the first sex chromosome), then the male gametes do not mature and vice versa. The difference between the various lines is in the stability of the nucleic acid modifier component (e.g., dCas9), and in the targeted genes. The stability is dictated by different tags that reduce or stabilize the dCas9. In some specific embodiments, the first sex chromosome of the genetically modified organism of the present disclosure comprises nucleic acid sequence encoding the dCas-KRAB-MeCP2, under the Tet-off promoter, and sequence encoding at least one gRNA targeting the Spem1 Jhdm2a Sun4 Sun5 Crem genes. Such genetically modified organism is illustrated by line #1, as shown in Table 4. In some alternative embodiments the first sex chromosome of the genetically modified organism of the present disclosure comprises nucleic acid sequence encoding the dCas-KRAB-MeCP2, under the Tet-off promoter, and sequence encoding at least one gRNA targeting the Spem1 gene. Such genetically modified organism is illustrated by line #2, as shown in Table 4, Examples 6 and 15. In some particular and non-limiting embodiments, such genetically modified organism may comprise the knock in insert as denoted by SEQ ID NO: 53.

In some further specific embodiments, the first sex chromosome of the genetically modified organism of the present disclosure comprises nucleic acid sequence encoding the dCas-KRAB-MeCP2, under the Tet-off promoter. The dCas is further regulated by the inclusion of the degron UbM. The genetically modified sex chromosome of this GMO organism further comprises a sequence encoding at least one gRNA targeting the Spem1 Jhdm2a Sun4 Sun5 Crem genes. Such genetically modified organism is illustrated by line #3, as shown in Table 4.

In some further specific embodiments, the first sex chromosome of the genetically modified organism of the present disclosure comprises nucleic acid sequence encoding the dCas-KRAB-MeCP2, under the Tet-off promoter. The dCas is further regulated by the inclusion of the degron UbD. The genetically modified sex chromosome of this GMO organism further comprises a sequence encoding at least one gRNA targeting the Spem1 Jhdm2a Sun4 Sun5 Crem genes. Such genetically modified organism is illustrated by line #4, as shown in Table 4. In some particular and non-limiting embodiments, such genetically modified organism may comprise the knock in insert as denoted by SEQ ID NO: 74.

In some further specific embodiments, the first sex chromosome of the genetically modified organism of the present disclosure comprises nucleic acid sequence encoding the dCas-KRAB-MeCP2, under the Tet-off promoter. The dCas is further regulated by the inclusion of the degron UbK. The genetically modified sex chromosome of this GMO organism further comprises a sequence encoding at least one gRNA targeting the Spem1 Jhdm2a Sun4 Sun5 Crem genes. Such genetically modified organism is illustrated by line #5, as shown in Table 4.

In some further specific embodiments, the first sex chromosome of the genetically modified organism of the present disclosure comprises nucleic acid sequence encoding the Cas9 under the Tet-off promoter. The genetically modified sex chromosome of this GMO organism further comprises a sequence encoding at least one gRNA targeting the Spem1 Jhdm2a Sun4 Sun5 Crem genes. Such genetically modified organism is illustrated by line #6, as shown in Table 4. In some particular and non-limiting embodiments, such genetically modified organism may comprise the knock in insert as denoted by SEQ ID NO: 79. It should be understood that in some embodiments, this line may only work as a GMO after crossing with lines 17-19. Tetracycline should be added during its production so that Cas9 does not damage the DNA. After crossing with a strain encoding a Cas9 inhibitor in the opposite gamete, the strain may be tested. The principle here is that throughout its expression, the Cas9 is inhibited in somatic cells due to the presence of the AcrII4A inhibitor provided by lines #17-19. Therefore, it does not cause any damage to the DNA. Only after separation, does it cleave the DNA and destroys the gamete.

Still further, the following lines #7-8 are similar in principle to lines 1-5, however, the promoter of dCas9 is constitutive. These two lines comprise degron in dCas9, specifically, the Degradation domain (DD). This domain protects from degradation of the dCas9 if Shield1 is added. Therefore, to produce single sex—the Shield1 should be added. In more specific embodiments, the first chromosome of the genetically modified organism of the present disclosure comprises nucleic acid sequence encoding the dCas-KRAB-MeCP2, under the CAGG promoter. The dCas is further regulated by the inclusion of the degron DD. The genetically modified sex chromosome of this GMO organism further comprises a sequence encoding at least one gRNA targeting the Spem1 Jhdm2a Sun4 Sun5 Crem genes. Such genetically modified organism is illustrated by line #7, as shown in Table 4. In some particular and non-limiting embodiments, such genetically modified organism may comprise the knock in insert as denoted by SEQ ID NO: 81. In some further specific embodiments, the first chromosome of the genetically modified organism of the present disclosure comprises nucleic acid sequence encoding the dCas-KRAB-MeCP2, under the CAGG promoter. The dCas is further regulated by the inclusion of the degron DD. The genetically modified sex chromosome of this GMO organism further comprises a sequence encoding at least one gRNA targeting the Spem1 gene. Such genetically modified organism is illustrated by line #8, as shown in Table 4. In some particular and non-limiting embodiments, such genetically modified organism may comprise the knock in insert as denoted by SEQ ID NO: 83.

Still further, Lines #9-10 rely on the stability of small interfering RNAs in the gamete after separation. The gamete having it cannot mature, whereas the gamete lacking it develops normally. The cassette is under regulation of the tet-off promoter. Therefore, to produce both sexes, tetracycline should be added. In some further specific embodiments, the first chromosome of the genetically modified organism of the present disclosure comprises nucleic acid sequence encoding at least one shRNA under the Tet-off promoter. The shRNA of this construct targets the Spem1 Jhdm2a Sun4 Sun5 Crem genes. Such genetically modified organism is illustrated by line #9, as shown in Table 4. In some further specific embodiments, the first sex chromosome of the genetically modified organism of the present disclosure comprises nucleic acid sequence encoding at least one shRNA under the Tet-off promoter. The shRNA of this construct targets the Spem1 gene. Such genetically modified organism is illustrated by line #10, as shown in Table 4.

In yet some further embodiments, lines #11-14 produce the dCas9 or Cas9 under the natural promoter of one of the gamete development genes. This promoter is active only at the late maturation stage of the gamete, and therefore does not harm somatic cells. Upon expression of the dCas9 or Cas9 in the gamete, the gamete development is halted, or the gamete is destroyed. This results in single sex gametes. The degron of dCas9 or Cas9 may be the DD that protects from degradation of the dCas9 if Shield1 is added.

In some further specific embodiments, the first chromosome of the genetically modified organism of the present disclosure comprises nucleic acid sequence encoding the dCas-KRAB-MeCP2, under the Spem1/Akap4 promoter. The dCas is further regulated by the inclusion of the degron DD. The genetically modified sex chromosome of this GMO organism further comprises a sequence encoding at least one gRNA targeting the Spem1 Jhdm2a Sun4 Sun5 Crem genes. Such genetically modified organism is illustrated by line #11, as shown in Table 4. In some particular and non-limiting embodiments, such genetically modified organism may comprise the knock in insert as denoted by SEQ ID NO: 85. In some further specific embodiments, the first chromosome of the genetically modified organism of the present disclosure comprises nucleic acid sequence encoding the dCas-KRAB-MeCP2, under the Spem1/Akap4 promoter. The dCas is further regulated by the inclusion of the degron DD. The genetically modified sex chromosome of this GMO organism further comprises a sequence encoding at least one gRNA targeting the Spem1 gene. Such genetically modified organism is illustrated by line #12, as shown in Table 4. In some particular and non-limiting embodiments, such genetically modified organism may comprise the knock in insert as denoted by SEQ ID NO: 87.

Still further, lines #15-16 are similar to lines #11-14, but the degron is different. “LID” is a domain that destabilizes the protein in the presence of Shield1. Therefore, to produce both sexes, Shield1 should be added. In some further specific embodiments, the first chromosome of the genetically modified organism of the present disclosure comprises nucleic acid sequence encoding the Cas9, under the Spem1/Akap4 promoter. The Cas9 is further regulated by the inclusion of the degron DD. The genetically modified sex chromosome of this GMO organism further comprises a sequence encoding at least one gRNA targeting the Spem1 Jhdm2a Sun4 Sun5 Crem genes. Such genetically modified organism is illustrated by line #13, as shown in Table 4. In some further specific embodiments, the first sex chromosome of the genetically modified organism of the present disclosure comprises nucleic acid sequence encoding the Cas9, under the Spem1/Akap4 promoter. The Cas9 is further regulated by the inclusion of the degron DD. The genetically modified sex chromosome of this GMO organism further comprises a sequence encoding at least one gRNA targeting the Spem1 gene. Such genetically modified organism is illustrated by line #14, as shown in Table 4. In some further specific embodiments, the first sex chromosome of the genetically modified organism of the present disclosure comprises nucleic acid sequence encoding the Cas9, under the Spem 1/Akap4 promoter. The Cas9 is further regulated by the inclusion of the degron LID. The genetically modified sex chromosome of this GMO organism further comprises a sequence encoding at least one gRNA targeting the Spem1 Jhdm2a Sun4 Sun5 Crem genes. Such genetically modified organism is illustrated by line #15, as shown in Table 4 . . . . In some further specific embodiments, the first sex chromosome of the genetically modified organism of the present disclosure comprises nucleic acid sequence encoding the Cas9, under the Spem1/Akap4 promoter. The Cas9 is further regulated by the inclusion of the degron LID. The genetically modified sex chromosome of this GMO organism further comprises a sequence encoding at least one gRNA targeting the Spem1 gene. Such genetically modified organism is illustrated by line #16, as shown in Table 4.

It should be noted that lines #17-19 may be crossed with lines 1-16 to provide an additional control on the dCas9 or Cas9 activity. The AcrIIA4 is an inhibitor of dCas9 and Cas9 protein. Its construction into the gamete that is desired to survive, may enhance the survival, as it inhibits the dCas9\Cas9 activity. The lines 1-16 engineered on the Y chromosome to produce only females, should be crossed with lines 17-19 engineered on the X chromosome (in case females are desired), and vice versa. The expression is different between the lines and on one of them, DD is added so that Shield 1 can be used to control the level of this inhibitor. If Shield1 is added. then the stability of the inhibitor is enhanced. However, despite the advantage of having an additional control of the Cas9, the resulting progeny is GMO because the AcrII4A inhibitor is inherited in the surviving sex (females). Thus, in some embodiments, the sex chromosome of the genetically modified homogametic organism of the present disclosure comprises nucleic acid sequence encoding the AcrIIA4, under the CAGG promoter. Such genetically modified organism is illustrated by line #17, as shown in Table 5.

In some other embodiments, the sex chromosome of the genetically modified homogametic organism of the present disclosure comprises nucleic acid sequence encoding the AcrIIA4, under the SV40 promoter. Such genetically modified organism is illustrated by line #18, as shown in Table 5. In some alternative embodiments, the sex chromosome of the genetically modified homogametic organism of the present disclosure comprises nucleic acid sequence encoding the AcrIIA4, under the CAGG promoter. The AcrIIA4 further comprises the degron DD. Such genetically modified organism is illustrated by line #19, as shown in Table 5. In some particular and non-limiting embodiments, such genetically modified organism may comprise the knock in insert as denoted by SEQ ID NO: 89.

Lines #20-27 rely on the principles of toxin-antitoxins. The toxin (dCas9) is more stable than the antitoxin (AcrII4A). Therefore, as long as both are expressed, no activity is detected. In this case −dCas9 does not inhibit transcription. However, if both are not expressed, after separation of the gametes, then the more stable toxin starts acting and repressing gene expression. Therefore, the gamete with the construct will survive, and the other will not. To save the other gamete, a degradation domain (DD) on the antitoxin can stabilize the anti-toxin and thus save both gametes. The Shield1 can be given at high doses for producing both sexes. It can be titrated at different concentrations to determine the most efficient stability required for the antitoxin to produce the best desired results. Different degrons are added to the dCas9 to determine optimal stability of it. Different targets are also combined to determine optimal construct.

In some further specific embodiments, the first sex chromosome of the genetically modified organism of the present disclosure comprises nucleic acid sequence encoding the dCas-KRAB-MeCP2, under the CAGG promoter. The first sex chromosome further comprises nucleic acid sequence encoding the AcrIIA4, that is further regulated by the inclusion of the DD degron sequence. The genetically modified sex chromosome of this GMO organism further comprises a sequence encoding at least one gRNA targeting the Spem1 Jhdm2a Sun4 Sun5 Crem genes. Such genetically modified organism is illustrated by line #20, as shown in Table 6. In some particular and non-limiting embodiments, such genetically modified organism may comprise the knock in insert as denoted by SEQ ID NO: 110.

In some further specific embodiments, the first sex chromosome of the genetically modified organism of the present disclosure comprises nucleic acid sequence encoding the dCas-KRAB-MeCP2, under the CAGG promoter. The first sex chromosome further comprises nucleic acid sequence encoding the AcrIIA4, that is further regulated by the inclusion of the DD degron sequence. The genetically modified sex chromosome of this GMO organism further comprises a sequence encoding at least one gRNA targeting the Spem1 gene. Such genetically modified organism is illustrated by line #21, as shown in Table 6. In some further specific embodiments, the first sex chromosome of the genetically modified organism of the present disclosure comprises nucleic acid sequence encoding the dCas-KRAB-MeCP2, under the CAGG promoter. The dCas is further regulated by the inclusion of the degron UbM. The first sex chromosome further comprises nucleic acid sequence encoding the AcrIIA4, that is further regulated by the inclusion of the DD degron sequence. The genetically modified sex chromosome of this GMO organism further comprises a sequence encoding at least one gRNA targeting the Spem1 Jhdm2a Sun4 Sun5 Crem genes. Such genetically modified organism is illustrated by line #22, as shown in Table 6. In some further specific embodiments, the first sex chromosome of the genetically modified organism of the present disclosure comprises nucleic acid sequence encoding the dCas-KRAB-MeCP2, under the CAGG promoter. The dCas is further regulated by the inclusion of the degron UbM. The first sex chromosome further comprises nucleic acid sequence encoding the AcrIIA4, that is further regulated by the inclusion of the DD degron sequence. The genetically modified sex chromosome of this GMO organism further comprises a sequence encoding at least one gRNA targeting the Spem1 gene. Such genetically modified organism is illustrated by line #23, as shown in Table 6.

In some further specific embodiments, the first sex chromosome of the genetically modified organism of the present disclosure comprises nucleic acid sequence encoding the dCas-KRAB-MeCP2, under the CAGG promoter. The dCas is further regulated by the inclusion of the degron UbD. The first sex chromosome further comprises nucleic acid sequence encoding the AcrIIA4, that is further regulated by the inclusion of the DD degron sequence. The genetically modified sex chromosome of this GMO organism further comprises a sequence encoding at least one gRNA targeting the Spem1 Jhdm2a Sun4 Sun5 Crem genes. Such genetically modified organism is illustrated by line #24, as shown in Table 6. In some further specific embodiments, the first sex chromosome of the genetically modified organism of the present disclosure comprises nucleic acid sequence encoding the dCas-KRAB-MeCP2, under the CAGG promoter. The dCas is further regulated by the inclusion of the degron UbD. The first sex chromosome further comprises nucleic acid sequence encoding the AcrIIA4, that is further regulated by the inclusion of the DD degron sequence. The genetically modified sex chromosome of this GMO organism further comprises a sequence encoding at least one gRNA targeting the Spem1 gene. Such genetically modified organism is illustrated by line #25, as shown in Table 6. In some further specific embodiments, the first sex chromosome of the genetically modified organism of the present disclosure comprises nucleic acid sequence encoding the dCas-KRAB-MeCP2, under the CAGG promoter. The dCas is further regulated by the inclusion of the degron UbK. The first sex chromosome further comprises nucleic acid sequence encoding the AcrIIA4, that is further regulated by the inclusion of the DD degron sequence. The genetically modified sex chromosome of this GMO organism further comprises a sequence encoding at least one gRNA targeting the Spem1 Jhdm2a Sun4 Sun5 Crem genes. Such genetically modified organism is illustrated by line #26, as shown in Table 6. In some further specific embodiments, the first sex chromosome of the genetically modified organism of the present disclosure comprises nucleic acid sequence encoding the dCas-KRAB-MeCP2, under the CAGG promoter. The dCas is further regulated by the inclusion of the degron UbK. The first sex chromosome further comprises nucleic acid sequence encoding the AcrIIA4, that is further regulated by the inclusion of the DD degron sequence. The genetically modified sex chromosome of this GMO organism further comprises a sequence encoding at least one gRNA targeting the Spem1 gene. Such genetically modified organism is illustrated by line #27, as shown in Table 6. Still further, in some embodiments, the disclosed lines, and specifically, lines #1-16 and 20-27, may be constructed either in the sex chromosome that characterizes the heterogametic organism (e.g., the y chromosome), or the chromosome that characterizes the homogametic chromosome (e.g., the X chromosome). It should be noted that these embodiments of the disclosed genetically modified organism are applicable for any eukaryotic organism disclosed by the present disclosure. For insertion in the Y or the X chromosomes, the disclosed exogeneous nucleic acid sequence may further comprise the appropriate homology arms. In some specific and non limiting embodiments, the exogeneous nucleic acid sequence is inserted into the Y chromosome. In yet some particular embodiments, the exogeneous nucleic acid sequence may be inserted at the uty gene. According to such embodiments, where the disclosed genetically modified organism is a mouse, the left homology arm may comprise the nucleic acid sequence as denoted by SEQ ID NO: 52, and the right homology arm may comprise the nucleic acid sequence as denoted by SEQ ID NO: 55. Still further, in some alternative embodiments, the exogeneous nucleic acid sequence is inserted into the X chromosome. In yet some particular embodiments, the exogeneous nucleic acid sequence may be inserted at the Hprt (hypoxanthine phosphoribosyltransferase 1) gene. According to such embodiments, where the disclosed genetically modified organism is a mouse, the left homology arm may comprise the nucleic acid sequence as denoted by SEQ ID NO: 120, and the right homology arm may comprise the nucleic acid sequence as denoted by SEQ ID NO: 121.

It should be understood that although the disclosed lines were exemplified, as a proof of concept in mammalian subjects, specifically in mice, the disclosed lines of genetically modified organisms may be applicable for any of the eukaryotic organisms disclosed by the present disclosure. Still further, it should be appreciated that the genetically modified organisms disclosed by the present disclosure, for example, those defined herein, are applicable for any aspect of the present disclosure, specifically, to any of the disclosed systems, products, cells, progeny, and methods as disclosed herein after.

A further aspect of the present disclosure relates to a system comprising at least one genetically engineered eukaryotic heterogametic organism and at least one homogametic eukaryotic organism. In some embodiments, the disclosed systems further comprise in addition to the genetically engineered heterogametic organism, also at least one homogametic organism. In some embodiments, the homogametic organism may be a Wild-type homogametic organism. In yet some other embodiments, the homogametic organism may be also genetically modified, specifically, a genetically engineered homogametic organism. In more specific embodiments, the heterogametic genetically engineered organism of the system of the present disclosure comprises: A first sex chromosome (a), comprising at least one exogenous nucleic acid sequence integrated therein. In more specific embodiments, the exogenous nucleic acid sequence comprises the following sequences:

In a first sequence (i), at least one nucleic acid sequence encoding at least one nucleic acids modifier component. In a second sequence (ii), at least one nucleic acid sequence encoding or forming at least one target recognition element for the at least one nucleic acids modifier component of (i). In some optional embodiments, the exogenous nucleic acid sequence may further comprise a third sequence (iii), at least one nucleic acid sequence encoding at least one inhibitor or repressor for the at least one nucleic acids modifier component of (i).

In some optional embodiments, the genetically engineered heterogametic organism of the invention may comprise exogenous nucleic acid sequences also in the second sex chromosome thereof. According to these optional embodiments, the genetically engineered eukaryotic heterogametic organism further comprises a second sex chromosome (b), comprising at least one of the following sequences: In a fourth sequence (iv), at least one exogenous nucleic acid sequence encoding at least one inhibitor or repressor for the at least one nucleic acids modifier component of (i); and as a fifth sequence (v), at least one endogenous or exogenous nucleic acid sequence encoding at least one transcription factor or repressor, that specifically bind at least one transcription regulatory element operably linked to the nucleic acid sequence of at least one of (i), (ii) and (iii).

It should be understood that in some optional embodiments, at least one of the first, second and the optional third nucleic acid sequences of (i), (ii) and the optional (iii), (iv) and/or (v), respectively, may be operably linked to at least one regulatory element. More specifically, such regulatory element may be at least one of: at least one inducible or repressible regulatory element [including inducible promoters (Tet-off), and also post translationally inducible elements, specifically, degrons (DD and LID)], at least one transcriptional regulatory element, at least one post-translationally regulatory element (degrons) and at least one temporally or spatially regulatory element.

As indicated above, the disclosed systems further comprise in addition to the genetically engineered heterogametic organism, also at least one homogametic organism. In some embodiments, the system of the invention comprises a homogametic Wild type organism, and any of the genetically engineered heterogametic organism described by the invention as detailed herein above. Still further, in some additional embodiments, the disclosed systems may comprise any of the heterogametic genetically engineered organisms disclosed by the present disclosure, and at least one homogametic genetically engineered organism. Specific embodiments for such genetically engineered homogametic organism may be any of the genetically engineered organisms described as lines #17 to #19. Still further, in some embodiments, the genetically engineered eukaryotic homogametic organism may comprise a second sex chromosome comprising at least one exogenous nucleic acid sequence encoding at least one inhibitor of said Cas9. In some optional embodiments, the Cas9 inhibitor may be at least one of AcrIIA4 and AcrIIA2.

In yet some further aspect, the invention provides at least one progeny or cell produced or prepared by at least one genetically engineered eukaryotic heterogametic organism provided by the invention, specifically, as disclosed above, or any product thereof.

In some specific embodiments, when the genetically engineered organism of the invention is a cow, it should be understood that the invention further encompasses any progeny of the genetically engineered cow of the invention (e.g., any progenies produced by fertilizing a wild type female cow with sperm cells provided by the genetically engineered male of the invention, or alternatively, fertilizing a genetically engineered female cow (for example in accordance with the lines #17 to #19), with sperm cells provided by the genetically engineered male of the invention), as well as any dairy product produced thereby, any leather, fur or meat product of any of the genetically engineered males or females or of progenies thereof. As noted above, the invention further encompasses the use of any of the genetically engineered organisms provided by the invention, as well as any tissue, organ, cell thereof and of any progeny thereof. It should be noted that the invention further encompasses any product produced from any of the genetically engineered organisms of the invention or of any progenies thereof.

In more specific embodiments, where the genetically engineered organism of the invention is an avian organism, specifically, a chicken, it should be understood that the invention pertains to any eggs, either fertilized or non-fertilized eggs laid by the genetically engineered hen (as being the heterogametic organism), and/or genetically engineered rooster of the present disclosure or by any progeny thereof. Still further, the invention encompasses any product produced by the genetically engineered chickens of the invention or by any progeny thereof or by any eggs produced by these genetically engineered chickens.

The term “fertilized egg” refers hereinafter to an egg laid by a hen wherein the hen has been mated by a rooster within two weeks, allowing deposit of male sperm into the female infundibulum and fertilization event to occur upon release of the ovum from the ovary. “Unhatched egg” as used herein, relates to an egg containing an embryo (also referred to herein as a fertile egg) within a structurally integral (not broken) shell. It should be understood that unhatched egg, as used herein refer either to an egg containing a vital avian embryo of the desired sex, or alternatively a non-vital embryo of the undesired sex.

The term “egg products” refers to any product/s obtained from eggs, from their different components or blends, once the shell and membranes have been removed and that are destined for human consumption, or any other use described herein. This term includes eggs that are removed from their shells for processing and convenience, for commercial, foodservice, and home use. These products can be classified as refrigerated liquid, frozen, and dried products.

They can be partially complemented by other food products or additives and can be found solid, concentrated, liquid, dried, crystallized, frozen, deep-frozen or coagulated. The possibilities in the use of egg products in accordance with the invention, are varied due to the techno-functional properties that they provide. Such properties may include foaming, emulsifying, and a unique color and flavor, which are important in several industrial products and processes, to name but a few, Confectionery, Bakery, Pastry, Dairy products, Ice creams, Drinks, Baby food, Creams and soups, Mayonnaise and sauces, Pasta, Ready cooked meals, Delicatessen, Pet food, Fish farming food, Cosmetic products, Glues (specifically, albumin), Tannery, pains, Pharmaceutical Industry. Still further, egg components and parts may also display useful properties and any uses thereof is also encompassed by the invention. More specifically, egg yolk and components thereof, may exhibit variety of properties such as, Flavouring, Coloring (by Xanthophyllis), Emulsifier capacity (by Lecithin, Lipoproteins LDL), Coagulant and binding substance (by Lipoproteins LDL and other proteins), Antioxidant (Phosvitin), Pharmaceutical uses (IgY, Cholesterol, Sialic acid). Egg white and its main protein, albumen may display Frother capacity, foam stabilizer (Lysozyme, Egg albumen), Anticrystallization (Egg mucin, Egg mucoid), Coagulant and binding substance (by Egg Albumin, Conalbumin), Preservatives (Lysozyme, Conalbumin), Rheological properties and Pharmaceutical properties. In some embodiments, any of the eggs of the invention as disclosed herein or any component, element part or product thereof may be used for cosmetic applications. More specifically, egg white produced from the eggs of the invention may be used as facial products, skin care, hair care and in lotions. Egg yolks produces from any of the eggs of the invention may be used in shampoos, conditioners and soaps. Cholesterol, lecithin and some of the egg's fatty acids may be used in skin care products, such as revitalizers, make-up foundations and lipstick. In yet some further embodiments, the eggs of the invention may be used in animal feed. The excellent nutrition of eggs enhances various pet foods. Egg white may be used as a protein reference in feeding laboratory animals. Eggshells produced from the eggs of the invention may be dried, crushed and used to fed to laying hens as a rich calcium source and high-quality protein source (from egg white left inside the shells). In yet some further embodiments, any of the eggs of the invention as disclosed herein or any component, element part or product thereof may be used for medical and pharmaceutical application. More specifically, fertile eggs provided by the invention may be used to manufacture vaccines (including influenza shots), as a source of purified protein and as an aid in the preservation of bull semen for artificial insemination.

Still further in some embodiments, any of the eggs of the invention as disclosed herein or any component, element part or product thereof may be used for nutraceutical application. More specifically, particular components purified and prepared from the eggs of the invention may be specifically applicable, in different products and processes. For example, lysozyme, an egg white protein, may be used as a food preservative and as an antimicrobial agent in pharmaceutical products. Avidin that is an egg white protein and biotin that is a vitamin found in egg white and, to a much greater extent, in egg yolk, may be prepared and purified from any of the eggs of the invention. Avidin-biotin technology in accordance with the invention may be used in various medical diagnostic applications such as immuno-assay, histopathology and gene probes. Sialic acid, an amido acid, that may be purified from any of the eggs of the invention, has been shown to inhibit certain stomach infections. Liposomes, fatty droplets found in eggs, are used as a controlled delivery mechanism for various drugs. Immunoglobulin yolk (IGY), a simple egg-yolk protein which has immunological properties, may be used as an anti-human-rotavirus (HRV) antibody in food products. Phosvitin, a phosphoprotein found in egg yolk, provides antioxidant benefits in food products. Choline, a B vitamin combined with lecithin in egg yolk, is important in brain development and is used to treat certain liver disorders. Eggs are one of the best food sources of choline. Ovolecithin, a phospholipid found in egg yolk, has a high proportion of phosphatidycholine and contains fatty acids-such as arachidonic acid (AA) and docosahexanoic acid (DHA), which have been shown to improve visual activity in infants and to improve fatty-acid status. Egg lecithin has both emulsifying and antioxidant properties and, beyond its usefulness in keeping the oil and vinegar of mayonnaise in suspension, it's used chiefly in medicine. Shell-membrane protein is being used experimentally to grow human skin fibroblasts (connective tissue cells) for severe-burn victims and in cosmetics. In yet some further embodiments, the invention further provides the use of eggshells prepared from any of the eggs laid by any genetically engineered chicken of the invention or by any progeny thereof, as a dietary source of calcium for humans and other mammals. In further embodiments, these eggshells may be used as a powdered, purified product in fortification of breads and confectioneries, fruit drinks, crackers, condiments. Egg shell calcium in accordance with the invention may be also used as oral phosphate binder in low phosphate diets for e.g. patients suffering from renal failure.

Still further, in some embodiments thereof, the invention provides the use of any protein or substance separated and/or purified from any of the eggs of the invention or from any element or component thereof. More specifically, such separated proteins can be used in food and pharmaceutical industry as is or after enzymatic modifications. In some embodiments, ovotransferrin that may be separated from any of the eggs of the invention, may be used as a metal transporter, antimicrobial, or anticancer agent, whereas lysozyme may be mainly used as a food preservative, and ovalbumin may be used as a nutrient supplement. Ovomucoid may be used to as an anticancer agent and ovomucin as a tumor suppression agent. Hydrolyzed peptides from these proteins may be also used for anticancer, metal binding, and antioxidant activities. Therefore, separation of egg white proteins from any of the eggs of the invention and the productions of bioactive peptides from egg white proteins are all are encompassed by the present invention.

Still further aspect of the invention relates to an in vivo or ex vivo population of gamete cells predominantly composed of one desired sex, or any composition or preparation thereof. It should be noted that in some embodiments, the population of cells is produced by at least one genetically engineered eukaryotic heterogametic organism as defined and provided by the present disclosure and/or the disclosed systems, specifically, as disclosed above. In some embodiment, the population may comprise at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% of gametes of a desired sex.

More specifically, the in vivo or ex vivo population of gamete cells may be a semen, oocytes or embryos. The term “semen”, also known as “seminal fluid”, refers to an organic bodily fluid created to contain spermatozoa. It is secreted by the gonads (sexual glands) and other sexual organs of male or hermaphroditic animals and can fertilize the female ovum. Semen is produced and originates from the seminal vesicle, which is located in the pelvis. The process that results in the discharge of semen from the urethral orifice is called ejaculation. In some embodiments of the disclosure the semen may be collected for cryopreservation until use (for future breeding). The seminal fluid, either fresh or cryopreserved, may be used in natural or non-natural fertilization of the female gamete as discussed herein after in connection with intracervical insemination (ICI), intrauterine insemination (IUI) and/or “in vitro fertilization” (or IVF). As indicated herein, the semen and any products thereof is composed predominantly of gamete cells of the desired sex.

As indicated above, in some embodiments, the in vivo or ex vivo population of gamete cells may be oocytes. The term “oocyte” or “egg cell” as used herein is the female reproductive cell, or female gamete cell. When egg and sperm fuse during fertilization, a diploid cell (the zygote) is formed, which rapidly grows into a new organism. Still further, the, the in vivo or ex vivo population of gamete cells may be included in an embryo. More specifically, the term “embryo” as used herein refers to the initial stage of development of a multicellular organism. In organisms that reproduce sexually, embryonic development is the part of the life cycle that begins just after fertilization of the female egg cell by the male sperm cell. The resulting fusion of these two cells produces a single-celled zygote that undergoes many cell divisions that produce cells known as blastomeres. The blastomeres are arranged as a solid ball that when reaching a certain size, called a morula, takes in fluid to create a cavity called a blastocoel. The structure is then termed a blastula, or a blastocyst in mammals. The mammalian blastocyst hatches before implantation into the endometrial lining of the womb. Once implanted the embryo will continue its development (through the next stages of gastrulation, neurulation, and organogenesis, wherein gastrulation is the formation of the three germ layers that will form all of the different parts of the body: neurulation forms the nervous system, and organogenesis is the development of all the various tissues and organs of the body).

A further aspect of the invention relates to a method for preparing gamete cell population predominantly composed of one desired sex. More specifically, the method comprising the step of obtaining gamete cell population from at least one genetically engineered eukaryotic heterogametic organism comprising: A first sex chromosome (a), comprising at least one exogenous nucleic acid sequence integrated therein. In more specific embodiments, the exogenous nucleic acid sequence comprises the following sequences:

In a first sequence (i), at least one nucleic acid sequence encoding at least one nucleic acids modifier component. In a second sequence (ii), at least one nucleic acid sequence encoding or forming at least one target recognition element for the at least one nucleic acids modifier component of (i). In some optional embodiments, the exogenous nucleic acid sequence may further comprise a third sequence (iii), at least one nucleic acid sequence encoding at least one inhibitor or repressor for the at least one nucleic acids modifier component of (i).

Optionally, at least one of the first, second and the optional third nucleic acid sequences of (i), (ii) and (iii), respectively, are operably linked to at least one regulatory element. More specifically, such regulatory element may be at least one of: at least one inducible or repressible regulatory element [e.g., inducible promoters (Tet-off), and also post translationally inducible elements, specifically, degrons (DD and LID)], at least one transcriptional regulatory element, at least one post-translationally regulatory element (degrons) and at least one temporally or spatially regulatory element.

In some optional embodiments, the genetically engineered heterogametic organism used by the methods of the invention may comprise exogenous nucleic acid sequences also in the second sex chromosome thereof. According to these optional embodiments, the genetically engineered eukaryotic heterogametic organism further comprises a second sex chromosome (b), comprising at least one of the following sequences: In a fourth sequence (iv), at least one exogenous nucleic acid sequence encoding at least one inhibitor or repressor for the at least one nucleic acids modifier component of (i); and as a fifth sequence (v), at least one endogenous or exogenous nucleic acid sequence encoding at least one transcription factor or repressor, that specifically bind at least one transcription regulatory element operably linked to the nucleic acid sequence of at least one of (i), (ii) and (iii).

It should be noted that optionally, the exogenous nucleic acid sequence/s are operably linked to at least one of: at least one inducible or repressible regulatory element, at least one transcriptional regulatory element, at least one post-translationally regulatory element and at least one temporally or spatially regulatory element.

In some specific embodiments of the methods of the invention, the gamete cell population is obtained from any of the genetically engineered eukaryotic heterogametic organism provided by the invention, specifically, any of the genetically engineered organisms as defined and disclosed by the present disclosure, as well as the systems disclosed herein.

In yet another aspect, the invention provides s methods for selecting a desired sex of a eukaryotic organism. More specifically, the method comprising the steps of: contacting at least one genetically engineered heterogametic organism, or any gamete cells obtained therefrom with at least one homogametic eukaryotic organism or gamete cells obtained therefrom, thereby obtaining a progeny predominantly composed of the one desired sex. More specifically, in some embodiments, the homogametic organism used in the disclosed methods, or gamete cells thereof, may be a Wild type homogametic organism. In yet some further alternative embodiments, the homogametic organism used in the disclosed methods, or gamete cells thereof, may be a genetically engineered organism or any gametes obtained therefrom. It should be understood that in some embodiments where the homogametic organism is contacted with the genetically engineered heterogametic organism, such contact may be also referred to herein as breeding, crossing and the like. Still further, in some embodiments, gamete cells obtained from the genetically engineered heterogametic organism may be contacted with the homogametic organism. Still further, in other embodiments, gamete cells of the homogametic organism may be contacted with the heterogametic organism. In yet some further embodiments, gamete cells of the genetically engineered heterogametic organism may be contacted with gamete cells of the homogametic organism. According to such embodiments, the fertilization may occur in vitro.

“Breeding” is sexual reproduction that produces offspring, usually animals or plants. It can only occur between a male and a female animal or plant. There are several options for such breeding. “Natural breeding” involves sexual contact between the animals, where the animals with the desirable characteristics are allowed to mate by natural means. Natural breeding requires minimum effort from the breeders. Mating can be ensured by placing the superior males together with the selected females in an enclosed space. Another breeding option is “artificial insemination” wherein a sperm is deliberately introduced into a female's cervix or uterine cavity for the purpose of achieving a pregnancy through in vivo fertilization by means other than sexual intercourse. It is a common practice in animal breeding, including dairy cattle and pigs. Artificial insemination may employ assisted reproductive technology, sperm donation and animal husbandry techniques. Artificial insemination techniques available include intracervical insemination (ICI) and intrauterine insemination (IUI).

“Intracervical insemination” (or ICI) is the method of artificial insemination which most closely simulates the natural ejaculation of semen by the penis into the vagina during sexual intercourse. It is painless and is the simplest, easiest and most common method of artificial insemination involving the introduction of unwashed or raw semen into the vagina at the entrance to the cervix, usually by means of a needleless syringe. Sperm used in ICI inseminations comprises the seminal fluid (no washing step is applied) so that raw semen from a donor may be used.

“Intrauterine insemination” (or IUI) involves injection of washed sperm directly into the uterus with a catheter. Insemination in this way means that the sperm do not have to swim through the cervix which is coated with a mucus layer. This layer of mucus can slow down the passage of sperm and can result in many sperm perishing before they can enter the uterus.

In some embodiments the contact between the homogametic organism and the transgenic heterogametic organism comprises an in vitro contact between gamete cells of the heterogametic and the homogametic organism. In some embodiments, such contact may be achieved by “in vitro fertilization” (or IVF). IVF is a process of fertilization where an egg is combined with sperm in vitro. The process involves harvesting unfertilized eggs directly from the animal and letting sperm fertilize them in a culture medium in a laboratory. After the fertilized egg (zygote) undergoes embryo culture for several days, it may be transferred into the uterus, with the intention of establishing a successful pregnancy.

In more specific embodiments, the genetically engineered eukaryotic heterogametic organism comprising:

A first sex chromosome (a), comprising at least one exogenous nucleic acid sequence integrated therein. In more specific embodiments, the exogenous nucleic acid sequence comprises the following sequences:

In a first sequence (i), at least one nucleic acid sequence encoding at least one nucleic acids modifier component. In a second sequence (ii), at least one nucleic acid sequence encoding or forming at least one target recognition element for the at least one nucleic acids modifier component of (i). In some optional embodiments, the exogenous nucleic acid sequence may further comprise a third sequence (iii), at least one nucleic acid sequence encoding at least one inhibitor or repressor for the at least one nucleic acids modifier component of (i).

In some optional embodiments, the genetically engineered heterogametic organism used by the methods of the invention may comprise exogenous nucleic acid sequences also in the second sex chromosome thereof. According to these optional embodiments, the genetically engineered eukaryotic heterogametic organism further comprises a second sex chromosome (b), comprising at least one of the following sequences: In a fourth sequence (iv), at least one exogenous nucleic acid sequence encoding at least one inhibitor or repressor for the at least one nucleic acids modifier component of (i); and as a fifth sequence (v), at least one endogenous or exogenous nucleic acid sequence encoding at least one transcription factor or repressor, that specifically bind at least one transcription regulatory element operably linked to the nucleic acid sequence of at least one of (i), (ii) and (iii).

In some optional embodiments, the at least one of the first, second and the optional third nucleic acid sequences of (i), (ii) and the optional (iii), (iv), and (v), respectively, may be operably linked to at least one regulatory element. More specifically, such regulatory element may be at least one of: at least one inducible or repressible regulatory element [for example, inducible or repressible promoters (Tet-off), and also post translationally inducible elements, specifically, degrons (DD and LID)], at least one transcriptional regulatory element, at least one post-translationally regulatory element (degrons) and at least one temporally or spatially regulatory element. In some embodiments of the genetically engineered organism used by the methods of the invention, the first sex chromosome (a), is a sex chromosome characterizing an undesired sex. Accordingly, the second sex chromosome (b), that may be either modified, or unmodified, is a chromosome characterizing a desired sex. In yet some further embodiments, the nucleic acids modifier component encoded by the nucleic acid sequence of the first sex chromosome (a) (i), of the genetically engineered organism used by the methods of the invention, may be at least one modifier protein or any fusion protein thereof. Alternatively, the modifier may be at least one modifier nucleic acid sequence. It should be appreciated that the first nucleic acid sequences may encode both, a protein-based modifier and a nucleic acid-based modifier. In some embodiments, the nucleic acid modifier is a protein. In yet some further embodiments, such modifier protein is a nuclease. More specifically, such nuclease according to some embodiments, may be at least one of: (i) a nuclease having a nucleolytic activity and/or a fusion protein thereof; (ii) a non-active nuclease and/or a fusion protein thereof. It should be understood that any modifier disclosed herein in connection with other aspects of the invention is also applicable in the present aspect.

In some embodiments, the nuclease encoded by the first nucleic acid sequence incorporated into the first sex chromosome of the genetically engineered heterogametic organism used by the methods of the invention may be an RNA guided nuclease. Accordingly, the first sext chromosome may therefore further comprise at least one target recognition element for such nuclease. In some embodiments, such recognition element is at least one ribonucleic acid guide (guide RNA) directed against at least one target sequence within at least one of coding and non-coding sequences, or any product/s thereof, of at least one gene involved in viability, stability and/or function of gamete cells of the organism. In certain embodiments, the at least one gene involved in viability, stability and/or function of gamete cells of the organism may be a gene expressed at a late stage of gametogenesis.

In some embodiments, the RNA guided DNA binding protein nuclease is any one of a clustered regularly interspaced short palindromic repeat (CRISPR) Class 2 or Class 1 system.

Still further, in some embodiments, the RNA guided DNA binding protein nuclease is any one of a clustered regularly interspaced short palindromic repeat (CRISPR) Class 2 or Class 1 system. In yet some further specific embodiments, the RNA guided DNA binding protein nuclease is a CRISPR-associated endonuclease 9 (Cas9) system.

In yet some further embodiments, the genetically engineered eukaryotic heterogametic organism used by the methods of the invention may comprise a transgenic nucleic acid sequence encoding an inhibitor of the modifier protein, specifically, nuclease. In more specific embodiments, such nuclease may be Cas9. Accordingly, an inhibitor of such at least one nuclease, may be an inhibitor of Cas9. In some specific embodiments, such Cas9 inhibitor may be at least one of anti-CRISPR (Acr) IIA4 (AcrIIA4) and AcrIIA2 AcrIIC3, AcrE1, AcrE2, AcrE3, AcrE4, AcrF1, AcrF2, AcrF3, AcrF4, AcrF5, AcrF6, AcrF7, AcrF8, AcrF9, AcrF10, AcrIIA1, AcrIIA2, AcrIIA3, AcrIIA4, AcrIIC1, AcrIIC2, AcrIIC3. It should be noted that the nucleic acid sequences of the genetically engineered eukaryotic heterogametic organism used by the methods of the invention, specifically, (i), (ii) and the optional (iii), may be operably linked to at least one regulatory element. In some embodiments, such elements may be an inducible and/or repressible element. In more specific embodiments, a repressible element (a), may be for example a repressible promoter such as the Tetracycline (Tet)-off, Alcohol dehydrogenase 1 (ADH1), GAL4/UAS, LexA/lexAop, and the Q system. In yet some further embodiments, an inducible or repressible element may be (b), at least one degron. In more specific embodiments, such degron may be at least one of: destabilization domain (DD), ligand-induced degradation (LID), and auxin-inducible degron (AID). Still further, in some embodiments, the regulatory element operably linked to the nucleic acid sequences of the genetically engineered heterogametic organism of the invention may be a post-translationally regulatory element, specifically, degron. Specifically, such degron may be any one of DD, UbK, UbD, and UbM. In yet some further embodiments, embodiments, the regulatory element operably linked to the nucleic acid sequences of the genetically engineered heterogametic organism of the invention may be at least one temporally or spatially regulatory element, specifically, a regulatory element that affects specific tissue or developmental stage. In more specific embodiments, such element may be the Spermatid maturation protein 1 (spem 1) promoter.

In some specific embodiments, the genetically engineered eukaryotic heterogametic organism used by the methods of the invention may comprise: a first sex chromosome comprising: (i), at least one nucleic acid sequence encoding at least one Cas9, or any fusion protein thereof; and (ii), at least one nucleic acid sequence encoding at least one gRNA directed against at least one target sequence.

In some optional embodiments, at least one of the nucleic acid sequences of (i) and (ii), may be operably linked to at least one of at least one inducible regulatory element, at least one transcriptional regulatory element, at least one post-translationally regulatory element and at least one temporally or spatially regulatory element. It should be noted that according to some embodiments, the first sex chromosome characterizes the undesired sex.

In some embodiments, wherein the desired sex is female, the genetically engineered eukaryotic heterogametic organism used by the disclosed methods may be a mammalian male comprising a Y chromosome comprising: (i), at least one nucleic acid sequence encoding at least one Cas9 having a nucleolytic activity and/or a fusion protein thereof; or at least one non-active Cas9 and/or fusion protein thereof; and (ii), at least one nucleic acid sequence encoding at least one gRNA directed against at least one target sequence. In some optional embodiments, the target sequence is at least one protospacer comprised within at least one of the Spem1, JmjC-containing H3K9 demethylase (Jhdm2a), SUN domain-containing protein 4 (Sun4), SUN domain-containing protein 5 (Sun5), Crem, Golgi-associated PDZ and coiled-coil motif-containing protein gene (Gopc), the hook microtubule tethering protein 1 (Hook1) and/or the TATA box-binding protein-like protein 1 (Tbpl1) genes, and/or any orthologs or homologs thereof. still further, in some optional embodiments, the nucleic acid sequences of (i) and (ii), may be operably linked to at least one of at least one inducible regulatory element, at least one transcriptional regulatory element, at least one post-translationally regulatory element and at least one temporally or spatially regulatory element.

In some particular and non-limiting embodiments, the genetically engineered eukaryotic heterogametic organism used by the methods of the present disclosure may comprise a first sex chromosome comprising: (i), at least one nucleic acid sequence encoding dCas9-KRAB-MeCP2 fusion protein under a Tet-off promoter; and (ii), at least one nucleic acid sequence encoding at least one gRNA directed against at least one target sequence within the Spem1 gene. It should be understood that the first sex chromosome that comprises the discussed exogeneous nucleic acid sequences, is the chromosome characterizing the undesired sex. It should be noted that particular embodiments for such genetically engineered organism are provided in the present disclosure by the genetically engineered organism of line #2 as disclosed in Table 4, and in Examples 6, and 15. Still further, in some other specific embodiments, the genetically engineered eukaryotic heterogametic organism used by the methods of the invention may comprise a first sex chromosome comprising: (i), at least one nucleic acid sequence encoding at least one Cas9, or any fusion protein thereof; (ii), at least one nucleic acid sequence encoding at least one gRNA directed against at least one target sequence; and (iii), at least one exogenous nucleic acid sequence encoding at least one inhibitor of said Cas9. In some optional embodiments, the Cas9 inhibitor may be at least one of AcrIIA4 and AcrIIA2. It should be further understood that the first sex chromosome that comprises the exogeneous nucleic acid sequence characterizes the desired sex. In some optional embodiments, at least one of the nucleic acid sequences of (i), (ii) and (iii), may be operably linked to at least one of at least one inducible regulatory element, at least one transcriptional regulatory element, at least one post-translationally regulatory element and at least one temporally or spatially regulatory element.

In some other specific embodiments, the genetically engineered eukaryotic heterogametic organism used by the methods of the invention may comprise:

A first sex chromosome (a), comprising the following sequences:

In a first sequence (i), at least one nucleic acid sequence encoding at least one Cas9, or any fusion protein thereof; and as the second sequence (ii), at least one nucleic acid sequence encoding at least one gRNA directed against at least one target sequence.

According to some optional embodiments, at least one of the nucleic acid sequences of (i) and (ii), are operably linked to at least one of at least one inducible regulatory element, at least one transcriptional regulatory element, at least one post-translationally regulatory element and at least one temporally or spatially regulatory element. It should be noted that this first sex chromosome, characterizes the undesired sex.

The genetically engineered heterogametic organism comprises a second sex chromosome (b), comprising at least one exogenous nucleic acid sequence encoding at least one inhibitor of said Cas9. According to optional embodiments, the Cas9 inhibitor may be at least one of AcrIIA4 and AcrIIA2, or at least one of AcrIIC3, AcrE1, AcrE2, AcrE3, AcrE4, AcrF1, AcrF2, AcrF3, AcrF4, AcrF5, AcrF6, AcrF7, AcrF8, AcrF9, AcrF10, AcrIIA1, AcrIIA2, AcrIIA3, AcrIIA4, AcrIIC1, AcrIIC2, AcrIIC3. It should be noted that the second sex chromosome characterizes the desired sex.

In yet some further embodiments, the genetically engineered eukaryotic heterogametic organism used by the method of the invention comprises:

A first sex chromosome (a), comprising the following sequences: (i), at least one nucleic acid sequence encoding at least one Cas9, or any fusion protein thereof; and (ii), at least one nucleic acid sequence encoding at least one gRNA directed against at least one target sequence.

It should be noted that at least one of the nucleic acid sequences of (i) and (ii), are operably linked to at least one transcription regulatory element. It should be noted that these regulatory elements being specific for a repressor endogenously encoded by a nucleic acid sequence comprised within the second sex chromosome. In yet some optional embodiments, at least one of the nucleic acid sequences of (i), (ii), is operably linked to at least one of: at least one inducible regulatory element, at least one transcriptional regulatory element, at least one post-translationally regulatory element and at least one temporally or spatially regulatory element. It should be noted that this first sex chromosome characterizes the undesired sex.

The genetically engineered organism of the invention further comprises a second sex chromosome (b), comprising at least one endogenous nucleic acid sequence encoding at least one repressor specific for the transcription regulatory element operably linked to at least one of said nucleic acid sequences of (i) and (ii). It should be noted that this second sex chromosome characterizes the desired sex.

Still further, in certain embodiments, the invention provides methods using a genetically engineered eukaryotic heterogametic organism that comprise:

A first sex chromosome (a) comprising: First (i), at least one nucleic acid sequence encoding at least one Cas9, or any fusion protein thereof; second (ii), at least one nucleic acid sequence encoding at least one gRNA directed against at least one target sequence; and third (iii), at least one nucleic acid sequence encoding at least one inhibitor of the Cas9. In some optional embodiments the Cas9 inhibitor is at least one of AcrIIA4 and AcrIIA2. In yet some further embodiments, the Cas9 inhibitor may be at least one of AcrIIC3, AcrE1, AcrE2, AcrE3, AcrE4, AcrF1, AcrF2, AcrF3, AcrF4, AcrF5, AcrF6, AcrF7, AcrF8, AcrF9, AcrF10, AcrIIA1, AcrIIA2, AcrIIA3, AcrIIA4, AcrIIC1, AcrIIC2, AcrIIC3. It should be noted that this third nucleic acid sequence is operably linked to at least one transcription regulatory element. Such regulatory element being specific for a transcription factor endogenously encoded by a nucleic acid sequence comprised within the second sex chromosome.

Optionally, at least one of the nucleic acid sequences of (i), (ii) and (iii), is operably linked to at least one of at least one inducible regulatory element, at least one transcriptional regulatory element, at least one post-translationally regulatory element and at least one temporally or spatially regulatory element. It should be noted that the first sex chromosome characterizes the undesired sex.

These genetically engineered organisms used by the methods of the present disclosure further comprises a second sex chromosome (b), comprising at least one endogenous nucleic acid sequence encoding at least one transcription factor specific for the transcription regulatory element operably linked to the nucleic acid sequences of (iii). It should be noted that the second sex chromosome characterizes the desired sex.

In yet some additional embodiments, the methods of the invention provide and use a genetically engineered eukaryotic heterogametic organism comprising a first sex chromosome comprising: (i), at least one nucleic acid sequence encoding at least one Cas9, or any fusion protein thereof; and (ii), at least one nucleic acid sequence encoding at least one gRNA directed against at least one target sequence. It should be noted that at least one of the nucleic acid sequences of (i) and (ii), is operably linked to at least one of at least one inducible regulatory element, at least one transcriptional regulatory element, at least one post-translationally regulatory element and at least one temporally or spatially regulatory element. It should be noted that this first sex chromosome characterizes the undesired sex.

In certain embodiments, the genetically engineered eukaryotic heterogametic organism used by the methods of the invention produces gamete cells predominantly composed of one desired sex. In some specific embodiments of the genetically engineered eukaryotic heterogametic organism used by the methods of the invention, in cases where the desired sex is of the homogametic organism (for example, female XX), and the undesired sex is of the heterogametic organism (for example a male XY), the first sex chromosome is a chromosome characterizing the heterogametic organism, that is the undesired sex (for example, the Y chromosome) and the second sex chromosome is a chromosome characterizing the homogametic organism (for example, the X chromosome).

In yet some alternative embodiments, where the desired sex is of the heterogametic organism (for example, male YX), and the undesired sex is of the homogametic organism (for example a male XX), the first sex chromosome is a chromosome characterizing the homogametic organism, that is the undesired sex (for example, the X chromosome) and the second sex chromosome is a chromosome characterizing the heterogametic organism (for example, the Y chromosome).

In some embodiments, the genetically engineered eukaryotic heterogametic organism used by the methods of the invention is of the biological kingdom Animalia.

Still further, in some embodiments, the genetically engineered eukaryotic heterogametic organism used by the methods of the invention may be any one of a non-human mammal, an avian, an insect, a fish, an amphibian, a reptile, a crustacean, a crab, a lobster, a snail, a clam, an octopus, a starfish, a sea-urchin, jellyfish, and worms.

In more specific embodiments, the genetically engineered eukaryotic heterogametic organism used by the methods of the invention may be a mammal, specifically, a non-human mammal. In some specific embodiment, such mammal may be at least one of a Cattle, domestic pig (swine, hog), sheep, horse, goat, alpaca, lama and Camels.

Still further, the methods of the invention may be applicable for mammalian organisms of the Order Artiodactyla, including members of the family Suidae, subfamily Suinae and Genus Sus, and members of the family Bovidae, subfamily Bovinae including ungulates, specifically, domestic cattle, bison, African buffalo, the water buffalo and the yak. Of particular interest in the present invention may be the domestic cattle being the most widespread species of the genus Bos, and are most commonly classified collectively as Bos taurus.

In yet some further specific embodiments, the genetically engineered eukaryotic heterogametic organism used by the methods of the invention is a mammal. In more specific embodiment, such mammal may be a domestic Cattle. According to such embodiments, the heterogametic organism is a male (YX). Thus, in cases (a), where the desired sex is female, the first sex chromosome is the Y chromosome, and the second sex chromosome is the X chromosome. The genetically engineered male used by the methods of the invention may therefore produce sperm predominantly composed of gamete cells comprising the X chromosome. In alternative embodiments, where the desired sex is male (b), the first sex chromosome as described by the invention herein above, is the X chromosome, and the second sex chromosome is the Y chromosome. According to such particular embodiments, such genetically engineered male produces sperm predominantly composed of gamete cells comprising the Y chromosome.

In yet some further embodiments, the genetically engineered eukaryotic heterogametic organism used by the methods of the invention is a mammal, in yet some further embodiments, such mammal is rodent.

In more particular embodiments, the methods of the invention provide and use a genetically engineered eukaryotic heterogametic rodent. In yet some further embodiments, such rodent is a mouse. Thus, according to some embodiments, the heterogametic organism is a male mouse, that carry both X and Y sex chromosomes. Therefore, in cases where the desired sex is female, the first sex chromosome of the genetically engineered muse of the invention is the Y chromosome, and the second sex chromosome is the X chromosome. In such case, the male produces sperm predominantly composed of gamete cells comprising the X chromosome.

In yet some alternative embodiments, where the desired sex is male, the first sex chromosome is the X chromosome, and the second sex chromosome is the Y chromosome. Accordingly, such male produces sperm predominantly composed of gamete cells comprising the Y chromosome. In more particular embodiments, the genetically engineered eukaryotic heterogametic organism provided and used by the methods of the invention may be a mammal as described above. In some specific embodiments in cases the desired sex is female (XX), the genetically engineered organism of the invention is the heterogametic organism, specifically, a male (YX). Such genetically engineered male comprises:

A Y chromosome (also referred by the invention as the first sex chromosome) comprising the following exogenous sequences: (i) at least one nucleic acid sequence encoding at least one Cas9, or any fusion protein thereof; and (ii), at least one nucleic acid sequence encoding at least one gRNA directed against at least one target sequence. Optionally, the target sequence targeted by the gRNAs may be at least one protospacer comprised within at least one of the JmjC-containing H3K9 demethylase (Jhdm2a), SUN domain-containing protein 4 (Sun4), SUN domain-containing protein 5 (Sun5), Spem1, Crem, Golgi-associated PDZ and coiled-coil motif-containing protein gene (Gopc), the hook microtubule tethering protein 1 (Hook1) and the TATA box-binding protein-like protein 1 (Tbpl1) genes, or any orthologs or homologs thereof.

In some optional embodiments, at least one of the nucleic acid sequences of (i) and (ii), is operably linked to at least one of at least one inducible regulatory element, at least one transcriptional regulatory element, at least one post-translationally regulatory element and at least one temporally or spatially regulatory element. An X chromosome (also referred to herein as the second sex chromosome) comprises at least one exogenous nucleic acid sequence encoding at least one inhibitor of the Cas9. In some optional embodiments, the Cas9 inhibitor is at least one of AcrIIA4 and AcrIIA2.

In yet some further particular embodiments, the genetically engineered eukaryotic heterogametic organism provided and used by the methods of the invention may be a mammal as described above. In some specific embodiments in cases the desired sex is female (XX), the genetically engineered organism of the invention is the heterogametic organism, specifically, a male (YX).

In some specific embodiments, the genetically engineered mammalian male used by the methods of the present disclosure may comprise a first sex chromosome comprising: (i), at least one nucleic acid sequence encoding at least one Cas9, or any fusion protein thereof; and (ii), at least one nucleic acid sequence encoding at least one gRNA directed against at least one target sequence.

In some optional embodiments, at least one of the nucleic acid sequences of (i) and (ii), may be operably linked to at least one of at least one inducible regulatory element, at least one transcriptional regulatory element, at least one post-translationally regulatory element and at least one temporally or spatially regulatory element. It should be noted that according to some embodiments, the first sex chromosome characterizes the undesired sex.

In some particular and non-limiting embodiments, the genetically engineered mammalian male used by the methods of the present disclosure may comprise a first sex chromosome comprising: (i), at least one nucleic acid sequence encoding dCas9-KRAB-MeCP2 fusion protein under a Tet-off promoter; and (ii), at least one nucleic acid sequence encoding at least one gRNA directed against at least one target sequence within the Spem1 gene. It should be understood that the first sex chromosome that comprises the discussed exogeneous nucleic acid sequences, is the chromosome characterizing the undesired sex. It should be noted that particular embodiments for such genetically engineered organism are provided in the present disclosure by the genetically engineered organism of line #2 as disclosed in Table 4, and in Examples 6, and 15.

In some specific embodiments, the genetically engineered mammalian male used by the methods of the present disclosure may comprise comprises:

A Y chromosome (also referred by the invention as the first sex chromosome) comprising the following exogenous sequences: (i), at least one nucleic acid sequence encoding Cas9 having a nucleolytic activity and/or a fusion protein thereof; and (ii) at least one nucleic acid sequence encoding at least one gRNA directed against at least one target sequence. Optionally, the target sequence is at least one protospacer comprised within at least one of Jhdm2a, Sun4, Sun5, Spem1, Crem, Gopc, Hook1, and the Tbpl1 genes or any orthologs or homologs thereof.

According to these embodiments, at least one of the nucleic acid sequences of (i) and (ii), is operably linked to at least one transcription regulatory element, such regulatory element being specific for a repressor endogenously encoded by a nucleic acid sequence comprised within the X chromosome. In yet some optional embodiments, at least one of the nucleic acid sequences of (i), (ii), is operably linked to at least one of: at least one inducible regulatory element, at least one transcriptional regulatory element, at least one post-translationally regulatory element and at least one temporally or spatially regulatory element. Still further, this genetically engineered male further comprises an X chromosome (also referred to herein as the second sex chromosome) comprising at least one endogenous nucleic acid sequence encoding at least one repressor specific for the transcription regulatory element operably linked to at least one of the nucleic acid sequences of (i) and (ii) in the Y chromosome as described above.

In yet some further specific embodiments, the genetically engineered eukaryotic heterogametic organism provided and used by the methods of the invention may be a mammal as described above. In some specific embodiments in cases the desired sex is female (XX), the genetically engineered organism of the invention is the heterogametic organism, specifically, a male (YX). Such genetically engineered male comprises:

A Y chromosome (also referred by the invention as the first sex chromosome) comprising the following exogenous sequences: (i), at least one nucleic acid sequence encoding Cas9 having a nucleolytic activity and/or a fusion protein thereof; (ii), at least one nucleic acid sequence encoding at least one gRNA directed against at least one target sequence. Optionally, the target sequence is at least one protospacer comprised within at least one of the Jhdm2a, Sun4, Sun5, Spem1, Crem, Gopc, Hook1, and the Tbpl1 genes or any orthologs or homologs thereof. The Y chromosome of this genetically engineered male may further comprise (iii), at least one nucleic acid sequence encoding at least one inhibitor of said Cas9, optionally, said Cas9 inhibitor is at least one of AcrIIA4 and AcrIIA2. It should be noted that this sequence is operably linked to at least one transcription regulatory element specific for a transcription factor endogenously encoded by a nucleic acid sequence comprised within the X chromosome. In some optional embodiments, at least one of the nucleic acid sequences of (i), (ii) and (iii), is operably linked to at least one of: at least one inducible regulatory element, at least one transcriptional regulatory element, at least one post-translationally regulatory element and at least one temporally or spatially regulatory element. The X chromosome of such genetically engineered male (also referred to herein as the second sex chromosome) comprises at least one endogenous nucleic acid sequence encoding at least one transcription factor specific for the transcription regulatory element operably linked to the nucleic acid sequences of (iii) in the Y chromosome.

In yet some further particular embodiments, the genetically engineered eukaryotic heterogametic organism provided and used by the methods of invention may be a mammal as described above. In some specific embodiments, in cases the desired sex is female (XX), the genetically engineered organism of the invention is the heterogametic organism, specifically, a male (YX). Such genetically engineered male comprises:

A Y chromosome (also referred by the invention as the first sex chromosome) comprising the following exogenous sequences: (i), at least one nucleic acid sequence encoding at least one Cas9 having a nucleolytic activity and/or a fusion protein thereof; (ii), at least one nucleic acid sequence encoding at least one gRNA directed against at least one target sequence. Optionally, the target sequence is at least one protospacer comprised within at least one of the Jhdm2a, Sun4, Sun5, Spem1, Crem, Gopc, Hook1, and the Tbpl1 genes or any orthologs or homologs thereof. It should be noted that at least one of said nucleic acid sequences of (i) and (ii), is operably linked to at least one of at least one of: at least one inducible regulatory element, at least one transcriptional regulatory element, at least one post-translationally regulatory element and at least one temporally or spatially regulatory element.

It yet some further embodiments, the genetically engineered eukaryotic heterogametic organism provided and used by the methods of the invention may be an avian organism. In more specific embodiments, such avian organism may be any one of a domesticated and an undomesticated bird. In yet some further embodiments, the genetically engineered eukaryotic heterogametic organism provided and used by the methods of the invention is a domesticated bird. In more specific embodiments, such bird is a chicken. In such case, the heterogametic organism is a female (ZW). Thus, in cases where the desired sex is female (ZW), the first sex chromosome is the Z chromosome, and the second sex chromosome is the W chromosome. Such female produces predominantly gamete cells comprising the W chromosome.

However, in cases where the desired sex is male (ZZ), the first sex chromosome is the W chromosome, and the second sex chromosome is the Z chromosome. In such case the genetically engineered hen provided by the invention produces predominantly gamete cells comprising the Z chromosome.

In yet some further specific embodiments, the genetically engineered eukaryotic heterogametic organism provided and used by the methods of the invention may be an organism of the biological kingdom Plantae.

In more specific embodiments, the genetically engineered eukaryotic heterogametic organism used by the methods of the invention may be a dioecious plant.

Still further, in some embodiments, the genetically engineered eukaryotic heterogametic organism used by the methods of the invention may be a dioecious plant, specifically, of the family Cannabaceae.

In some embodiments, the plant of the family Cannabaceae is any one of Cannabis and Humulus. In yet some further embodiments, the methods of the invention are particularly applicable for the preparation of a population of eukaryotic organisms predominantly composed of one desired sex, or any cell or product produced therefrom.

It should be understood that the invention further encompasses any construct, nucleic acid cassette, prepared by the invention and used for the creation of the genetically engineered organisms of the invention, as well as any vector or vehicle comprising such cassettes. The invention further encompasses any host cell comprising any of the constructs or cassettes of the invention, as disclosed herein by the example.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should be understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Experimental Procedures

### Mice Construction:

Mice with knock-in DNA cassettes described in Tables 4, 5 and 6, are constructed according to standard protocols by a commercial company, as described below.

### Uty and Hprt Genes

The uty gene (NCBI Reference Sequence: NM_009484.3, is located on mouse chromosome Y. Twenty-seven exons have been identified, with the ATG start codon in exon 1 and the TAA stop codon in exon 27. The sequence targeted by the targeting vector for insertion of the transgene, specifically, the nucleic acid sequence encoding the nucleic acid modifier of the present disclosure and at least one target recognition element, resides within intron 2 of the uty gene. The entire sequence of the mouse uty gene that includes a UTR region, intron 1, exon 2, intron 2 and exon 3 of the mouse uty gene, comprises the nucleic acid sequence as denoted by SEQ ID NO: 107. In some specific embodiments, the left homology arm (LHA) used in the disclosed constructs, as denoted by SEQ ID NO: 52, comprises sequences of the UTR, intron 1, exon 2, and part of intron 2 of the mouse uty gene, where the right homology arm (RHA) as denoted by SEQ ID NO: 55, contains most of intron 2 and part of exon 3 of the mouse uty gene. The target intron 2 of the mouse uty gene is also denoted herein by SEQ ID NO: 108).

Still further, in some alternative embodiments that require insertion of the transgene to the X chromosome, the Hprt (hypoxanthine phosphoribosyltransferase 1) of Mus musculus ID: MGI: 96217, NCBI Gene: 15452, ENSMUSG00000025630, may be used as a target. In some specific embodiments, the left homology arm (LHA) used in the disclosed constructs, as denoted by SEQ ID NO: 120, the right homology arm (RHA) as denoted by SEQ ID NO: 121. The target intron 2 of the mouse Hprt gene is also denoted herein by SEQ ID NO: 122).

### Construction of Uty Targeting Vector

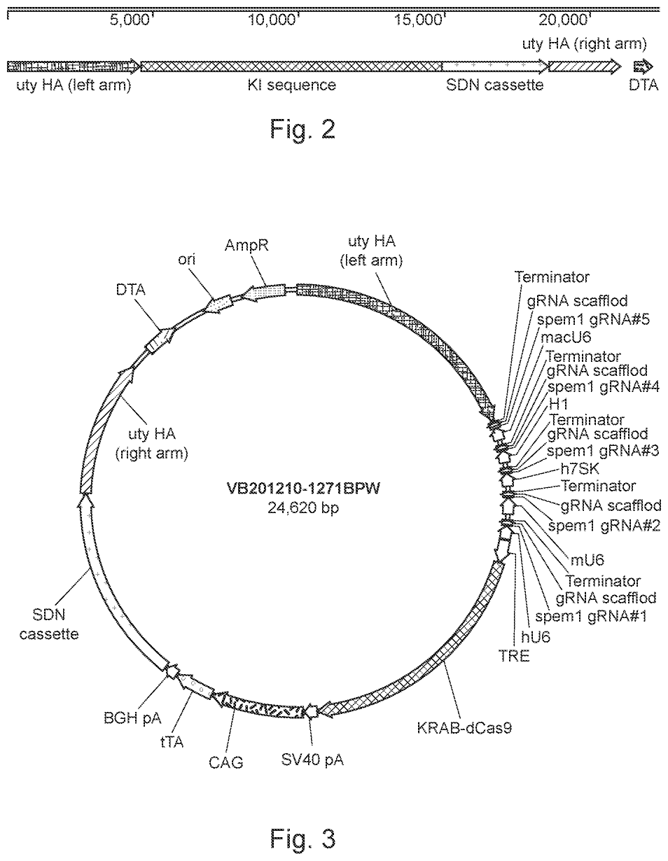

Mouse genomic fragments containing homology arms (HAs) were amplified from Bacterial Artificial Chromosome (BAC) clones by using high fidelity Taq DNA polymerase and were sequentially assembled into a targeting vector together with recombination sites and selection markers as schematically illustrated in FIG. 2.

All targeting vectors were synthesized by VectorBuilder. The targeting vector utilized for mouse construction contains a Neo cassette for positive selection allowing for the subsequent excision of the Neo cassette through SDN (Self-deleting Neo Cassette) (FIG. 2). A non-limiting example for the SDN cassette used in the present disclosure comprises the nucleic acid sequence as denoted by SEQ ID NO: 54. Additionally, the vector includes DTA (diphtheria toxin A) for negative selection and homology arms (HAs) necessary for efficient recombination into the uty gene, which is located on chromosome Y. The knockin (KI) sequence was inserted in the reverse orientation into intron 2 of the mouse uty gene.

### Establishment of KI ES Cells

The uty targeting vector (linearized and purified) was transfected into C57BL/6N ES cells according to cyagen's standard electroporation procedures. The transfected ES cells were subject to G418 selection (200 μg/mL) 24 hours post electroporation. 185 G418 resistant clones were picked and amplified in 96-well plates. Two copies of 96-well plates were made, one copy was frozen down and stored at −80° C. and the other copy of the 96-well plates was used for DNA isolation and subsequence PCR screening for homologous recombination. Selected PCR positive clones were further verified by Southern blot analysis.

### PCR Conditions

### 3′ Arm PCR (Includes Part of the Neo Cassette and the Right Homology Arm):

### Primers:

F1:
5′-CCTGAATGAACTGCAAGACGAGG-3′
as denoted by SEQ ID NO: 90.
R1:
5′-GAGTTCCAGGCCAGTCTATGGTC-3′
as denoted by SEQ ID NO: 91.

### Expected PCR Product:

Wildtype: N.A.
Targeted: 3993 bp

### Reaction Mix:

### Component x1

ES cell genomic DNA 1.5 μL
Forward primer (10 μM) 0.6 μL
Reverse primer (10 μM) 0.6 μL
dNTPs (2.5 mM) 1.8 μL
5× LongAmp Taq Reaction 3 μL
LongAmp Taq DNA Polymerase 0.9 μL
DMSO 0.5 μL
ddH2O 6.1 μL
Total 15 μL

### Cycling Condition:

Step Temp. Time Cycles
Initial denaturation 94° C. 3 min
Denaturation 94° C. 30 s
Annealing 60° C. 30 s 35×
Extension 65° C. 200 s
Additional extension 65° C. 10 min

### KI PCR (Includes Part of the Left Homology Arm and Part of the KI):

### Primers for Mice of Line #2:

F2:
5′-GGTTGTTGCAGCCTCTTGTGA-3′
as denoted by SEQ ID NO: 92.
R2:
5′-CGCCCCAACCTCATTGTGA-3′
as denoted by SEQ ID NO: 93.

### Expected PCR Product:

Wildtype: N.A.
Targeted: 410 bp

### Neo-L PCR (Includes Part of the KI and Part of the Neo Cassette):

### Primers for Mice of Line #2:

F3:
5′-CTTAGACATGCTCCCAGCCGA-3′
as denoted by SEQ ID NO: 94.
R3:
5′-GGCCCACAACAGCACCATTG-3′
as denoted by SEQ ID NO: 95.

### Expected PCR Product:

Wildtype: N.A.
Targeted: 493 bp
   Wildtype PCR (Includes a Wild Type Construct within the Homology Arms):

F4:
5′-GGTTGTTGCAGCCTCTTGTGAT-3′
as denoted by SEQ ID NO: 96.
R4:
5′-TGGTAAAACTACCCTGTTTCCTTTC-3′
as denoted by SEQ ID NO: 97.

### Expected PCR Product:

Wildtype: 348 bp
Targeted: N.A.

### Reaction Mix for KI, Neo-L and Wildtype PCR:

### Component x1

ES cell genomic DNA 1.5 μL
Forward primer (10 μM) 1 μL
Reverse primer (10 μM) 1 μL
P112 Taq DNA Polymerase 12.5 μL
ddH2O 9 μL
Total 25 μL

### Cycling Condition for KI, Neo-L and Wildtype PCR:

Step Temp. Time Cycles
Initial denaturation 94° C. 3 min
Denaturation 94° C. 30 s
Annealing 62° C. 35 s 35×
Extension 72° C. 35 s
Additional extension 72° C. 5 min
Storage temperature 25° C.

### Southern Blot Analysis

The genomic DNA was digested with each of the following restriction enzymes:

KpnI (which digests within the KI and the 3′ arm) and hybridized using a Neo probe, as denoted by SEQ ID NO: 106.

ScaI (which digests within the 5′ arm and the homology sequence in wildtype or the 5′ arm and KI homology arm in the targeted allele) and hybridized using a 5′ arm probe.

AvrII (which digests within the homology sequence the 3′ arm in wildtype or KI homology arm and the 3′ arm in the targeted allele) and hybridized using a 3′ arm probe.

The following probes were used for Southern blot analysis:
Neo probe: F: Neo-F primer as denoted by SEQ ID NO: 104.
R: Neo-R primer, as denoted by SEQ ID NO: 105.

5′ arm probe:
F:
CAGGGATAACCTTTGTGAGGGACTGTT,
as denoted by SEQ ID NO: 98.
R:
AAGTTCCCAGAATCGGTGCTCCTT
as denoted by SEQ ID NO: 99.
3′ arm probe:
F:
GAAATAGAGGGAGGAGAGGGGAAATG,
as denoted by SEQ ID NO: 100.
R:
GGGAATACTTTTCATTAACCTGTACTTGCT,
as denoted by SEQ ID NO: 101.

For TKN-201123-CAA-01-TAC (prepared using plasmid 1 (FIG. 3) for the preparation of line #2 (Table 4), the PCR screening identified forty-one potential targeted clones, from among which six were expanded and further characterized by Southern blot analysis. All of the six expanded clones were confirmed to be correctly targeted.

### Animal Generation

Targeted ES cell were injected into C57BL/6 albino embryos, and then re-implanted into CD-1 pseudo-pregnant females. Founder animals were identified by their coat color and their germline transmission is confirmed by breeding with C57BL/6 females and subsequent genotyping of the offspring. The SDN cassette (also denoted by SEQ ID NO: 54) is self-deleted in germ cells so the offspring are Neo cassette-free. Male s targeted mice were generated from targeted ES clones as final deliverables for this project.

### Determining the Sex of the Offspring:

Pregnancy is monitored daily. Sex of offspring was determined by PCR of the Y chromosome as detailed below on DNA extracted from the animal's tail on day 1. Sex was further confirmed at day 7 and at weaning by observing the genitals.

TABLE 1

| | | | SEQ | | |
|---|---|---|---|---|---|
| | | | ID | Expected PCR | |
| | Primer | Sequence | NO: | Product: | |
| PCR1 | TKIE100- | GGTTGTTGCAGCCTCTTGTGA | 102 | Wildtype: | Targeted: |
| | F1 | | | N.A. | 410 bp |
| | TKIE100- | CGCCCCAACCTCATTGTGA | 93 | | |
| | R1 | | | | |
| PCR2 | TKIE100- | CTTAGACATGCTCCCAGCCGA | 94 | Wildtype: | Targeted: |
| | F3 | | | N.A. | 431 bp |
| | TKIE100- | AACTGTTTCATTTCCCCTCTCCT | 103 | | |
| | R2 | | | | |
| PCR3 | TKIE100- | GGTTGTTGCAGCCTCTTGTGAT | 96 | Wildtype: | Targeted: |
| | F4 | | | 348 bp | N.A. |
| | TKIE100- | TGGTAAAACTACCCTGTTTCCTTTC | 97 | | |
| | R4 | | | | |

N.A. = Not applicable

TABLE 2A
Reaction Mix:

| Component | x1 | |
|---|---|---|
| Mouse genomic DNA | 1.5 | μl |
| Forward primer (10 μM) | 1.0 | μl |
| Reverse primer (10 μM) | 1.0 | μl |
| Premix Taq Polymerase | 12.5 | μl |
| ddH₂O | 9.0 | μl |
| Total | 25.0 | μl |

TABLE 2B
Cycling Condition:

| Step | Temp. | | Time | | Cycles |
|---|---|---|---|---|---|
| Initial denaturation | 94 | ° C. | 3 | min | |
| Denaturation | 94 | ° C. | 30 | s | |
| Annealing | 62 | ° C. | 35 | s | 33 x |
| Extension | 72 | ° C. | 35 | s | |
| Additional extension | 72 | ° C. | 5 | min | |

TABLE 3
Interpolation of PCRs 1-3:

| | | | Interpolation | Interpolation |
|---|---|---|---|---|
| PCR1 | PCR2 | PCR3 | for KI | for sex |
| Positive | Positive | Negative | KI | Male |
| Positive | Negative | Negative | KI suspected | Male |
| Negative | Positive | Negative | KI suspected | Male |
| Negative | Negative | Negative | WT | Female |
| Negative | Negative | Positive | WT | Male |

### Determining the Sex of the Gametes:

Semen from representative from each genetically engineered mouse line is extracted. This semen is stained with nucleic acid-specific fluorophore, such as Hoechst 33342. It is then analyzed using a flow cytometry machine as described (Garner et al, Methods Mol Biol. 2013; 927:279-95). It is expected that the control mice will have a 1:1 ratio of X to Y chromosome-containing gametes, whereas the genetically engineered mice will have a biased ratio of gametes. This sperm is used alternatively to fertilize ova ex vivo and determine the karyotype and sex of the developing zygote.

### Example 1

Preparation of a Male Mouse that Produces Only Female Gametes-Production of GMO-Female Offspring of the Desired Sex

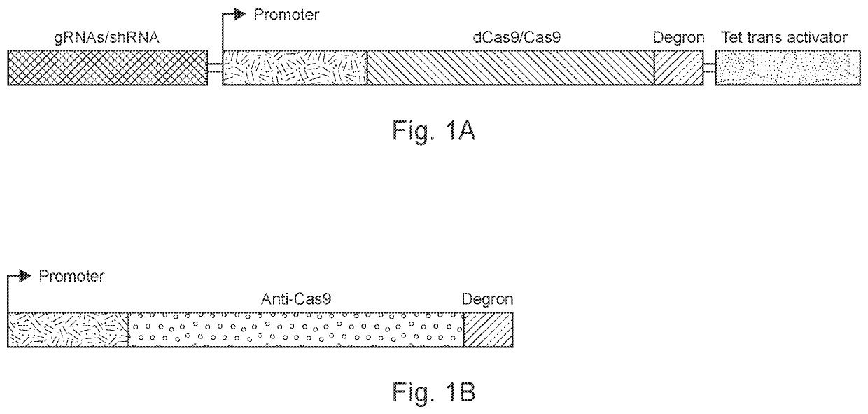

Mice are genetically engineered to encode a functional dCas9 system and guide RNAs on their Y chromosome. In some embodiments, the X chromosome of the male (the heterogametic organism) encodes an anti-dCas9 protein (AcrII4A) under a constitutive promoter. In other embodiments as illustrated in FIG. 1B, the anti-dCas9 protein (AcrII4A) under a constitutive promoter is placed in the X chromosome of another organism (either a homogametic or heterogametic, specifically, male or female). The guides repress genes that are essential for sperm morphogenesis and motility (e.g., Jhdm2a, Sun4, Sun5, Spem1, Crem, Gopc, Hook1, and the Tbpl1 genes). Table 7 discloses additional potential target genes. These genes are repressed only in the Y gametes, as these gametes do not have the anti-dCas9. Thus, only X gametes are produced in this male, as they are protected by the anti-Cas9. This is a simple model for a male mouse that produces only female gametes. However, the female offspring is GMO because their X chromosome encodes an anti-CRISPR protein.

The dCas9 in the above construct is controlled by a Tet-off promoter. Thus, the parental mouse lines are able to produce male progeny for future breeding upon supplement of doxycycline in their drinking water. The supplement of doxycycline constantly represses the dCas9 by preventing binding of the tTA transcription factor to the promoter, thereby inhibiting gene expression. Therefore, the Y-chromosome containing gamete is preserved. For producing female-only progeny, doxycycline is removed from the water.

It should be understood that genetically engineered organisms (e.g., mice) that are modified in both in the heterogametic sex chromosome (e.g., Y, to carry the Cas9), and in the homogametic sex chromosome (X chromosome), will produce gamete cells (e.g., sperm) mainly composed of X chromosome. These gamete cells carry the anti-Cas9, and therefore are GMO. In case the modification is performed in two organisms, for example, in case the female sex is desired, the Y chromosome of the heterogametic organism (male) is modified and carry the Cas9, for example, under an inducible promoter, and the X chromosome of the heterogametic organism (female) is modified to carry the anti-Cas, for example, under constitutive promoter. F1 male offspring of such crossing (e.g. between the male of any of lines #1-16, to female of lines #17-19) that can be created under doxycycline treatment carry the transgene in both sex chromosomes (Y with Cas9 and X with the anti-Cas9), and will produce gamete cells (e.g., sperm), mainly composed of X chromosome. Gamete cells that carry the Y chromosome may be produced only under doxycycline treatment.

The genetic system composed of two cassettes that are introduced into X or Y chromosomes, is represented schematically in FIG. 1A-1B. More specifically, FIG. 1A generally illustrates the mice of strains #1-#16), and FIG. 1B, illustrates the mice of lines #17-#19.

### Example 2

### System Producing a Non-GMO Female-Only Progeny, Using an X-Encoded Repressor

To generate a non-GMO female offspring, genetically engineered mice that encode a functional CRISPR-Cas9 system and guide RNAs under a Tet-off promoter are generated. In these mice, a sequence repressed by an X-encoded repressor, such as Nkrf, is cloned upstream of the CRISPR-Cas9 system on the Y chromosome. Thus, the toxin (CRISPR-Cas9 system and guide RNAs) is repressed as long as the X chromosome is present. Upon separation of the X chromosome, the Y gametes are exterminated.

### Example 3

### System Producing a Non-GMO Female-Only Progeny, Using an X-Encoded Transcription Factor

In yet a further system for creating a non-GMO offspring of the desired sex, genetically engineered mice that comprise a functional CRISPR-Cas9 system and guide RNAs under a Tet-off promoter as well as an anti-CRISPR protein under an X-encoded transcription factor promoter is encoded on the same cassette on the Y-chromosome, are generated. An X-encoded transcription factor that activates the antitoxin, such as Sox3, Foxp3, Tfe3, are used. Thus, the antitoxin is expressed as long as the X chromosome is present. Once the X is separated, the toxin is not repressed, and the Y encoding gametes are exterminated.

In all the above cases, for producing males of these mice, doxycycline is used in the drinking water of the mice to constantly repress the Cas9 toxin. For producing female-only progeny, doxycycline is removed from the water.

Semen is extracted from the engineered mice and test it for producing female-only zygotes.

### Example 4

### System Producing a Non-GMO Female-Only Progeny

A system producing a non-GMO female-only progeny is generated. In this system, the Y chromosome of mice is genetically engineered to encode a functional dCas9 and guide RNAs. This cassette is driven by a sperm-specific gene (e.g., Spem1), which is expressed only in the gamete stage. Thus, dCas9-mediated repression of genes required for spermatogenesis only occurs in gametes carrying the Y-chromosome, and not in gametes carrying the X chromosome. This male thus produces only females, and since the X chromosome is unmodified, the resulting progeny is non-GMO. It should be understood that other targets may be applicable in the present disclosure, for example, any one of Spem1, Sun5, Sun4, Hlfnt—all are testis specific as the table indicates. There are obviously many more, and combinations thereof.

In some embodiments, the dCas9 is controlled at the protein level by a synthetic chemical. The dCas9 is fused to a Ligand-induced degradation (LID) domain that leads to the entire protein degradation in the presence of the synthetic chemical Shield1. Thus, the parental mouse line is able to produce male progeny for future breeding upon supplement of Shield1 in their drinking water. The supplement of Shield1 constantly degrades the dCas9 and therefore, the Y-chromosome containing gamete is preserved. For producing female-only progeny, Shield1 is removed from the water. Examples for such genetically modified organism is provided by Lines #11 to #16 of Table 4.

The semen is extracted from the engineered mice and tested for producing female-only zygotes in vitro. In parallel, the mice are bred, and the sex of the resulting progeny is examined. It is expected that only females will be born, with normal litter size and normal fertility.

### Example 5

Optimizing the Expression Level of the dCas9 Relative to the Anti-dCas9

The separation of the chromosomes during meiosis may leave some dCas-9 in the cytoplasm of the X gametes and anti-dCas9 in the cytoplasm of the Y gametes. It is therefore required to optimize the levels of the dCas9 and anti-dCas9 so that anti-dCas9 is sufficient to repress dCas9 in the spermatocytes where it is encoded, but is at a minimal level, so that upon gamete segregation its effect will diminish. In this respect, it is noteworthy that there is an extensive timeframe for the dCas9 and anti-dCas9 to degrade/dilute as the process of gamete maturation from the time it separates from its counter-sex chromosome is ˜15 days (in mice; this timeframe is longer in cows, goats, pigs and other larger mammals). In order to optimize the levels of the dCas9 and the anti-dCas9, different promoters are constructed upstream of the anti-dCas9 to find the ideal combination resulting in highest fertility and sex selection. Several degradation domains (degrons) are also constructed fused to the dCas9 and to the antitoxin in order to change its stability until finding the optimal combination. The targets of repression should also be selected experimentally, including the type of targets and the number of gRNA targeting them. Some gRNAs are known from the literature to be essential for spermatogenesis, however, the setting in which they were tested is different than current one, and therefore optimization in this respect is also required.

Table 4 details the different dCas9 strains that are produced for optimizing the systems of the invention.

Table 5 details the different Anti-dCas9 strains that are produced for optimizing the systems of the invention.

Table 6 details the combined dCas9-Anti-dCas9 strains that are produced for optimizing the systems of the invention.

TABLE 4
Cas9 strains

| | | | | | |
|---|---|---|---|---|---|
| dCas9 | | | | | |
| strains | | Cas | dCas9 | | |
| Line # | Promoter | enzyme | degron | gRNAs/shRNA | Comments |

Y chromosome engineering - for producing females; X - for producing males.

| | | | | | |
|---|---|---|---|---|---|
| 1 | Tet-off | dCas9- | No | Full array: Spem1 | For crossing with |
| | | KRAB- | | Jhdm2a Sun4 Sun5 | antitoxin |
| | | MeCP2 | | Crem | or standalone. |
| 2 | Tet-off | dCas9- | No | Partial array: Spem1 | For crossing with |
| | | KRAB- | | | antitoxin |
| | | MeCP2 | | | or standalone. |
| 3 | Tet-off | dCas9- | UbM | Full array: Spem1 | For crossing with |
| | | KRAB- | | Jhdm2a Sun4 Sun5 | antitoxin |
| | | MeCP2 | | Crem | or standalone. |
| 4 | Tet-off | dCas9- | UbD | Full array: Spem1 | For crossing with |
| | | KRAB- | | Jhdm2a Sun4 Sun5 | antitoxin |
| | | MeCP2 | | Crem | or standalone. |
| 5 | Tet-off | dCas9- | UbK | Full array: Spem1 | For crossing with |
| | | KRAB- | | Jhdm2a Sun4 Sun5 | antitoxin |
| | | MeCP2 | | Crem | or standalone. |
| 6 | Tet-off | Cas9 | No | Full array: Spem1 | For crossing with |
| | | | | Jhdm2a Sun4 Sun5 | antitoxin. |
| | | | | Crem | |
| 7 | CAGG | dCas9- | DD | Full array: Spem1 | Shield1 produces |
| | | KRAB- | | Jhdm2a Sun4 Sun5 Crem | single sex. |
| | | MeCP2 | | | |
| 8 | CAGG | dCas9- | DD | Partial array: Spem1 | Shield1 produces |
| | | KRAB- | | | single sex. |
| | | MeCP2 | | | |
| 9 | Tet-off | None | No | shRNA targeting spem1 | Tet produces both sexes. |
| | | | | Jhdm2a Sun4 Sun5 | |
| | | | | Crem | |
| 10 | Tet-off | None | No | shRNA targeting spem1 | Tet produces both sexes. |
| 11 | Spem1/ | dCas9- | DD | Full array: Spem1 | Shield1 produces |
| | Akap4 | KRAB- | | Jhdm2a Sun4 Sun5 | single sex. |
| | | MeCP2 | | Crem | |
| 12 | Spem1/ | dCas9- | DD | Partial array: Spem1 | Shield1 produces |
| | Akap4 | KRAB- | | | single sex. |
| | | MeCP2 | | | |
| 13 | Spem1/ | Cas9 | DD | Full array: Spem1 | Shield1 produces |
| | Akap4 | | | Jhdm2a Sun4 Sun5 | single sex. |
| | | | | Crem | |
| 14 | Spem1/ | Cas9 | DD | Partial array: Spem1 | Shield1 produces |
| | Akap4 | | | | single sex. |
| 15 | Spem1/ | Cas9 | LID | Full array: Spem1 | Shield1 produces both |
| | Akap4 | | | Jhdm2a Sun4 Sun5 | sexes. |
| | | | | Crem | |
| 16 | Spem1/ | Cas9 | LID | Partial array: Spem1 | Shield1 produces both |
| | Akap4 | | | | sexes. |

TABLE 5
Anti-dCas9 strains

| | | | | |
|---|---|---|---|---|
| | | | Degron (N | |
| # | Promoter | Anti-Cas | terminus) | Comments |

For female only - construct on X; for males only - on Y.

| | | | | |
|---|---|---|---|---|
| 17 | CAGG | AcrIIA4 | No | |
| | (strong) | | | |
| 18 | SV40 | AcrIIA4 | No | |
| | (medium) | | | |
| 19 | CAGG | AcrIIA4 | DD | Shield1-controlled |
| | | | | (Titrate in mice |
| | | | | drinking water). |

TABLE 6
Combined dCas9-Anti-dCas9 strains

| | | | | | | | |
|---|---|---|---|---|---|---|---|
| Line | | Cas | dCas9 | | Antitoxin | | |
| # | Promoter | enzyme | degron | Antitoxin | degron | gRNAs | Comments |

Y chromosome engineering - for producing females; X - for producing males.

| | | | | | | | |
|---|---|---|---|---|---|---|---|
| 20 | CAGG | dCas9- | No | AcrII4A | DD | Full array: | Shield1 for both |
| | | KRAB- | | | | Spem1 | sexes. Titrate |
| | | MeCP2 | | | | Jhdm2a Sun4 | Shield1. |
| | | | | | | Sun5 Crem | |
| 21 | CAGG | dCas9- | No | AcrII4A | DD | Partial array: | Shield1 for both |
| | | KRAB- | | | | Spem1 | sexes. Titrate |
| | | MeCP2 | | | | | Shield1. |
| 22 | CAGG | dCas9- | UbM | AcrII4A | DD | Full array: | Shield1 for both |
| | | KRAB- | | | | Spem1 | sexes. Titrate |
| | | MeCP2 | | | | Jhdm2a Sun4 | Shield1. |
| | | | | | | Sun5 Crem | |
| 23 | CAGG | dCas9- | UbM | AcrII4A | DD | Partial array: | Shield1 for both |
| | | KRAB- | | | | Spem1 | sexes. Titrate |
| | | MeCP2 | | | | | Shield1. |
| 24 | CAGG | dCas9- | UbD | AcrII4A | DD | Full array: | Shield1 for both |
| | | KRAB- | | | | Spem1 | sexes. Titrate |
| | | MeCP2 | | | | Jhdm2a Sun4 | Shield1. |
| | | | | | | Sun5 Crem | |
| 25 | CAGG | dCas9- | UbD | AcrII4A | DD | Partial array: | Shield1 for both |
| | | KRAB- | | | | Spem1 | sexes. Titrate |
| | | MeCP2 | | | | | Shield1. |
| 26 | CAGG | dCas9- | UbK | AcrII4A | DD | Full array: | Shield1 for both |
| | | KRAB- | | | | Spem1 | sexes. Titrate |
| | | MeCP2 | | | | Jhdm2a Sun4 | Shield1. |
| | | | | | | Sun5 Crem | |
| 27 | CAGG | dCas9- | UbK | AcrII4A | DD | Partial array: | Shield1 for both |
| | | KRAB | | | | Spem1 | sexes. Titrate |
| | | | | | | | Shield1. |

Lines #1-5 all rely on the same principle. The Y gamete separates from the X gamete. Therefore, after separation, only the engineered gamete produces the dCas9. Because it produces it at an essential stage of gamete formation and targets genes essential for gamete cell formation, stability, viability and function, the gamete cannot mature. When the Y gamete contains the construct-then the male gametes do not mature and vice versa. The difference between the different lines is in the stability of dCas9, and in the genes targeted by the dCas9. The stability is dictated by different tags that reduce or stabilize the dCas9 (listed in the column “dCas9 degron”). The target genes are listed in the column “gRNAs/shRNA. Tetracycline addition results in silencing of the dCas9, and therefore produces both sexes.

Specifically, line #2 has been partially constructed and F0 mice show significant bias toward female production, as also disclosed in Examples 6 and 15.

Line #6 can only work as a GMO after crossing with lines 17-19. Tetracycline should be added during its production so that Cas9 does not damage the DNA. After crossing with a strain encoding a Cas9 inhibitor in the opposite gamete, the strain may be tested. The principle here is that throughout its expression, the Cas9 is inhibited in somatic cells due to the presence of the AcrII4A inhibitor. Therefore, it does not cause any damage to the DNA. Only after separation, does it cleave the DNA and destroys the gamete.

Lines #7-8_are similar in principle to lines 1-5. However, the promoter of dCas9 is constitutive. The degron of dCas9 is “Degradation domain—DD”. This domain protects from degradation of the dCas9 if Shield1 is added. Therefore, to produce single sex—the Shield1 should be added.

Lines #9-10 rely on the stability of small interfering RNAs in the gamete after separation. The gamete having it cannot mature, whereas the gamete lacking it develops normally. The cassette is under regulation of the tet-off promoter. Therefore, to produce both sexes, tetracycline should be added.

Lines #11-14 produce the dCas9 or Cas9 (more aggressive-cleaves DNA, not only represses transcription) under the natural promoter of one of the gamete development genes. This promoter is active only at the late maturation stage of the gamete, and therefore does not harm somatic cells. Upon expression of the dCas9 or Cas9 in the gamete, the gamete development is halted, or the gamete is destroyed. This results in single sex gametes. The degron of dCas9 or Cas9 is “Degradation domain—DD”. This domain protects from degradation of the dCas9 if Shield1 is added. Therefore, to produce single sex—the Shield1 should be added.

Lines #15-16 are similar to lines #11-14, but the degron is different. “LID” is a domain that destabilizes the protein in the presence of Shield1. Therefore, to produce both sexes, Shield1 should be added.

Lines #17-19 may be crossed with lines 1-16 to provide an additional control on the dCas9 or Cas9 activity. The AcrIIA4 is an inhibitor of dCas9 and Cas9 protein. Its construction into the gamete that is desired to survive, may enhance the survival, as it inhibits the dCas9\Cas9 activity. The lines 1-16 engineered on the Y chromosome to produce only females, should be crossed with lines 17-19 engineered on the X chromosome (in case females are desired), and vice versa. The expression is different between the lines and on one of them, DD is added so that Shield1 can be used to control the level of this inhibitor. If Shield1 is added. then the stability of the inhibitor is enhanced. However, despite the advantage of having an additional control of the Cas9, the resulting progeny is GMO because the AcrII4A inhibitor is inherited in the surviving sex (females).

Lines #20-27 rely on the principles of toxin-antitoxins. The toxin (dCas9) is more stable than the antitoxin (AcrII4A). Therefore, as long as both are expressed, no activity is detected. In this case −dCas9 does not inhibit transcription. However, if both are not expressed, after separation of the gametes, then the more stable toxin starts acting and repressing gene expression. Therefore, the gamete with the construct will survive, and the other will not. To save the other gamete, a degradation domain (DD) on the antitoxin can stabilize the anti-toxin and thus save both gametes. The Shield1 can be given at high doses for producing both sexes. It can be titrated at different concentrations to determine the most efficient stability required for the antitoxin to produce the best desired results. Different degrons are added to the dCas9 to determine optimal stability of it. Different targets are also combined to determine optimal construct.

TABLE 7
Gene list

| Gene | | Expression | | Phenotype in knockout mice | | |
|---|---|---|---|---|---|---|
| name | Specificity | Timeframe | Process | Testis size | Testis weight | Testis Histology |
| Gopc | Testis- | | Acrosome | Normal | Normal | Normal |
| | High | | | | | |
| | Kidney- | | | | | |
| | intermediate | | | | | |
| | Cerebellum - | | | | | |
| | low | | | | | |
| Akap4 | Highly in | | Tail | Normal | Normal | Normal |
| | Testis | | Formation | | | |
| | Some | | (Flagella) | | | |
| | reports in | | | | | |
| | CRC cells | | | | | |
| Sepp1 | Widely | | Tail | Normal | Normal | Normal |
| | expressed | | Formation | | | |
| | | | (Flagella)- | | | |
| | | | Selenium | | | |
| | | | transporter | | | |
| Vdac3 | Widely | | Tail | | | |
| | expressed | | Formation- | | | |
| | | | Expressed in | | | |
| | | | mitochnodria | | | |
| Tekt4 | Testis and | postnatal | Tail | Normal | Normal | Normal |
| | liver | days 16-18 | Formation | | | |
| | (Expressed | with a | (Flagella)- | | | |
| | in human | burst of | | | | |
| | pancreas) | expression | | | | |
| | | at day 18 | | | | |
| | | (18 d) | | | | |
| Tekt2 | Testis and | | Tail | | | |
| | liver | | Formation | | | |
| | | | (Flagella)- | | | |
| H1fnt | Testis | postnatal | Nuclear | Normal | Normal | Normal |
| | specific | days 21 | Condensation | | | |
| | | (continuous | | | | |
| | | until adult) | | | | |
| Prm2 | | step 7-15 | | Normal | Normal | Normal |
| | | spermatids | | | | |
| | | Many | | | | |
| | | mammals | | | | |
| | | express | | | | |
| | | only a | | | | |
| Prm1 | | single | | | | |
| | | protamine | | | | |
| | | gene | | | | |
| Tnp2 | | | | | | |
| Tnp1 | | | | | | |
| Crem | | | | Normal | Reduced | Normal |
| Sun4 | Testis | present in | Manchette | Normal | Normal | Normal |
| | specific | post- | formation | | | |
| | | meiotic | Nuclear | | | |
| | | cells. | remodeling | | | |
| | | SUN4 first | | | | |
| | | appears in | | | | |
| | | spermatids | | | | |
| | | where it | | | | |
| | | localizes | | | | |
| | | to the | | | | |
| | | nuclear | | | | |
| | | pole distal | | | | |
| | | to the | | | | |
| | | acrosome | | | | |
| Sun5 | Testis | Secondary | anchoring | Normal | Normal | Normal |
| | specific | spermatocytes | sperm head | | | |
| | | | to the tail | | | |
| Spag16 | | | Flagella | Trend | Normal | Some |
| | | | fromation | towards | | abnormalities |
| | | | | smaller | | observed |
| | | | | size | | |
| Meig1 | Testis and | 3 Meig1 | Elongation | Normal | Normal | Normal |
| | lungs | transcripts ~d | and | | | |
| | | 16-20 | condensation | | | |
| | | (one at d 6) | | | | |
| IFT88 | Testis liver | spermatocytes | Flagella | | | |
| | and heart | of stage | formation | | | |
| | | II-III | | | | |
| Hook1 | | | Manchette | | | |
| | | | development | | | |
| Tbpl1 | Testis and | Step 6-7 | Spermiogenic | Reduced | Reduced | Normal |
| | Brain | spermatids | arrest | | | |
| Tpap | | d 16-low | Spermiogenic | 45% | | Abnormal |
| | | d 20-high | arrest | decrease | | |
| Spem1 | Testis | Highly | Sperm | Normal | Normal | Normal |
| | specific | conserved | formation/ | | | |
| | | | maturation | | | |
| Jhdm2a | | d 18 | post- | Reduced | Reduced | Abnormal |
| | | | meiotic | | | |
| | | | chromatin | | | |
| | | | condensation | | | |
| | | | defects | | | |
| | | | leading to | | | |
| | | | morphogenic | | | |
| | | | defects | | | |
| Piwil1 | | Highly | Spermiogenic | Reduced | Reduced | Abnormal |
| | | conserved | arrest | | | |

| Gene | Phenotype in knockout mice | | | References/ |
|---|---|---|---|---|
| name | Sperm Count | Fertility | Comments | Credibility |
| Gopc | Reduced | infertile | no overt | Proc Natl Acad Sci |
| | | | abnormalities | U S A. 2002 Aug. |
| | | | in KO mice | 20; 99(17): 11211-6. |
| | | | | Epub 2002 Jul. 30 |
| | | | | 13 manuscripts- High |
| | | | | credibility (10) |
| Akap4 | Normal/ | Infertile | Short tail | Dev Biol. 2002 Aug. |
| | Reduced | | | 15; 248(2): 331-42. |
| | | | | 34 manuscripts (10) |
| Sepp1 | | Partial | 2 pregnancies | Biol Reprod. 2005 |
| | | Infertile | observed out | July; 73(1): 201-11. |
| | | | of 23 matings | Epub 2005 Mar. 2. |
| | | | (no litter | J Biol Chem. 2003 |
| | | | survived) | Apr. 18; |
| | | | Reduced | 278(16): 13640-6. |
| | | | Sperm | Epub 2003 Feb. 6. |
| | | | motility | (10) |
| Vdac3 | | Infertile | Pregnancies | J Biol Chem. 2001 Oct. |
| | | | observed | 19; 276(42): 39206-12. |
| | | | (plugs) but no | Epub 2001 Aug. 15. |
| | | | pups | |
| | | | Reduced | |
| | | | Sperm | |
| | | | motility | |
| Tekt4 | Normal | Reduced | Reduced | FASEB J. 2007 |
| | | fertility | Sperm | April; 21(4): 1013-25. |
| | | | motility | Epub 2007 Jan. 23. |
| | | | | (10) |
| Tekt2 | | | No data on | J Cell Biol. 2008 May |
| | | | knockout | 19; 181(4): 595-603. |
| | | | mice | doi: |
| | | | | 10.1083/jcb.200711160. |
| | | | | Epub 2008 May 12. |
| | | | | (10) |
| H1fnt | Normal | Infertile | | Mol Cell Biol. 2005 |
| | | | | August; 25(16): 7107-19. |
| | | | | (10) |
| Prm2 | Normal | Infertile | Prm2 can be | Nat Genet. 2001 |
| | | | targeted by | May; 28(1): 82-6. |
| | | | CRISPR | Sci Rep. 2016 Nov. |
| | | | Severe | 11; 6:36764. doi: |
| | | | morphological | 10.1038/srep36764. |
| | | | defects | (10) |
| | | | Immotile | |
| Prm1 | | Fertile | | Nat Genet. 2001 |
| | | | | May; 28(1): 82-6. |
| | | | | (10) |
| Tnp2 | | | No data on | |
| | | | knockout | |
| | | | mice | |
| Tnp1 | | | No data on | |
| | | | knockout | |
| | | | mice | |
| Crem | 0 (No | Infertile | | Nature. 1996 Mar. |
| | sperm | | | 14; 380(6570): 159-62. |
| | count) | | | (10) |
| Sun4 | Normal- | Infertile | | Dev Biol. 2015 Nov. |
| | No | | | 15; 407(2): 321-30. doi: |
| | mature | | | 10.1016/j.ydbio.2015.09.010. |
| | sperms | | | Epub 2015 Sep. 28. |
| | | | | (10) |
| Sun5 | Normal- | Infertile | Reduced | Elife. 2017 Sep. 25; 6. |
| | No | | motility | pii: e28199. doi: |
| | mature | | | 10.7554/eLife.28199. |
| | sperms | | | (10) |
| Spag16 | Normal- | Infertile | Reduced | Biol Reprod. 2006 |
| | No | | motility | April; 74(4): 751-9. Epub |
| | mature | | | 2005 Dec. 28 |
| | sperms | | | (10) |
| Meig1 | Normal | Infertile | MEIG1 is not | Proc Natl Acad Sci |
| | | | necessary for | U S A. 2009 Oct. |
| | | | meiosis, but | 6; 106(40): 17055-60. |
| | | | is required | doi: |
| | | | for the | 10.1073/pnas.0906414106. |
| | | | completion of | Epub 2009 Sep. 17 |
| | | | spermiogenesis | (10) |
| IFT88 | 1/350 | Infertile | Immotile | Mol Biol Cell. 2015 |
| | sperm | | completely | Dec. 1; 26(24): 4358-72. |
| | compared | | | doi: |
| | to WT | | | 10.1091/mbc.E15-08-0578. |
| | | | | Epub 2015 Sep. 30. |
| | | | | (10) |
| Hook1 | | | | Biol Reprod. 1988 |
| | | | | March; 38(2): 385-401 |
| | | | | (10) |
| Tbpl1 | None | Infertile | Probably die | Mol Cell. 2001 |
| | | | from | March; 7(3): 509-15. |
| | | | apoptosis | (10) |
| Tpap | | | Probably die | Science 2002: |
| | | | from | Vol. 298, Issue 5600, |
| | | | apoptosis | pp. 1999-2002 |
| | | | Regulator of | DOI: |
| | | | Prm1 and | 10.1126/science.1074632 |
| | | | Prm2 but not | (10) |
| | | | PIWI and | |
| | | | CREM | |
| Spem1 | Reduced | Infertile | | Proc Natl Acad Sci |
| | | | | U S A. 2007 Apr. |
| | | | | 17; 104(16): 6852-7. |
| | | | | Epub 2007 Apr. 10 |
| | | | | (10) |
| Jhdm2a | Reduced | Infertile | Appears to be | Nature. 2007 Nov. |
| | | | a master | 1; 450(7166): 119-23. |
| | | | regulator via | Epub 2007 Oct. 17. |
| | | | control of Prm | (10) |
| | | | and Tpn | |
| Piwil1 | None | Infertile | A master | |
| | | | regulator | |
| | | | together with | |
| | | | MIWI and | |
| | | | MILI | |
| | | | (controls | |
| | | | miRNAs) | |

### Example 6

### Preparation of Non-GMO Genetically Engineered Strain #2

The inventors constructed a plasmid for the preparation of the genetically engineered strain #2 for producing of progenies of the desired sex.

The construct for the preparation of a genetically engineered male that carry on the Y chromosome thereof nucleic acid sequences encoding the dCas9-KRAB under the Tet-off inducible promoter. The plasmid VB201210-1271bpw that comprise the dCas9-KRAB and five gRNAs directed at the Spem1 gene (Spem1-gRNA #1 (SEQ ID NO: 65), Spem1-gRNA #2 (SEQ ID NO: 63), Spem1-gRNA #3 (SEQ ID NO: 61), Spem1-gRNA #4 (SEQ ID NO: 59), Spem1-gRNA #5 (SEQ ID NO: 57)) that affects the maturation of the sperm. The insert is flanked by a right and a left homology arms that are specific for the target location (the uty gene) as shown in FIG. 3. Insertion of the cassette into chromosome Y results in transcription inhibition of the Spem1 gene and disturbed spermatogenesis of gametes that carry the Y transgene, while gametes that carry the X chromosome are not affected. As indicated above, doxycycline constantly represses the dCas9 by preventing binding of the tTA transcription factor to the promoter, thereby inhibiting gene expression. Therefore, for producing male progeny, doxycycline is added to the water and then the Y-chromosome containing gamete is preserved.

More specifically, a mouse uty knocking (KI) model in C57BL/6N mice was created as described in Experimental procedures. The elected target insertion gene is the uty gene (NCBI Reference Sequence: NM_009484.3), located on mouse chromosome Y. Twenty-seven exons have been identified, with the ATG start codon in exon 1 and the TAA stop codon in exon 27. Mouse genomic fragments containing homology arms (HAs) were amplified from BAC clones by using high fidelity Taq DNA polymerase and were sequentially assembled into a targeting vector together with recombination sites and selection markers as illustrated in the targeting vector of FIG. 2, the knock in (KI) sequence was inserted into intron 2 of mouse uty gene in the reverse orientation. The targeting vector includes an SDN (Self-deleting Neo Cassette) to allow the excision of the Neo cassette. It should be noted that the SDN may be also referred to in the present disclosure as SDA (self detection anchor). DTA is used for negative selection. The Sequence of the final targeting vectors of this genetically engineered male is denoted by SEQ ID NO: 51, the left homology arm is denoted by SEQ ID NO: 52, the KI transgene sequence is denoted by SEQ ID NO: 53, the SDN (Self-deleting Neo Cassette) is denoted by SEQ ID NO: 54, the right homology arm is denoted by SEQ ID NO: 55, and the DTA sequence is denoted by SEQ ID NO: 56. More specifically, the KI transgene sequence comprises a sequence encoding the spem1 gRNA #5, as denoted by SEQ ID NO: 57, U6 promoter sequence as denoted by SEQ ID NO: 58, a sequence encoding the spem1 gRNA #4, as denoted by SEQ ID NO: 59, the H1 promoter sequence as denoted by SEQ ID NO: 60, a sequence encoding the spem1 gRNA #3, as denoted by SEQ ID NO: 61, the h7SK promoter sequence as denoted by SEQ ID NO: 62, a sequence encoding the spem1 gRNA #2 as denoted by SEQ ID NO: 63, the mouse U6 promoter sequence as denoted by SEQ ID NO: 64, a sequence encoding the spem1 gRNA #1 as denoted by SEQ ID NO: 65, the human U6 promoter sequence as denoted by SEQ ID NO: 66, the tetracycline responsive element as denoted by SEQ ID NO: 67, a sequence encoding the KRAB domain as denoted by SEQ ID NO: 68, a sequence encoding the dCas9 as denoted by SEQ ID NO: 69, the CAG promoter sequence as denoted by SEQ ID NO: 70, the tTa sequence as denoted by SEQ ID NO: 71.

ES cells of C57BL/6N were used for gene targeting, and KI (knock-in) ES cells were established as described in the experimental procedures, using linearized vectors (as illustrated in FIG. 2). About 8-15 targeted ES cells were injected into the albino B6N embryos with using the Tubroknockout technology and then transplanted them into the CD-1 pseudo-pregnant mice to generate F0 mice with 100% chimerism.

Founder animals are identified by their coat color, their germline transmission was confirmed by breeding with B6N females and subsequent genotyping of the offspring. The SDN cassette is self-deleted in germ cells, so the offspring are Neo cassette-free. Male targeted mice were generated from targeted ES clones as final deliverables for this project.

### Example 7

### Preparation of Non-GMO Transgenic Strain #4

The inventors construct a further plasmid for the preparation of the genetically engineered strain #4 for producing of progenies of the desired sex.

The construct for the preparation of a genetically engineered male that carry on the Y chromosome thereof nucleic acid sequences encoding the dCas9-KRAB fused to a UbD degron under the Tet-off inducible promoter.

The plasmid pIY1001 comprises the dCas9-KRAB, UbD degron and five gRNAs directed at the Spem1 (SEQ ID NO: 116), Jhdm2a (SEQ ID NO: 115), Sun4 (SEQ ID NO: 114), Sun5 (SEQ ID NO: 113) and Crem (SEQ ID NO: 112) genes that affect the maturation and functionality of the gamete. The insert is flanked by a right and a left homology arms that are specific for the target location (the uty gene) as shown in FIG. 2. Insertion of the cassette into chromosome Y results in transcription inhibition of the Spem1, Jhdm2a, Sun4, Sun5 and Crem genes. Doxycycline constantly represses the dCas9 by preventing binding of the tTA transcription factor to the promoter, thereby inhibiting gene expression. Therefore, for producing male progeny, doxycycline is added to the water and the Y-chromosome containing gamete is preserved.

The mouse uty knockin (KI) model in C57BL/6N mice is next created as described above in Example 6 for the mice of line #2. Mouse genomic fragments containing homology arms are assembled into the targeting vector together with recombination sites and selection markers as illustrated in the targeting vector of FIG. 2, the KI sequence was inserted into intron 2 of mouse uty gene in the reverse orientation. The targeting vector includes an SDN (Self-deleting Neo Cassette) to allow the excision of the Neo cassette. DTA is used for negative selection. The Sequence of the final targeting vectors of this genetically engineered male is denoted by SEQ ID NO: 72, the left homology arm is denoted by SEQ ID NO: 73, the KI transgene sequence is denoted by SEQ ID NO: 74, the SDN (Self-deleting Neo Cassette) is denoted by SEQ ID NO: 75, the right homology arm is denoted by SEQ ID NO: 76, and the DTA sequence is denoted by SEQ ID NO: 77.

ES cells of C57BL/6N procedures, using linearized vectors. Targeted ES cell are injected into C57BL/6 albino embryos, and then re-implanted into CD-1 pseudo-pregnant females. Founder animals are identified by their coat color, their germline transmission is confirmed by breeding with C57BL/6 females and subsequent genotyping of the offspring. The SDN cassette is self-deleted in germ cells, so the offspring are Neo cassette-free. Male targeted mice are generated from targeted ES clones as final deliverables for this project.

### Example 8

### Preparation of GMO Genetically Engineered Strain #6

The inventors construct a further plasmid for the preparation of the genetically engineered strain #6 for producing of progenies of the desired sex.

The construct for the preparation of a genetically engineered male that carry on the Y chromosome thereof nucleic acid sequences encoding the Cas9 under the Tet-off inducible promoter. The Cas9 used herein is the Streptococcus pyogenes M1 GAS Cas9 having protein id: AAK33936.1, that comprises the amino acid sequence as denoted by SEQ ID NO: 48. In some embodiments, the disclosed Cas9 is encoded by the nucleic acid sequence as denoted by SEQ ID NO: 47. Additional alternative embodiments may use the Cas9 adapted for mammalian expression, for example the one comprising the amino acid sequence as denoted by SEQ ID NO: 50, encoded by SEQ D NO: 49. The plasmid pIY1001 comprises the Cas9 and five gRNAs directed at the Spem1, Jhdm2a, Sun4, Sun5 and Crem genes that affect the maturation and functionality of the gamete. The insert is flanked by a right and a left homology arms that are specific for the target location (the uty gene) as shown in FIG. 2. Insertion of the cassette into chromosome Y results in transcription inhibition of the Spem1 (SEQ ID NO: 116), Jhdm2a (SEQ ID NO: 115), Sun4 (SEQ ID NO: 114), Sun5 (SEQ ID NO: 113) and Crem (SEQ ID NO: 112). Doxycycline constantly represses the dCas9 by preventing binding of the tTA transcription factor to the promoter, thereby inhibiting gene expression. Therefore, for producing male progeny, doxycycline is added to the water and the Y-chromosome containing gamete is preserved.

The mouse uty knockin (KI) model in C57BL/6 mice is next created as described above in Example 6 for the mice of line #2. Mouse genomic fragments containing homology arms are assembled into the targeting vector together with recombination sites and selection markers as illustrated in the targeting vector of FIG. 2, the KI sequence is inserted into intron 2 of mouse uty gene in the reverse orientation. The targeting vector includes an SDN (Self-deleting Neo Cassette) to allow the excision of the Neo cassette. DTA is used for negative selection. The Sequence of the final targeting vectors of this genetically engineered male is denoted by SEQ ID NO: 78, the left homology arm is denoted by SEQ ID NO: 73, the KI transgene sequence is denoted by SEQ ID NO: 79, the SDN (Self-deleting Neo Cassette) is denoted by SEQ ID NO: 75, the right homology arm is denoted by SEQ ID NO: 76, and the DTA sequence is denoted by SEQ ID NO: 77.

ES cells of C57BL/6 are used for gene targeting, and KI (knock-in) ES cells are established as described in the experimental procedures, using linearized vectors (as illustrated in FIG. 2). Targeted ES cell are injected into C57BL/6 albino embryos, and then re-implanted into CD-1 pseudo-pregnant females. Founder animals are identified by their coat color, their germline transmission is confirmed by breeding with C57BL/6 females, and subsequent genotyping of the offspring. The SDN cassette is self-deleted in germ cells, so the offspring are Neo cassette-free. Male targeted mice are generated from targeted ES clones as final deliverables for this project. Mice of the above lines may be crossed with homogametic mice (female) of lines #17-#19, that carry the anti-Cas transgene on their X chromosome, thereby producing GMO female that carry the anti-Cas9 provided by the X chromosome of the female of lines #17-19. Doxycycline treatment provides production of male GMO progenies (and/or semen) that carry the dCas9 or Cas9 on the Y chromosome and the anti-Cas9 on the X chromosome.

### Example 9

### Preparation of Non-GMO Genetically Engineered Strain #7

The inventors construct a further plasmid for the preparation of the genetically engineered strain #7 for producing of progenies of the desired sex.

The construct for the preparation of a genetically engineered male, carry on the Y chromosome thereof nucleic acid sequences encoding the dCas9-KRAB fused to “Degradation domain” (DD) degron under the CAGG promoter for constitutive expression (as denoted by SEQ ID NO: 70). The DD domain protects from degradation of the dCas9 if Shield1 is added.

The plasmid pIY1001 comprises the dCas9-KRAB, DD degron and five gRNAs directed at the Spem1 (SEQ ID NO: 116), Jhdm2a (SEQ ID NO: 115), Sun4 (SEQ ID NO: 114), Sun5 (SEQ ID NO: 113) and Crem (SEQ ID NO: 112) that affect the maturation and functionality of the gamete. The insert is flanked by a right and a left homology arms that are specific for the target location (the uty gene) as shown in FIG. 2. Insertion of the cassette into chromosome Y results in transcription inhibition of the Spem1, Jhdm2a, Sun4, Sun5 and Crem genes when Shield1 is added. The mouse uty knock in (KI) model in C57BL/6 mice is next created as described above in Example 6. Mouse genomic fragments containing homology arms are assembled into the targeting vector together with recombination sites and selection markers as illustrated in the targeting vector of FIG. 2, the KI sequence is inserted into intron 2 of mouse uty gene in the reverse orientation. The targeting vector includes an SDN (Self-deleting Neo Cassette) to allow the excision of the Neo cassette. DTA is used for negative selection. The Sequence of the final targeting vectors of this genetically engineered male is denoted by SEQ ID NO: 80, the left homology arm is denoted by SEQ ID NO: 73, the KI transgene sequence is denoted by SEQ ID NO: 81, the SDN (Self-deleting Neo Cassette) is denoted by SEQ ID NO: 75, the right homology arm is denoted by SEQ ID NO: 76, and the DTA sequence is denoted by SEQ ID NO: 77.

ES cells of C57BL/6 are used for gene targeting, and KI (knock-in) ES cells are established as described in the experimental procedures, using linearized vectors (as illustrated in FIG. 2). Targeted ES cell are injected into C57BL/6 albino embryos, and then re-implanted into CD-1 pseudo-pregnant females. Founder animals are identified by their coat color, their germline transmission is confirmed by breeding with C57BL/6 females and subsequent genotyping of the offspring. The SDN cassette is self-deleted in germ cells, so the offspring are Neo cassette-free. Male targeted mice are generated from targeted ES clones as final deliverables for this project.

### Example 10

### Preparation of Non-GMO Genetically Engineered Strain #8

The inventors construct a further plasmid for the preparation of the genetically engineered strain #8 for producing of progenies of the desired sex.

The construct for the preparation of a genetically engineered male that carry on the Y chromosome thereof nucleic acid sequences encoding the dCas9-KRAB fused to “Degradation domain” (DD) degron under the CAGG promoter for constitutive expression as described in Example 9 for strain #7. The DD domain protects from degradation of the dCas9 if Shield1 is added.

The plasmid pIY1001 comprises the dCas9-KRAB, DD degron and five gRNAs directed at the Spem1 gene (Spem1-gRNA #1 (SEQ ID NO: 65), Spem1-gRNA #2 (SEQ ID NO: 63), Spem1-gRNA #3 (SEQ ID NO: 61), Spem1-gRNA #4 (SEQ ID NO: 59), Spem1-gRNA #5 (SEQ ID NO: 57)) that affects the maturation of the sperm. The insert is flanked by a right and a left homology arms that are specific for the target location (the uty gene) as shown in FIG. 2. Insertion of the cassette into chromosome Y results in transcription inhibition of the Spem1 gene when Shield1 is added and disturbed spermatogenesis of gametes that carry the Y transgene, while gametes that carry the X chromosome are not affected.

The mouse uty knockin (KI) model in C57BL/6 mice is next created as described above in Example 6. Mouse genomic fragments containing homology arms are assembled into the targeting vector together with recombination sites and selection markers as illustrated in the targeting vector of FIG. 2, the KI sequence is inserted into intron 2 of mouse uty gene in the reverse orientation. The targeting vector includes an SDN (Self-deleting Neo Cassette) to allow the excision of the Neo cassette. DTA is used for negative selection. The Sequence of the final targeting vectors of this genetically engineered male is denoted by SEQ ID NO: 82, the left homology arm is denoted by SEQ ID NO: 73, the KI transgene sequence is denoted by SEQ ID NO: 83, the SDN (Self-deleting Neo Cassette) is denoted by SEQ ID NO: 75, the right homology arm is denoted by SEQ ID NO: 76, and the DTA sequence is denoted by SEQ ID NO: 77.

ES cells of C57BL/6 are used for gene targeting, and KI (knock-in) ES cells are established as described in the experimental procedures, using linearized vectors (as illustrated in FIG. 2). Targeted ES cell are injected into C57BL/6 albino embryos, and then re-implanted into CD-1 pseudo-pregnant females. Founder animals are identified by their coat color, their germline transmission is confirmed by breeding with C57BL/6 females and subsequent genotyping of the offspring. The SDN cassette is self-deleted in germ cells so the offspring are Neo cassette-free. Male targeted mice are generated from targeted ES clones as final deliverables for this project.

### Example 11

### Preparation of Non-GMO Genetically Engineered Strain #11

The inventors construct a further plasmid for the preparation of the genetically engineered strain #11 for producing of progenies of the desired sex.

The construct for the preparation of a genetically engineered male that carry on the Y chromosome thereof nucleic acid sequences encoding the dCas9-KRAB fused to “Degradation domain” (DD) degron under the natural promoter of the gamete development gene Spem1/Akap4. This promoter is active only at the late maturation stage of the gamete, and therefore does not harm somatic cells. In addition, the DD domain protects from degradation of the dCas9 if Shield1 is added.

The plasmid pIY1001 comprises the dCas9-KRAB, DD degron and five gRNAs directed at the Spem1 (SEQ ID NO: 116), Jhdm2a (SEQ ID NO: 115), Sun4 (SEQ ID NO: 114), Sun5 (SEQ ID NO: 113) and Crem (SEQ ID NO: 112) genes that affect the maturation and functionality of the gamete. The insert is flanked by a right and a left homology arms that are specific for the target location (the uty gene) as shown in FIG. 2. Insertion of the cassette into chromosome Y results in transcription inhibition of the Spem1, Jhdm2a, Sun4, Sun5 and Crem genes.

The mouse uty knockin (KI) model in C57BL/6 mice is next created as described above in Example 6, for the mice of line #2. Mouse genomic fragments containing homology arms are assembled into the targeting vector together with recombination sites and selection markers as illustrated in the targeting vector of FIG. 2, the KI sequence is inserted into intron 2 of mouse uty gene in the reverse orientation. The targeting vector includes an SDN (Self-deleting Neo Cassette) to allow the excision of the Neo cassette. DTA is used for negative selection. The Sequence of the final targeting vectors of this genetically engineered male is denoted by SEQ ID NO: 84, the left homology arm is denoted by SEQ ID NO: 73, the KI transgene sequence is denoted by SEQ ID NO: 85, the SDN (Self-deleting Neo Cassette) is denoted by SEQ ID NO: 75, the right homology arm is denoted by SEQ ID NO: 76, and the DTA sequence is denoted by SEQ ID NO: 77. ES cells of C57BL/6 are used for gene targeting, and KI (knock-in) ES cells are established as described in the experimental procedures, using linearized vectors (as illustrated in FIG. 2). Targeted ES cell are injected into C57BL/6 albino embryos, and then re-implanted into CD-1 pseudo-pregnant females. Founder animals are identified by their coat color, their germline transmission is confirmed by breeding with C57BL/6 females and subsequent genotyping of the offspring. The SDN cassette is self-deleted in germ cells, so the offspring are Neo cassette-free. Male targeted mice are generated from targeted ES clones as final deliverables for this project.

### Example 12

### Preparation of Non-GMO Genetically Engineered Strain #12

The inventors construct a further plasmid for the preparation of the genetically engineered strain #12 for producing of progenies of the desired sex.

The construct for the preparation of a genetically engineered male that carry on the Y chromosome thereof nucleic acid sequences encoding the dCas9-KRAB fused to “Degradation domain” (DD) degron under the natural promoter of the gamete development gene Spem1/Akap4. This promoter is active only at the late maturation stage of the gamete, and therefore does not harm somatic cells. In addition, the DD domain protects from degradation of the dCas9 if Shield1 is added.

The plasmid pIY1001 comprises the dCas9-KRAB, DD degron and five gRNAs directed at five gRNAs directed at the Spem1 gene (Spem1-gRNA #1 (SEQ ID NO: 65), Spem1-gRNA #2 (SEQ ID NO: 63), Spem1-gRNA #3 (SEQ ID NO: 61), Spem1-gRNA #4 (SEQ ID NO: 59), Spem1-gRNA #5 (SEQ ID NO: 57)) that affects the maturation of the sperm. The insert is flanked by a right and a left homology arms that are specific for the target location (the uty gene) as shown in FIG. 2. Insertion of the cassette into chromosome Y results in transcription inhibition of the Spem1 gene and disturbed spermatogenesis of gametes that carry the Y transgene, while gametes that carry the X chromosome are not affected.

The mouse uty knockin (KI) model in C57BL/6 mice is next created as described above in Example 6. Mouse genomic fragments containing homology arms are assembled into the targeting vector together with recombination sites and selection markers as illustrated in the targeting vector of FIG. 2, the KI sequence is inserted into intron 2 of mouse uty gene in the reverse orientation. The targeting vector includes an SDN (Self-deleting Neo Cassette) to allow the excision of the Neo cassette. DTA is used for negative selection. The Sequence of the final targeting vectors of this genetically engineered male is denoted by SEQ ID NO: 86, the left homology arm is denoted by SEQ ID NO: 73 the KI transgene sequence is denoted by SEQ ID NO: 87, the SDN (Self-deleting Neo Cassette) is denoted by SEQ ID NO: 75, the right homology arm is denoted by SEQ ID NO: 76, and the DTA sequence is denoted by SEQ ID NO: 77.

ES cells of C57BL/6 are used for gene targeting, and KI (knock-in) ES cells are established as described in the experimental procedures, using linearized vectors (as illustrated in FIG. 2). Targeted ES cell are injected into C57BL/6 albino embryos, and then re-implanted into CD-1 pseudo-pregnant females. Founder animals are identified by their coat color, their germline transmission is confirmed by breeding with C57BL/6 females and subsequent genotyping of the offspring. The SDN cassette is self-deleted in germ cells, so the offspring are Neo cassette-free. Male targeted mice are generated from targeted ES clones as final deliverables for this project.

### Example 13

### Preparation of GMO Genetically Engineered Strain #19

The inventors construct a further plasmid for the preparation of the genetically engineered strain #19 for producing of progenies of the desired sex.

The construct for the preparation of a genetically engineered female that carry on the X chromosome thereof nucleic acid sequences encoding the anti-Cas9 inhibitor (AcrIIA4, as denoted by SEQ ID NO: 118) fused to “Degradation domain” (DD, as denoted by SEQ ID NO: 117) degron under the CAGG promoter for constitutive expression as described in Example 10 for strain #7. The DD domain protects from degradation of the dCas9 if Shield1 is added.

The plasmid pIY1001 comprises the AcrIIA4 and the DD degron. The insert is flanked by a right and a left homology arms that are specific for the target location (Hprt exon 2 on the X chromosome). Insertion of the cassette into chromosome X results in inhibition of Cas9 or dCas9 gene. Therefore, line #19 may be crossed with lines 1-16 to provide an additional control on the dCas9 or Cas9 activity.

The mouse hprt knock in (KI) model in C57BL/6 female mice is next created as described above in Example 6. Mouse genomic fragments containing homology arms are assembled into the targeting vector together with recombination sites and selection. The KI sequence is inserted into exon 2 of mouse hprt gene in the reversed orientation. The targeting vector includes an SDN (Self-deleting Neo Cassette) to allow the excision of the Neo cassette. DTA is used for negative selection. The Sequence of the final targeting vectors of this genetically engineered male is denoted by SEQ ID NO: 88, the left homology arm is denoted by SEQ ID NO: 120, the KI transgene sequence is denoted by SEQ ID NO: 89, the SDN (Self-deleting Neo Cassette) is denoted by SEQ ID NO: 75, the right homology arm is denoted by SEQ ID NO: 121, and the DTA sequence is denoted by SEQ ID NO: 77.

Targeted ES cell are injected into C57BL/6 albino embryos, and then re-implanted into CD-1 pseudo-pregnant females. Founder animals are identified by their coat color, their germline transmission is confirmed by breeding with either of C57BL/6 males of line #1-16 and subsequent genotyping of the offspring. The SDN cassette is self-deleted in germ cells, so the offspring are Neo cassette-free. Female targeted mice are generated from targeted ES clones as final deliverables for this project.

### Example 14

### Preparation of GMO Genetically Engineered Strain #20

The inventors next constructed a plasmid that contain both, the toxin (dCas9-KRAB) and the anti-toxin (AcrII4A-DD) for the preparation of the genetically engineered strains for production of progenies of the desired sex. More specifically, a genetically engineered male that carry on the Y chromosome thereof nucleic acid sequences encoding the dCas9-KRAB under the CAGG promoter for constitutive expression, and the following gRNAs directed at the Spem1 (SEQ ID NO: 116), Jhdm2a (SEQ ID NO: 115), Sun4 (SEQ ID NO: 114), Sun5 (SEQ ID NO: 113) and Crem (SEQ ID NO: 112) genes that affect the maturation and functionality of the gamete. The insert is flanked by a right and a left homology arms that are specific for the target location (the uty gene). Nucleic acid sequences encoding the antitoxin AcrII4A fused to a destabilizing domain (DD) that leads to the entire protein degradation in the absence of the synthetic chemical Shield1, was also included in the construct. Insertion of the cassette into chromosome Y resulted in transcription inhibition of the Spem1, Jhdm2a, Sun4, Sun5 and Crem genes, however, the co-expression of the antitoxin AcrII4A, protects the Y-gametes. Still further, since the dCas9 is stable (e.g., expressed under a strong constitutive promoter), it may be present in the X gametes, while the antitoxin (degraded by the degron) is not stable in these gametes. After meiosis, dCas9 eliminates X gametes due to loss of inactivation by the antitoxin. However, in Y-gametes, dCas9 is still inactivated by the anti-dCas9 AcrII4A. More specifically, the dCas9 presence in the X gametes with only negligible amounts of the antitoxin AcrII4A, results in distribution of gametes that carry the X chromosome, while the Y gametes that co-express the antitoxin AcrII4A, are protected. Therefore, gametes that carry the Y chromosome that also includes the anti-dCas9 AcrII4A, will survive, while X gametes that are exposed to the presence of dCas9 with only negligible amounts of antitoxin AcrII4A, are destroyed. The parental mouse line is able to produce both male and female progeny upon supplement of Shield1 in their drinking water, that stabilizes the antitoxin AcrII4A, such that the presence of the antitoxin in the separated X gametes is sufficient for protecting against the dCas9.

The mouse uty knockin (KI) model in C57BL/6 mice is next created as described above in Example 6, for the mice of line #2. Mouse genomic fragments containing homology arms are assembled into the targeting vector together with recombination sites and selection markers as illustrated in the targeting vector of FIG. 2, the KI sequence was inserted into intron 2 of mouse uty gene in the reverse orientation. The Sequence of the final targeting vectors of this genetically engineered male is denoted by SEQ ID NO: 119, the left homology arm is denoted by SEQ ID NO: 109, the KI transgene sequence is denoted by SEQ ID NO: 110, the SDN (Self-deleting Neo Cassette) is denoted by SEQ ID NO: 75, the right homology arm is denoted by SEQ ID NO: 111, and the DTA sequence is denoted by SEQ ID NO: 77. More specifically, the destabilizing domain (DD) sequence as denoted by SEQ ID NO: 117, a sequence encoding the AcrIIA4 as denoted by SEQ ID NO: 118.

### Example 15

### Genetic Sex Biasing in Mammals and Productions of Non-GMO Progeny of the Desired Sex

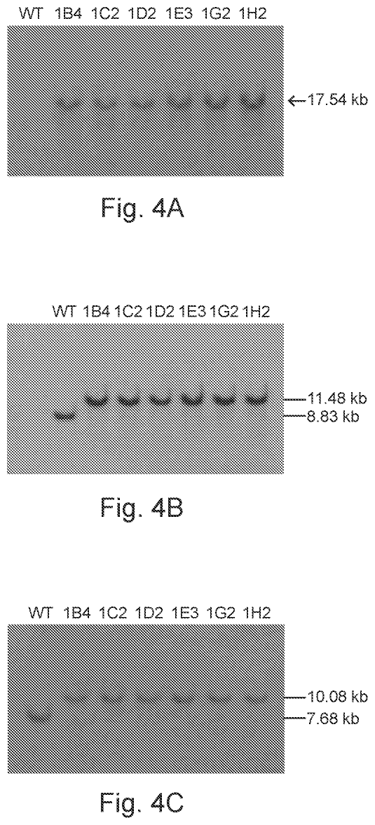

As a proof of concept for the inventor's approach, single-sex mouse progeny that retains a reproductive reservoir of males and females were produced. For developing mice that produce only females, the inventors used the BL6 self-sustained mouse line that produces males and females at an equal ratio. Using this mouse line, genetically modified males were generated by encoding on the Y chromosome CRISPR guide RNAs (gRNAs) that target one (Spem1) (named line #2, as per the dCAS9 strains; Table 4). The selected target genes were all shown to be essential for mouse spermatogenesis. As can be seen in FIG. 4, the inventors have successfully generated ES cell clones. The mESC were then used to generate F0 mice using a proprietary technology resulting in little to no chimerism of the developing mouse [Ukai H, et al., Nature Protocols 12:2513-2530 (2017)]. Line #2, which targeted only 1 gene, showed normal testis and epididymis morphology and normal sperm (Table 8).

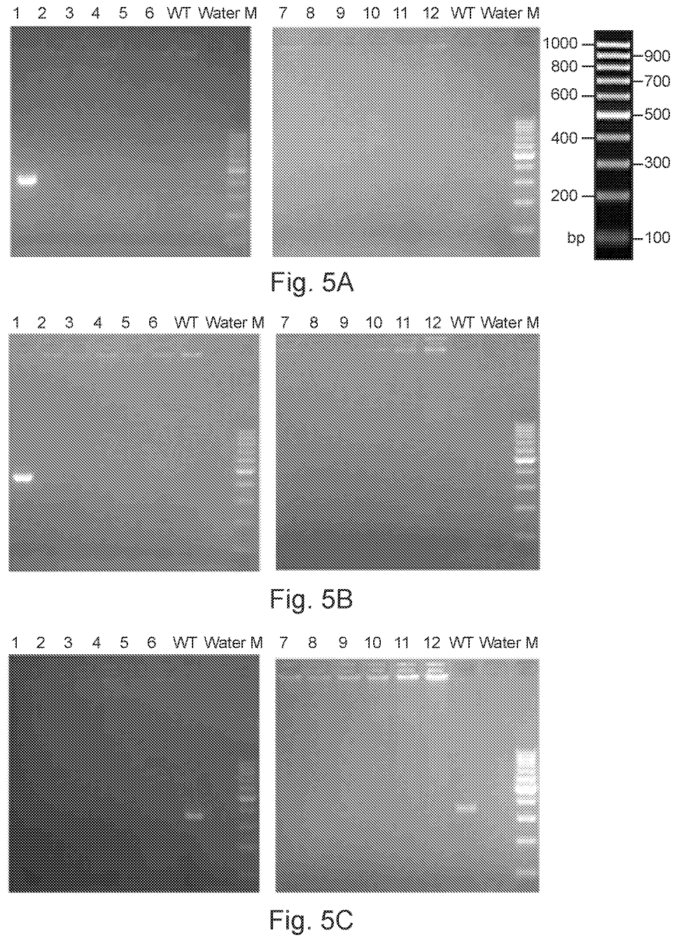

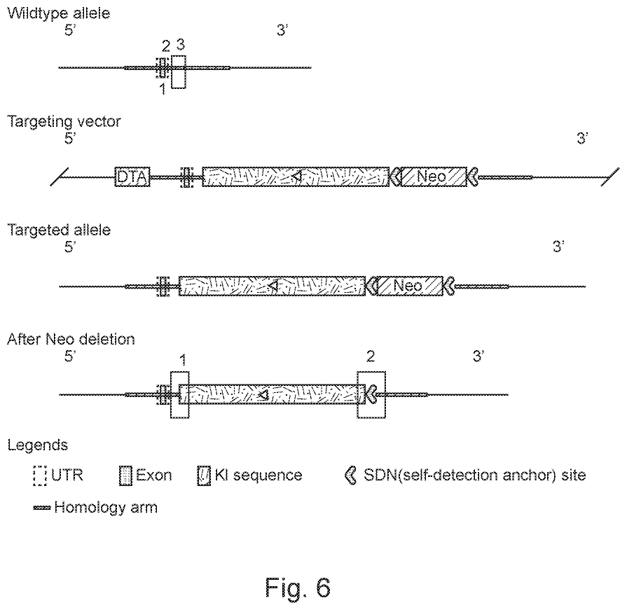

The inventors crossed the F0 line #2 males with wild type C57BL/6N females. Two litters of 6 pups each were obtained, with a total of 11 females (91.7%) and 1 male (8.3%) (Table 9). Genetic examination of the sex of the pups is shown in FIG. 5 and was performed as illustrated in FIG. 6. This result suggested that the genetically engineered male mice bias the sex ratio significantly. To obtain results with better statistical power, and to determine the tetracycline switch efficiency, the inventors divided F0 males into two groups: 18 received tetracycline at a concentration of 2 mg/ml in their drinking water for 5 weeks before mating, and 4 received regular water. Sperm from these mice was taken for in vitro fertilization of wild type C57BL/6N females.

Sperm from the untreated mice produced 4 litters totaling 35 pups (average of ˜8.7 pups per litter). Sperm from the 18 tetracycline-treated mice produced 18 litters totaling 152 pups (average of ˜8.4 pups per litter) (Table 10 and Table 11). Remarkably, the untreated sperm produced 32 females (91.4%), and only 3 males (8.6%), as determined by PCR (Table 11). The sperm from the treated mice produced 125 females (82.2%) and 27 males (17.8%) (Table 11). These results show, with high statistical significance (P<0.00001; a two-tailed binomial test, assuming an equal male-to-female ratio) that the untreated sperm biases the sex ratio toward females. They also demonstrate with high statistical significance (P<0.001; a two-tailed binomial test, assuming the female:male ratio of the untreated sperm) that the tetracycline treated sperm reduces this bias. Importantly, all of the 125 F1 females lacked the transgene, as determined by PCR. The negative PCR thus confirm that genetic crossover between the Y chromosome and the X chromosome did not occur in these females, and that they are all non-GMO. All the males had a positive PCR amplification of the cassette confirming the presence of the transgene on their Y chromosome, as expected. These results demonstrate, for the first time, a genetic sex biasing in mammals resulting in non-GMO progeny of the desired sex.

TABLE 8
Characteristics of F0 line 2 males
Line #2: TKN-201123-CAA-01, pDonor-DTA-mUty_LA-5x
gRNA-TRE > KRAB: dCas9-CAG > tTA-SDN-mUty_RA

| Project | Mouse | | | Sperm volume | Sperm diffusion |
|---|---|---|---|---|---|
| ID | ID | Testis | Epididymis | judgment | status |
| TKIE100 | 2 | Normal | Normal | Normal | Good diffusion |
| | 3 | Normal | Small | Scarce | General diffusion |
| | | | | | and sperm clumping |
| | 4 | Normal | Normal | Slightly less | Good diffusion |
| | 5 | Normal | Normal | Slightly less | Good diffusion |

TABLE 9
Summary of F1 sex identification from crossing
F0 line #2 males with BL6-line females

| F1 Date of birth | #of F1 ♀ | #of F1 ♂ |
|---|---|---|
| 2022 Jul. 23 | 5 | 1 |
| 2022 Aug. 24 | 6 | 0 |

TABLE 10
F1 sex identification from crossing F0 line #2 males
with BL6-line females treated or untreated with tetracycline

| | | positive F0 | | | | | | Number of |
|---|---|---|---|---|---|---|---|---|
| | | male (DOB | Number of | Number of | Number of | Number of | Number of | positive |
| | | 2022 Nov. | total | male | female | positive | positive | PCR3 (WT Y- |
| Group | Clone | 22, KI) | F1 mice | F1 mice | F1 mice | PCR1 (KI) | PCR2 (KI) | chromosome) |
| Untreated | 1E3 | F0-1 | 6 | 0 | 6 | 0 | 0 | 0 |
| | 1E3 | F0-2 | 15 | 1 | 14 | 1 | 1 | 0 |
| | 1E3 | F0-3 | 10 | 2 | 8 | 2 | 2 | 0 |
| | 1E3 | F0-5 | 4 | 0 | 4 | 0 | 0 | 0 |
| Tet treated | 1G2 | F0-8 | 8 | 2 | 6 | 2 | 2 | 0 |
| | 1G2 | F0-9 | 7 | 3 | 4 | 3 | 3 | 0 |
| | 1G2 | F0-10 | 8 | 2 | 6 | 2 | 2 | 0 |
| | 1G2 | F0-7 | 18 | 5 | 13 | 5 | 4 | 0 |
| | 1C2 | F0-1 | 7 | 1 | 6 | 1 | 1 | 0 |
| | 1C2 | F0-2 | 12 | 0 | 12 | 0 | 0 | 0 |
| | 1C2 | F0-3 | 16 | 1 | 15 | 1 | 1 | 0 |
| | 1C2 | F0-4 | 7 | 0 | 7 | 0 | 0 | 0 |
| | 1C2 | F0-5 | 5 | 1 | 4 | 1 | 1 | 0 |
| | 1C2 | F0-6 | 8 | 2 | 6 | 2 | 1 | 0 |
| | 1E3 | F0-8 | 2 | 0 | 2 | 0 | 0 | 0 |
| | 1E3 | F0-9 | 3 | 0 | 3 | 0 | 0 | 0 |
| | 1E3 | F0-10 | 6 | 0 | 6 | 0 | 0 | 0 |
| | 1E3 | F0-11 | 10 | 1 | 9 | 1 | 1 | 0 |
| | 1E3 | F0-12 | 7 | 1 | 6 | 1 | 1 | 0 |
| | 1E3 | F0-13 | 4 | 1 | 3 | 1 | 1 | 0 |
| | 1E3 | F0-15 | 10 | 3 | 7 | 3 | 3 | 0 |
| | 1E3 | F0-16 | 14 | 4 | 10 | 4 | 4 | 0 |

TABLE 11
Summary of F1 sex identification from crossing F0 line #2
males with BL6-line females treated or untreated with tetracycline

| | Total | Total | Total | Average |
|---|---|---|---|---|
| Summary | F1 mice | F1 male | F1 female | litter size |
| Untreated | 35 | 3 | 32 | 8.75 |
| Tet treated | 152 | 27 | 125 | 8.44 |

1.-60. (canceled)

61. A genetically engineered eukaryotic heterogametic organism comprising:
(a) a first sex chromosome comprising at least one exogenous nucleic acid sequence comprising:
(i) at least one nucleic acid sequence encoding at least one nucleic acids modifier component; and
(ii) at least one nucleic acid sequence encoding or forming at least one target recognition element for said at least one nucleic acids modifier component;
wherein said exogenous nucleic acid sequence optionally further comprise:
(iii) at least one nucleic acid sequence encoding at least one inhibitor or repressor for said at least one nucleic acids modifier component of (i);
optionally, said genetically engineered eukaryotic heterogametic organism further comprises:
(b) a second sex chromosome comprising at least one of:
(iv) at least one exogenous nucleic acid sequence encoding at least one inhibitor or repressor for said at least one nucleic acids modifier component of (i); and/or
(v) at least one endogenous or exogenous nucleic acid sequence encoding at least one transcription factor and/or repressor, that specifically bind at least one transcription regulatory element operably linked to the nucleic acid sequence of at least one of (i), (ii) and (iii).

62. The genetically engineered eukaryotic heterogametic organism according to claim 61, wherein at least one of:
(A) at least one of said nucleic acid sequences of (i), (ii), (iii), (iv) and/or (v) are operably linked to at least one of: at least one inducible or repressible regulatory element, at least one transcriptional regulatory element, at least one post-translationally regulatory element and at least one temporally or spatially regulatory element;
(B) wherein said nucleic acids modifier component is at least one of: at least one modifier protein or any fusion protein thereof, and at least one modifier nucleic acid sequence, optionally, said modifier protein is a nuclease, said nuclease is at least one of: (i) a nuclease having a nucleolytic activity and/or a fusion protein thereof; (ii) a non-active nuclease and/or a fusion protein thereof;
(C) wherein the nuclease is an RNA guided nuclease, and wherein said at least one target recognition element is at least one ribonucleic acid guide (guide RNA) directed against at least one target sequence within coding and/or non-coding sequences, and/or any product/s thereof, of at least one gene involved in viability, stability and/or function of gamete cells of said organism;
(D) wherein said at least one gene involved in viability, stability and/or function of gamete cells of said organism is expressed at a late stage of gametogenesis;
(E) wherein at least one of:
(a) said RNA guided DNA binding protein nuclease is a CRISPR-associated endonuclease 9 (Cas9) system; and/or
(b) said inhibitor of said at least one nucleic acids modifier protein, is an inhibitor of Cas9, optionally, said Cas9 inhibitor is anti-CRISPR (Acr) IIA4 (AcrIIA4); and
(F) wherein at least one of:
(I) wherein said inducible or repressible regulatory element is at least one of:
(a) a repressible promoter, said repressible promoter is at least one of Tetracycline (Tet)-off, Alcohol dehydrogenase 1 (ADH1), GAL4/UAS, LexA/lexAop, and the Q system;
(b) at least one degron, wherein said degron is at least one of: destabilization domain (DD), ligand-induced degradation (LID), and/or auxin-inducible degron (AID);
(II) wherein said post-translationally regulatory element is a degron, said degron is any one of DD, UbK, UbD, and/or UbM; and
(III) wherein said at least one temporally or spatially regulatory element is the Spermatid maturation protein 1 (spem 1) promoter.

63. The genetically engineered eukaryotic heterogametic organism according to claim 61, wherein at least one of:
(A) the heterogametic organism comprising a first sex chromosome comprising:
(i) at least one nucleic acid sequence encoding at least one Cas9 having a nucleolytic activity and/or a fusion protein thereof; or at least one non-active Cas9 and/or a fusion protein thereof; and
(ii) at least one nucleic acid sequence encoding at least one gRNA directed against at least one target sequence; wherein said first sex chromosome characterizes the undesired sex;
optionally, at least one of said nucleic acid sequences of (i) and (ii), are operably linked to at least one of at least one inducible regulatory element, at least one transcriptional regulatory element, at least one post-translationally regulatory element and at least one temporally or spatially regulatory element;
(B) the heterogametic organism comprising a first sex chromosome comprising:
(i) at least one nucleic acid sequence encoding a catalytically dead Cas9 (dCas9)-Krüppel associated box (KRAB) domain-methyl CpG-binding protein 2 (MeCP2) (dCas9-KRAB-MeCP2) fusion protein under a Tet-off promoter; and
(ii) at least one nucleic acid sequence encoding at least one gRNA directed against at least one target sequence within coding and/or non-coding sequences, and/or any product/s thereof, of at least one gene involved in viability, stability and/or function of gamete cells of said organism; and
(C) the heterogametic organism comprising a first sex chromosome comprising:
(i) at least one nucleic acid sequence encoding dCas9-KRAB-MeCP2 fusion protein under a Tet-off promoter; and
(ii) at least one nucleic acid sequence encoding at least one gRNA directed against at least one target sequence within the spermatid maturation 1 (Spem1) gene; wherein said first sex chromosome characterizes the undesired sex.

64. The genetically engineered eukaryotic heterogametic organism according to claim 61, wherein:
(A) the heterogametic organism comprising:
(a) a first sex chromosome comprising:
(i) at least one nucleic acid sequence encoding at least one Cas9 having a nucleolytic activity and/or a fusion protein thereof; or at least one non-active Cas9 and/or any fusion protein thereof; and
(ii) at least one nucleic acid sequence encoding at least one gRNA directed against at least one target sequence;
wherein at least one of said nucleic acid sequences of (i) and/or (ii), are operably linked to at least one transcription regulatory element, said regulatory element being specific for a repressor endogenously encoded by a nucleic acid sequence comprised within the second sex chromosome;
and wherein said first sex chromosome characterizes the undesired sex;
optionally, at least one of said nucleic acid sequences of (i), (ii), is operably linked to at least one of at least one inducible regulatory element, at least one transcriptional regulatory element, at least one post-translationally regulatory element and at least one temporally or spatially regulatory element; and
(b) a second sex chromosome comprising at least one endogenous nucleic acid sequence encoding at least one repressor specific for the transcription regulatory element operably linked to at least one of said nucleic acid sequences of (i) and (ii); wherein said second sex chromosome characterizes the desired sex; or
(B) the heterogametic organism comprising:
(a) a first sex chromosome comprising:
(i) at least one nucleic acid sequence encoding at least one Cas9 having a nucleolytic activity and/or a fusion protein thereof; or at least one non-active Cas9 and/or any fusion protein thereof;
(ii) at least one nucleic acid sequence encoding at least one gRNA directed against at least one target sequence; and
(iii) at least one nucleic acid sequence encoding at least one inhibitor of said Cas9, optionally, said Cas9 inhibitor is AcrIIA4; wherein said sequence is operably linked to at least one transcription regulatory element, said regulatory element being specific for a transcription factor endogenously encoded by a nucleic acid sequence comprised within the second sex chromosome;
wherein said first sex chromosome characterizes the undesired sex;
optionally, at least one of said nucleic acid sequences of (i), (ii) and (iii), is operably linked to at least one of at least one inducible regulatory element, at least one transcriptional regulatory element, at least one post-translationally regulatory element and at least one temporally or spatially regulatory element; and
(b) a second sex chromosome comprising at least one endogenous nucleic acid sequence encoding at least one transcription factor specific for the transcription regulatory element operably linked to the nucleic acid sequences of (iii); said second sex chromosome characterizes the desired sex.

65. The genetically engineered eukaryotic heterogametic organism according to claim 61, wherein at least one of:
(A) said organism produces gamete cells predominantly composed of said one desired sex;
(B) said eukaryotic heterogametic organism is of the biological kingdom Animalia;
(C) said organism is any one of a non-human mammal, an avian, an insect, a fish, an amphibian, a reptile, a crustacean, a crab, a lobster, a snail, a clam, an octopus, a starfish, a sea-urchin, jellyfish, and worms;
(D) the desired sex is of the homogametic organism and the undesired sex is of the heterogametic organism, and wherein said first sex chromosome is a chromosome characterizing the heterogametic organism and said second sex chromosome is a chromosome characterizing the homogametic organism; and
(E) said mammal is at least one of Cattle, domestic pig (swine, hog), sheep, horse, goat, alpaca, lama and Camels.

66. The genetically engineered eukaryotic heterogametic organism according to claim 61, wherein said organism is a mammalian organism, and wherein:
(A) said mammal is a domestic Cattle, wherein said heterogametic organism is a male, and wherein one of:
(a) the desired sex is female, said first sex chromosome is the Y chromosome, said second sex chromosome is the X chromosome, and said male produces sperm predominantly composed of gamete cells comprising X chromosome; or
(b) the desired sex is male, said first sex chromosome is the X chromosome, said second sex chromosome is the Y chromosome, and said male produces sperm predominantly composed of gamete cells comprising Y chromosome; or
(B) said mammal is rodent, wherein said heterogametic rodent is a male, and wherein one of:
(a) the desired sex is female, said first sex chromosome is the Y chromosome, said second sex chromosome is the X chromosome, and said male produces sperm predominantly composed of gamete cells comprising X chromosome; or
(b) the desired sex is male, said first sex chromosome is the X chromosome, said second sex chromosome is the Y chromosome, and said male produces sperm predominantly composed of gamete cells comprising Y chromosome.

67. The genetically engineered eukaryotic heterogametic organism according to claim 61, wherein the heterogametic organism is a mammal selected from: mammal is at least one of Cattle, domestic pig (swine, hog), sheep, horse, goat, alpaca, lama and Camels, and wherein:
(A) wherein the desired sex is female, and wherein said genetically engineered male is a mammalian male comprising a Y chromosome comprising:
(i) at least one nucleic acid sequence encoding at least one Cas9 having a nucleolytic activity and/or a fusion protein thereof; or at least one non-active Cas9 and/or fusion protein thereof; and
(ii) at least one nucleic acid sequence encoding at least one gRNA directed against at least one target sequence, optionally, said target sequence is at least one protospacer comprised within at least one of: the Spem1, JmjC-containing H3K9 demethylase (Jhdm2a), SUN domain-containing protein 4 (Sun4), SUN domain-containing protein 5 (Sun5), Crem, Golgi-associated PDZ and coiled-coil motif-containing protein gene (Gopc), the hook microtubule tethering protein 1 (Hook1) and/or the TATA box-binding protein-like protein 1 (Tbpl1) genes, and/or any orthologs or homologs thereof;
optionally, at least one of:
(I) said nucleic acid sequences of (i) and (ii), is operably linked to at least one of at least one inducible regulatory element, at least one transcriptional regulatory element, at least one post-translationally regulatory element and at least one temporally or spatially regulatory element; and
(II) wherein said Y chromosome of said mammalian male comprises:
(a) at least one nucleic acid sequence encoding dCas9-KRAB-MeCP2 fusion protein under a Tet-off promoter; and
(b) at least one nucleic acid sequence encoding at least one gRNA directed against at least one target sequence within the Spem 1 gene; and wherein said first sex chromosome is the Y chromosome characterizing the undesired sex;
(B) wherein the desired sex is female and wherein said genetically engineered male is a mammalian male comprising:
(a) a Y chromosome comprising:
(i) at least one nucleic acid sequence encoding at least one Cas9 having a nucleolytic activity and/or a fusion protein thereof; or at least one non-active Cas9 and/or any fusion protein thereof; and
(ii) at least one nucleic acid sequence encoding at least one gRNA directed against at least one target sequence, optionally, said target sequence is at least one protospacer comprised within at least one of Jhdm2a, Sun4, Sun5, Spem1, Crem, Gopc, Hook1, and/or the Tbpl1 genes and/or any orthologs or homologs thereof; wherein at least one of said nucleic acid sequences of (i) and (ii), is operably linked to at least one transcription regulatory element, said regulatory element being specific for a repressor endogenously encoded by a nucleic acid sequence comprised within the X chromosome;
optionally, at least one of said nucleic acid sequences of (i), (ii), is further operably linked to at least one of at least one inducible regulatory element, at least one transcriptional regulatory element, at least one post-translationally regulatory element and at least one temporally or spatially regulatory element; and
(b) an X chromosome comprising at least one endogenous nucleic acid sequence encoding at least one repressor specific for the transcription regulatory element operably linked to at least one of said nucleic acid sequences of (i) and (ii) in said Y chromosome; and
(C) wherein the desired sex is female and wherein said genetically engineered male is a mammalian male comprising:
(a) a Y chromosome comprising:
(i) at least one nucleic acid sequence encoding at least one Cas9 having a nucleolytic activity and/or a fusion protein thereof; or at least one non-active Cas9 and/or any fusion protein thereof;
(ii) at least one nucleic acid sequence encoding at least one gRNA directed against at least one target sequence, optionally, said target sequence is at least one protospacer comprised within at least one of the Jhdm2a, Sun4, Sun5, Spem1, Crem, Gopc, Hook1, and the Tbpl1 genes or any orthologs or homologs thereof; and
(iii) at least one nucleic acid sequence encoding at least one inhibitor of said Cas9, optionally, said Cas9 inhibitor AcrIIA4; wherein said sequence is operably linked to at least one transcription regulatory element specific for a transcription factor endogenously encoded by a nucleic acid sequence comprised within the X chromosome;
optionally, at least one of said nucleic acid sequences of (i), (ii) and (iii), is further operably linked to at least one of: at least one inducible regulatory element, at least one transcriptional regulatory element, at least one post-translationally regulatory element and at least one temporally or spatially regulatory element; and
(b) an X chromosome comprising at least one endogenous nucleic acid sequence encoding at least one transcription factor specific for the transcription regulatory element operably linked to the nucleic acid sequences of (iii) in said Y chromosome.

68. The genetically engineered eukaryotic heterogametic organism according to claim 61, wherein one of:
(A) said organism is an avian organism being a domesticated or an undomesticated bird, and wherein said domesticated bird is a chicken and wherein said heterogametic organism is a female (ZW), and wherein one of:
(a) the desired sex is female, said first sex chromosome is the Z chromosome, said second sex chromosome is the W chromosome, and said female produces predominantly gamete cells comprising one W chromosomes; or
(b) the desired sex is male, said first sex chromosome is the W chromosome, said second sex chromosome is the Z chromosome, and said female produces predominantly gamete cells comprising Z chromosome; or
(B) wherein said organism is of the biological kingdom Plantae, and wherein at least one of:
(I) said organisms is a dioecious plant; and
(II) said dioecious plant is of the family Cannabaceae.

69. A system comprising at least one genetically engineered eukaryotic heterogametic organism according to claim 61, and at least one homogametic eukaryotic organism, wherein said heterogametic organism comprising:
(a) a first sex chromosome comprising at least one exogenous nucleic acid sequence integrated therein, said exogenous nucleic acid sequence comprising:
(i) at least one nucleic acid sequence encoding at least one nucleic acids modifier component; and
(ii) at least one nucleic acid sequence encoding or forming at least one target recognition element for said at least one nucleic acids modifier component;
wherein said exogenous nucleic acid sequence optionally further comprise:
(iii) at least one nucleic acid sequence encoding at least one inhibitor or repressor for said at least one nucleic acids modifier component of (i);
optionally, at least one of said nucleic acid sequences of (i), (ii) and (iii), are operably linked to at least one of: at least one inducible or repressible regulatory element, at least one transcriptional regulatory element, at least one post-translationally regulatory element and at least one temporally or spatially regulatory element;
optionally, said genetically engineered eukaryotic heterogametic organism further comprises:
(b) a second sex chromosome comprising at least one of:
(iv) at least one exogenous nucleic acid sequence encoding at least one inhibitor or repressor for said at least one nucleic acids modifier component of (i); and
(v) at least one endogenous or exogenous nucleic acid sequence encoding at least one transcription factor or repressor, that specifically bind at least one transcription regulatory element operably linked to the nucleic acid sequence of at least one of (i), (ii) and (iii); optionally, said exogenous nucleic acid sequence of (iv) and/or (v) is operably linked to at least one of: at least one inducible regulatory element, at least one transcriptional regulatory element, at least one post-translationally regulatory element and at least one temporally or spatially regulatory element, optionally, wherein said homogametic organism is a Wild type organism: or a genetically engineered organism, said genetically engineered eukaryotic homogametic organism comprises a second sex chromosome comprising at least one exogenous nucleic acid sequence encoding at least one inhibitor of said at least one nucleic acids modifier component of (i).

70. At least one progeny or cell produced or prepared by at least one genetically engineered eukaryotic heterogametic organism as define according to claim 61, or the system comprising said organism, or any product of said progeny or cell.

71. An in vivo or ex vivo population of gamete cells predominantly composed of one desired sex, or any composition or preparation thereof, wherein said population of cells is produced by at least one genetically engineered eukaryotic heterogametic organism as defined according to claim 61, or the system comprising said organism.

72. A method for preparing gamete cell population predominantly composed of one desired sex, the method comprising the step of obtaining gamete cell population from at least one genetically engineered eukaryotic heterogametic organism comprising:
(a) a first sex chromosome comprising at least one exogenous nucleic acid sequence integrated therein, said exogenous nucleic acid sequence comprising:
(i) at least one nucleic acid sequence encoding at least one nucleic acids modifier component, or any fusion protein thereof; and
(ii) at least one nucleic acid sequence encoding or forming at least one target recognition element for said at least one nucleic acids modifier component;
wherein said exogenous nucleic acid sequence optionally further comprise:
(iii) at least one nucleic acid sequence encoding at least one inhibitor or repressor for said at least one nucleic acids modifier component of (i);
optionally, at least one of said nucleic acid sequences of (i), (ii) and (iii), are operably linked to at least one of: at least one inducible or repressible regulatory element, at least one transcriptional regulatory element, at least one post-translationally regulatory element and at least one temporally or spatially regulatory element;
optionally, said genetically engineered eukaryotic heterogametic organism further comprises:
(b). a second sex chromosome comprising at least one of:
(iv) at least one exogenous nucleic acid sequence encoding at least one inhibitor and/or repressor for said at least one nucleic acids modifier component of (i); and
(v) at least one endogenous or exogenous nucleic acid sequence encoding at least one transcription factor or repressor, that specifically bind at least one transcription regulatory element operably linked to the nucleic acid sequence of at least one of (i), (ii) and (iii);
optionally, said exogenous nucleic acid sequence of (iv) and/or (v) is operably linked to at least one of: at least one inducible regulatory element, at least one transcriptional regulatory element, at least one post-translationally regulatory element and at least one temporally or spatially regulatory element.

73. A method for selecting a desired sex of a eukaryotic organism, the method comprising the steps of: contacting at least one genetically engineered heterogametic organism or gamete cell/s thereof with at least one homogametic eukaryotic organism or gamete cell/s thereof, thereby obtaining a progeny of a desired sex and/or a progeny population predominantly composed of said one desired sex, said genetically engineered eukaryotic heterogametic organism comprising:
(a) a first sex chromosome comprising at least one exogenous nucleic acid sequence integrated therein, said exogenous nucleic acid sequence comprising:
(i) at least one nucleic acid sequence encoding at least one nucleic acids modifier component, or any fusion protein thereof; and
(ii) at least one nucleic acid sequence encoding or forming at least one target recognition element for said at least one nucleic acids modifier component;
wherein said exogenous nucleic acid sequence optionally further comprise:
(iii) at least one nucleic acid sequence encoding at least one inhibitor or repressor for said at least one nucleic acids modifier component of (i);
optionally, said genetically engineered eukaryotic heterogametic organism further comprises:
(b) a second sex chromosome comprising at least one of:
(iv) at least one exogenous nucleic acid sequence encoding at least one inhibitor or repressor for said at least one nucleic acids modifier component of (i); and
(v) at least one endogenous or exogenous nucleic acid sequence encoding at least one transcription factor or repressor, that specifically bind at least one transcription regulatory element operably linked to the nucleic acid sequence of at least one of (i), (ii) and (iii).

74. The method according to claim 73, wherein at least one of:
(A) at least one of said nucleic acid sequences of (i), (ii), (iii), (iv) and (v), are operably linked to at least one of: at least one inducible or repressible regulatory element, at least one transcriptional regulatory element, at least one post-translationally regulatory element and at least one temporally or spatially regulatory element;
(B) said nucleic acids modifier component is at least one of: at least one modifier protein or any fusion protein thereof, and at least one modifier nucleic acid sequence, said modifier protein of said genetically engineered eukaryotic heterogametic organism is a nuclease, said nuclease is at least one of: (i) a nuclease having a nucleolytic activity and/or a fusion protein thereof; (ii) a non-active nuclease and/or a fusion protein thereof;
(C) the nuclease is an RNA guided nuclease, and wherein said at least one target recognition element is at least one guide RNA directed against at least one target sequence within coding and/or non-coding sequences, and/or any product/s thereof, of at least one gene involved in viability, stability and/or function of gamete cells of said organism;
(D) said at least one gene involved in viability, stability and/or function of gamete cells of said organism is expressed at a late stage of gametogenesis;
(E) wherein at least one of:
(a) said RNA guided DNA binding protein nuclease is a CRISPR-associated endonuclease 9 (Cas9) system; and/or
(b) said inhibitor of said at least one nucleic acids modifier protein, is an inhibitor of Cas9, optionally, said Cas9 inhibitor is the anti-CRISPR (Acr) IIA4 (AcrIIA4); and
(F) wherein at least one of:
(I) said inducible or repressible regulatory element is at least one of:
(a) a repressible promoter, said repressible promoter is Tet-off, ADH1, GAL4/UAS, LexA/lexAop, and/or the Q system;
(b) at least one degron, wherein said degron is at least one of: DD, LID, and/or AID;
(II) wherein said post-translationally regulatory element is a degron, said degron is any one of DD, UbK, UbD, and UbM; and
(III) wherein said at least one temporally or spatially regulatory element is the Spermatid maturation protein 1 (spem 1) promoter.

75. The method according to claim 73, wherein at least one of:
(A) said genetically engineered eukaryotic heterogametic organism comprises a first sex chromosome comprising:
(i) at least one nucleic acid sequence encoding at least one Cas9 having a nucleolytic activity and/or a fusion protein thereof; or at least one non-active Cas9 and/or any fusion protein thereof; and
(ii) at least one nucleic acid sequence encoding at least one gRNA directed against at least one target sequence;
optionally, at least one of said nucleic acid sequences of (i) and (ii), are operably linked to at least one of at least one inducible regulatory element, at least one transcriptional regulatory element, at least one post-translationally regulatory element and at least one temporally or spatially regulatory element, wherein said first sex chromosome characterizes the undesired sex;
(B) wherein said first sex chromosome comprises:
(i) at least one nucleic acid sequence encoding dCas9-KRAB-MeCP2 fusion protein under a Tet-off promoter; and
(ii) at least one nucleic acid sequence encoding at least one gRNA directed against at least one target sequence within coding and/or non-coding sequences, and/or any product/s thereof, of at least one gene involved in viability, stability and/or function of gamete cells of said organism; and wherein said first sex chromosome characterizes the undesired sex; and
(C) wherein said first sex chromosome comprises:
(i) at least one nucleic acid sequence encoding dCas9-KRAB-MeCP2 fusion protein under a Tet-off promoter; and
(ii) at least one nucleic acid sequence encoding at least one gRNA directed against at least one target sequence within the Spem1 gene; wherein said first sex chromosome characterizes the undesired sex.

76. The method according to claim 73, wherein one of:
(A) said genetically engineered eukaryotic heterogametic organism comprises:
(a) a first sex chromosome comprising:
(i) at least one nucleic acid sequence encoding at least one Cas9 having a nucleolytic activity and/or a fusion protein thereof; or at least one non-active Cas9 and/or any fusion protein thereof; and
(ii) at least one nucleic acid sequence encoding at least one gRNA directed against at least one target sequence;
wherein at least one of said nucleic acid sequences of (i) and (ii), are operably linked to at least one transcription regulatory element, said regulatory element being specific for a repressor endogenously encoded by a nucleic acid sequence comprised within the second sex chromosome; said first sex chromosome characterizes the undesired sex; optionally, at least one of said nucleic acid sequences of (i), (ii), is further operably linked to at least one of at least one inducible regulatory element, at least one transcriptional regulatory element, at least one post-translationally regulatory element and at least one temporally or spatially regulatory element; and
(b) a second sex chromosome comprising at least one endogenous nucleic acid sequence encoding at least one repressor specific for the transcription regulatory element operably linked to at least one of said nucleic acid sequences of (i) and (ii); said second sex chromosome characterizes the desired sex; or
(B) wherein said genetically engineered eukaryotic heterogametic organism comprises:
(a) a first sex chromosome comprising:
(i) at least one nucleic acid sequence encoding at least one Cas9 having a nucleolytic activity and/or a fusion protein thereof; or at least one non-active Cas9 and/or any fusion protein thereof;
(ii) at least one nucleic acid sequence encoding at least one gRNA directed against at least one target sequence; and
(iii) at least one nucleic acid sequence encoding at least one inhibitor of said Cas9, optionally, said Cas9 inhibitor is AcrIIA4; wherein said sequence is operably linked to at least one transcription regulatory element, said regulatory element being specific for a transcription factor endogenously encoded by a nucleic acid sequence comprised within the second sex chromosome;
Wherein said first sex chromosome characterizes the undesired sex;
optionally, at least one of said nucleic acid sequences of (i), (ii) and (iii), is operably further linked to at least one of at least one inducible regulatory element, at least one transcriptional regulatory element, at least one post-translationally regulatory element and at least one temporally or spatially regulatory element; and
(b) a second sex chromosome comprising at least one endogenous nucleic acid sequence encoding at least one transcription factor specific for the transcription regulatory element operably linked to the nucleic acid sequences of (iii); said second sex chromosome characterizes the desired sex.

77. The method according to claim 73, wherein at least one of:
(A) said organism produces gamete cells predominantly composed of said single desired sex;
(B) the desired sex is of the homogametic organism and the undesired sex is of the heterogametic organism, and wherein said first sex chromosome is a chromosome characterizing the heterogametic organism and said second sex chromosome is a chromosome characterizing the homogametic organism;
(C) said eukaryotic heterogametic organism is of the biological kingdom Animalia;
(D) said organism is any one of a non-human mammal, an avian, an insect, a fish, an amphibian, a reptile, a crustacean, a crab, a lobster, a snail, a clam, an octopus, a starfish, a sea-urchin, jellyfish, and worms;
(E) said mammal is at least one of: Cattle, domestic pig (swine, hog), sheep, horse, goat, alpaca, lama and Camels, wherein said heterogametic organism is a male, and wherein one of:
(a) the desired sex is female, said first sex chromosome is the Y chromosome, said second sex chromosome is the X chromosome, and said male produces sperm predominantly composed of gamete cells comprising X chromosome; or
(b) the desired sex is male, said first sex chromosome is the X chromosome, and said second sex chromosome is the Y chromosome, and said male produces sperm predominantly composed of gamete cells comprising Y chromosome; and
(F) said mammal is rodent, wherein said heterogametic organism is a male, and wherein one of:
(a) the desired sex is female, said first sex chromosome is the Y chromosome, and said second sex chromosome is the X chromosome, and said male produces sperm predominantly composed of gamete cells comprising X chromosome; or
(b) the desired sex is male, said first sex chromosome is the X chromosome, and said second sex chromosome is the Y chromosome, and said male produces sperm predominantly composed of gamete cells comprising Y chromosome.

78. The method according to claim 73, wherein the organism is a mammalian subject, and wherein on of:
(A) the desired sex is female and wherein said genetically engineered male, is a mammalian male comprising a Y chromosome comprising:
(i) at least one nucleic acid sequence encoding at least one Cas9 having a nucleolytic activity and/or a fusion protein thereof; or at least one non-active Cas9 and/or any fusion protein thereof; and
(ii) at least one nucleic acid sequence encoding at least one gRNA directed against at least one target sequence, optionally, said target sequence is at least one protospacer comprised within at least one of the Spem1, JmjC-containing H3K9 demethylase (Jhdm2a), SUN domain-containing protein 4 (Sun4), SUN domain-containing protein 5 (Sun5), Crem, Golgi-associated PDZ and coiled-coil motif-containing protein gene (Gopc), the hook microtubule tethering protein 1 (Hook1) and/or the TATA box-binding protein-like protein 1 (Tbpl1) genes, and/or any orthologs or homologs thereof; optionally, at least one of:
(I) said nucleic acid sequences of (i) and (ii), is operably linked to at least one of at least one inducible regulatory element, at least one transcriptional regulatory element, at least one post-translationally regulatory element and at least one temporally or spatially regulatory element; and
(II) said mammalian genetically engineered male comprises a Y chromosome comprising:
(i) at least one nucleic acid sequence encoding dCas9-KRAB-MeCP2 fusion protein under a Tet-off promoter; and
(ii) at least one nucleic acid sequence encoding at least one gRNA directed against at least one target sequence within the Spem1 gene.
(B) wherein the desired sex is female and wherein said genetically engineered male comprises:
(a) a Y chromosome comprising:
(i) at least one nucleic acid sequence encoding at least one Cas9 having a nucleolytic activity and/or a fusion protein thereof; or at least one non-active Cas9 and/or any fusion protein thereof; and
(ii) at least one nucleic acid sequence encoding at least one gRNA directed against at least one target sequence, optionally, said target sequence is at least one protospacer comprised within at least one of Jhdm2a, Sun4, Sun5, Spem1, Crem, Gopc, Hook1, and/or the Tbpl1 genes and/or any orthologs or homologs thereof;
wherein at least one of said nucleic acid sequences of (i) and (ii), is operably linked to at least one transcription regulatory element, said regulatory element being specific for a repressor endogenously encoded by a nucleic acid sequence comprised within the X chromosome;
optionally, at least one of said nucleic acid sequences of (i), (ii), is operably linked to at least one of at least one inducible regulatory element, at least one transcriptional regulatory element, at least one post-translationally regulatory element and at least one temporally or spatially regulatory element; and
(b) an X chromosome comprising at least one endogenous nucleic acid sequence encoding at least one repressor specific for the transcription regulatory element operably linked to at least one of said nucleic acid sequences of (i) and (ii) in said Y chromosome; or
(C) the desired sex is female and wherein said genetically engineered male comprises:
(a) a Y chromosome comprising:
(i) at least one nucleic acid sequence encoding at least one Cas9 having a nucleolytic activity and/or a fusion protein thereof; or at least one non-active Cas9 and/or any fusion protein thereof;
(ii) at least one nucleic acid sequence encoding at least one gRNA directed against at least one target sequence, optionally, said target sequence is at least one protospacer comprised within at least one of the Jhdm2a, Sun4, Sun5, Spem1, Crem, Gopc, Hook, and the Tbpl1 genes or any orthologs or homologs thereof; and
(iii) at least one nucleic acid sequence encoding at least one inhibitor of said Cas9, optionally, said Cas9 inhibitor is AcrIIA4; wherein said sequence is operably linked to at least one transcription regulatory element specific for a transcription factor endogenously encoded by a nucleic acid sequence comprised within the X chromosome;
optionally, at least one of said nucleic acid sequences of (i), (ii) and (iii), is operably linked to at least one of: at least one inducible regulatory element, at least one transcriptional regulatory element, at least one post-translationally regulatory element and at least one temporally or spatially regulatory element; and
(b) an X chromosome comprising at least one endogenous nucleic acid sequence encoding at least one transcription factor specific for the transcription regulatory element operably linked to the nucleic acid sequences of (iii) in said Y chromosome.

79. The method according to claim 73, wherein one of:
(A) said wherein said avian organism is any one of a domesticated and an undomesticated bird, wherein said heterogametic organism is a female (ZW), and wherein one of:
(a) the desired sex is female, said first sex chromosome is the Z chromosome, said second sex chromosome is the W chromosome, and said female produces predominantly gamete cells comprising W chromosomes; or
(b) the desired sex is male, said first sex chromosome is the W chromosome, and said second sex chromosome is the Z chromosome, and said female produces predominantly gamete cells comprising Z chromosome, optionally, wherein said domesticated bird is a chicken; or
(B) said organism is of the biological kingdom Plantae, and wherein at least one of:
(a) said organisms is a dioecious plant; and/or
(b) said dioecious plant is of the family Cannabaceae.

80. The method according to claim 73, for the preparation of a population of eukaryotic organisms predominantly composed of a single desired sex, or any cell or product produced therefrom.
